(19) United States
(12) Patent Application Publication
(10) Pub. No.: US 2025/0353852 A1
(43) Pub. Date: Nov. 20, 2025

(54) COMPOUNDS AND COMPOSITIONS THAT INHIBIT PIKFYVE

(71) Applicant: Kineta, Inc., Seattle, WA (US)

(72) Inventors: Gnanasambandam Kumaravel, Lexington, MA (US); Madeline Macdonnell, Brighton, MA (US); Hairuo Peng, Needham, MA (US)

(21) Appl. No.: 18/717,144

(22) PCT Filed: Dec. 7, 2022
(86) PCT No.: PCT/US2022/052130

§ 371 Date: Jun. 6, 2024

Related U.S. Application Data
(60) Provisional application No. 63/287,177, filed on Dec. 8, 2021.

(51) Int. Cl. C07D 487/04; A61K 31/5377; A61K 31/5386; C07D 519/00

(52) U.S. Cl. CPC C07D 487/04; A61K 31/5377; A61K 31/5386; C07D 519/00

(57) ABSTRACT

Pyrazolo[1,5-a]pyrimidines are disclosed. These compounds may be useful in the treatment of neurological disorder, including frontotemporal dementia, chronic traumatic encephalopathy, Alzheimer's disease, limbic-predominant age-related TDP-43 encephalopathy, or frontotemporal lobar degeneration. Further, the invention features a method of inhibiting toxicity in a cell related to a protein TDP-43 or C9orf72. The compounds of the invention, alone or in combination with other pharmaceutically active agents, can be used for treating or preventing neurological disorders.

### FIELD OF THE INVENTION

The invention relates to pyrazolo[1,5-a]pyrimidines and their use in the treatment of neurological disorders in patients, such as human patients.

### BACKGROUND

An incomplete understanding of the molecular perturbations that cause disease, as well as a limited arsenal of robust model systems, has contributed to a failure to generate successful disease-modifying therapies against common and progressive neurological disorders, such as ALS and FTD. Progress is being made on many fronts to find agents that can arrest the progress of these disorders. However, the present therapies for most, if not all, of these diseases provide very little relief. Accordingly, a need exists to develop therapies that can alter the course of neurodegenerative diseases. More generally, a need exists for better methods and compositions for the treatment of neurodegenerative diseases in order to improve the quality of the lives of those afflicted by such diseases.

### SUMMARY

TDP-43 is a nuclear DNA/RNA binding protein involved in RNA splicing. Under pathological cell stress, TDP-43 translocates to the cytoplasm and aggregates into stress granules and related protein inclusions. These phenotypes are hallmarks of degenerating motor neurons and are found in 97% of all ALS cases. The highly penetrant nature of this pathology indicates that TDP-43 is broadly involved in both familial and sporadic ALS. Additionally, TDP-43 mutations that promote aggregation are linked to higher risk of developing ALS, suggesting protein misfolding and aggregation act as drivers of toxicity. TDP-43 toxicity can be recapitulated in yeast models, where the protein induces a viability deficit and localizes to stress granules.

In an aspect, the invention features a compound, or pharmaceutically acceptable salt thereof, of Formula I:

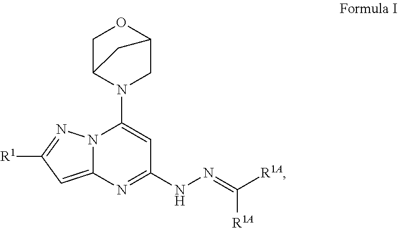

or pharmaceutically acceptable salt thereof,
where:
R¹ is optionally substituted C₂-C₉ heteroaryl; and
each R^{1A} is independently H, optionally substituted C₁-C₆ alkyl, optionally substituted C₆-C₁₀ aryl, or optionally substituted C₂-C₉ heteroaryl; and the remaining R^{1B} is optionally substituted C₁-C₆ alkyl, optionally substituted C₆-C₁ aryl, or optionally substituted C₂-C₉ heteroaryl.

In some embodiments, one R^{1A} is hydrogen, and the remaining R^{1A} is optionally substituted C₆-C₁₀ aryl. In some embodiments, R¹ is pyrid-4-yl.

In another aspect, the invention provides a compound having the structure:

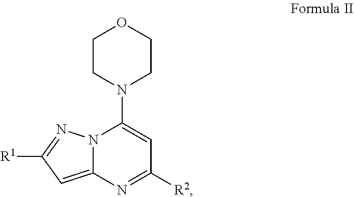

or pharmaceutically acceptable salt thereof,
where:
R¹ is optionally substituted pyridin-4-yl;
R² is optionally substituted C₂-C₉ heterocyclyl, optionally substituted C₁-C₆ alkyl, optionally substituted C₁-C₆ alkenyl, optionally substituted pyridin-2-yl, optionally substituted pyridin-3-yl, optionally substituted pyrimidn-4-yl, optionally substituted thiadiazolyl, optionally substituted oxadiazolyl, optionally substituted dialkylamino, optionally substituted 6-oxo-1,5-dihydropyridazin-1-yl, optionally substituted pyrazinyl, fluoro, cyano, optionally substituted pyrazol-3-yl, optionally substituted pyrazol-5-yl, optionally substituted oxazole, optionally substituted N-tetrahydropyranopyrazolyl, optionally substituted N-tetrahydroindazolyl, optionally substituted C₁-C₆ heteroalkyl, optionally substituted C₆-C₁₀ aryl C₁-C₆ alkyl, optionally substituted C₆-C₁₀ aryl C₁-C₆ alkenyl, optionally substituted C₆-C₁₀ aryl C₁-C₆ heteroalkyl, CONH₂, —CONH—NHR^{1A},

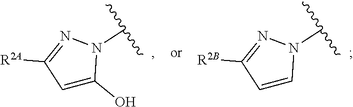

R^{1A} is H or optionally substituted C₂-C₁₀ acyl;
R^{2A} is optionally substituted aryl, optionally substituted C₁-C₆ alkyl, optionally substituted C₂-C₅ heteroaryl, or optionally substituted C₃-C₆ cycloalkyl; and
R^{2B} is pyridizin-4-yl, phenyl substituted with fluoro or methoxy, piperidinyl optionally substituted with C₁-C₆ alkyl, optionally substituted pyrimidin-5-yl, optionally substituted pyridin-2-yl, optionally substituted pyridine-3-yl, optionally substituted C₃ carbocyclyl, azetidin-3-yl, optionally substituted C₁-C₆ hydroxyalkyl, or C₃ heteroalkyl.

In some embodiments, the R² is an optionally substituted C₂-C₉ heterocyclyl. In some embodiments, R² is optionally substituted azetidine-3-yl or optionally substituted azetidine-1-yl. In some embodiments, R² is optionally substituted piperazin-1-yl or optionally substituted piperidin-1-yl. In some embodiments, R² is optionally substituted morpholin-1-yl. In some embodiments, R² is optionally substituted pyrrolidine-2-yl. In some embodiments, R² is an optionally substituted C₁-C₆ alkyl. In some embodiments, R² is —CONH—NHR^{1A}. In some embodiments, R^{1A} is optionally substituted C₂-C₁₀ acyl. In some embodiments, R² is an optionally substituted pyridin-2-yl. In some embodiments, R² is an optionally substituted pyridin-3-yl. In some embodiments, R² is an optionally substituted pyrimidin-4-yl. In some embodiments, R² is an optionally substituted C₆-C₁₀ aryl C₁-C₆ alkyl. In some embodiments, R² is an optionally substituted C₁-C₆ alkenyl. In some embodiments, R² is an optionally substituted C₆-C₁₀ aryl C₁-C₆ alkenyl. In some embodiments, R² is an optionally substituted thiadiazolyl. In some embodiments, R² is an optionally substituted oxadiazolyl. In some embodiments, R² is an optionally substituted 6-oxo-1,5-dihydropyridazin-1-yl. In some embodiments, R² is an optionally substituted dialkylamino. In some embodiments, R² is an optionally substituted pyrazinyl. In some embodiments, R² is an optionally substituted pyrazol-3-yl. In some embodiments, R² is an optionally substituted pyrazol-5-yl. In some embodiments, R² is an optionally substituted oxazolyl. In some embodiments, R² is an optionally substituted N-tetrahydropyranopyrazolyl. In some embodiments, R² is an optionally substituted N-tetrahydroindazolyl. In some embodiments, R² is an optionally substituted imidazolyl. In some embodiments, R² is an optionally substituted C₁-C₆ heteroalkyl. In some embodiments, R² is an optionally substituted C₆-C₁₀ aryl C₁-C₆ heteroalkyl.

In some embodiments, R² is substituted with oxo. In some embodiments, R² is substituted with optionally substituted phenyl. In some embodiments, R² is substituted with optionally substituted benzyl. In some embodiments, R² is substituted with optionally substituted phenoxy. In some embodiments, R² is substituted with 4-fluorophenoxy or 3-fluorophenoxy. In some embodiments, R² is substituted with optionally substituted amino. In some embodiments, R² is substituted with ═NH. In some embodiments, R² is substituted with optionally substituted C₁-C₆ alkyl. In some embodiments, R² is substituted with methyl. In some embodiments, R² is substituted with halo. In some embodiments, R² is substituted with bromo. In some embodiments, R² is substituted with optionally substituted C₁-C₆ heteroalkyl. In some embodiments, R² is substituted with methoxy. In some embodiments, R² is substituted with optionally substituted pyridin-3-yl. In some embodiments, R² is substituted with optionally substituted C₂-C₉ heterocyclyl. In some embodiments, R² is substituted with optionally substituted piperidin-3-yl or optionally substituted 1,2,3,6-tetrahydropyridin-3-yl. In some embodiments, R² is substituted with hydroxyl. In some embodiments, R² is substituted with nitro.

In some embodiments, R² is

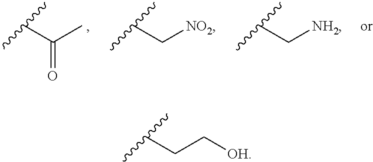

In some embodiments, R² is

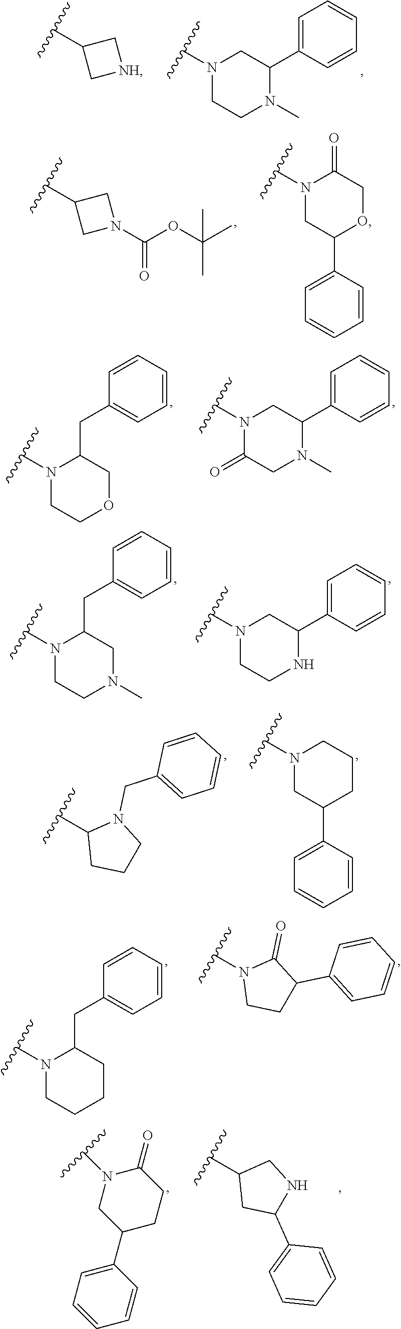

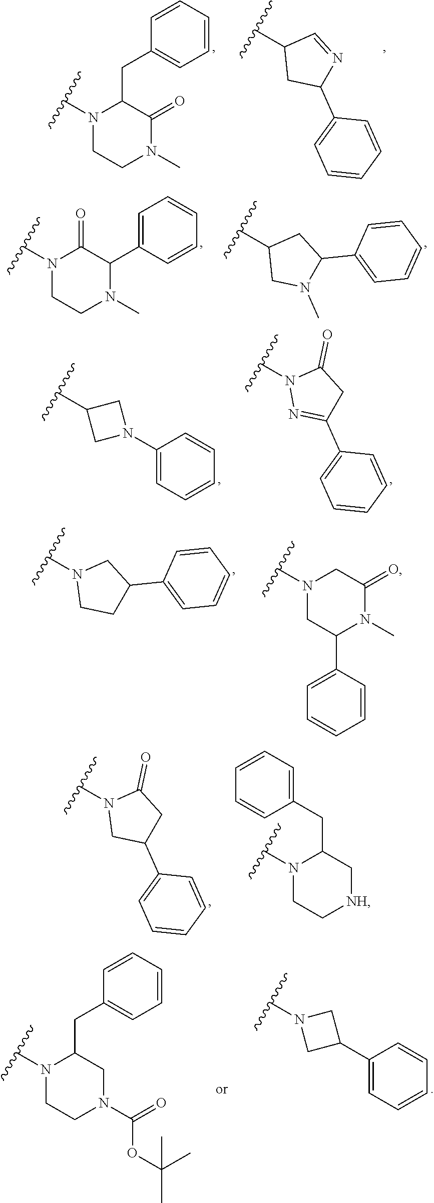

In some embodiments, R² is

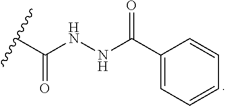 [SMILES: *C(=O)NNC(=O)c1ccccc1]

In some embodiments, R² is

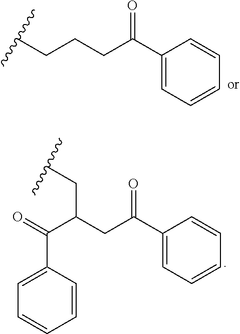 [SMILES: *CCCC(=O)c1ccccc1 ; *CC(CC(=O)c1ccccc1)C(=O)c1ccccc1]

In some embodiments, R² is

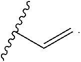 [SMILES: *C=C]

In some embodiments, R² is

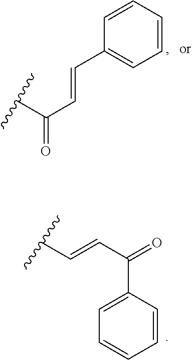 [SMILES: *C(=O)/C=C/c1ccccc1 ; */C=C/C(=O)c1ccccc1]

In some embodiments, R² is

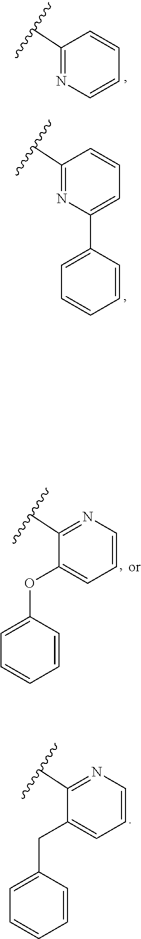 [SMILES: *c1ccccn1 ; *c1cccc(-c2ccccc2)n1 ; *c1ncccc1Oc1ccccc1 ; *c1ncccc1Cc1ccccc1]

In some embodiments, R² is

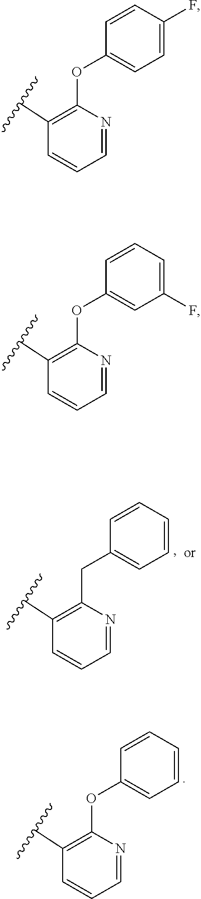 [SMILES: *c1cccnc1Oc1ccc(F)cc1 ; *c1cccnc1Oc1cccc(F)c1 ; *c1cccnc1Cc1ccccc1 ; *c1cccnc1Oc1ccccc1]

In some embodiments, R² is

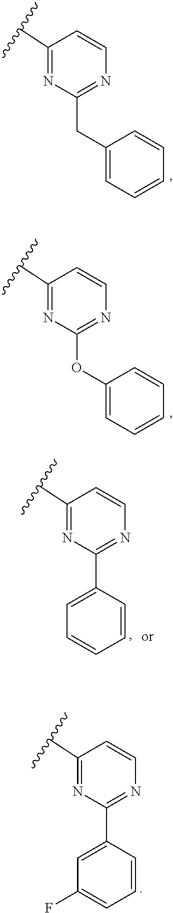 [SMILES: *c1ccnc(Cc2ccccc2)n1 ; *c1ccnc(Oc2ccccc2)n1 ; *c1ccnc(-c2ccccc2)n1 ; *c1ccnc(-c2cccc(F)c2)n1]

In some embodiments, R² is

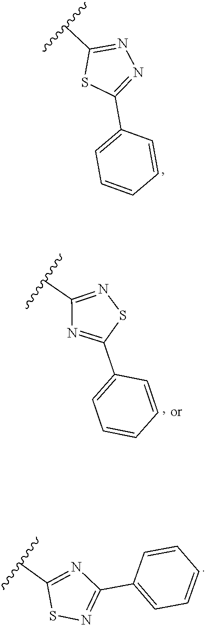 [SMILES: *c1nnc(-c2ccccc2)s1 ; *c1nsc(-c2ccccc2)n1 ; *c1nc(-c2ccccc2)ns1]

In some embodiments, R² is

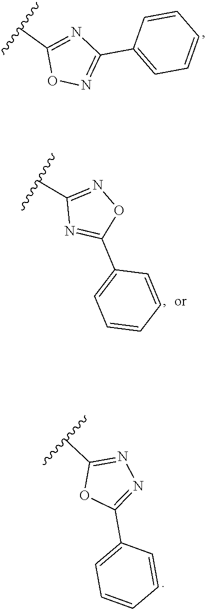 [SMILES: *c1nc(-c2ccccc2)no1 ; *c1noc(-c2ccccc2)n1 ; *c1nnc(-c2ccccc2)o1]

In some embodiments, R² is

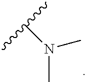 [SMILES: *N(C)C]

In some embodiments, R² is

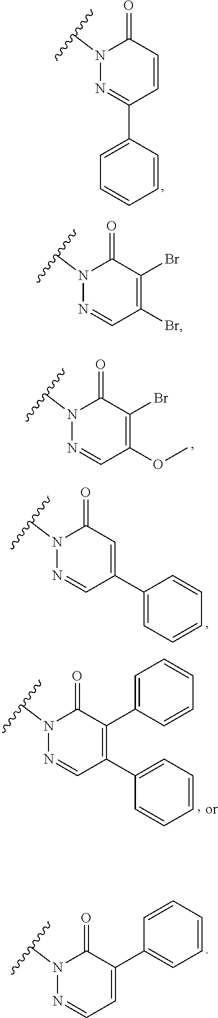

In some embodiments, R² is

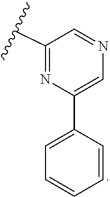 [SMILES: *c1cncc(-c2ccccc2)n1]

In some embodiments, R² is

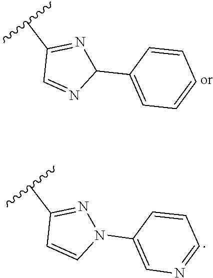 [SMILES: *C1=NC(c2ccccc2)N=C1 ; *c1ccn(-c2cccnc2)n1]

In some embodiments, R² is

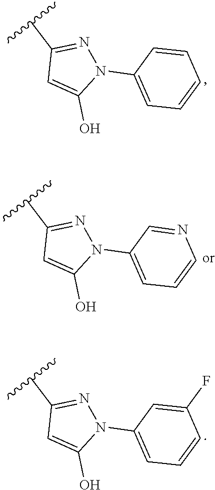 [SMILES: *c1cc(O)n(-c2ccccc2)n1 ; *c1cc(O)n(-c2cccnc2)n1 ; *c1cc(O)n(-c2cccc(F)c2)n1]

In some embodiments, R² is

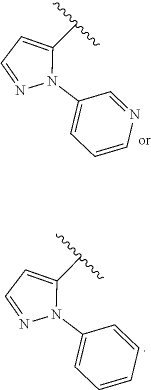 [SMILES: *c1ccnn1-c1cccnc1 ; *c1ccnn1-c1ccccc1]

In some embodiments, R² is

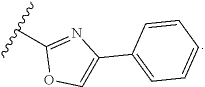 [SMILES: *c1nc(-c2ccccc2)co1]

In some embodiments, R² is

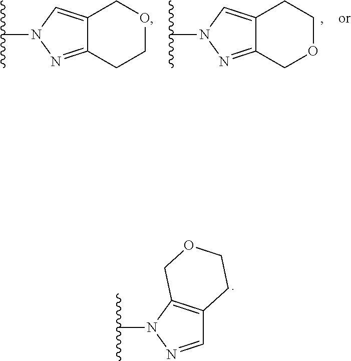

In some embodiments, R² is

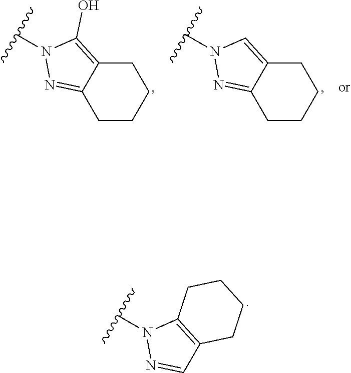 [SMILES: *n1nc2c(c1O)CCCC2 ; *n1cc2c(n1)CCCC2 ; *n1ncc2c1CCCC2]

In some embodiments, R² is

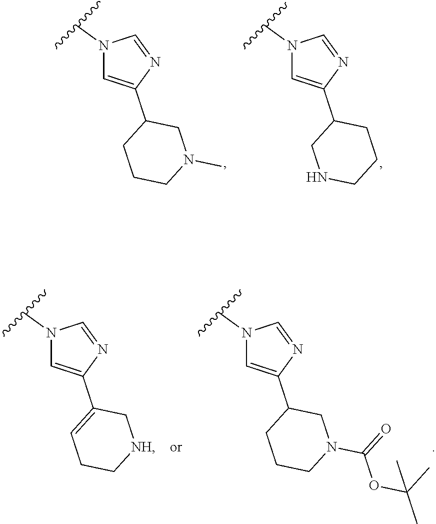 [SMILES: *n1cnc(C2CCCN(C)C2)c1 ; *n1cnc(C2CCCNC2)c1 ; *n1cnc(C2=CCCNC2)c1 ; *n1cnc(C2CCCN(C(=O)OC(C)(C)C)C2)c1]

In some embodiments, R² is,

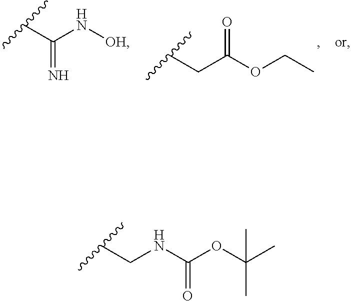

In some embodiments, R² is

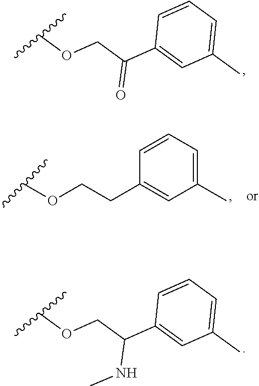 [SMILES: *OCC(=O)c1cccc(C)c1 ; *OCCc1cccc(C)c1 ; *OCC(NC)c1cccc(C)c1]

In some embodiments, R² is

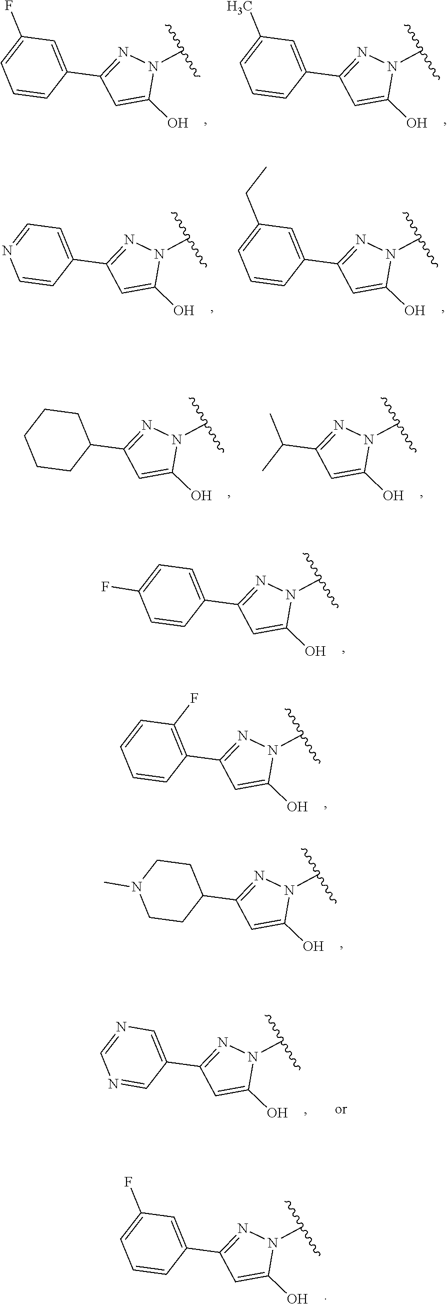

In some embodiments, R² is

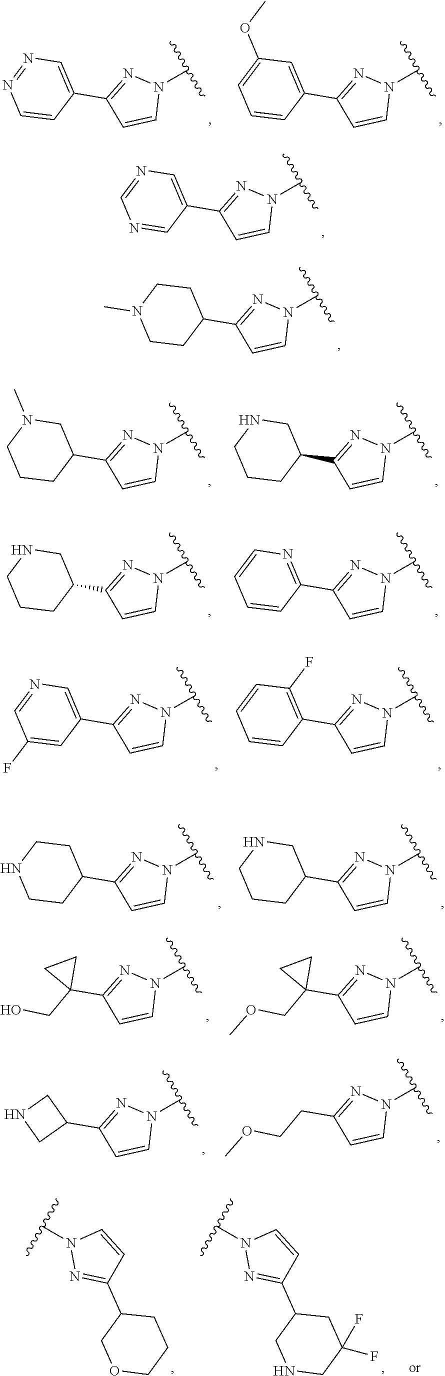

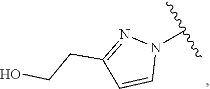 [SMILES: *n1ccc(CCO)n1]

In yet another aspect, the invention provides a compound having the structure:

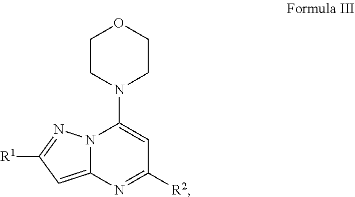

or a pharmaceutically acceptable salt thereof,
where:
R¹ is optionally substituted C₁₋₆ alkenyl, optionally substituted C₁-C₆ hydroxyalkyl, C₁-C₆ alkyl substituted with dialkyl amino, hydrogen, or optionally substituted C₂-C₉ heterocyclyl; and
R² is optionally substituted pyrazol-1-yl, optionally substituted pyrazol-3-yl, optionally substituted N-tetrahydropyranopyrazolyl, C₆-C₁₀ aryl optionally substituted with optionally substituted C₂-C₉ heteroaryl, or optionally substituted pyrimidin-4-yl.

In some embodiments, R¹ is an optionally substituted C₁-C₆ alkenyl. In some embodiments, R¹ is an optionally substituted C₁-C₆ hydroxyalkyl. In some embodiments, R¹ is an optionally substituted C₂-C₉ heterocyclyl. In some embodiments, R¹ is a C₁-C₆ alkyl substituted with dialkyl amino.

In some embodiments, R¹ is

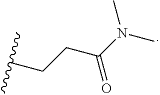 [SMILES: *CCC(=O)N(C)C]

In some embodiments, R¹ is

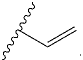 [SMILES: *C=C]

In some embodiments, R¹ is

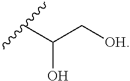 [SMILES: *C(O)CO]

In some embodiments, R¹ is

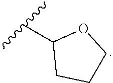 [SMILES: *C1CCCO1]

In some embodiments, R¹ is.

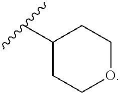 [SMILES: *C1CCOCC1]

In some embodiments, R¹ is

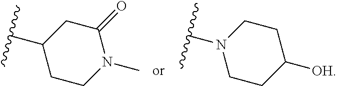

In some embodiments, R is an optionally substituted pyrazol-3-yl. In some embodiments, R² is an optionally substituted pyrazol-1-yl. In some embodiments, R² is an optionally substituted N-tetrahydropyranopyrazolyl. In some embodiments, R² is an optionally substituted pyrimidin-4-yl. In some embodiments, R² is substituted with optionally substituted phenyl. In some embodiments, R² is substituted with 3-methoxy-phenyl. In some embodiments, R² is substituted with 2-fluorophenyl. In some embodiments, R² is substituted with pyrimidin-3-yl. In some embodiments, R² is substituted with pyrazol-1-yl. In some embodiments, R² is phenyl substituted with optionally substituted pyrazol-3-yl.

In some embodiments, R² is

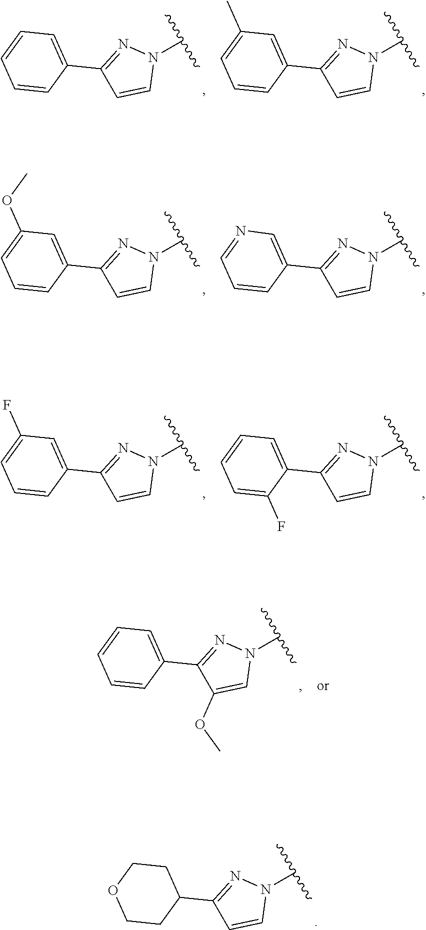

In some embodiments, R² is

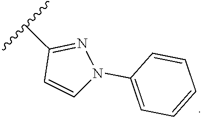 [SMILES: *c1ccn(-c2ccccc2)n1]

In some embodiments, R² is

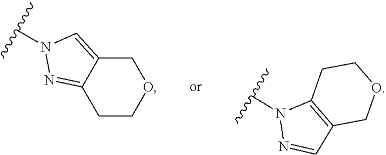 [SMILES: *n1cc2c(n1)CCOC2 ; "or" ; *n1ncc2c1CCOC2]

In some embodiments, R² is

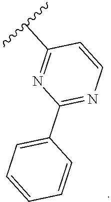 [SMILES: *c1ccnc(-c2ccccc2)n1]

In some embodiments, R² is R² is

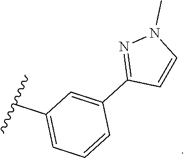 [SMILES: *c1cccc(-c2ccn(C)n2)c1]

In still another aspect, the invention features a compound, or pharmaceutically acceptable salt thereof, having the structure of any one of compound 1-152 in Table 1, or a pharmaceutically acceptable salt thereof.

In an aspect, the invention features a pharmaceutical composition comprising any of the foregoing compounds and a pharmaceutically acceptable excipient.

In an aspect, the invention features a method of treating a neurological disorder (e.g., frontotemporal dementia (FTLD-TDP), chronic traumatic encephalopathy, ALS, Alzheimer's disease, limbic-predominant age-related TDP-43 encephalopathy (LATE), or frontotemporal lobar degeneration) in a subject in need thereof. This method includes administering an effective amount of any of the foregoing compounds or pharmaceutical compositions.

In an aspect, the invention features a method of inhibiting toxicity in a cell (e.g., mammalian neural cell) related to a protein (e.g., TDP-43 or C₉orf72). This method includes administering an effective amount of any of the foregoing compounds or pharmaceutical compositions.

In an aspect, the invention features a method of treating a TDP-43-associated disorder or C₉orf72-associated disorder (e.g., FTLD-TDP, chronic traumatic encephalopathy, ALS, Alzheimer's disease, LATE, or frontotemporal lobar degeneration) in a subject in need thereof. This method includes administering to the subject an effective amount of a compound described herein or a pharmaceutical composition containing one or more compounds described herein.

In another aspect, the invention features a method of inhibiting PIKfyve in a cell expressing PIKfyve protein, the method including contacting the cell with any of the foregoing compounds, or a pharmaceutically acceptable salt thereof.

In another aspect, the invention features a method of treating a neurological disorder in a patient, such as a human patient, identified as likely to benefit from treatment with a compound of the invention on the basis of TDP-43 toxicity. In this aspect, the method may include (i) determining that the patient exhibits, or is prone to develop, TDP-43 toxicity, and (ii) providing to the patient a therapeutically effective amount of a compound of the invention. In some embodiments, the patient has previously been determined to exhibit, or to be prone to developing, TDP-43 toxicity, and the method includes providing to the patient a therapeutically effective amount of a compound of the invention. The susceptibility of the patient to developing TDP-43 aggregation may be determined, e.g., by determining whether the patient expresses a mutant isoform of TDP-43 containing a mutation that is associated with TDP-43 aggregation and toxicity, such as a mutation selected from Q331K, M337V, Q343R, N345K, R361 S, and N390D. This may be performed, for example, by determining the amino acid sequence of a TDP-43 isoform isolated from a sample obtained from the patient or by determining the nucleic acid sequence of a TDP-43 gene isolated from a sample obtained from the patient. In some embodiments, the method includes the step of obtaining the sample from the patient.

In an additional aspect, the invention features a method of treating a neurological disorder in a patient, such as a human patient, identified as likely to benefit from treatment with a compound of the invention on the basis of TDP-43 expression. In this aspect, the method includes (i) determining that the patient expresses a mutant form of TDP-43 having a mutation associated with TDP-43 aggregation (e.g., a mutation selected from Q331K, M337V, Q343R, N345K, R³⁶¹S, and N390D), and (ii) providing to the patient a therapeutically effective amount of a compound of the invention. In some embodiments, the patient has previously been determined to express a mutant form of TDP-43 having a mutation associated with TDP-43 aggregation, such as a Q331K, M337V, Q343R, N345K, R361 S, or N390D mutation, and the method includes providing to the patient a therapeutically effective amount of a compound of the invention.

In another aspect, the invention features a method of determining whether a patient (e.g., a human patient) having a neurological disorder is likely to benefit from treatment with a compound of the invention by (i) determining whether the patient exhibits, or is prone to develop, TDP-43 aggregation and (ii) identifying the patient as likely to benefit from treatment with a compound of the invention if the patient exhibits, or is prone to develop, TDP-43 aggregation. In some embodiments, the method further includes the step of (iii) informing the patient whether he or she is likely to benefit from treatment with a compound of the invention. The susceptibility of the patient to developing TDP-43 aggregation may be determined, e.g., by determining whether the patient expresses a mutant isoform of TDP-43 containing a mutation that is associated with TDP-43 aggregation and toxicity, such as a mutation selected from Q331K, M337V, Q343R, N345K, R³⁶¹S, and N390D. This may be performed, for example, by determining the amino acid sequence of a TDP-43 isoform isolated from a sample obtained from the patient or by determining the nucleic acid sequence of a TDP-43 gene isolated from a sample obtained from the patient. In some embodiments, the method includes the step of obtaining the sample from the patient.

In another aspect, the invention features a method of determining whether a patient (e.g., a human patient) having a neurological disorder is likely to benefit from treatment with a compound of the invention by (i) determining whether the patient expresses a TDP-43 mutant having a mutation associated with TDP-43 aggregation (e.g., a mutation selected from Q331K, M337V, Q343R, N345K, R³⁶¹S, and N390D) and (ii) identifying the patient as likely to benefit from treatment with a compound of the invention if the patient expresses a TDP-43 mutant. In some embodiments, the method further includes the step of (iii) informing the patient whether he or she is likely to benefit from treatment with a compound of the invention. The TDP-43 isoform expressed by the patient may be assessed, for example, by isolated TDP-43 protein from a sample obtained from the patient and sequencing the protein using molecular biology techniques described herein or known in the art. In some embodiments, the TDP-43 isoform expressed by the patient is determined by analyzing the patient's genotype at the TDP-43 locus, for example, by sequencing the TDP-43 gene in a sample obtained from the patient. In some embodiments, the method includes the step of obtaining the sample from the patient.

In some embodiments of any of the above aspects, the compound of the invention is provided to the patient by administration of the compound of the invention to the patient. In some embodiments, the compound of the invention is provided to the patient by administration of a prodrug that is converted in vivo to the compound of the invention.

In some embodiments of any of the above aspects, the neurological disorder is a neuromuscular disorder, such as a neuromuscular disorder selected from amyotrophic lateral sclerosis, congenital myasthenic syndrome, congenital myopathy, cramp fasciculation syndrome, Duchenne muscular dystrophy, glycogen storage disease type II, hereditary spastic paraplegia, inclusion body myositis, Isaac's Syndrome, Kearns-Sayre syndrome, Lambert-Eaton myasthenic syndrome, mitochondrial myopathy, muscular dystrophy, myasthenia gravis, myotonic dystrophy, peripheral neuropathy, spinal and bulbar muscular atrophy, spinal muscular atrophy, Stiff person syndrome, Troyer syndrome, and Guillain-Barre syndrome. In some embodiments, the neurological disorder is amyotrophic lateral sclerosis.

In some embodiments of any of the above aspects, the neurological disorder is selected from frontotemporal degeneration (also referred to as frontotemporal lobar degeneration and frontotemporal dementia), Alzheimer's disease, Parkinson's disease, dementia with Lewy Bodies, corticobasal degeneration, progressive supranuclear palsy, dementia parkinsonism ALS complex of Guam, Huntington's disease, Inclusion body myopathy with early-onset Paget disease and frontotemporal dementia (IBMPFD), sporadic inclusion body myositis, myofibrillar myopathy, dementia pugilistica, chronic traumatic encephalopathy, Alexander disease, and hereditary inclusion body myopathy.

In some embodiments, the neurological disorder is amyotrophic lateral sclerosis, and following administration of the compound of the invention to the patient, the patient exhibits one or more, or all, of the following responses:
(i) an improvement in condition as assessed using the amyotrophic lateral sclerosis functional rating scale (ALSFRS) or the revised ALSFRS (ALSFRS-R), such as an improvement in the patient's ALSFRS or ALSFRS-R score within one or more days, weeks, or months following administration of the compound of the invention (e.g., an improvement in the patient's ALSFRS or ALSFRS-R score within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the compound of the invention to the patient, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, weeks, 46 weeks, 47 weeks, 48 weeks, or more, following the initial administration of the compound of the invention to the patient);
(ii) an increase in slow vital capacity, such as an increase in the patient's slow vital capacity within one or more days, weeks, or months following administration of the compound of the invention (e.g., an increase in the patient's slow vital capacity within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the compound of the invention to the patient, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the compound of the invention to the patient);
(iii) a reduction in decremental responses exhibited by the patient upon repetitive nerve stimulation, such as a reduction that is observed within one or more days, weeks, or months following administration of the compound of the invention (e.g., a reduction that is observed within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the compound of the invention to the patient, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the compound of the invention to the patient);
(iv) an improvement in muscle strength, as assessed, for example, by way of the Medical Research Council muscle testing scale (as described, e.g., in Jagtap et al., Ann. Indian. Acad. Neurol. 17:336-339 (2014), the disclosure of which is incorporated herein by reference as it pertains to measuring patient response to neurological disease treatment), such as an improvement that is observed within one or more days, weeks, or months following administration of the compound of the invention (e.g., an improvement that is observed within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the compound of the invention to the patient, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the compound of the invention to the patient);
(v) an improvement in quality of life, as assessed, for example, using the amyotrophic lateral sclerosis-specific quality of life (ALS-specific QOL) questionnaire, such as an improvement in the patient's quality of life that is observed within one or more days, weeks, or months following administration of the compound of the invention (e.g., an improvement in the subject's quality of life that is observed within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the compound of the invention to the patient, such as within 1 day, 2 days, 3 days, 4 days, 5 days, 6 days, 7 days, 2 weeks, 3 weeks, 4 weeks, weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the compound of the invention to the patient);
(vi) a decrease in the frequency and/or severity of muscle cramps, such as a decrease in cramp frequency and/or severity within one or more days, weeks, or months following administration of the compound of the invention (e.g., a decrease in cramp frequency and/or severity within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the compound of the invention to the patient, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the compound of the invention to the patient); and/or
(vii) a decrease in TDP-43 aggregation, such as a decrease in TDP-43 aggregation within one or more days, weeks, or months following administration of the compound of the invention (e.g., a decrease in TDP-43 aggregation within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the compound of the invention to the patient, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the compound of the invention to the patient.

### Chemical Terms

It is to be understood that the terminology employed herein is for the purpose of describing particular embodiments and is not intended to be limiting.

Those skilled in the art will appreciate that certain compounds described herein can exist in one or more different isomeric (e.g., stereoisomers, geometric isomers, tautomers) and/or isotopic (e.g., in which one or more atoms has been substituted with a different isotope of the atom, such as hydrogen substituted for deuterium) forms. Unless otherwise indicated or clear from context, a depicted structure can be understood to represent any such isomeric or isotopic form, individually or in combination.

In some embodiments, one or more compounds depicted herein may exist in different tautomeric forms. As will be clear from context, unless explicitly excluded, references to such compounds encompass all such tautomeric forms. In some embodiments, tautomeric forms result from the swapping of a single bond with an adjacent double bond and the concomitant migration of a proton. In certain embodiments, a tautomeric form may be a prototropic tautomer, which is an isomeric protonation states having the same empirical formula and total charge as a reference form. Examples of moieties with prototropic tautomeric forms are ketone—enol pairs, amide—imidic acid pairs, lactam—lactim pairs, amide—imidic acid pairs, enamine—imine pairs, and annular forms where a proton can occupy two or more positions of a heterocyclic system, such as, 1H- and 3H-imidazole, 1H—, 2H- and 4H-1,2,4-triazole, 1H- and 2H-isoindole, and 1H- and 2H-pyrazole. In some embodiments, tautomeric forms can be in equilibrium or sterically locked into one form by appropriate substitution. In certain embodiments, tautomeric forms result from acetal interconversion, e.g., the interconversion illustrated in the scheme below:

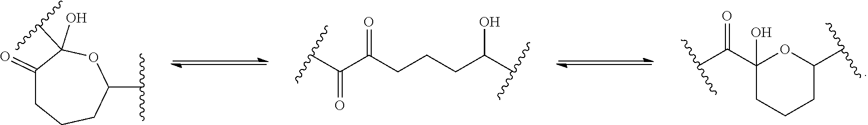 [SMILES: *C1CCCC(=O)C(*)(O)O1 ; *C(=O)C(=O)CCCC(*)O ; *C(=O)C1(O)CCCC(*)O1]

Those skilled in the art will appreciate that, in some embodiments, isotopes of compounds described herein may be prepared and/or utilized in accordance with the present invention. “Isotopes” refers to atoms having the same atomic number but different mass numbers resulting from a different number of neutrons in the nuclei. For example, isotopes of hydrogen include tritium and deuterium. In some embodiments, an isotopic substitution (e.g., substitution of hydrogen with deuterium) may alter the physiciochemical properties of the molecules, such as metabolism and/or the rate of racemization of a chiral center.

As is known in the art, many chemical entities (in particular many organic molecules and/or many small molecules) can adopt a variety of different solid forms such as, for example, amorphous forms and/or crystalline forms (e.g., polymorphs, hydrates, solvates, etc). In some embodiments, such entities may be utilized in any form, including in any solid form. In some embodiments, such entities are utilized in a particular form, for example in a particular solid form.

In some embodiments, compounds described and/or depicted herein may be provided and/or utilized in salt form.

In certain embodiments, compounds described and/or depicted herein may be provided and/or utilized in hydrate or solvate form.

At various places in the present specification, substituents of compounds of the present disclosure are disclosed in groups or in ranges. It is specifically intended that the present disclosure include each and every individual subcombination of the members of such groups and ranges. For example, the term “C₁-C₆ alkyl” is specifically intended to individually disclose methyl, ethyl, C₃ alkyl, C₄ alkyl, C₅ alkyl, and C₆ alkyl. Furthermore, where a compound includes a plurality of positions at which substitutes are disclosed in groups or in ranges, unless otherwise indicated, the present disclosure is intended to cover individual compounds and groups of compounds (e.g., genera and subgenera) containing each and every individual subcombination of members at each position.

Herein a phrase of the form “optionally substituted X” (e.g., optionally substituted alkyl) is intended to be equivalent to “X, where X is optionally substituted” (e.g., “alkyl, where said alkyl is optionally substituted”). It is not intended to mean that the feature “X” (e.g. alkyl) per se is optional.

The term “acyl,” as used herein, represents a hydrogen or an alkyl group, as defined herein that is attached to a parent molecular group through a carbonyl group, as defined herein, and is exemplified by formyl (i.e., a carboxyaldehyde group), acetyl, trifluoroacetyl, propionyl, and butanoyl. Exemplary unsubstituted acyl groups include from 1 to 6, from 1 to 11, or from 1 to 21 carbons.

The term “alkyl,” as used herein, refers to a branched or straight-chain monovalent saturated aliphatic hydrocarbon radical of 1 to 20 carbon atoms (e.g., 1 to 16 carbon atoms, 1 to 10 carbon atoms, or 1 to 6 carbon atoms). An alkylene is a divalent alkyl group.

The term “alkenyl,” as used herein, alone or in combination with other groups, refers to a straight-chain or branched hydrocarbon residue having a carbon-carbon double bond and having 2 to 20 carbon atoms (e.g., 2 to 16 carbon atoms, 2 to 10 carbon atoms, 2 to 6, or 2 carbon atoms).

The term “alkynyl,” as used herein, alone or in combination with other groups, refers to a straight-chain or branched hydrocarbon residue having a carbon-carbon triple bond and having 2 to 20 carbon atoms (e.g., 2 to 16 carbon atoms, 2 to 10 carbon atoms, 2 to 6, or 2 carbon atoms).

The term “amino,” as used herein, represents —N(R^{N1})₂, where each R^{N}1 is, independently, H, OH, NO2, N(R^{N2})₂, SO₂OR^{N2}, SO₂RN², SOR^{N2}, an N-protecting group, alkyl, alkoxy, aryl, arylalkyl, cycloalkyl, acyl (e.g., acetyl, trifluoroacetyl, or others described herein), where each of these recited R^{N}1 groups can be optionally substituted; or two R^{N}1 combine to form an alkylene or heteroalkylene, and where each R^{N2} is, independently, H, alkyl, or aryl. The amino groups of the invention can be an unsubstituted amino (i.e., —NH₂) or a substituted amino (i.e., —N(R^{N1})₂). An amino group, having one R¹ are H and the other R^{N}1 as a non-H group, may be referred to as a monosubstituted amino. For example, when one R^{N}1 is H, and the other R^{N}1 is optionally substituted alkyl, the resulting amino group is an optionally substituted monoalkylamino. When both R^{N}1 groups are independently optionally substituted alkyls, the resulting amino group is an optionally substituted dialkylamino.

The term “aryl,” as used herein, refers to an aromatic mono- or polycarbocyclic radical of 6 to 12 carbon atoms having at least one aromatic ring. Examples of such groups include, but are not limited to, phenyl, naphthyl, 1,2,3,4-tetrahydronaphthyl, 1,2-dihydronaphthyl, indanyl, and 1H-indenyl.

The term “arylalkyl,” as used herein, represents an alkyl group substituted with an aryl group. Exemplary unsubstituted arylalkyl groups are from 7 to 30 carbons (e.g., from 7 to 16 or from 7 to 20 carbons, such as C₆₋₁₀ aryl C₁-C₆ alkyl, C₆₋₁₀ aryl C₁-C₁₀ alkyl, or C₆₋₁₀ aryl C₁-C₂₀ alkyl), such as, benzyl and phenethyl. In some embodiments, the akyl and the aryl each can be further substituted with 1, 2, 3, or 4 substituent groups as defined herein for the respective groups.

The term “arylalkenyl,” as used herein, represents an alkenyl group substituted with an aryl group. Exemplary unsubstituted aryl alkenyl groups are from 7 to 30 carbons (e.g., from 7 to 16 or from 7 to 20 carbons, such as C₆₋₁₀ aryl C₁-C₆ alkyl, C₆₋₁₀ aryl C₁-C₁₀ alkyl, or C₆₋₁₀ aryl C₁-C₂₀ alkyl), such as, 2-phenyl-ethenyl. In some embodiments, the alkenyl and the aryl each can be further substituted with 1, 2, 3, or 4 substituent groups as defined herein for the respective groups.

The term “arylheteroalkyl,” as used herein, represents an heteroalkyl group substituted with an aryl group. Exemplary unsubstituted heteroarylalkyl groups are from 7 to 30 carbons (e.g., from 7 to 16 or from 7 to 20 carbons, such as C₆₋₁₀ heteroaryl C₁-C₆ alkyl, C₆₋₁₀ heteroaryl C₁-C₁₀ alkyl, or C₆₋₁₀ heteroaryl C₁-C₂₀ alkyl), such as benzyloxy. In some embodiments, the akyl and the aryl each can be further substituted with 1, 2, 3, or 4 substituent groups as defined herein for the respective groups.

The term “azido,” as used herein, represents a —N₃ group.

The term “cyano,” as used herein, represents a CN group.

The terms “carbocyclyl,” as used herein, refer to a non-aromatic C₃-C₁₂ monocyclic, bicyclic, or tricyclic structure in which the rings are formed by carbon atoms. Carbocyclyl structures include cycloalkyl groups and unsaturated carbocyclyl radicals.

The term “cycloalkyl,” as used herein, refers to a saturated, non-aromatic, monovalent mono- or polycarbocyclic radical of three to ten, preferably three to six carbon atoms. This term is further exemplified by radicals such as cyclopropyl, cyclobutyl, cyclopentyl, cyclohexyl, cycloheptyl, norbornyl, and adamantyl.

The term “halo,” as used herein, means a fluorine (fluoro), chlorine (chloro), bromine (bromo), or iodine (iodo) radical.

The term “heteroalkyl,” as used herein, refers to an alkyl group, as defined herein, in which one or more of the constituent carbon atoms have been replaced by nitrogen, oxygen, or sulfur. In some embodiments, the heteroalkyl group can be further substituted with 1, 2, 3, or 4 substituent groups as described herein for alkyl groups. Examples of heteroalkyl groups are an “alkoxy” which, as used herein, refers alkyl-O— (e.g., methoxy and ethoxy). A heteroalkylene is a divalent heteroalkyl group.

The term “heteroalkenyl,” as used herein, refers to an alkenyl group, as defined herein, in which one or more of the constituent carbon atoms have been replaced by nitrogen, oxygen, or sulfur. In some embodiments, the heteroalkenyl group can be further substituted with 1, 2, 3, or 4 substituent groups as described herein for alkenyl groups. Examples of heteroalkenyl groups are an “alkenoxy” which, as used herein, refers alkenyl-O—.

The term “heteroalkynyl,” as used herein, refers to an alkynyl group, as defined herein, in which one or more of the constituent carbon atoms have been replaced by nitrogen, oxygen, or sulfur. In some embodiments, the heteroalkynyl group can be further substituted with 1, 2, 3, or 4 substituent groups as described herein for alkynyl groups. Examples of heteroalkynyl groups are an “alkynoxy” which, as used herein, refers alkynyl-O—.

The term “heteroaryl,” as used herein, refers to an aromatic mono- or polycyclic radical of 5 to 12 atoms having at least one aromatic ring, and containing one, two, three, or four ring heteroatoms selected from N, O, and S, with the remaining ring atoms being C₂. One or two ring carbon atoms of the heteroaryl group may be replaced with a carbonyl group. Examples of heteroaryl groups are pyridyl, pyrazoyl, benzooxazolyl, benzoimidazolyl, benzothiazolyl, imidazolyl, oxaxolyl, and thiazolyl.

The term “heteroarylalkyl,” as used herein, represents an alkyl group substituted with a heteroaryl group. Exemplary unsubstituted heteroarylalkyl groups are from 7 to 30 carbons (e.g., from 7 to 16 or from 7 to 20 carbons, such as C₂-C₉ heteroaryl C₁-C₆ alkyl, C₂-C₉ heteroaryl C₁-C₁₀ alkyl, or C₂-C₉ heteroaryl C₁-C₂₀ alkyl). In some embodiments, the akyl and the heteroaryl each can be further substituted with 1, 2, 3, or 4 substituent groups as defined herein for the respective groups.

The term “heterocyclyl,” as used herein, denotes a mono- or polycyclic radical having 3 to 12 atoms having at least one ring containing one, two, three, or four ring heteroatoms selected from N, O or S, where no ring is aromatic. Examples of heterocyclyl groups include, but are not limited to, morpholinyl, thiomorpholinyl, furyl, piperazinyl, piperidinyl, pyranyl, pyrrolidinyl, tetrahydropyranyl, tetrahydrofuranyl, and 1,3-dioxanyl.

The term “heterocyclylalkyl,” as used herein, represents an alkyl group substituted with a heterocyclyl group. Exemplary unsubstituted heterocyclylalkyl groups are from 7 to 30 carbons (e.g., from 7 to 16 or from 7 to 20 carbons, such as C₂-C₉ heterocyclyl C₁-C₆ alkyl, C₂-C₉ heterocyclyl C₁-C₁₀ alkyl, or C₂-C₉ heterocyclyl C₁-C₂₀ alkyl). In some embodiments, the akyl and the heterocyclyl each can be further substituted with 1, 2, 3, or 4 substituent groups as defined herein for the respective groups.

The term “hydroxyl,” as used herein, represents an —OH group.

The term “N-protecting group,” as used herein, represents those groups intended to protect an amino group against undesirable reactions during synthetic procedures. Commonly used N-protecting groups are disclosed in Greene, “Protective Groups in Organic Synthesis,” 3^{rd} Edition (John Wiley & Sons, New York, 1999). N-protecting groups include acyl, aryloyl, or carbamyl groups such as formyl, acetyl, propionyl, pivaloyl, t-butylacetyl, 2-chloroacetyl, 2-bromoacetyl, trifluoroacetyl, trichloroacetyl, phthalyl, o-nitrophenoxyacetyl, α-chlorobutyryl, benzoyl, 4-chlorobenzoyl, 4-bromobenzoyl, 4-nitrobenzoyl, and chiral auxiliaries such as protected or unprotected D, L or D, L-amino acids such as alanine, leucine, and phenylalanine; sulfonyl-containing groups such as benzenesulfonyl, and p-toluenesulfonyl; carbamate forming groups such as benzyloxycarbonyl, p-chlorobenzyloxycarbonyl, p-methoxybenzyloxycarbonyl, p-nitrobenzyloxycarbonyl, 2-nitrobenzyloxycarbonyl, p-bromobenzyloxycarbonyl, 3,4-dimethoxybenzyloxycarbonyl, 3,5-dimethoxybenzyloxycarbonyl, 2,4-dimethoxybenzyloxycarbonyl, 4-methoxybenzyloxycarbonyl, 2-nitro-4,5-dimethoxybenzyloxycarbonyl, 3,4,5-trimethoxybenzyloxycarbonyl, 1-(p-biphenylyl)-1-methylethoxycarbonyl, α,α-dimethyl-3,5-dimethoxybenzyloxycarbonyl, benzhydryloxy carbonyl, t-butyloxycarbonyl, diisopropylmethoxycarbonyl, isopropyloxycarbonyl, ethoxycarbonyl, methoxycarbonyl, allyloxycarbonyl, 2,2,2,-trichloroethoxycarbonyl, phenoxycarbonyl, 4-nitrophenoxy carbonyl, fluorenyl-9-methoxycarbonyl, cyclopentyloxycarbonyl, adamantyloxycarbonyl, cyclohexyloxycarbonyl, and phenylthiocarbonyl, arylalkyl groups such as benzyl, triphenylmethyl, and benzyloxymethyl, and silyl groups, such as trimethylsilyl. Preferred N-protecting groups are alloc, formyl, acetyl, benzoyl, pivaloyl, t-butylacetyl, alanyl, phenylsulfonyl, benzyl, t-butyloxycarbonyl (Boc), and benzyloxycarbonyl (Cbz).

The term “nitro,” as used herein, represents an NO₂ group.

The term “oxyheteroaryl,” as used herein, represents a heteroaryl group having at least one endocyclic oxygen atom.

The term “oxyheterocyclyl,” as used herein, represents a heterocyclyl group having at least one endocyclic oxygen atom.

The term “primary imine,” as used herein, represents a ═NH group.

The term “thiol,” as used herein, represents an —SH group.

The alkyl, alkenyl, alkynyl, heteroalkyl, heteroalkenyl, heteroalkynyl, carbocyclyl (e.g., cycloalkyl), aryl, heteroaryl, and heterocyclyl groups may be substituted or unsubstituted. When substituted, there will generally be 1 to 4 substituents present, unless otherwise specified. Substituents include, for example: aryl (e.g., substituted and unsubstituted phenyl), carbocyclyl (e.g., substituted and unsubstituted cycloalkyl), halo (e.g., fluoro), hydroxyl, oxo, heteroalkyl (e.g., substituted and unsubstituted methoxy, ethoxy, or thioalkoxy), heteroaryl, heterocyclyl, amino (e.g., NH₂ or mono- or dialkyl amino), azido, cyano, nitro, or thiol. Aryl, carbocyclyl (e.g., cycloalkyl), heteroaryl, and heterocyclyl groups may also be substituted with alkyl (unsubstituted and substituted such as arylalkyl (e.g., substituted and unsubstituted benzyl)).

Compounds of the invention can have one or more asymmetric carbon atoms and can exist in the form of optically pure enantiomers, mixtures of enantiomers such as, for example, racemates, optically pure diastereoisomers, mixtures of diastereoisomers, diastereoisomeric racemates or mixtures of diastereoisomeric racemates. The optically active forms can be obtained for example by resolution of the racemates, by asymmetric synthesis or asymmetric chromatography (chromatography with a chiral adsorbent or eluant). That is, certain of the disclosed compounds may exist in various stereoisomeric forms. Stereoisomers are compounds that differ only in their spatial arrangement. Enantiomers are pairs of stereoisomers whose mirror images are not superimposable, most commonly because they contain an asymmetrically substituted carbon atom that acts as a chiral center. “Enantiomer” means one of a pair of molecules that are mirror images of each other and are not superimposable. Diastereomers are stereoisomers that are not related as mirror images, most commonly because they contain two or more asymmetrically substituted carbon atoms and represent the configuration of substituents around one or more chiral carbon atoms. Enantiomers of a compound can be prepared, for example, by separating an enantiomer from a racemate using one or more well-known techniques and methods, such as, for example, chiral chromatography and separation methods based thereon. The appropriate technique and/or method for separating an enantiomer of a compound described herein from a racemic mixture can be readily determined by those of skill in the art. “Racemate” or “racemic mixture” means a compound containing two enantiomers, where such mixtures exhibit no optical activity; i.e., they do not rotate the plane of polarized light. “Geometric isomer” means isomers that differ in the orientation of substituent atoms in relationship to a carbon-carbon double bond, to a cycloalkyl ring, or to a bridged bicyclic system. Atoms (other than H) on each side of a carbon-carbon double bond may be in an E (substituents are on opposite sides of the carbon-carbon double bond) or Z (substituents are oriented on the same side) configuration. “R,” “S,” “S*,” “R*,” “E,” “Z,” “cis,” and “trans,” indicate configurations relative to the core molecule. Certain of the disclosed compounds may exist in atropisomeric forms. Atropisomers are stereoisomers resulting from hindered rotation about single bonds where the steric strain barrier to rotation is high enough to allow for the isolation of the conformers. The compounds of the invention may be prepared as individual isomers by either isomer-specific synthesis or resolved from an isomeric mixture. Conventional resolution techniques include forming the salt of a free base of each isomer of an isomeric pair using an optically active acid (followed by fractional crystallization and regeneration of the free base), forming the salt of the acid form of each isomer of an isomeric pair using an optically active amine (followed by fractional crystallization and regeneration of the free acid), forming an ester or amide of each of the isomers of an isomeric pair using an optically pure acid, amine or alcohol (followed by chromatographic separation and removal of the chiral auxiliary), or resolving an isomeric mixture of either a starting material or a final product using various well known chromatographic methods. When the stereochemistry of a disclosed compound is named or depicted by structure, the named or depicted stereoisomer is at least 60%, 70%, 80%, 90%, 99% or 99.9%) by weight relative to the other stereoisomers. When a single enantiomer is named or depicted by structure, the depicted or named enantiomer is at least 60%, 70%, 80%, 90%, 99% or 99.9% by weight optically pure. When a single diastereomer is named or depicted by structure, the depicted or named diastereomer is at least 60%, 70%, 80%, 90%, 99% or 99.9% by weight pure. Percent optical purity is the ratio of the weight of the enantiomer or over the weight of the enantiomer plus the weight of its optical isomer. Diastereomeric purity by weight is the ratio of the weight of one diastereomer or over the weight of all the diastereomers. When the stereochemistry of a disclosed compound is named or depicted by structure, the named or depicted stereoisomer is at least 60%, 70%, 80%, 90%, 99% or 99.9% by mole fraction pure relative to the other stereoisomers. When a single enantiomer is named or depicted by structure, the depicted or named enantiomer is at least 60%, 70%, 80%, 90%, 99% or 99.9% by mole fraction pure. When a single diastereomer is named or depicted by structure, the depicted or named diastereomer is at least 60%, 70%, 80%, 90%, 99% or 99.9% by mole fraction pure. Percent purity by mole fraction is the ratio of the moles of the enantiomer or over the moles of the enantiomer plus the moles of its optical isomer. Similarly, percent purity by moles fraction is the ratio of the moles of the diastereomer or over the moles of the diastereomer plus the moles of its isomer. When a disclosed compound is named or depicted by structure without indicating the stereochemistry, and the compound has at least one chiral center, it is to be understood that the name or structure encompasses either enantiomer of the compound free from the corresponding optical isomer, a racemic mixture of the compound or mixtures enriched in one enantiomer relative to its corresponding optical isomer. When a disclosed compound is named or depicted by structure without indicating the stereochemistry and has two or more chiral centers, it is to be understood that the name or structure encompasses a diastereomer free of other diastereomers, a number of diastereomers free from other diastereomeric pairs, mixtures of diastereomers, mixtures of diastereomeric pairs, mixtures of diastereomers in which one diastereomer is enriched relative to the other diastereomer(s) or mixtures of diastereomers in which one or more diastereomer is enriched relative to the other diastereomers. The invention embraces all of these forms.

### Definitions

In this application, unless otherwise clear from context, (i) the term “a” may be understood to mean “at least one”; (ii) the term “or” may be understood to mean “and/or”; (iii) the terms “comprising” and “including” may be understood to encompass itemized components or steps whether presented by themselves or together with one or more additional components or steps; and (iv) the terms “about” and “approximately” may be understood to permit standard variation as would be understood by those of ordinary skill in the art; and (v) where ranges are provided, endpoints are included.

As used herein, the term “administration” refers to the administration of a composition (e.g., a compound, a complex or a preparation that includes a compound or complex as described herein) to a subject or system. Administration to an animal subject (e.g., to a human) may be by any appropriate route. For example, in some embodiments, administration may be bronchial (including by bronchial instillation), buccal, enteral, interdermal, intra-arterial, intradermal, intragastric, intramedullary, intramuscular, intranasal, intraperitoneal, intrathecal, intravenous, intraventricular, mucosal, nasal, oral, rectal, subcutaneous, sublingual, topical, tracheal (including by intratracheal instillation), transdermal, vaginal and vitreal.

As used herein, the term “animal” refers to any member of the animal kingdom. In some embodiments, “animal” refers to humans, at any stage of development. In some embodiments, “animal” refers to non-human animals, at any stage of development. In some embodiments, the non-human animal is a mammal (e.g., a rodent, a mouse, a rat, a rabbit, a monkey, a dog, a cat, a sheep, cattle, a primate, and/or a pig). In some embodiments, animals include, but are not limited to, mammals, birds, reptiles, amphibians, fish, and/or worms. In some embodiments, an animal may be a transgenic animal, genetically-engineered animal, and/or a clone.

As used herein, the terms “approximately” and “about” are each intended to encompass normal statistical variation as would be understood by those of ordinary skill in the art as appropriate to the relevant context. In certain embodiments, the terms “approximately” or “about” each refer to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of a stated value, unless otherwise stated or otherwise evident from the context (e.g., where such number would exceed 100% of a possible value).

Two events or entities are “associated” with one another, as that term is used herein, if the presence, level and/or form of one is correlated with that of the other. For example, a particular entity (e.g., polypeptide) is considered to be associated with a particular disease, disorder, or condition, if its presence, level and/or form correlates with incidence of and/or susceptibility of the disease, disorder, or condition (e.g., across a relevant population).

As used herein, the terms “benefit” and “response” are used interchangeably in the context of a subject, such as a human subject undergoing therapy for the treatment of a neurological disorder, for example, amyotrophic lateral sclerosis, frontotemporal degeneration (also referred to as frontotemporal lobar degeneration and frontotemporal dementia), Alzheimer's disease, Parkinson's disease, dementia with Lewy Bodies, corticobasal degeneration, progressive supranuclear palsy, dementia parkinsonism ALS complex of Guam, Huntington's disease, Inclusion body myopathy with early-onset Paget disease and frontotemporal dementia (IBMPFD), sporadic inclusion body myositis, myofibrillar myopathy, dementia pugilistica, chronic traumatic encephalopathy, Alexander disease, and hereditary inclusion body myopathy. The terms “benefit” and “response” refer to any clinical improvement in the subject's condition. Exemplary benefits in the context of a subject undergoing treatment for a neurological disorder using the compositions and methods described herein (e.g., in the context of a human subject undergoing treatment for a neurological disorder described herein, such as amyotrophic lateral sclerosis, with a FYVE-type zinc finger containing phosphoinositide kinase (PIKfyve) inhibitor described herein, such as an inhibitory small molecule, antibody, antigen-binding fragment thereof, or interfering RNA molecule) include the slowing and halting of disease progression, as well as suppression of one or more symptoms associated with the disease. Particularly, in the context of a patient (e.g., a human patient) undergoing treatment for amyotrophic lateral sclerosis with a compound of the invention, examples of clinical “benefits” and “responses” are (i) an improvement in the subject's condition as assessed using the amyotrophic lateral sclerosis functional rating scale (ALSFRS) or the revised ALSFRS (ALSFRS-R) following administration of the compound of the invention, such as an improvement in the subject's ALSFRS or ALSFRS-R score within one or more days, weeks, or months following administration of the compound of the invention (e.g., an improvement in the subject's ALSFRS or ALSFRS-R score within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the compound of the invention to the subject, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, weeks, 46 weeks, 47 weeks, 48 weeks, or more, following the initial administration of the compound of the invention to the subject); (ii) an increase in the subject's slow vital capacity following administration of the compound of the invention, such as an increase in the subject's slow vital capacity within one or more days, weeks, or months following administration of the compound of the invention (e.g., an increase in the subject's slow vital capacity within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the compound of the invention to the subject, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the compound of the invention to the subject); (iii) a reduction in decremental responses exhibited by the subject upon repetitive nerve stimulation, such as a reduction that is observed within one or more days, weeks, or months following administration of the compound of the invention (e.g., a reduction that is observed within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the compound of the invention to the subject, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the compound of the invention to the subject); (iv) an improvement in the subject's muscle strength, as assessed, for example, by way of the Medical Research Council muscle testing scale (as described, e.g., in Jagtap et al., Ann. Indian. Acad. Neurol. 17:336-339 (2014), the disclosure of which is incorporated herein by reference as it pertains to measuring patient response to neurological disease treatment), such as an improvement that is observed within one or more days, weeks, or months following administration of the compound of the invention (e.g., an improvement that is observed within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the compound of the invention to the subject, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, weeks, 46 weeks, 47 weeks, 48 weeks, or more, following the initial administration of the compound of the invention to the subject); (v) an improvement in the subject's quality of life, as assessed, for example, using the amyotrophic lateral sclerosis-specific quality of life (ALS-specific QOL) questionnaire, such as an improvement in the subject's quality of life that is observed within one or more days, weeks, or months following administration of the compound of the invention (e.g., an improvement in the subject's quality of life that is observed within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the compound of the invention to the subject, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the compound of the invention to the subject); and (vi) a decrease in the frequency and/or severity of muscle cramps exhibited by the subject, such as a decrease in cramp frequency and/or severity within one or more days, weeks, or months following administration of the compound of the invention (e.g., a decrease in cramp frequency and/or severity within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the compound of the invention to the subject, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the compound of the invention to the subject).

As used herein, the term “dosage form” refers to a physically discrete unit of an active compound (e.g., a therapeutic or diagnostic agent) for administration to a subject. Each unit contains a predetermined quantity of active agent. In some embodiments, such quantity is a unit dosage amount (or a whole fraction thereof) appropriate for administration in accordance with a dosing regimen that has been determined to correlate with a desired or beneficial outcome when administered to a relevant population (i.e., with a therapeutic dosing regimen). Those of ordinary skill in the art appreciate that the total amount of a therapeutic composition or compound administered to a particular subject is determined by one or more attending physicians and may involve administration of multiple dosage forms.

As used herein, the term “dosing regimen” refers to a set of unit doses (typically more than one) that are administered individually to a subject, typically separated by periods of time. In some embodiments, a given therapeutic compound has a recommended dosing regimen, which may involve one or more doses. In some embodiments, a dosing regimen comprises a plurality of doses each of which are separated from one another by a time period of the same length; in some embodiments, a dosing regimen comprises a plurality of doses and at least two different time periods separating individual doses. In some embodiments, all doses within a dosing regimen are of the same unit dose amount. In some embodiments, different doses within a dosing regimen are of different amounts. In some embodiments, a dosing regimen comprises a first dose in a first dose amount, followed by one or more additional doses in a second dose amount different from the first dose amount. In some embodiments, a dosing regimen comprises a first dose in a first dose amount, followed by one or more additional doses in a second dose amount same as the first dose amount In some embodiments, a dosing regimen is correlated with a desired or beneficial outcome when administered across a relevant population (i.e., is a therapeutic dosing regimen).

In the practice of the methods of the present invention, an “effective amount” of any one of the compounds of the invention or a combination of any of the compounds of the invention or a pharmaceutically acceptable salt thereof, is administered via any of the usual and acceptable methods known in the art, either singly or in combination.

The term “pharmaceutical composition,” as used herein, represents a composition containing a compound described herein formulated with a pharmaceutically acceptable excipient, and manufactured or sold with the approval of a governmental regulatory agency as part of a therapeutic regimen for the treatment of disease in a mammal. Pharmaceutical compositions can be formulated, for example, for oral administration in unit dosage form (e.g., a tablet, capsule, caplet, gelcap, or syrup); for topical administration (e.g., as a cream, gel, lotion, or ointment); for intravenous administration (e.g., as a sterile solution free of particulate emboli and in a solvent system suitable for intravenous use); or in any other pharmaceutically acceptable formulation.

A “pharmaceutically acceptable excipient,” as used herein, refers any ingredient other than the compounds described herein (for example, a vehicle capable of suspending or dissolving the active compound) and having the properties of being substantially nontoxic and non-inflammatory in a patient. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspensing or dispersing agents, sweeteners, and waters of hydration. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E, vitamin C, and xylitol.

As used herein, the term “pharmaceutically acceptable salt” means any pharmaceutically acceptable salt of the compound of formula (I). For example pharmaceutically acceptable salts of any of the compounds described herein include those that are within the scope of sound medical judgment, suitable for use in contact with the tissues of humans and animals without undue toxicity, irritation, allergic response and are commensurate with a reasonable benefit/risk ratio. Pharmaceutically acceptable salts are well known in the art. For example, pharmaceutically acceptable salts are described in: Berge et al., J. Pharmaceutical Sciences 66:1-19, 1977 and in Pharmaceutical Salts: Properties, Selection, and Use, (Eds. P. H. Stahl and C. G. Wermuth), Wiley-VCH, 2008. The salts can be prepared in situ during the final isolation and purification of the compounds described herein or separately by reacting a free base group with a suitable organic acid.

The compounds of the invention may have ionizable groups so as to be capable of preparation as pharmaceutically acceptable salts. These salts may be acid addition salts involving inorganic or organic acids or the salts may, in the case of acidic forms of the compounds of the invention be prepared from inorganic or organic bases. Frequently, the compounds are prepared or used as pharmaceutically acceptable salts prepared as addition products of pharmaceutically acceptable acids or bases. Suitable pharmaceutically acceptable acids and bases and methods for preparation of the appropriate salts are well-known in the art. Salts may be prepared from pharmaceutically acceptable non-toxic acids and bases including inorganic and organic acids and bases.

The terms “PIKfyve” and “FYVE-type zinc finger containing phosphoinositide kinase” are used interchangeably herein and refer to the enzyme that catalyzes phosphorylation of phosphatidylinositol 3-phosphate to produce phosphatidylinositol 3,5-bisphosphate, for example, in human subjects. The terms “PIKfyve” and “FYVE-type zinc finger containing phosphoinositide kinase” refer not only to wild-type forms of PIKfyve, but also to variants of wild-type PIKfyve proteins and nucleic acids encoding the same. The gene encoding PIKfyve can be accessed under NCBI Reference Sequence No. NG_021188.1. Exemplary transcript sequences of wild-type form of human PIKfyve can be accessed under NCBI Reference Sequence Nos. NM_015040.4, NM_152671.3, and NM_001178000.1. Exemplary protein sequences of wild-type form of human PIKfyve can be accessed under NCBI Reference Sequence Nos. NP_055855.2, NP_689884.1, and NP_001171471.1.

As used herein, the term “PIKfyve inhibitor” refers to substances, such as compounds of Formula I. Inhibitors of this type may, for example, competitively inhibit PIKfyve activity by specifically binding the PIKfyve enzyme (e.g., by virtue of the affinity of the inhibitor for the PIKfyve active site), thereby precluding, hindering, or halting the entry of one or more endogenous substrates of PIKfyve into the enzyme's active site. Additional examples of PIKfyve inhibitors that suppress the activity of the PIKfyve enzyme include substances that may bind PIKfyve at a site distal from the active site and attenuate the binding of endogenous substrates to the PIKfyve active site by way of a change in the enzyme's spatial conformation upon binding of the inhibitor. In addition to encompassing substances that modulate PIKfyve activity, the term “PIKfyve inhibitor” refers to substances that reduce the concentration and/or stability of PIKfyve mRNA transcripts in vivo, as well as those that suppress the translation of functional PIKfyve enzyme.

The term “pure” means substantially pure or free of unwanted components (e.g., other compounds and/or other components of a cell lysate), material defilement, admixture or imperfection.

Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, and valerate salts. Representative alkali or alkaline earth metal salts include sodium, lithium, potassium, calcium, and magnesium, as well as nontoxic ammonium, quaternary ammonium, and amine cations, including, but not limited to ammonium, tetramethylammonium, tetraethylammonium, methylamine, dimethylamine, trimethylamine, triethylamine, and ethylamine.

A variety of clinical indicators can be used to identify a patient as “at risk” of developing a particular neurological disease. Examples of patients (e.g., human patients) that are “at risk” of developing a neurological disease, such as amyotrophic lateral sclerosis, frontotemporal degeneration, Alzheimer's disease, Parkinson's disease, dementia with Lewy Bodies, corticobasal degeneration, progressive supranuclear palsy, dementia parkinsonism ALS complex of Guam, Huntington's disease, Inclusion body myopathy with early-onset Paget disease and frontotemporal dementia (IBMPFD), sporadic inclusion body myositis, myofibrillar myopathy, dementia pugilistica, chronic traumatic encephalopathy, Alexander disease, and hereditary inclusion body myopathy, include (i) subjects exhibiting or prone to exhibit aggregation of TAR-DNA binding protein (TDP)-43, and (ii) subjects expressing a mutant form of TDP-43 containing a mutation associated with TDP-43 aggregation and toxicity, such as a mutation selected from Q331K, M337V, Q343R, N345K, R³⁶¹S, and N390D. Subjects that are “at risk” of developing amyotrophic lateral sclerosis may exhibit one or both of these characteristics, for example, prior to the first administration of a PIKfyve inhibitor in accordance with the compositions and methods described herein.

As used herein, the terms “TAR-DNA binding protein-43” and “TDP-43” are used interchangeably and refer to the transcription repressor protein involved in modulating HIV-1 transcription and alternative splicing of the cystic fibrosis transmembrane conductance regulator (CFTR) pre-mRNA transcript, for example, in human subjects. The terms “TAR-DNA binding protein-43” and “TDP-43” refer not only to wild-type forms of TDP-43, but also to variants of wild-type TDP-43 proteins and nucleic acids encoding the same. The amino acid sequence and corresponding mRNA sequence of a wild-type form of human TDP-43 are provided under NCBI Reference Sequence Nos. NM_007375.3 and NP_031401.1, respectively.

The terms “TAR-DNA binding protein-43” and “TDP-43” as used herein include, for example, forms of the human TDP-43 protein that have an amino acid sequence that is at least 85% identical to the amino acid sequence of NCBI Reference Sequence No. NP_031401.1 (e.g., 85%, 86%, 87%, 88%, 89%, 90%, 91%, 92%, 93%, 94%, 95%, 96%, 97%, 98%, 99%, 99.9%, or 100% identical to the amino acid sequence of NCBI Reference Sequence No. NP_031401.1) and/or forms of the human TDP-43 protein that contain one or more substitutions, insertions, and/or deletions (e.g., one or more conservative and/or nonconservative amino acid substitutions, such as up to 5, 10, 15, 20, 25, or more, conservative or nonconservative amino acid substitutions) relative to a wild-type TDP-43 protein. For instance, patients that may be treated for a neurological disorder as described herein, such as amyotrophic lateral sclerosis, frontotemporal degeneration, Alzheimer's disease, Parkinson's disease, dementia with Lewy Bodies, corticobasal degeneration, progressive supranuclear palsy, dementia parkinsonism ALS complex of Guam, Huntington's disease, Inclusion body myopathy with early-onset Paget disease and frontotemporal dementia (IBMPFD), sporadic inclusion body myositis, myofibrillar myopathy, dementia pugilistica, chronic traumatic encephalopathy, Alexander disease, and hereditary inclusion body myopathy, include human patients that express a form of TDP-43 having a mutation associated with elevated TDP-43 aggregation and toxicity, such as a mutation selected from Q331K, M337V, Q343R, N345K, R³⁶¹S, and N390D. Similarly, the terms “TAR-DNA binding protein-43” and “TDP-43” as used herein include, for example, forms of the human TDP-43 gene that encode an mRNA transcript having a nucleic acid sequence that is at least 85% identical to the nucleic acid sequence of NCBI Reference Sequence No. NM_007375.3 (e.g., 85%, 86%, 87%, 88%, 89%, 90%, 91%, 92%, 93%, 94%, 95%, 96%, 97%, 98%, 99%, 99.9%, or 100% identical to the amino acid sequence of NCBI Reference Sequence No. NM_007375.3).

As used herein, the term “subject” refers to any organism to which a composition in accordance with the invention may be administered, e.g., for experimental, diagnostic, prophylactic, and/or therapeutic purposes. Typical subjects include any animal (e.g., mammals such as mice, rats, rabbits, non-human primates, and humans). A subject may seek or be in need of treatment, require treatment, be receiving treatment, be receiving treatment in the future, or be a human or animal who is under care by a trained professional for a particular disease or condition.

A “therapeutic regimen” refers to a dosing regimen whose administration across a relevant population is correlated with a desired or beneficial therapeutic outcome.

The term “therapeutically effective amount” means an amount that is sufficient, when administered to a population suffering from or susceptible to a disease, disorder, and/or condition in accordance with a therapeutic dosing regimen, to treat the disease, disorder, and/or condition. In some embodiments, a therapeutically effective amount is one that reduces the incidence and/or severity of, and/or delays onset of, one or more symptoms of the disease, disorder, and/or condition. Those of ordinary skill in the art will appreciate that the term “therapeutically effective amount” does not in fact require successful treatment be achieved in a particular individual. Rather, a therapeutically effective amount may be that amount that provides a particular desired pharmacological response in a significant number of subjects when administered to patients in need of such treatment. It is specifically understood that particular subjects may, in fact, be “refractory” to a “therapeutically effective amount.” To give but one example, a refractory subject may have a low bioavailability such that clinical efficacy is not obtainable. In some embodiments, reference to a therapeutically effective amount may be a reference to an amount as measured in one or more specific tissues (e.g., a tissue affected by the disease, disorder or condition) or fluids (e.g., blood, saliva, serum, sweat, tears, urine, etc). Those of ordinary skill in the art will appreciate that, in some embodiments, a therapeutically effective amount may be formulated and/or administered in a single dose. In some embodiments, a therapeutically effective amount may be formulated and/or administered in a plurality of doses, for example, as part of a dosing regimen.

### BRIEF DESCRIPTION OF THE DRAWINGS

FIG. 1 is a scheme showing an approach to generation of a control TDP-43 yeast model (FAB1 TDP-43). A control yeast TDP-43 model was generated by integrating the human TDP-43 gene and the GAL1 promoter into the yeast genome. The yeast ortholog of human PIKFYVE is FAB1.

FIG. 2 is a scheme showing an approach to generation of a humanized PIKFYVE TDP-43 yeast model (PIKFYVE TDP-43). FAB1 gene through homologous recombination with a G418 resistance cassette (fab1::G418R) (FIG. 2). PIKFYVE was cloned downstream of the GPD promoter harbored on a URA3-containing plasmid and introduced into the fab1::G418R ura3 strain. The pGAL1-TDP-43 construct was then introduced into the “humanized” yeast strain and assessed for cytotoxicity.

FIG. 3 is a histogram generated from the flow cytometry-based viability assay of FAB1 TDP-43.

FIG. 4 is a histogram generated from the flow cytometry-based viability assay of PIKFYVE TDP-43. Upon induction of TDP-43, there was a marked increase in inviable cells (rightmost population), with a more pronounced effect in PIKFYVE TDP-43 than in FAB1 TDP-43 strain (see FIG. 3).

FIG. 5 is an overlay of histograms generated from the flow cytometry-based viability assay of FAB1 TDP-43 in the presence of APY0201.

FIG. 6 is an overlay of histograms generated from the flow cytometry-based viability assay of PIKFYVE TDP-43 in the presence of APY0201.

FIG. 7 is a scatter plot comparing cytoprotection efficacy in PIKFYVE TDP-43 to PIKfyve inhibitory activity of test compounds.

### DETAILED DESCRIPTION

The present invention features compositions and methods for treating neurological disorders, such as amyotrophic lateral sclerosis and other neuromuscular disorders, as well as frontotemporal degeneration, Alzheimer's disease, Parkinson's disease, dementia with Lewy Bodies, corticobasal degeneration, progressive supranuclear palsy, dementia parkinsonism ALS complex of Guam, Huntington's disease, Inclusion body myopathy with early-onset Paget disease and frontotemporal dementia (IBMPFD), sporadic inclusion body myositis, myofibrillar myopathy, dementia pugilistica, chronic traumatic encephalopathy, Alexander disease, and hereditary inclusion body myopathy among others. Particularly, the invention provides inhibitors of FYVE-type zinc finger containing phosphoinositide kinase (PIKfyve), that may be administered to a patient (e.g., a human patient) so as to treat or prevent a neurological disorder, such as one or more of the foregoing conditions. In the context of therapeutic treatment, the PIKfyve inhibitor may be administered to the patient to alleviate one or more symptoms of the disorder and/or to remedy an underlying molecular pathology associated with the disease, such as to suppress or prevent aggregation of TAR-DNA binding protein (TDP)-43.

The disclosure herein is based, in part, on the discovery that PIKfyve inhibition modulates TDP-43 aggregation in cells. Suppression of TDP-43 aggregation exerts beneficial effects in patients suffering from a neurological disorder. Many pathological conditions have been correlated with TDP-43-promoted aggregation and toxicity, such as amyotrophic lateral sclerosis, frontotemporal degeneration, Alzheimer's disease, Parkinson's disease, dementia with Lewy Bodies, corticobasal degeneration, progressive supranuclear palsy, dementia parkinsonism ALS complex of Guam, Huntington's disease, IBMPFD, sporadic inclusion body myositis, myofibrillar myopathy, dementia pugilistica, chronic traumatic encephalopathy, Alexander disease, and hereditary inclusion body myopathy. Without being limited by mechanism, by administering an inhibitor of PIKfyve, patients suffering from diseases associated with TDP-43 aggregation and toxicity may be treated, for example, due to the suppression of TDP-43 aggregation induced by the PIKfyve inhibitor.

Patients that are likely to respond to PIKfyve inhibition as described herein include those that have or are at risk of developing TDP-43 aggregation, such as those that express a mutant form of TDP-43 associated with TDP-43 aggregation and toxicity in vivo. Examples of such mutations in TDP-43 that have been correlated with elevated TDP-43 aggregation and toxicity include Q331K, M337V, Q343R, N345K, R361 S, and N390D, among others. The compositions and methods described herein thus provide the additional clinical benefit of enabling the identification of patients that are likely to respond to PIKfyve inhibitor therapy, as well as processes for treating these patients accordingly.

The sections that follow provide a description of exemplary PIKfyve inhibitors that may be used in conjunction with the compositions and methods disclosed herein. The sections below additionally provide a description of various exemplary routes of administration and pharmaceutical compositions that may be used for delivery of these substances for the treatment of a neurological disorder.

### PIKfyve Inhibitors

Exemplary PIKfyve inhibitors described herein include a compound of Formula I:

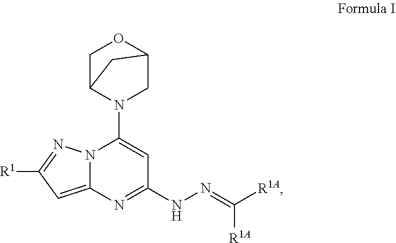

or pharmaceutically acceptable salt thereof,
where:
R¹ is optionally substituted C₂-C₉ heteroaryl; and
each R^{1A} is independently H, optionally substituted C₁-C₆ alkyl, optionally substituted C₆-C₁₀ aryl, or optionally substituted C₂-C₉ heteroaryl; and the remaining R^{1B} is optionally substituted 01-C₆ alkyl, optionally substituted C₆-C₁₀ aryl, or optionally substituted C₂-C₉ heteroaryl.

Exemplary PIKfyve inhibitors described herein include a compound of Formula II:

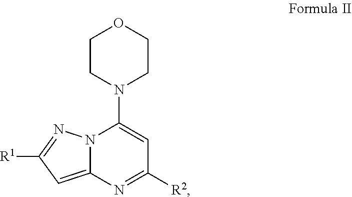

or pharmaceutically acceptable salt thereof,
where:
R¹ is optionally substituted pyridin-4-yl;
R² is optionally substituted C₂-C₉ heterocyclyl, optionally substituted C₁-C₆ alkyl, optionally substituted C₁-C₆ alkenyl, optionally substituted pyridin-2-yl, optionally substituted pyridin-3-yl, optionally substituted pyrimidn-4-yl, optionally substituted thiadiazolyl, optionally substituted oxadiazolyl, optionally substituted dialkylamino, optionally substituted 6-oxo-1,5-dihydropyridazin-1-yl, optionally substituted pyrazinyl, fluoro, cyano, optionally substituted pyrazol-3-yl, optionally substituted pyrazol-5-yl, optionally substituted oxazole, optionally substituted N-tetrahydropyranopyrazolyl, optionally substituted N-tetrahydroindazolyl, optionally substituted C₁-C₆ heteroalkyl, optionally substituted C₆-C₁₀ aryl C₁-C₆ alkyl, optionally substituted C₆-C₁₀ aryl C₁-C₆ alkenyl, optionally substituted C₆-C₁₀ aryl C₁-C₆ heteroalkyl, CONH₂, —CONH—NHR^{1A},

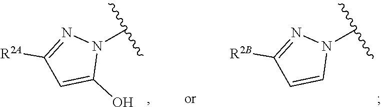

R^{1A} is H or optionally substituted C₂-C₁₀ acyl;
R^{2A} is optionally substituted aryl, optionally substituted C₁-C₆ alkyl, optionally substituted C₂-C₅ heteroaryl, or optionally substituted C₃-C₆ cycloalkyl; and
R^{2B} is pyridizin-4-yl, phenyl substituted with fluoro or methoxy, piperidinyl optionally substituted with C₁-C₆ alkyl, optionally substituted pyrimidin-5-yl, optionally substituted pyridin-2-yl, optionally substituted pyridine-3-yl, optionally substituted C₃ carbocyclyl, azetidin-3-yl, optionally substituted C₁-C₆ hydroxyalkyl, or C₃ heteroalkyl.

Exemplary PIKFyve inhibitors described herein include a compound of Formula III:

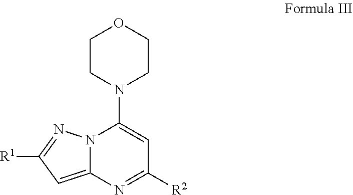

where:
R¹ is optionally substituted C₁₋₆ alkenyl, optionally substituted C₁-C₆ hydroxyalkyl, C₁-C₆ alkyl substituted with dialkyl amino, hydrogen, or optionally substituted C₂-C₉ heterocyclyl; and
R² is optionally substituted pyrazol-1-yl, optionally substituted pyrazol-3-yl, C₆-C₁₀ aryl optionally substituted with optionally substituted C₂-C₉ heteroaryl, optionally substituted N-tetrahydropyranopyrazolyl, or optionally substituted pyrimidin-4-yl.

PIKfyve inhibitors described herein include any one of the compounds in Table 1.

TABLE 1
Compounds of the Invention.

| # | Structure |
|---|---|
| 1 | 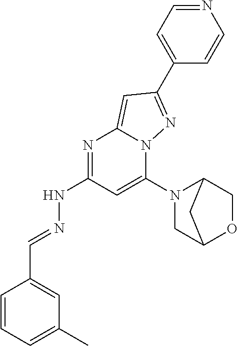 [SMILES: Cc1cccc(/C=N/Nc2cc(N3CC4CC3CO4)n3nc(-c4ccncc4)cc3n2)c1] |
| 2 | 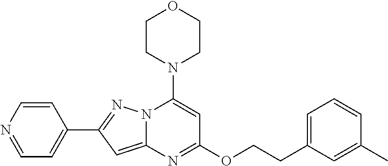 [SMILES: Cc1cccc(CCOc2cc(N3CCOCC3)n3nc(-c4ccncc4)cc3n2)c1] |
| 3 | 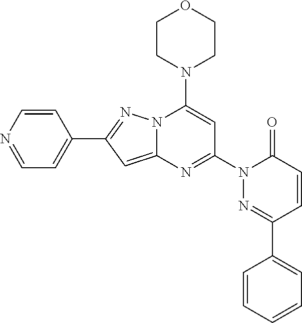 [SMILES: O=c1ccc(-c2ccccc2)nn1-c1cc(N2CCOCC2)n2nc(-c3ccncc3)cc2n1] |
| 4 | 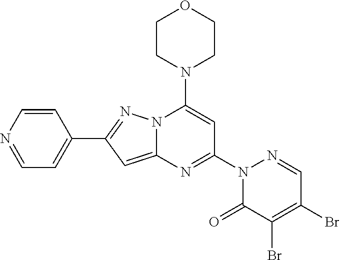 [SMILES: O=c1c(Br)c(Br)cnn1-c1cc(N2CCOCC2)n2nc(-c3ccncc3)cc2n1] |
| 5 | 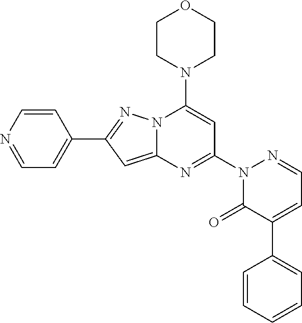 [SMILES: O=c1c(-c2ccccc2)ccnn1-c1cc(N2CCOCC2)n2nc(-c3ccncc3)cc2n1] |
| 6 | 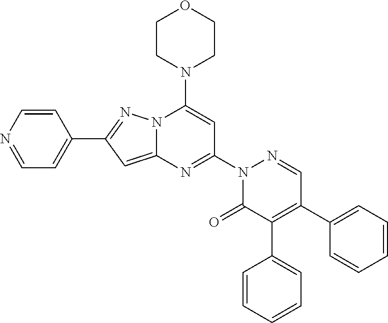 [SMILES: O=c1c(-c2ccccc2)c(-c2ccccc2)cnn1-c1cc(N2CCOCC2)n2nc(-c3ccncc3)cc2n1] |
| 7 | 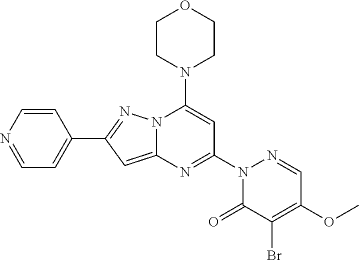 [SMILES: COc1cnn(-c2cc(N3CCOCC3)n3nc(-c4ccncc4)cc3n2)c(=O)c1Br] |
| 8 | 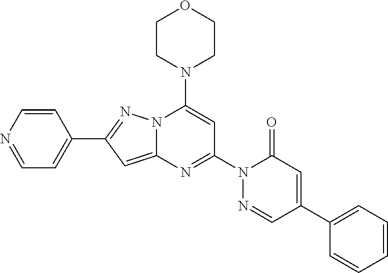 [SMILES: O=c1cc(-c2ccccc2)cnn1-c1cc(N2CCOCC2)n2nc(-c3ccncc3)cc2n1] |
| 9 | 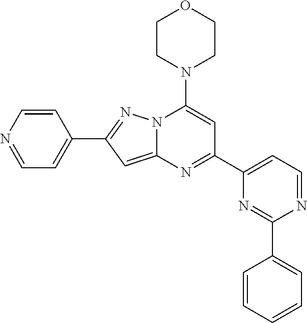 [SMILES: c1ccc(-c2nccc(-c3cc(N4CCOCC4)n4nc(-c5ccncc5)cc4n3)n2)cc1] |
| 10 | 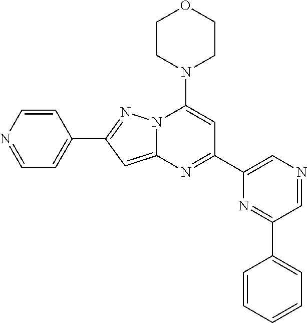 [SMILES: c1ccc(-c2cncc(-c3cc(N4CCOCC4)n4nc(-c5ccncc5)cc4n3)n2)cc1] |
| 11 | 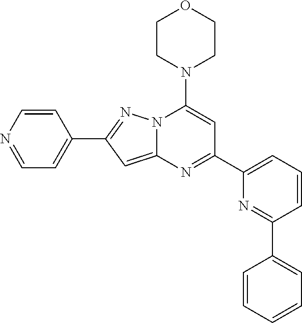 [SMILES: c1ccc(-c2cccc(-c3cc(N4CCOCC4)n4nc(-c5ccncc5)cc4n3)n2)cc1] |
| 12 | 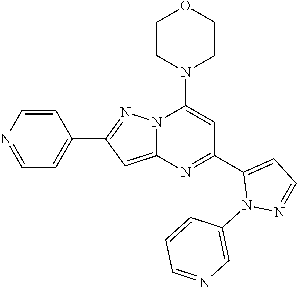 [SMILES: c1cncc(-n2nccc2-c2cc(N3CCOCC3)n3nc(-c4ccncc4)cc3n2)c1] |
| 13 | 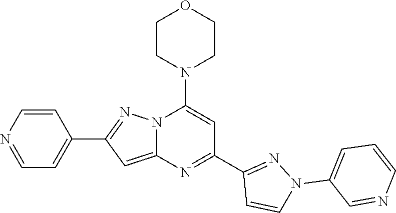 [SMILES: c1cncc(-n2ccc(-c3cc(N4CCOCC4)n4nc(-c5ccncc5)cc4n3)n2)c1] |
| 14 | 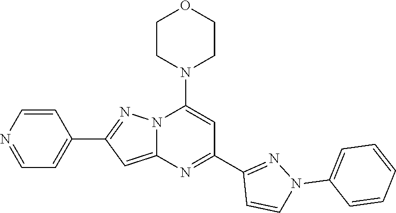 [SMILES: c1ccc(-n2ccc(-c3cc(N4CCOCC4)n4nc(-c5ccncc5)cc4n3)n2)cc1] |
| 15 | 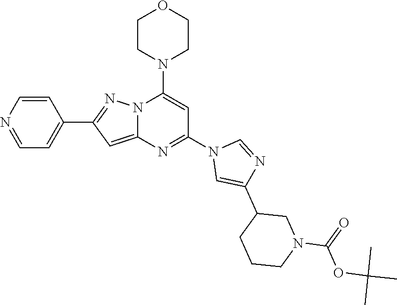 [SMILES: CC(C)(C)OC(=O)N1CCCC(c2cn(-c3cc(N4CCOCC4)n4nc(-c5ccncc5)cc4n3)cn2)C1] |
| 16 | 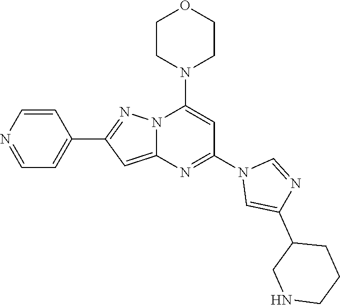 [SMILES: c1cc(-c2cc3nc(-n4cnc(C5CCCNC5)c4)cc(N4CCOCC4)n3n2)ccn1] |
| 17 | 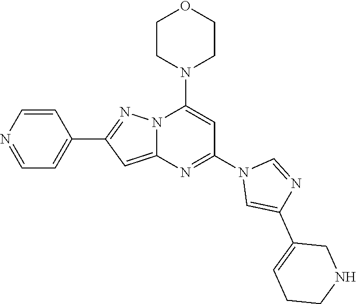 [SMILES: C1=C(c2cn(-c3cc(N4CCOCC4)n4nc(-c5ccncc5)cc4n3)cn2)CNCC1] |
| 18 | 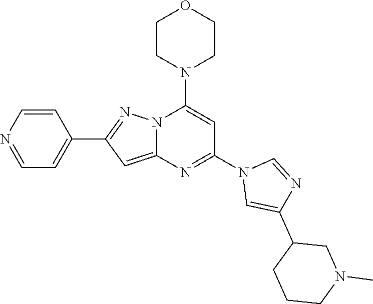 [SMILES: CN1CCCC(c2cn(-c3cc(N4CCOCC4)n4nc(-c5ccncc5)cc4n3)cn2)C1] |
| 19 | 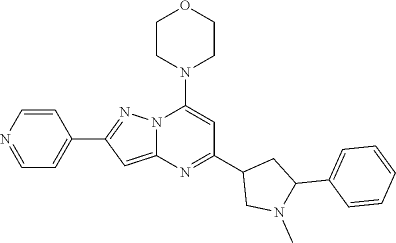 [SMILES: CN1CC(c2cc(N3CCOCC3)n3nc(-c4ccncc4)cc3n2)CC1c1ccccc1] |
| 20 | 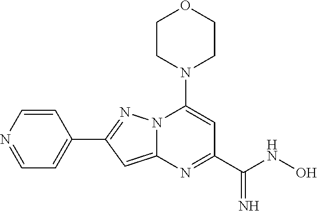 [SMILES: N=C(NO)c1cc(N2CCOCC2)n2nc(-c3ccncc3)cc2n1] |
| 21 | 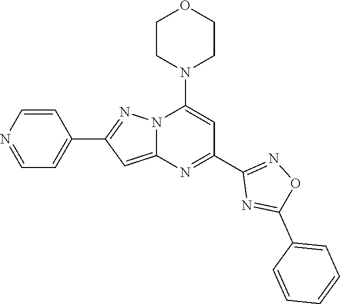 [SMILES: c1ccc(-c2nc(-c3cc(N4CCOCC4)n4nc(-c5ccncc5)cc4n3)no2)cc1] |
| 22 | 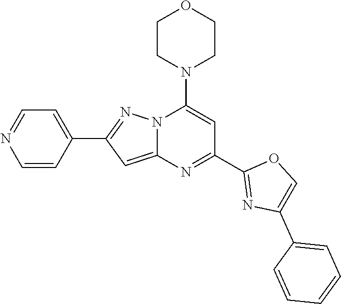 [SMILES: c1ccc(-c2coc(-c3cc(N4CCOCC4)n4nc(-c5ccncc5)cc4n3)n2)cc1] |
| 23 | 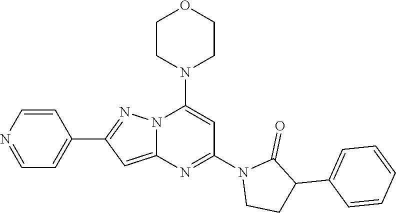 [SMILES: O=C1C(c2ccccc2)CCN1c1cc(N2CCOCC2)n2nc(-c3ccncc3)cc2n1] |
| 24 | 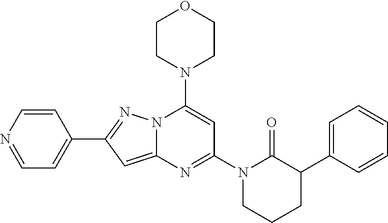 [SMILES: O=C1C(c2ccccc2)CCCN1c1cc(N2CCOCC2)n2nc(-c3ccncc3)cc2n1] |
| 25 | 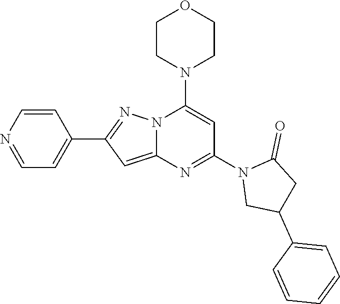 [SMILES: O=C1CC(c2ccccc2)CN1c1cc(N2CCOCC2)n2nc(-c3ccncc3)cc2n1] |
| 26 | 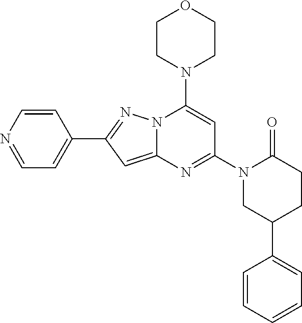 [SMILES: O=C1CCC(c2ccccc2)CN1c1cc(N2CCOCC2)n2nc(-c3ccncc3)cc2n1] |
| 27 | 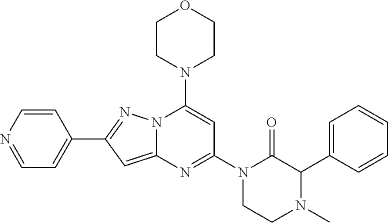 [SMILES: CN1CCN(c2cc(N3CCOCC3)n3nc(-c4ccncc4)cc3n2)C(=O)C1c1ccccc1] |
| 28 | 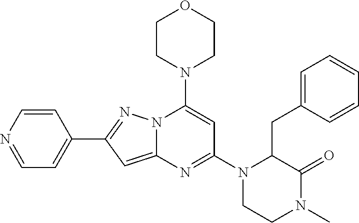 [SMILES: CN1CCN(c2cc(N3CCOCC3)n3nc(-c4ccncc4)cc3n2)C(Cc2ccccc2)C1=O] |
| 29 | 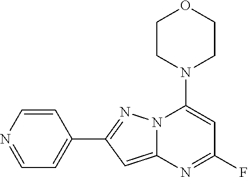 [SMILES: Fc1cc(N2CCOCC2)n2nc(-c3ccncc3)cc2n1] |
| 30 | 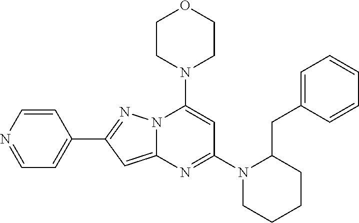 [SMILES: c1ccc(CC2CCCCN2c2cc(N3CCOCC3)n3nc(-c4ccncc4)cc3n2)cc1] |
| 31 | 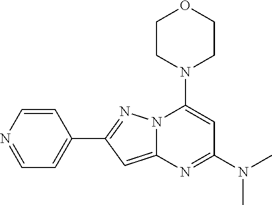 [SMILES: CN(C)c1cc(N2CCOCC2)n2nc(-c3ccncc3)cc2n1] |
| 32 | 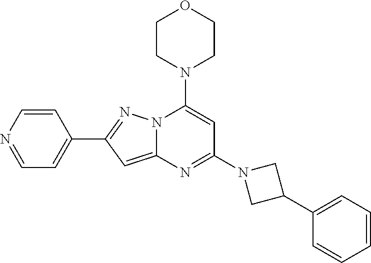 [SMILES: c1ccc(C2CN(c3cc(N4CCOCC4)n4nc(-c5ccncc5)cc4n3)C2)cc1] |
| 33 | 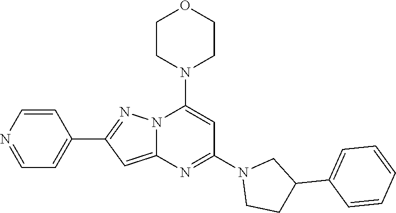 [SMILES: c1ccc(C2CCN(c3cc(N4CCOCC4)n4nc(-c5ccncc5)cc4n3)C2)cc1] |
| 34 | 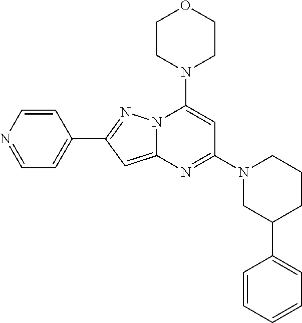 [SMILES: c1ccc(C2CCCN(c3cc(N4CCOCC4)n4nc(-c5ccncc5)cc4n3)C2)cc1] |
| 35 | 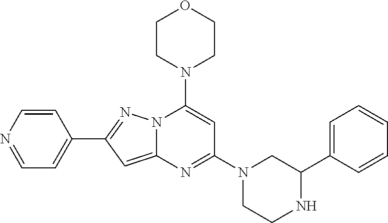 [SMILES: c1ccc(C2CN(c3cc(N4CCOCC4)n4nc(-c5ccncc5)cc4n3)CCN2)cc1] |
| 36 | 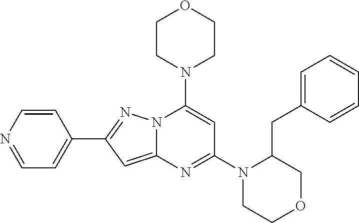 [SMILES: c1ccc(CC2COCCN2c2cc(N3CCOCC3)n3nc(-c4ccncc4)cc3n2)cc1] |
| 37 | 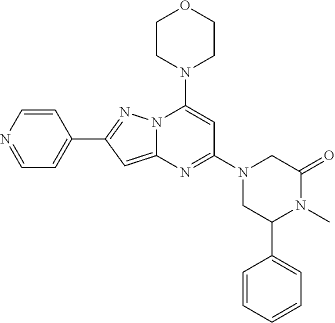 [SMILES: CN1C(=O)CN(c2cc(N3CCOCC3)n3nc(-c4ccncc4)cc3n2)CC1c1ccccc1] |
| 38 | 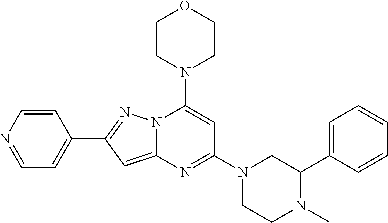 [SMILES: CN1CCN(c2cc(N3CCOCC3)n3nc(-c4ccncc4)cc3n2)CC1c1ccccc1] |
| 39 | 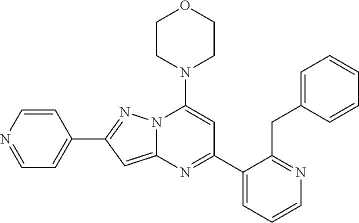 [SMILES: c1ccc(Cc2ncccc2-c2cc(N3CCOCC3)n3nc(-c4ccncc4)cc3n2)cc1] |
| 40 | 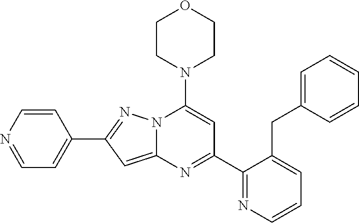 [SMILES: c1ccc(Cc2cccnc2-c2cc(N3CCOCC3)n3nc(-c4ccncc4)cc3n2)cc1] |
| 41 | 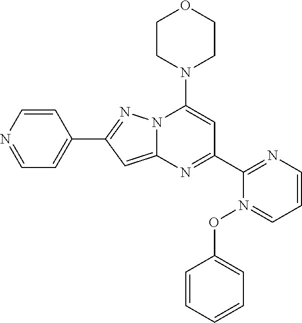 |
| 42 | 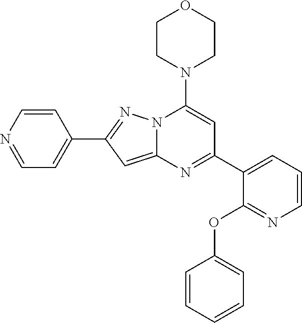 [SMILES: c1ccc(Oc2ncccc2-c2cc(N3CCOCC3)n3nc(-c4ccncc4)cc3n2)cc1] |
| 43 | 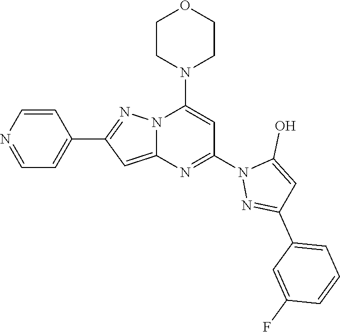 [SMILES: Oc1cc(-c2cccc(F)c2)nn1-c1cc(N2CCOCC2)n2nc(-c3ccncc3)cc2n1] |
| 44 | 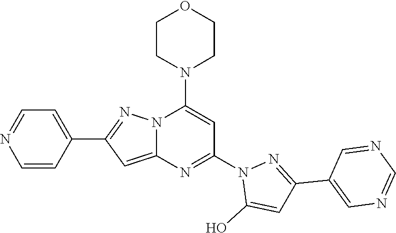 [SMILES: Oc1cc(-c2cncnc2)nn1-c1cc(N2CCOCC2)n2nc(-c3ccncc3)cc2n1] |
| 45 | 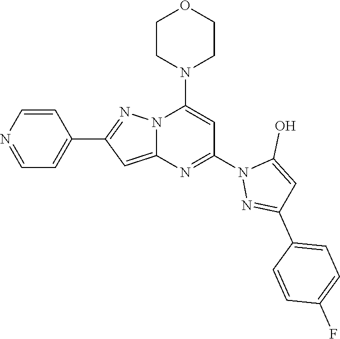 [SMILES: Oc1cc(-c2ccc(F)cc2)nn1-c1cc(N2CCOCC2)n2nc(-c3ccncc3)cc2n1] |
| 46 | 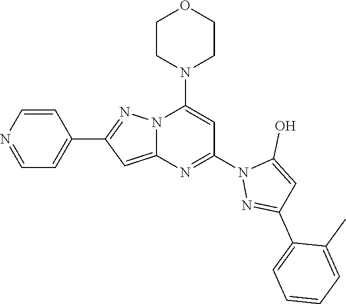 [SMILES: Cc1ccccc1-c1cc(O)n(-c2cc(N3CCOCC3)n3nc(-c4ccncc4)cc3n2)n1] |
| 47 | 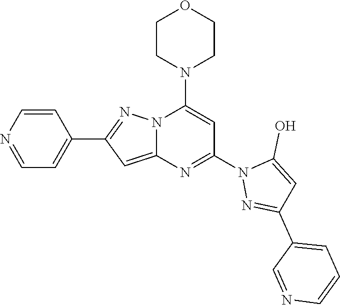 [SMILES: Oc1cc(-c2cccnc2)nn1-c1cc(N2CCOCC2)n2nc(-c3ccncc3)cc2n1] |
| 48 | 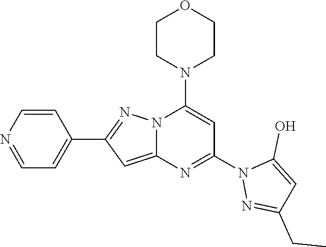 [SMILES: CCc1cc(O)n(-c2cc(N3CCOCC3)n3nc(-c4ccncc4)cc3n2)n1] |
| 49 | 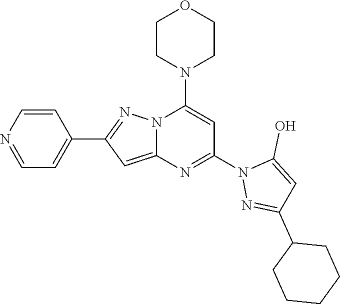 [SMILES: Oc1cc(C2CCCCC2)nn1-c1cc(N2CCOCC2)n2nc(-c3ccncc3)cc2n1] |
| 50 | 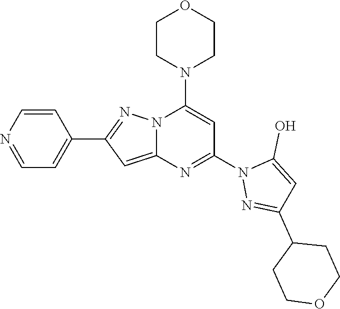 [SMILES: Oc1cc(C2CCOCC2)nn1-c1cc(N2CCOCC2)n2nc(-c3ccncc3)cc2n1] |
| 51 | 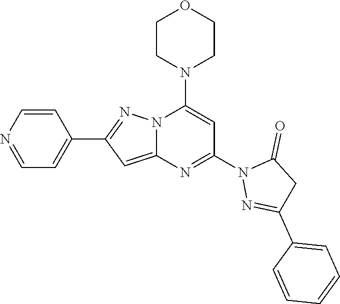 [SMILES: O=C1CC(c2ccccc2)=NN1c1cc(N2CCOCC2)n2nc(-c3ccncc3)cc2n1] |
| 52 | 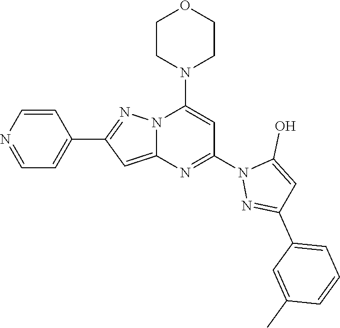 [SMILES: Cc1cccc(-c2cc(O)n(-c3cc(N4CCOCC4)n4nc(-c5ccncc5)cc4n3)n2)c1] |
| 53 | 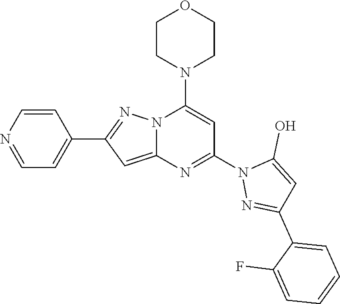 [SMILES: Oc1cc(-c2ccccc2F)nn1-c1cc(N2CCOCC2)n2nc(-c3ccncc3)cc2n1] |
| 54 | 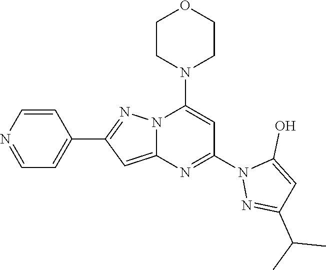 [SMILES: CC(C)c1cc(O)n(-c2cc(N3CCOCC3)n3nc(-c4ccncc4)cc3n2)n1] |
| 55 | 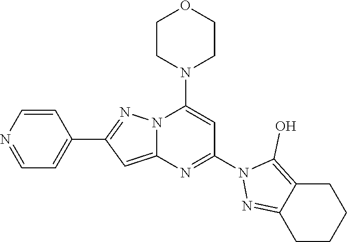 [SMILES: Oc1c2c(nn1-c1cc(N3CCOCC3)n3nc(-c4ccncc4)cc3n1)CCCC2] |
| 56 | 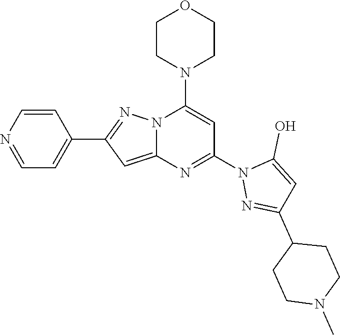 [SMILES: CN1CCC(c2cc(O)n(-c3cc(N4CCOCC4)n4nc(-c5ccncc5)cc4n3)n2)CC1] |
| 57 | 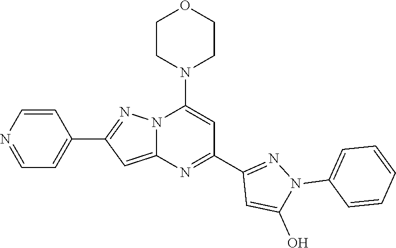 [SMILES: Oc1cc(-c2cc(N3CCOCC3)n3nc(-c4ccncc4)cc3n2)nn1-c1ccccc1] |
| 58 | 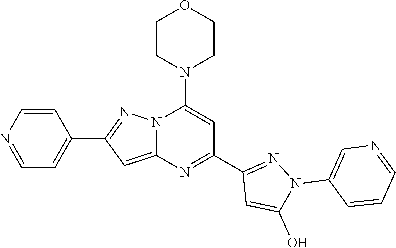 [SMILES: Oc1cc(-c2cc(N3CCOCC3)n3nc(-c4ccncc4)cc3n2)nn1-c1cccnc1] |
| 59 | 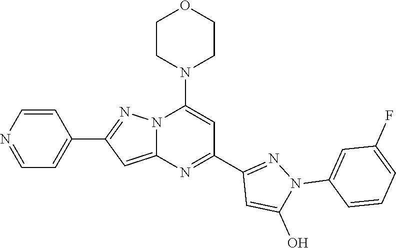 [SMILES: Oc1cc(-c2cc(N3CCOCC3)n3nc(-c4ccncc4)cc3n2)nn1-c1cccc(F)c1] |
| 60 | 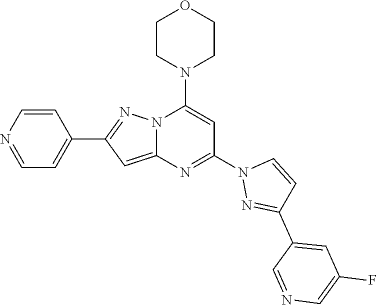 [SMILES: Fc1cncc(-c2ccn(-c3cc(N4CCOCC4)n4nc(-c5ccncc5)cc4n3)n2)c1] |
| 61 | 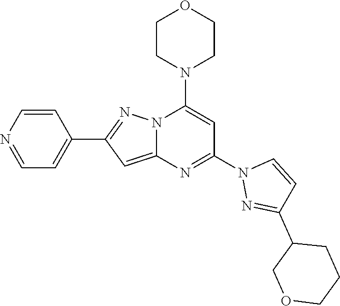 [SMILES: c1cc(-c2cc3nc(-n4ccc(C5CCCOC5)n4)cc(N4CCOCC4)n3n2)ccn1] |
| 62 | 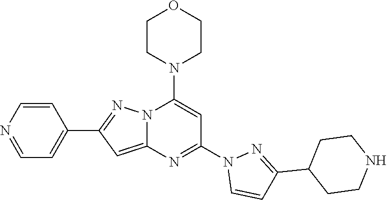 [SMILES: c1cc(-c2cc3nc(-n4ccc(C5CCNCC5)n4)cc(N4CCOCC4)n3n2)ccn1] |
| 63 | 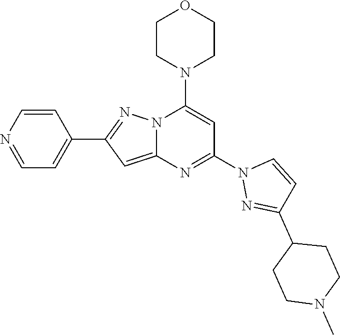 [SMILES: CN1CCC(c2ccn(-c3cc(N4CCOCC4)n4nc(-c5ccncc5)cc4n3)n2)CC1] |
| 64 | 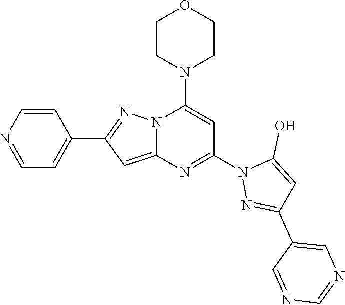 [SMILES: Oc1cc(-c2cncnc2)nn1-c1cc(N2CCOCC2)n2nc(-c3ccncc3)cc2n1] |
| 65 | 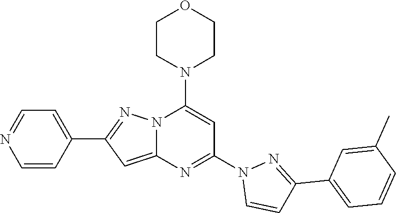 [SMILES: Cc1cccc(-c2ccn(-c3cc(N4CCOCC4)n4nc(-c5ccncc5)cc4n3)n2)c1] |
| 66 | 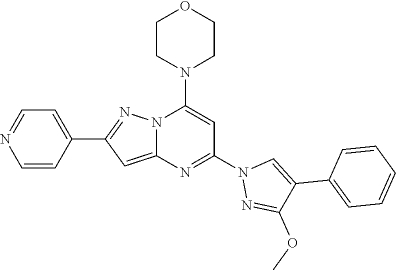 [SMILES: COc1nn(-c2cc(N3CCOCC3)n3nc(-c4ccncc4)cc3n2)cc1-c1ccccc1] |
| 67 | 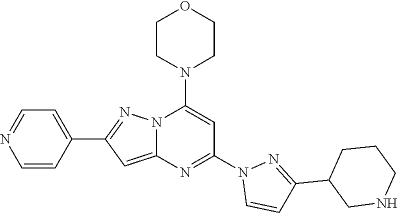 [SMILES: c1cc(-c2cc3nc(-n4ccc(C5CCCNC5)n4)cc(N4CCOCC4)n3n2)ccn1] |
| 68 | 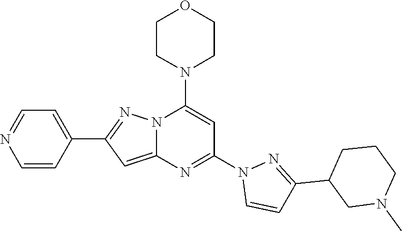 [SMILES: CN1CCCC(c2ccn(-c3cc(N4CCOCC4)n4nc(-c5ccncc5)cc4n3)n2)C1] |
| 69 | 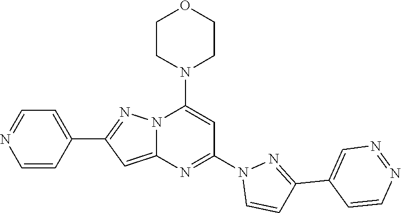 [SMILES: c1cc(-c2cc3nc(-n4ccc(-c5ccnnc5)n4)cc(N4CCOCC4)n3n2)ccn1] |
| 70 | 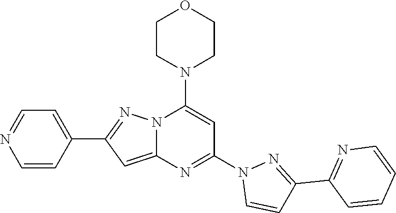 [SMILES: c1ccc(-c2ccn(-c3cc(N4CCOCC4)n4nc(-c5ccncc5)cc4n3)n2)nc1] |
| 71 | 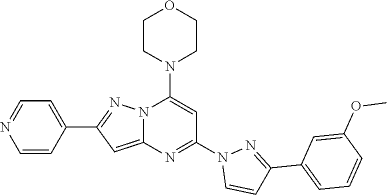 [SMILES: COc1cccc(-c2ccn(-c3cc(N4CCOCC4)n4nc(-c5ccncc5)cc4n3)n2)c1] |
| 72 | 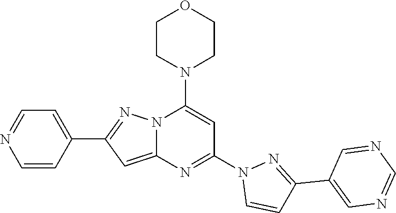 [SMILES: c1cc(-c2cc3nc(-n4ccc(-c5cncnc5)n4)cc(N4CCOCC4)n3n2)ccn1] |
| 73 | 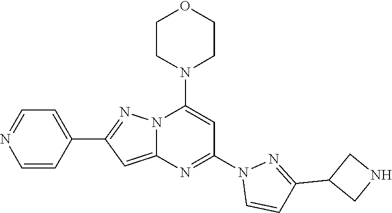 [SMILES: c1cc(-c2cc3nc(-n4ccc(C5CNC5)n4)cc(N4CCOCC4)n3n2)ccn1] |
| 74 | 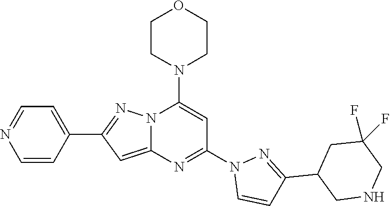 [SMILES: FC1(F)CNCC(c2ccn(-c3cc(N4CCOCC4)n4nc(-c5ccncc5)cc4n3)n2)C1] |
| 75 | 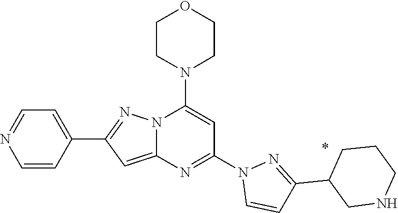 [SMILES: c1cc(-c2cc3nc(-n4ccc(C5CCCNC5)n4)cc(N4CCOCC4)n3n2)ccn1] |
| 76 | 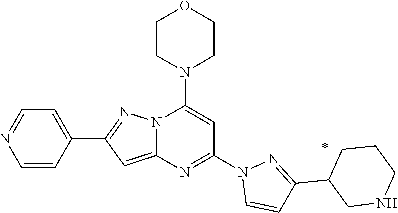 [SMILES: c1cc(-c2cc3nc(-n4ccc(C5CCCNC5)n4)cc(N4CCOCC4)n3n2)ccn1] |
| 77 | 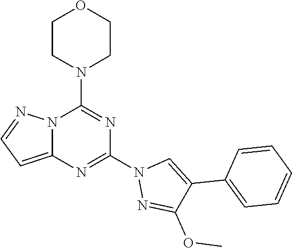 [SMILES: COc1nn(-c2nc(N3CCOCC3)n3nccc3n2)cc1-c1ccccc1] |
| 78 | 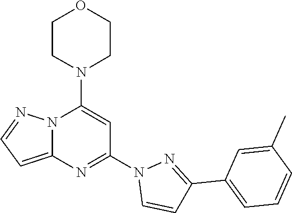 [SMILES: Cc1cccc(-c2ccn(-c3cc(N4CCOCC4)n4nccc4n3)n2)c1] |
| 80 | 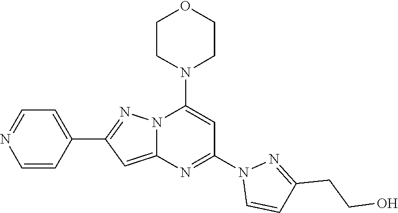 [SMILES: OCCc1ccn(-c2cc(N3CCOCC3)n3nc(-c4ccncc4)cc3n2)n1] |
| 81 | 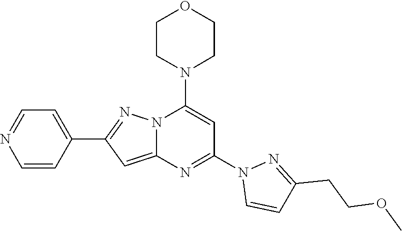 [SMILES: COCCc1ccn(-c2cc(N3CCOCC3)n3nc(-c4ccncc4)cc3n2)n1] |
| 82 | 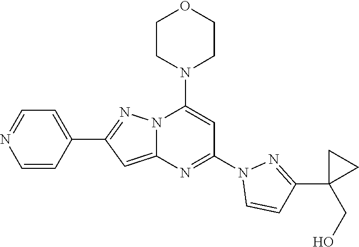 [SMILES: OCC1(c2ccn(-c3cc(N4CCOCC4)n4nc(-c5ccncc5)cc4n3)n2)CC1] |
| 83 | 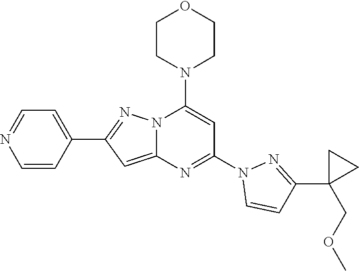 [SMILES: COCC1(c2ccn(-c3cc(N4CCOCC4)n4nc(-c5ccncc5)cc4n3)n2)CC1] |
| 84 | 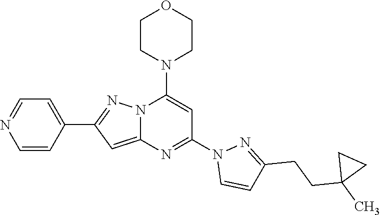 [SMILES: CC1(CCc2ccn(-c3cc(N4CCOCC4)n4nc(-c5ccncc5)cc4n3)n2)CC1] |
| 85 | 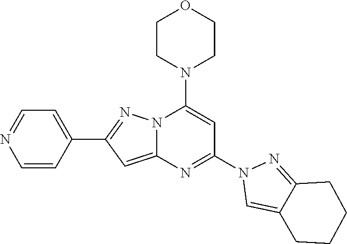 [SMILES: c1cc(-c2cc3nc(-n4cc5c(n4)CCCC5)cc(N4CCOCC4)n3n2)ccn1] |
| 86 | 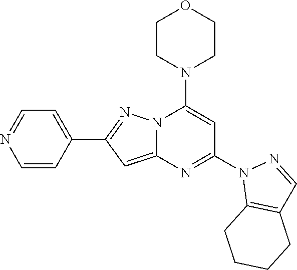 [SMILES: c1cc(-c2cc3nc(-n4ncc5c4CCCC5)cc(N4CCOCC4)n3n2)ccn1] |
| 87 | 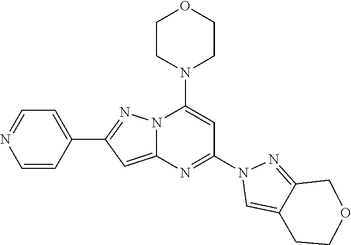 [SMILES: c1cc(-c2cc3nc(-n4cc5c(n4)COCC5)cc(N4CCOCC4)n3n2)ccn1] |
| 88 | 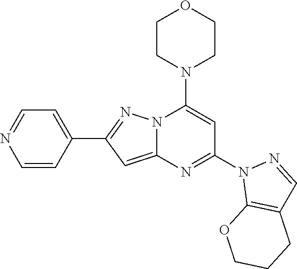 [SMILES: c1cc(-c2cc3nc(-n4ncc5c4OCCC5)cc(N4CCOCC4)n3n2)ccn1] |
| 89 | 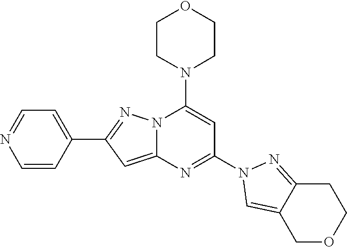 [SMILES: c1cc(-c2cc3nc(-n4cc5c(n4)CCOC5)cc(N4CCOCC4)n3n2)ccn1] |
| 90 | 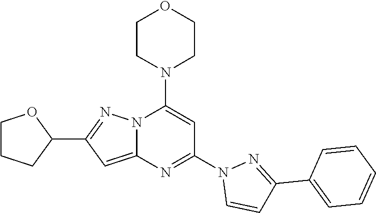 [SMILES: c1ccc(-c2ccn(-c3cc(N4CCOCC4)n4nc(C5CCCO5)cc4n3)n2)cc1] |
| 91 | 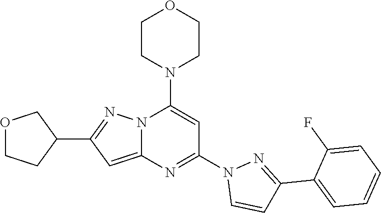 [SMILES: Fc1ccccc1-c1ccn(-c2cc(N3CCOCC3)n3nc(C4CCOC4)cc3n2)n1] |
| 92 | 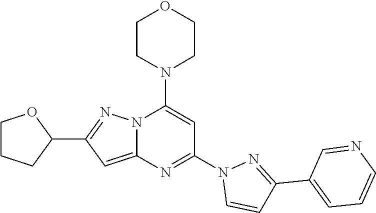 [SMILES: c1cncc(-c2ccn(-c3cc(N4CCOCC4)n4nc(C5CCCO5)cc4n3)n2)c1] |
| 93 | 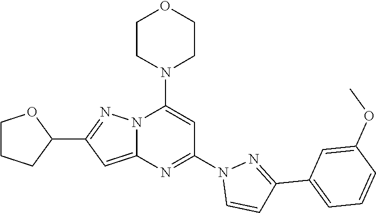 [SMILES: COc1cccc(-c2ccn(-c3cc(N4CCOCC4)n4nc(C5CCCO5)cc4n3)n2)c1] |
| 94 | 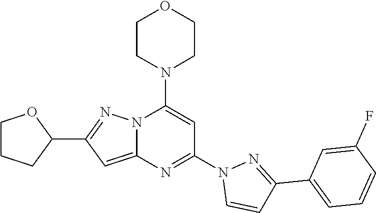 [SMILES: Fc1cccc(-c2ccn(-c3cc(N4CCOCC4)n4nc(C5CCCO5)cc4n3)n2)c1] |
| 95 | 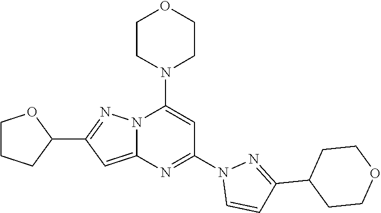 [SMILES: c1cn(-c2cc(N3CCOCC3)n3nc(C4CCCO4)cc3n2)nc1C1CCOCC1] |
| 96 | 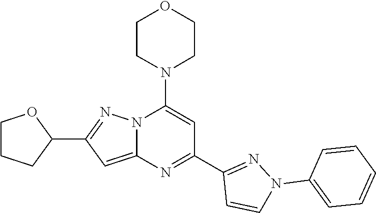 [SMILES: c1ccc(-n2ccc(-c3cc(N4CCOCC4)n4nc(C5CCCO5)cc4n3)n2)cc1] |
| 97 | 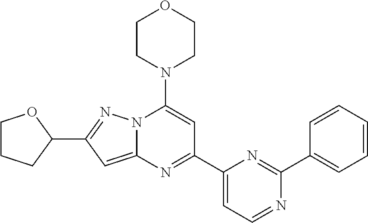 [SMILES: c1ccc(-c2nccc(-c3cc(N4CCOCC4)n4nc(C5CCCO5)cc4n3)n2)cc1] |
| 98 | 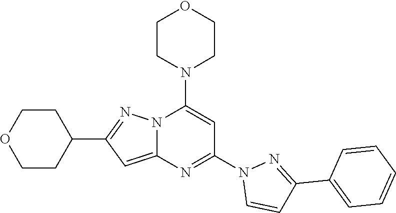 [SMILES: c1ccc(-c2ccn(-c3cc(N4CCOCC4)n4nc(C5CCOCC5)cc4n3)n2)cc1] |
| 99 | 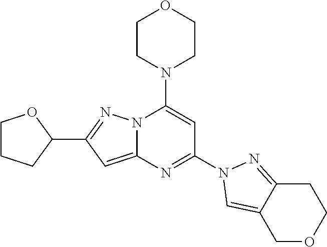 [SMILES: c1c(-n2cc3c(n2)CCOC3)nc2cc(C3CCCO3)nn2c1N1CCOCC1] |
| 100 | 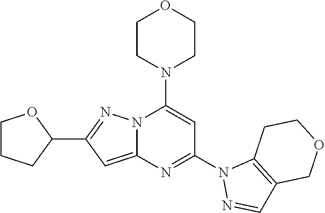 [SMILES: c1nn(-c2cc(N3CCOCC3)n3nc(C4CCCO4)cc3n2)c2c1COCC2] |
| 101 | 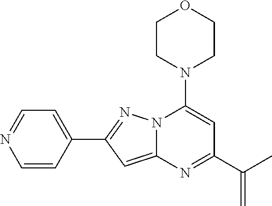 [SMILES: C=C(C)c1cc(N2CCOCC2)n2nc(-c3ccncc3)cc2n1] |
| 102 | 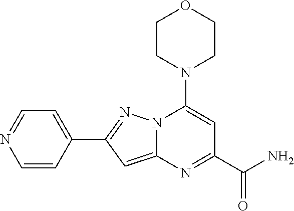 [SMILES: NC(=O)c1cc(N2CCOCC2)n2nc(-c3ccncc3)cc2n1] |
| 103 | 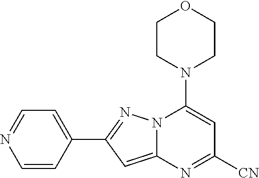 [SMILES: N#Cc1cc(N2CCOCC2)n2nc(-c3ccncc3)cc2n1] |
| 104 | 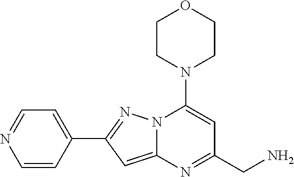 [SMILES: NCc1cc(N2CCOCC2)n2nc(-c3ccncc3)cc2n1] |
| 105 | 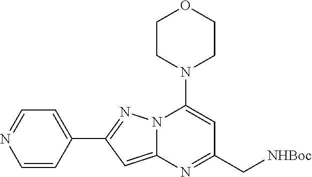 [SMILES: CC(C)(C)OC(=O)NCc1cc(N2CCOCC2)n2nc(-c3ccncc3)cc2n1] |
| 106 | 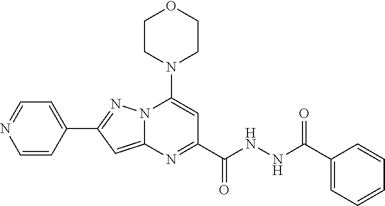 [SMILES: O=C(NNC(=O)c1cc(N2CCOCC2)n2nc(-c3ccncc3)cc2n1)c1ccccc1] |
| 107 | 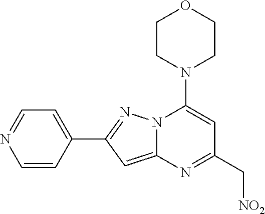 [SMILES: O=[N+]([O-])Cc1cc(N2CCOCC2)n2nc(-c3ccncc3)cc2n1] |
| 108 | 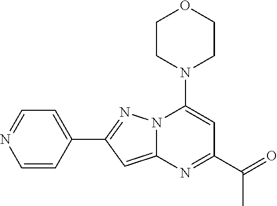 [SMILES: CC(=O)c1cc(N2CCOCC2)n2nc(-c3ccncc3)cc2n1] |
| 109 | 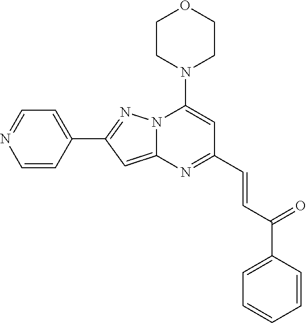 [SMILES: O=C(/C=C/c1cc(N2CCOCC2)n2nc(-c3ccncc3)cc2n1)c1ccccc1] |
| 110 | 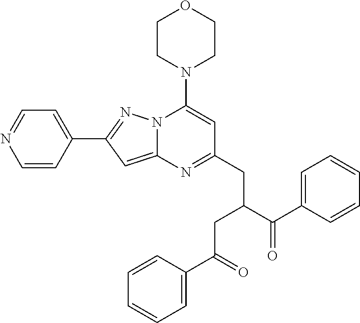 [SMILES: O=C(CC(Cc1cc(N2CCOCC2)n2nc(-c3ccncc3)cc2n1)C(=O)c1ccccc1)c1ccccc1] |
| 111 | 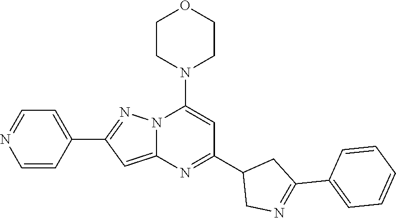 [SMILES: c1ccc(C2=NCC(c3cc(N4CCOCC4)n4nc(-c5ccncc5)cc4n3)C2)cc1] |
| 112 | 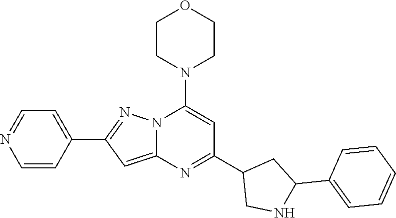 [SMILES: c1ccc(C2CC(c3cc(N4CCOCC4)n4nc(-c5ccncc5)cc4n3)CN2)cc1] |
| 113 | 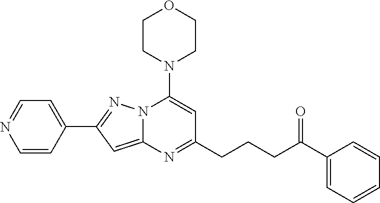 [SMILES: O=C(CCCc1cc(N2CCOCC2)n2nc(-c3ccncc3)cc2n1)c1ccccc1] |
| 114 | 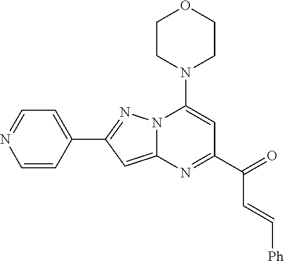 [SMILES: O=C(/C=C/c1ccccc1)c1cc(N2CCOCC2)n2nc(-c3ccncc3)cc2n1] |
| 115 | 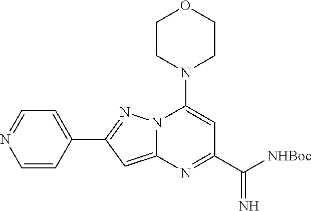 [SMILES: CC(C)(C)OC(=O)NC(=N)c1cc(N2CCOCC2)n2nc(-c3ccncc3)cc2n1] |
| 116 | 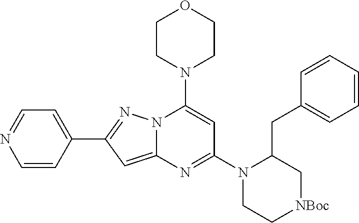 [SMILES: CC(C)(C)OC(=O)N1CCN(c2cc(N3CCOCC3)n3nc(-c4ccncc4)cc3n2)C(Cc2ccccc2)C1] |
| 117 | 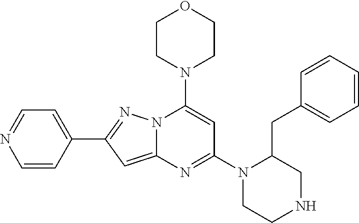 [SMILES: c1ccc(CC2CNCCN2c2cc(N3CCOCC3)n3nc(-c4ccncc4)cc3n2)cc1] |
| 118 | 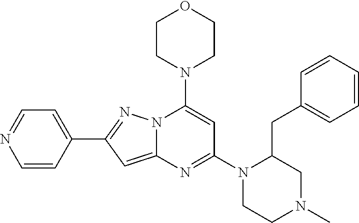 [SMILES: CN1CCN(c2cc(N3CCOCC3)n3nc(-c4ccncc4)cc3n2)C(Cc2ccccc2)C1] |
| 119 | 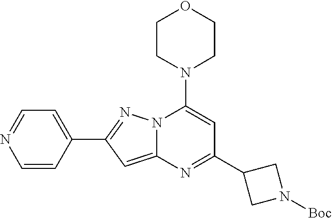 [SMILES: CC(C)(C)OC(=O)N1CC(c2cc(N3CCOCC3)n3nc(-c4ccncc4)cc3n2)C1] |
| 120 | 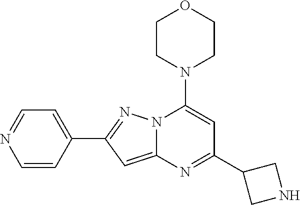 [SMILES: c1cc(-c2cc3nc(C4CNC4)cc(N4CCOCC4)n3n2)ccn1] |
| 121 | 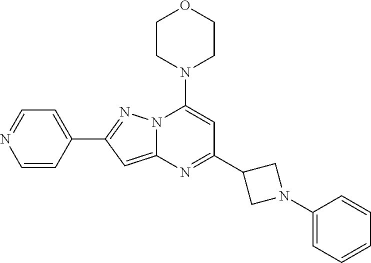 [SMILES: c1ccc(N2CC(c3cc(N4CCOCC4)n4nc(-c5ccncc5)cc4n3)C2)cc1] |
| 122 | 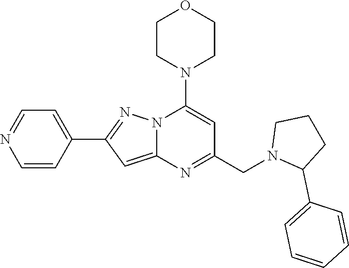 [SMILES: c1ccc(C2CCCN2Cc2cc(N3CCOCC3)n3nc(-c4ccncc4)cc3n2)cc1] |
| 123 | 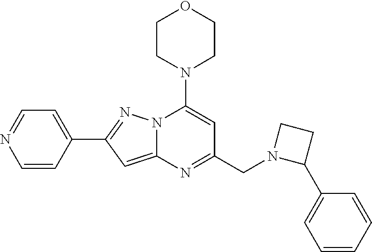 [SMILES: c1ccc(C2CCN2Cc2cc(N3CCOCC3)n3nc(-c4ccncc4)cc3n2)cc1] |
| 124 | 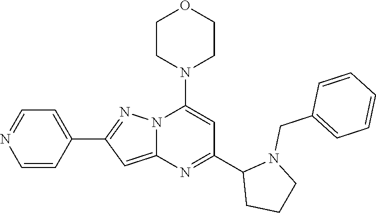 [SMILES: c1ccc(CN2CCCC2c2cc(N3CCOCC3)n3nc(-c4ccncc4)cc3n2)cc1] |
| 125 | 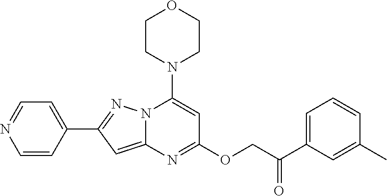 [SMILES: Cc1cccc(C(=O)COc2cc(N3CCOCC3)n3nc(-c4ccncc4)cc3n2)c1] |
| 126 | 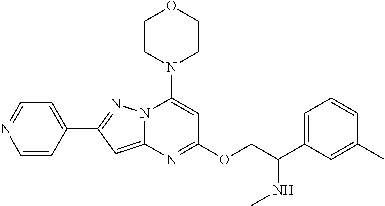 [SMILES: CNC(COc1cc(N2CCOCC2)n2nc(-c3ccncc3)cc2n1)c1cccc(C)c1] |
| 127 | 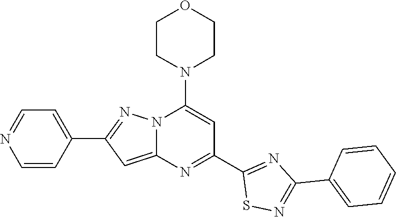 [SMILES: c1ccc(-c2nsc(-c3cc(N4CCOCC4)n4nc(-c5ccncc5)cc4n3)n2)cc1] |
| 128 | 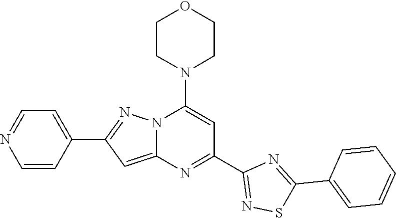 [SMILES: c1ccc(-c2nc(-c3cc(N4CCOCC4)n4nc(-c5ccncc5)cc4n3)ns2)cc1] |
| 129 | 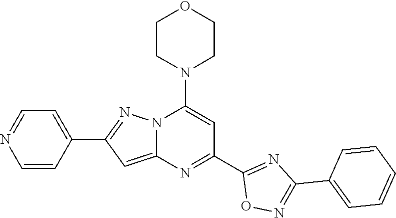 [SMILES: c1ccc(-c2noc(-c3cc(N4CCOCC4)n4nc(-c5ccncc5)cc4n3)n2)cc1] |
| 130 | 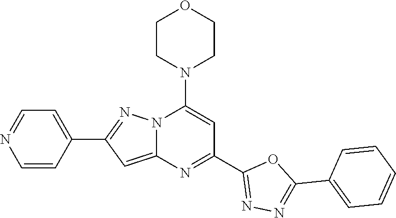 [SMILES: c1ccc(-c2nnc(-c3cc(N4CCOCC4)n4nc(-c5ccncc5)cc4n3)o2)cc1] |
| 131 | 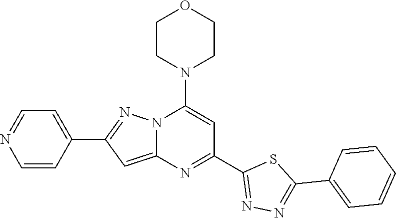 [SMILES: c1ccc(-c2nnc(-c3cc(N4CCOCC4)n4nc(-c5ccncc5)cc4n3)s2)cc1] |
| 132 | 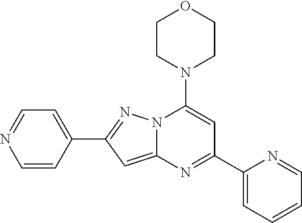 [SMILES: c1ccc(-c2cc(N3CCOCC3)n3nc(-c4ccncc4)cc3n2)nc1] |
| 133 | 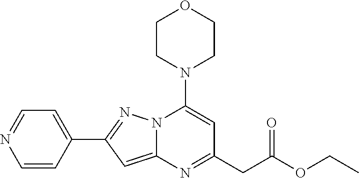 [SMILES: CCOC(=O)Cc1cc(N2CCOCC2)n2nc(-c3ccncc3)cc2n1] |
| 134 | 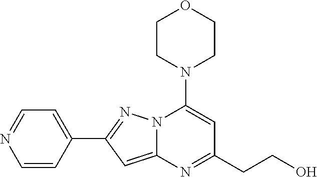 [SMILES: OCCc1cc(N2CCOCC2)n2nc(-c3ccncc3)cc2n1] |
| 135 | 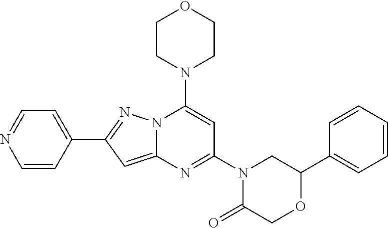 [SMILES: O=C1COC(c2ccccc2)CN1c1cc(N2CCOCC2)n2nc(-c3ccncc3)cc2n1] |
| 136 | 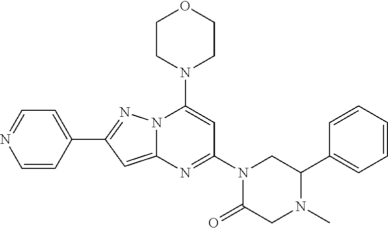 [SMILES: CN1CC(=O)N(c2cc(N3CCOCC3)n3nc(-c4ccncc4)cc3n2)CC1c1ccccc1] |
| 137 | 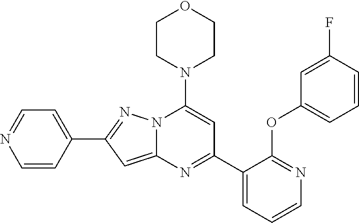 [SMILES: Fc1cccc(Oc2ncccc2-c2cc(N3CCOCC3)n3nc(-c4ccncc4)cc3n2)c1] |
| 138 | 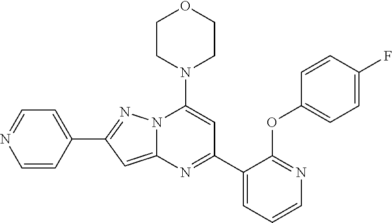 [SMILES: Fc1ccc(Oc2ncccc2-c2cc(N3CCOCC3)n3nc(-c4ccncc4)cc3n2)cc1] |
| 139 | 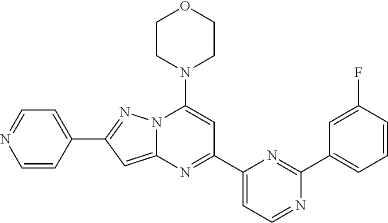 [SMILES: Fc1cccc(-c2nccc(-c3cc(N4CCOCC4)n4nc(-c5ccncc5)cc4n3)n2)c1] |
| 140 | 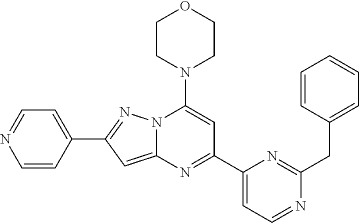 [SMILES: c1ccc(Cc2nccc(-c3cc(N4CCOCC4)n4nc(-c5ccncc5)cc4n3)n2)cc1] |
| 141 | 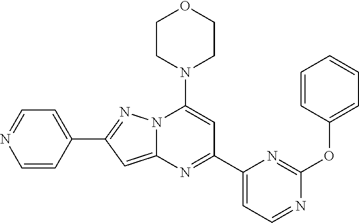 [SMILES: c1ccc(Oc2nccc(-c3cc(N4CCOCC4)n4nc(-c5ccncc5)cc4n3)n2)cc1] |
| 142 | 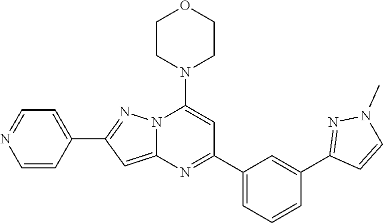 [SMILES: Cn1ccc(-c2cccc(-c3cc(N4CCOCC4)n4nc(-c5ccncc5)cc4n3)c2)n1] |
| 143 | 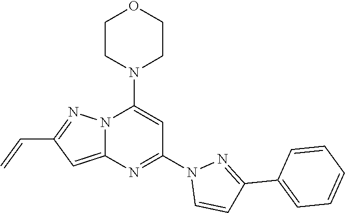 [SMILES: C=Cc1cc2nc(-n3ccc(-c4ccccc4)n3)cc(N3CCOCC3)n2n1] |
| 144 | 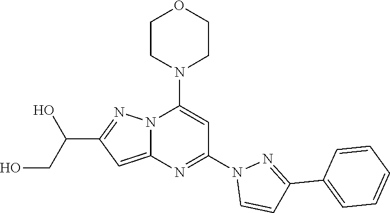 [SMILES: OCC(O)c1cc2nc(-n3ccc(-c4ccccc4)n3)cc(N3CCOCC3)n2n1] |
| 145 | 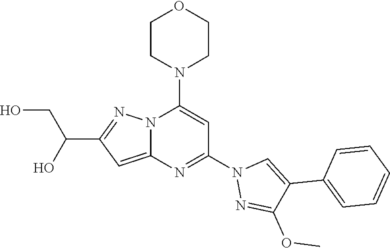 [SMILES: COc1nn(-c2cc(N3CCOCC3)n3nc(C(O)CO)cc3n2)cc1-c1ccccc1] |
| 146 | 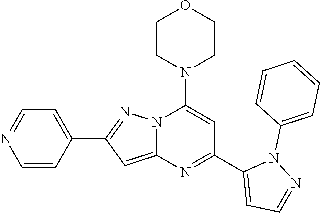 [SMILES: c1ccc(-n2nccc2-c2cc(N3CCOCC3)n3nc(-c4ccncc4)cc3n2)cc1] |
| 147 | 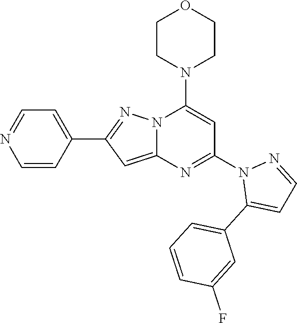 [SMILES: Fc1cccc(-c2ccnn2-c2cc(N3CCOCC3)n3nc(-c4ccncc4)cc3n2)c1] |
| 149 | 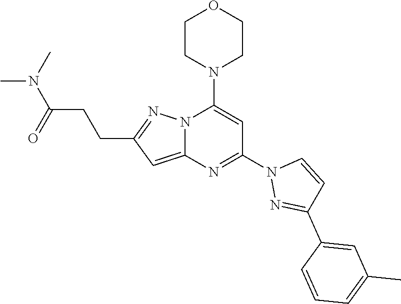 |
| 150 | 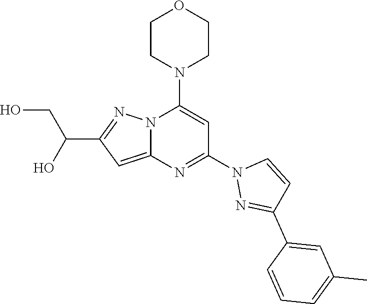 [SMILES: Cc1cccc(-c2ccn(-c3cc(N4CCOCC4)n4nc(C(O)CO)cc4n3)n2)c1] |
| 151 | 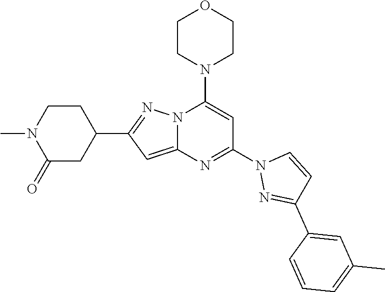 |
| 152 | 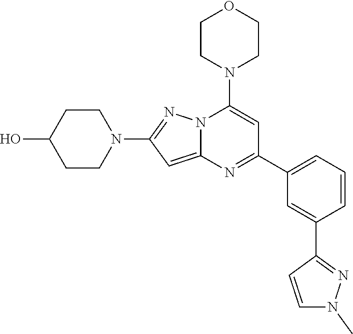 [SMILES: Cn1ccc(-c2cccc(-c3cc(N4CCOCC4)n4nc(N5CCC(O)CC5)cc4n3)c2)n1] |

### Methods of Treatment

### Suppression of PIKfyve Activity and TDP-43 Aggregation to Treat Neurological Disorders

Using the compositions and methods described herein, a patient suffering from a neurological disorder may be administered a PIKfyve inhibitor, such as a small molecule described herein, so as to treat the disorder and/or to suppress one or more symptoms associated with the disorder. Exemplary neurological disorders that may be treated using the compositions and methods described herein are, without limitation, amyotrophic lateral sclerosis, frontotemporal degeneration, Alzheimer's disease, Parkinson's disease, dementia with Lewy Bodies, corticobasal degeneration, progressive supranuclear palsy, dementia parkinsonism ALS complex of Guam, Huntington's disease, IBMPFD, sporadic inclusion body myositis, myofibrillar myopathy, dementia pugilistica, chronic traumatic encephalopathy, Alexander disease, and hereditary inclusion body myopathy, as well as neuromuscular diseases such as congenital myasthenic syndrome, congenital myopathy, cramp fasciculation syndrome, Duchenne muscular dystrophy, glycogen storage disease type II, hereditary spastic paraplegia, inclusion body myositis, Isaac's Syndrome, Kearns-Sayre syndrome, Lambert-Eaton myasthenic syndrome, mitochondrial myopathy, muscular dystrophy, myasthenia gravis, myotonic dystrophy, peripheral neuropathy, spinal and bulbar muscular atrophy, spinal muscular atrophy, Stiff person syndrome, Troyer syndrome, and Guillain-Barré syndrome.

The present disclosure is based, in part, on the discovery that PIKfyve inhibitors, such as the agents described herein, are capable of attenuating TDP-43 toxicity. TDP-43-promoted toxicity has been associated with various neurological diseases. The discovery that PIKfyve inhibitors modulate TDP-43 aggregation provides an important therapeutic benefit. Using a PIKfyve inhibitor, such as a PIKfyve inhibitor described herein, a patient suffering from a neurological disorder or at risk of developing such a condition may be treated in a manner that remedies an underlying molecular etiology of the disease. Without being limited by mechanism, the compositions and methods described herein can be used to treat or prevent such neurological conditions, for example, by suppressing the TDP-43 aggregation that promotes pathology.

Additionally, the compositions and methods described herein provide the beneficial feature of enabling the identification and treatment of patients that are likely to respond to PIKfyve inhibitor therapy. For example, in some embodiments, a patient (e.g., a human patient suffering from or at risk of developing a neurological disease described herein, such as amyotrophic lateral sclerosis) is administered a PIKfyve inhibitor if the patient is identified as likely to respond to this form of treatment. Patients may be identified as such on the basis, for example, of susceptibility to TDP-43 aggregation. In some embodiments, the patient is identified is likely to respond to PIKfyve inhibitor treatment based on the isoform of TDP-43 expressed by the patient. For example, patients expressing TDP-43 isoforms having a mutation selected from Q331K, M337V, Q343R, N345K, R³⁶¹S, and N390D, among others, are more likely to develop TDP-43-promoted aggregation and toxicity relative to patients that do not express such isoforms of TDP-43. Using the compositions and methods described herein, a patient may be identified as likely to respond to PIKfyve inhibitor therapy on the basis of expressing such an isoform of TDP-43, and may subsequently be administered a PIKfyve inhibitor so as to treat or prevent one or more neurological disorders, such as one or more of the neurological disorders described herein.

### Assessing Patient Response

A variety of methods known in the art and described herein can be used to determine whether a patient having a neurological disorder (e.g., a patient at risk of developing TDP-43 aggregation, such as a patient expressing a mutant form of TDP-43 having a mutation associated with elevated TDP-43 aggregation and toxicity, for example, a mutation selected from Q331K, M337V, Q343R, N345K, R³⁶¹S, and N390D) is responding favorably to PIKfyve inhibition. For example, successful treatment of a patient having a neurological disease, such as amyotrophic lateral sclerosis, with a PIKfyve inhibitor described herein may be signaled by:
(i) an improvement in condition as assessed using the amyotrophic lateral sclerosis functional rating scale (ALSFRS) or the revised ALSFRS (ALSFRS-R), such as an improvement in the patient's ALSFRS or ALSFRS-R score within one or more days, weeks, or months following administration of the PIKfyve inhibitor (e.g., an improvement in the patient's ALSFRS or ALSFRS-R score within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the PIKfyve inhibitor to the patient, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the PIKfyve inhibitor to the patient);
(ii) an increase in slow vital capacity, such as an increase in the patient's slow vital capacity within one or more days, weeks, or months following administration of the PIKfyve inhibitor (e.g., an increase in the patient's slow vital capacity within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the PIKfyve inhibitor to the patient, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the PIKfyve inhibitor to the patient);
(iii) a reduction in decremental responses exhibited by the patient upon repetitive nerve stimulation, such as a reduction that is observed within one or more days, weeks, or months following administration of the PIKfyve inhibitor (e.g., a reduction that is observed within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the PIKfyve inhibitor to the patient, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the PIKfyve inhibitor to the patient);
(iv) an improvement in muscle strength, as assessed, for example, by way of the Medical Research Council muscle testing scale (as described, e.g., in Jagtap et al., Ann. Indian. Acad. Neurol. 17:336-339 (2014), the disclosure of which is incorporated herein by reference as it pertains to measuring patient response to neurological disease treatment), such as an improvement that is observed within one or more days, weeks, or months following administration of the PIKfyve inhibitor (e.g., an improvement that is observed within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the PIKfyve inhibitor to the patient, such as within 1 day, 2 days, 3 days, 4 days, 5 days, 6 days, 7 days, 2 weeks, 3 weeks, 4 weeks, weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the PIKfyve inhibitor to the patient);
(v) an improvement in quality of life, as assessed, for example, using the amyotrophic lateral sclerosis-specific quality of life (ALS-specific QOL) questionnaire, such as an improvement in the patient's quality of life that is observed within one or more days, weeks, or months following administration of the PIKfyve inhibitor (e.g., an improvement in the subject's quality of life that is observed within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the PIKfyve inhibitor to the patient, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the PIKfyve inhibitor to the patient);
(vi) a decrease in the frequency and/or severity of muscle cramps, such as a decrease in cramp frequency and/or severity within one or more days, weeks, or months following administration of the PIKfyve inhibitor (e.g., a decrease in cramp frequency and/or severity within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the PIKfyve inhibitor to the patient, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the PIKfyve inhibitor to the patient); and/or
(vii) a decrease in TDP-43 aggregation, such as a decrease in TDP-43 aggregation within one or more days, weeks, or months following administration of the PIKfyve inhibitor (e.g., a decrease in TDP-43 aggregation within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the PIKfyve inhibitor to the patient, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, weeks, 46 weeks, 47 weeks, 48 weeks, or more, following the initial administration of the PIKfyve inhibitor to the patient.

### Combination Formulations and Uses Thereof

The compounds of the invention can be combined with one or more therapeutic agents. In particular, the therapeutic agent can be one that treats or prophylactically treats any neurological disorder described herein.

### Combination Therapies

A compound of the invention can be used alone or in combination with other agents that treat neurological disorders or symptoms associated therewith, or in combination with other types of treatment to treat, prevent, and/or reduce the risk of any neurological disorders. In combination treatments, the dosages of one or more of the therapeutic compounds may be reduced from standard dosages when administered alone. For example, doses may be determined empirically from drug combinations and permutations or may be deduced by isobolographic analysis (e.g., Black et al., Neurology 65:S3-S6, 2005). In this case, dosages of the compounds when combined should provide a therapeutic effect.

### Pharmaceutical Compositions

The compounds of the invention are preferably formulated into pharmaceutical compositions for administration to human subjects in a biologically compatible form suitable for administration in vivo. Accordingly, in another aspect, the present invention provides a pharmaceutical composition comprising a compound of the invention in admixture with a suitable diluent, carrier, or excipient.

The compounds of the invention may be used in the form of the free base, in the form of salts, solvates, and as prodrugs. All forms are within the scope of the invention. In accordance with the methods of the invention, the described compounds or salts, solvates, or prodrugs thereof may be administered to a patient in a variety of forms depending on the selected route of administration, as will be understood by those skilled in the art. The compounds of the invention may be administered, for example, by oral, parenteral, buccal, sublingual, nasal, rectal, patch, pump, ortransdermal administration and the pharmaceutical compositions formulated accordingly. Parenteral administration includes intravenous, intraperitoneal, subcutaneous, intramuscular, transepithelial, nasal, intrapulmonary, intrathecal, rectal, and topical modes of administration. Parenteral administration may be by continuous infusion over a selected period of time.

A compound of the invention may be orally administered, for example, with an inert diluent or with an assimilable edible carrier, or it may be enclosed in hard or soft shell gelatin capsules, or it may be compressed into tablets, or it may be incorporated directly with the food of the diet. For oral therapeutic administration, a compound of the invention may be incorporated with an excipient and used in the form of ingestible tablets, buccal tablets, troches, capsules, elixirs, suspensions, syrups, and wafers.

A compound of the invention may also be administered parenterally. Solutions of a compound of the invention can be prepared in water suitably mixed with a surfactant. Dispersions can also be prepared in glycerol, liquid polyethylene glycols, DMSO and mixtures thereof with or without alcohol, and in oils. Under ordinary conditions of storage and use, these preparations may contain a preservative to prevent the growth of microorganisms. Conventional procedures and ingredients for the selection and preparation of suitable formulations are described, for example, in Remington's Pharmaceutical Sciences (2003, 20^{th} ed.) and in The United States Pharmacopeia: The National Formulary (USP 24 NF19), published in 1999.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that may be easily administered via syringe.

Compositions for nasal administration may conveniently be formulated as aerosols, drops, gels, and powders. Aerosol formulations typically include a solution or fine suspension of the active substance in a physiologically acceptable aqueous or non-aqueous solvent and are usually presented in single or multidose quantities in sterile form in a sealed container, which can take the form of a cartridge or refill for use with an atomizing device. Alternatively, the sealed container may be a unitary dispensing device, such as a single dose nasal inhaler or an aerosol dispenser fitted with a metering valve which is intended for disposal after use. Where the dosage form comprises an aerosol dispenser, it will contain a propellant, which can be a compressed gas, such as compressed air or an organic propellant, such as fluorochlorohydrocarbon. The aerosol dosage forms can also take the form of a pump-atomizer. Compositions suitable for buccal or sublingual administration include tablets, lozenges, and pastilles, where the active ingredient is formulated with a carrier, such as sugar, acacia, tragacanth, gelatin, and glycerine. Compositions for rectal administration are conveniently in the form of suppositories containing a conventional suppository base, such as cocoa butter.

The compounds of the invention may be administered to an animal, e.g., a human, alone or in combination with pharmaceutically acceptable carriers, as noted herein, the proportion of which is determined by the solubility and chemical nature of the compound, chosen route of administration, and standard pharmaceutical practice.

### Dosages

The dosage of the compounds of the invention, and/or compositions comprising a compound of the invention, can vary depending on many factors, such as the pharmacodynamic properties of the compound; the mode of administration; the age, health, and weight of the recipient; the nature and extent of the symptoms; the frequency of the treatment, and the type of concurrent treatment, if any; and the clearance rate of the compound in the animal to be treated. One of skill in the art can determine the appropriate dosage based on the above factors. The compounds of the invention may be administered initially in a suitable dosage that may be adjusted as required, depending on the clinical response. In general, satisfactory results may be obtained when the compounds of the invention are administered to a human at a daily dosage of, for example, between 0.05 mg and 3000 mg (measured as the solid form). Dose ranges include, for example, between 10-1000 mg.

Alternatively, the dosage amount can be calculated using the body weight of the patient. For example, the dose of a compound, or pharmaceutical composition thereof, administered to a patient may range from 0.1-50 mg/kg.

The following examples are meant to illustrate the invention. They are not meant to limit the invention in any way.

### Examples

Abbreviations:

| | |
|---|---|
| 3A-MS | 3 Å Molecular sieves |
| ACN | Acetonitrile |
| (Boc)₂O | Di-tert-butyl dicarbonate |
| Conc. | Concentrated |
| DCM | Dichloromethane |
| DIAD | Diisopropyl azodicarboxylate |
| DIBAL-H | Diisobutylaluminium hydride |
| DIPEA | Diisopropyl ethylamine |
| DMAc | N,N-Dimethylacetamide |
| DMAP | 4-Dimethylaminopyridine |
| DME | Dimethoxyethane |
| DMF | N,N-Dimethylformamide |
| DMF-DMA | N,N-Dimethylformamide dimethyl acetal |
| DMSO | Dimethyl sulfoxide |
| DPPA | Diphenylphosphoryl azide |
| EA | Ethyl acetate |
| EDCl | 1-Ethyl-3-(3-dimethylaminopropyl)carbodiimide |
| HATU | (1-[Bis(dimethylamino)methylene]-1H-1,2,3- |
| | triazolo[4,5-b]pyridinium 3-oxide hexafluorophosphate |
| HOBt | Hydroxybenzotriazole |
| LAH | Lithium aluminum hydride |
| LiHMDS | Lithium bis(trimethylsily)amide |
| mW | microwave |
| NMO | N-Methylmorpholine N-oxide |
| NMP | N-Methyl-2-pyrrolidone |
| NMR | s: singlet, d: doublet, t: triplet, q: quartet, hept: heptet, |
| | m: multiplet, dd: doublet-of-doublet, dt: doublet-of-triplet |
| PCC | Pyridinium chlorochromate |
| PCy₃ | Tricyclohexylphosphine |
| Pd(dppf)Cl₂ | [1,1′-Bis(diphenylphosphino)ferrocene]dichloropalla- |
| | dium(II) |
| Pd₂(dba)₃ | Tris(dibenzylideneacetone)dipalladium(0) |
| prep-HPLC | preparative-scale high performance liquid chromatography |
| Prep-TLC | Preparative-thin layer chromatography |
| RT | Room temperature |
| RT | Room temperature |
| TEA | Triethylamine |
| TFA | Trifluoroacetic acid |
| THF | Tetrahydrofuran |
| TMEDA | Tetramethylethylenediamine |
| Xantphos | 4,5-Bis(diphenylphosphino)-9,9-dimethylxanthene |
| X-Phos | 2-Dicyclohexylphosphino-2′,4′,6′-triisopropylbiphenyl; |

### Synthesis of (E)-5-(5-(2-(3-methylbenzylidene)hydrazinyl)-2-(pyridin-4-yl)pyrazolo[1,5-a]pyrimidin-7-yl)-2-oxa-5-azabicyclo[2.2.1]heptane (Compound 1)

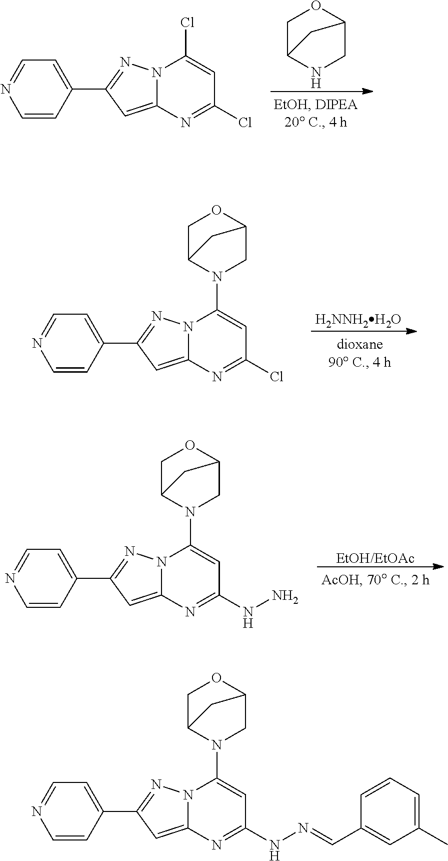

### Step 1: Synthesis of 5-(5-chloro-2-(pyridin-4-yl)pyrazolo[1,5-a]pyrimidin-7-yl)-2-oxa-5-azabicyclo[2.2.1]heptane

To a solution of 5,7-dichloro-2-(pyridin-4-yl)pyrazolo[1,5-a]pyrimidine (200 mg, 0.76 mmol), 2-oxa-5-azabicyclo[2.2.1]heptane (75 mg, 0.76 mmol) in ethanol (15 mL) was added diisopropylethylamine (98 mg, 0.76 mmol) at 0° C. Then mixture was allowed to warm up to 25° C. and stirred for 4h. It was concentrated and the residue was subjected to silica gel column chromatography (dichloromethane:/methanol=20:1) to obtain 5-(5-chloro-2-(pyridin-4-yl)pyrazolo[1,5-a]pyrimidin-7-yl)-2-oxa-5-azabicyclo[2.2.1]heptane (190 mg) as white solid. LCMS (ESI) m/z: 328.1 [M+H]+.

### Step 2: Synthesis of 5-(5-hydrazinyl-2-(pyridin-4-yl)pyrazolo[1,5-a]pyrimidin-7-yl)-2-oxa-5-azabicyclo[2.2.1]heptane

To a solution of 5-(5-chloro-2-(pyridin-4-yl)pyrazolo[1,5-a]pyrimidin-7-yl)-2-oxa-5-azabicyclo[2.2.1]heptane (190 mg, 0.588 mmol) in dioxane (9 mL) was added hydrazine hydrate (3 mL). Then the mixture was heated to 90° C. and stirred for 4h. It was concentrated and the residue was subjected to silica gel column chromatography (dichloromethane:/methanol=20:1) to obtain 5-(5-hydrazinyl-2-(pyridin-4-yl)pyrazolo[1,5-a]pyrimidin-7-yl)-2-oxa-5-azabicyclo[2.2.1]heptane (160 mg) as white solid. LCMS (ESI) m/z: 324.1 [M+H]+.

### Step 3: Synthesis of (E)-5-(5-(2-(3-methylbenzylidene)hydrazinyl)-2-(pyridin-4-yl)pyrazolo[1,5-a]pyrimidin-7-yl)-2-oxa-5-azabicyclo[2.2.1]heptane

To a solution of 5-(5-hydrazinyl-2-(pyridin-4-yl)pyrazolo[1,5-a]pyrimidin-7-yl)-2-oxa-5-azabicyclo[2.2.1]heptane (160 mg, 0.689 mmol) and 3-methylbenzaldehyde (91 mg, 0.758 mmol) in ethyl acetate (10 mL) and ethanol (10 mL) was added a drop of acetic acid. Then the mixture was heated to 70° C. and stirred for 2h. It was concentrated and the residue was subjected to prep-HPLC (SunFire C18, 4.6*50 mm, 3.5 um column Xbridge C18 3.5 μm 4.6×50 mm column. The elution system used was a gradient of 5%-95% over 1.5 min at 2 ml/min and the solvent was acetonitrile/0.01% aqueous NH₄HCO₃.) to obtain (E)-5-(5-(2-(3-methylbenzylidene)hydrazinyl)-2-(pyridin-4-yl)pyrazolo[1,5-a]pyrimidin-7-yl)-2-oxa-5-azabicyclo[2.2.1]heptane (111 mg, 38%) as light-yellow solid. ¹H NMR (400 MHz, DMSO-d₆) δ 11.06 (s, 1H), 8.65 (d, J=6.0 Hz, 2H), 8.02 (s, 1H), 7.91 (dd, J=4.6, 1.4 Hz, 2H), 7.64-7.42 (m, 2H), 7.31 (t, J=7.6 Hz, 1H), 7.18 (d, J=7.5 Hz, 1H), 6.65 (s, 1H), 6.02 (s, 2H), 4.77 (s, 1H), 4.13-3.84 (m, 3H), 3.69 (d, J=10.0 Hz, 1H), 2.36 (s, 3H), 2.10 (d, J=8.3 Hz, 1H), 1.99 (d, J=9.7 Hz, 1H). LCMS (ESI) m/z: 426.1 [M+H]+.

### Synthesis of 4-(5-(3-methylphenethoxy)-2-(pyridin-4-yl)pyrazolo[1,5-a]pyrimidin-7-yl)morpholine (Compound 2)

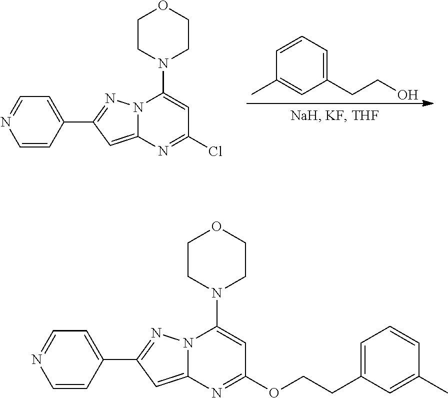

To a solution of 2-(m-tolyl)ethan-1-ol (0.21 g, 1.52 mmol) in tetrahydrofuran (5 mL) was added sodium hydride (0.05 g, 1.33 mmol) at 0° C. and the mixture was stirred at 25° C. for 0.5h. A solution of 4-(5-chloro-2-(pyridin-4-yl)pyrazolo[1,5-a]pyrimidin-7-yl)morpholine (0.2 g, 0.63 mmol) in THE and potassium fluoride (0.092 g, 1.58 mmol) were then added to the mixture at 25° C. The mixture was heated to 110° C. and stirred for 17 h. The mixture was then filtered to remove the solids and the filtrate was concentrated and the residue was subjected to prep-HPLC (Boston C18 21*250 mm 10 μm column. The mobile phase was acetonitrile/10 mM formic acid aqueous solution) to obtain 4-(5-(3-methylphenethoxy)-2-(pyridin-4-yl)pyrazolo[1,5-a]pyrimidin-7-yl)morpholine as white solid.(62.9 mg, 24%)¹H NMR (400 MHz, DMSO-d6) δ 8.67 (d, J=5.0 Hz, 2H), 8.46 (s, 1H), 7.93 (d, J=4.7 Hz, 2H), 7.21 (t, J=7.7 Hz, 1H), 7.15 (s, 1H), 7.12 (d, J=7.0 Hz, 1H), 7.05 (d, J=7.3 Hz, 1H), 6.99 (s, 1H), 5.86 (s, 1H), 4.53 (t, J=6.8 Hz, 2H), 3.83 (bs, 4H), 3.70 (bs, 4H), 3.02 (t, J=6.7 Hz, 2H), 2.30 (s, 3H).; LCMS (ESI) m/z: 416.3 [M+H]+.

### Synthesis of 2-(7-morpholino-2-(pyridin-4-yl)pyrazolo[1,5-a]pyrimidin-5-yl)-6-phenylpyridazin-3(2H)-one (Compound 3)

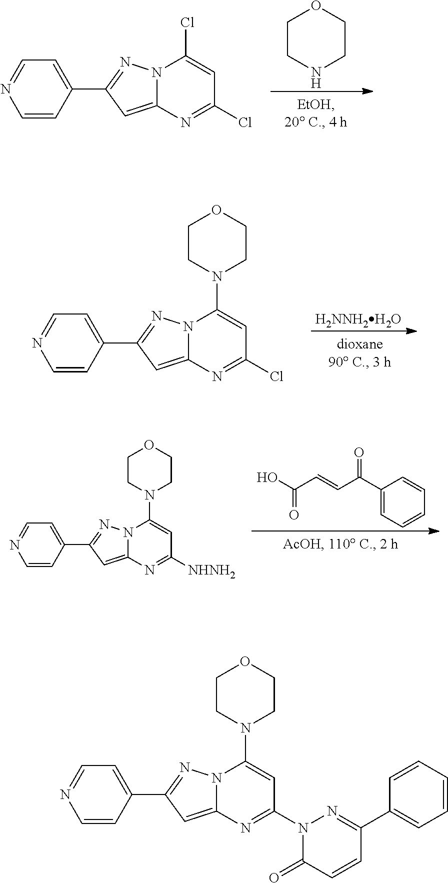

### Step 1: Synthesis of 4-(5-chloro-2-(pyridin-4-yl)pyrazolo[1,5-a]pyrimidin-7-yl)morpholine

To a solution of 5,7-dichloro-2-(pyridin-4-yl)pyrazolo[1,5-a]pyrimidine (514 mg, 1.95 mmol) in ethanol (25 mL) was added morpholine (339 mg, 3.893 mmol) at 0° C. Then the mixture was warmed up and stirred for another 4 h at 25° C. The mixture was concentrated, the resultant precipitate was collected by filtration and vacuum dried to obtain 4-(5-chloro-2-(pyridin-4-yl)pyrazolo[1,5-a]pyrimidin-7-yl)morpholine (505 mg) as light-yellow solid. LCMS (ESI) m/z: 316.2 [M+H]+.

### Step 2: Synthesis of 4-(5-hydrazinyl-2-(pyridin-4-yl)pyrazolo[1,5-a]pyrimidin-7-yl)morpholine

To a solution of 4-(5-chloro-2-(pyridin-4-yl)pyrazolo[1,5-a]pyrimidin-7-yl)morpholine (515 mg, 1.634 mmol) in dioxane (8 mL) was added hydrazine hydrate (2 mL). Then the mixture was heated to 90° C. and stirred for 4h. It was concentrated, the residue was slurred in ethanol and resultant precipitate was collected by filtration. The solids were vacuum dried to obtain 4-(5-hydrazinyl-2-(pyridin-4-yl)pyrazolo[1,5-a]pyrimidin-7-yl)morpholine (410 mg) as white solid. LCMS (ESI) m/z: 312.1 [M+H]+.

### Step 3: Synthesis of 2-(7-morpholino-2-(pyridin-4-yl)pyrazolo[1,5-a]pyrimidin-5-yl)-6-phenylpyridazin-3(2H)-one

A solution of 4-(5-hydrazinyl-2-(pyridin-4-yl)pyrazolo[1,5-a]pyrimidin-7-yl)morpholine (200 mg, 0.643 mmol) and (E)-4-oxo-4-phenylbut-2-enoic acid (113 mg, 0.643 mmol) in acetic acid (5 mL) was heated to 110° C. and stirred for 2 h. The mixture was concentrated, and residue was subjected to prep-HPLC (SunFire C18, 4.6*50 mm, 3.5 um column Xbridge C18 3.5 μm 4.6×50 mm column. The elution system used was a gradient of 5%-95% over 1.5 min at 2 ml/min and the solvent was acetonitrile/0.01% aqueous HCOOH) to obtain 2-(7-morpholino-2-(pyridin-4-yl)pyrazolo[1,5-a]pyrimidin-5-yl)-6-phenylpyridazin-3(2H)-one (30.2 mg, 10%) as white solid. ¹H NMR (400 MHz, DMSO-d₆) δ 8.73 (d, J=5.9 Hz, 2H), 8.23 (d, J=9.9 Hz, 1H), 8.04 (d, J=6.0 Hz, 2H), 7.93 (dd, J=7.8, 1.8 Hz, 2H), 7.51 (d, J=7.2 Hz, 3H), 7.39 (s, 1H), 7.26 (d, J=9.9 Hz, 1H), 6.78 (s, 1H), 3.92 (d, J=5.2 Hz, 4H), 3.88 (d, J=5.2 Hz, 4H). LCMS (ESI) m/z: 452.1 [M+H]+.

### Syntheses of 4,5-dibromo-2-(7-morpholino-2-(pyridin-4-yl)pyrazolo[1,5-a]pyrimidin-5-yl)pyridazin-3(2H)-one (Compound 4) and 2-(7-morpholino-2-(pyridin-4-yl)pyrazolo[1,5-a]pyrimidin-5-yl)-4-phenylpyridazin-3(2H)-one (Compound 5)

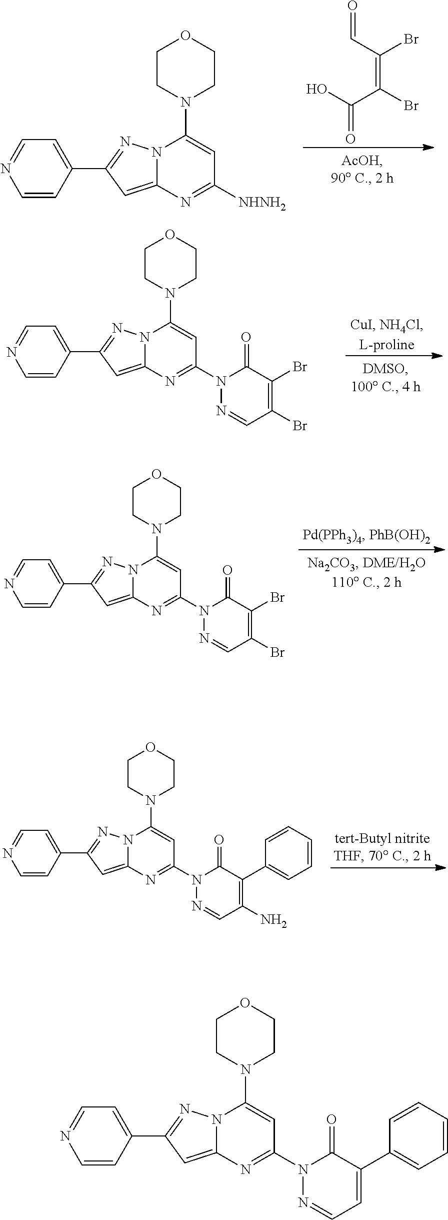

### Step 1: Synthesis of 4,5-dibromo-2-(7-morpholino-2-(pyridin-4-yl)pyrazolo[1,5-a]pyrimidin-5-yl)pyridazin-3(2H)-one

A solution of 4-(5-hydrazinyl-2-(pyridin-4-yl)pyrazolo[1,5-a]pyrimidin-7-yl)morpholine (1.62 g, 5.21 mmol), and (Z)-2,3-dibromo-4-oxobut-2-enoic acid (917 mg, 5.21 mmol) in acetic acid (10 mL) was heated to 90° C. and stirred for 2h. It was concentrated under reduced pressure and the residue was subjected to silica gel column chromatography (dichloromethane:/methanol=20:1) to obtain 4,5-dibromo-2-(7-morpholino-2-(pyridin-4-yl)pyrazolo[1,5-a]pyrimidin-5-yl)pyridazin-3(2H)-one(1.308 g) as yellow solid.

¹H NMR (400 MHz, DMSO-d₆) δ 8.72 (d, J=6.0 Hz, 2H), 8.35 (s, 1H), 8.03 (d, J=6.0 Hz, 2H), 7.38 (s, 1H), 6.75 (s, 1H), 3.91 (d, J=4.8 Hz, 4H), 3.87 (d, J=4.8 Hz, 4H); LCMS (ESI) m/z: 533.8 [M+H]+.

### Step 2: Synthesis of 5-amino-4-bromo-2-(7-morpholino-2-(pyridin-4-yl)pyrazolo[1,5-a]pyrimidin-5-yl)pyridazin-3(2H)-one

To a solution of 4,5-dibromo-2-(7-morpholino-2-(pyridin-4-yl)pyrazolo[1,5-a]pyrimidin-5-yl)pyridazin-3(2H)-one (1.308 g, 2.463 mmol), ammonium chloride (522 mg, 9.854 mmol) and L-proline (113 mg, 0.985 mmol) in DMSO (10 mL) was added copper iodide (94 mg, 0.493 mmol). The mixture was heated to 100° C. and stirred for 4h. It was concentrated and the residue was subjected to prep-HPLC (SunFire C18, 4.6*50 mm, 3.5 um column Xbridge C18 3.5 μm 4.6×50 mm column. The elution system used was a gradient of 5%-95% over 1.5 min at 2 ml/min and the solvent was acetonitrile/0.01% aqueous NH₄HCO₃) to obtain 5-amino-4-bromo-2-(7-morpholino-2-(pyridin-4-yl)pyrazolo[1,5-a]pyrimidin-5-yl)pyridazin-3(2H)-one (230 mg) was isolated as yellow solid. ¹H NMR (400 MHz, DMSO-d₆) δ 8.72 (s, 2H), 8.02 (d, J=4.1 Hz, 2H), 7.72 (s, 1H), 7.32 (s, 1H), 7.13 (bs, 2H), 6.64 (s, 1H), 3.87 (s, 8H); LCMS (ESI) m/z: 469.0 [M+H]+. The regioisomer 4-amino-5-bromo-2-(7-morpholino-2-(pyridin-4-yl)pyrazolo[1,5-a]pyrimidin-5-yl)pyridazin-3(2H)-one was also isolated as yellow solid during this step.

### Step 3: Synthesis of 5-amino-2-(7-morpholino-2-(pyridin-4-yl)pyrazolo[1,5-a]pyrimidin-5-yl)-4-phenylpyridazin-3(2H)-one

To a solution of 5-amino-4-bromo-2-(7-morpholino-2-(pyridin-4-yl)pyrazolo[1,5-a]pyrimidin-5-yl)pyridazin-3(2H)-one (225 mg, 0.481 mmol), phenylboronic acid (117 mg, 0.961 mmol) and sodium carbonate (aqueous, 2N, 1.0 mL) in dimethoxyethane (10 mL) was added tetrakis(triphenylphosphine)palladium(0) (56 mg, 0.048 mmol). Then the mixture was heated to 110° C. and stirred for 2h. It was concentrated and the residue was subjected to prep-HPLC (SunFire C18, 4.6*50 mm, 3.5 um column Xbridge C18 3.5 μm 4.6×50 mm column. The elution system used was a gradient of 5%-95% over 1.5 min at 2 ml/min and the solvent was acetonitrile/0.01% aqueous NH₄HCO₃) to obtain 5-amino-2-(7-morpholino-2-(pyridin-4-yl)pyrazolo[1,5-a]pyrimidin-5-yl)-4-phenylpyridazin-3(2H)-one (110 mg) as brown solid. LCMS (ESI) m/z: 467.1 [M+H]+.

### Step 4: Synthesis of 2-(7-morpholino-2-(pyridin-4-yl)pyrazolo[1,5-a]pyrimidin-5-yl)-4-phenylpyridazin-3(2H)-one

To a solution of 5-amino-2-(7-morpholino-2-(pyridin-4-yl)pyrazolo[1,5-a]pyrimidin-5-yl)-4-phenylpyridazin-3(2H)-one (110 mg, 0.235 mmol) in tetrahydrofuran (10 mL) was added tert-butyl nitrite (194 mg, 1.88 mmol). The mixture was heated to 70° C. and stirred for 2h. It was concentrated and the residue was subjected to prep-HPLC (SunFire C18, 4.6*50 mm, 3.5 um column Xbridge C18 3.5 μm 4.6×50 mm column. The elution system used was a gradient of 5%-95% over 1.5 min at 2 ml/min and the solvent was acetonitrile/0.01% aqueous HCOOH) to obtain 2-(7-morpholino-2-(pyridin-4-yl)pyrazolo[1,5-a]pyrimidin-5-yl)-4-phenylpyridazin-3(2H)-one (7.8 mg, 7.4%) as light-yellow solid. ¹H NMR (400 MHz, DMSO-d₆) δ 8.72 (d, J=5.3 Hz, 2H), 8.17 (d, J=4.2 Hz, 1H), 8.04 (d, J=5.8 Hz, 2H), 7.88 (dd, J=6.5, 3.0 Hz, 2H), 7.74 (d, J=4.2 Hz, 1H), 7.64-7.44 (m, 3H), 7.37 (s, 1H), 6.77 (s, 1H), 3.92-3.88 (m, 8H); LCMS (ESI) m/z: 452.1 [M+H]+.

### Synthesis of 2-(7-morpholino-2-(pyridin-4-yl)pyrazolo[1,5-a]pyrimidin-5-yl)-4,5-diphenylpyridazin-3(2H)-one (Compound 6) and 4-bromo-5-methoxy-2-(7-morpholino-2-(pyridin-4-yl)pyrazolo[1,5-a]pyrimidin-5-yl)pyridazin-3(2H)-one (Compound 7)

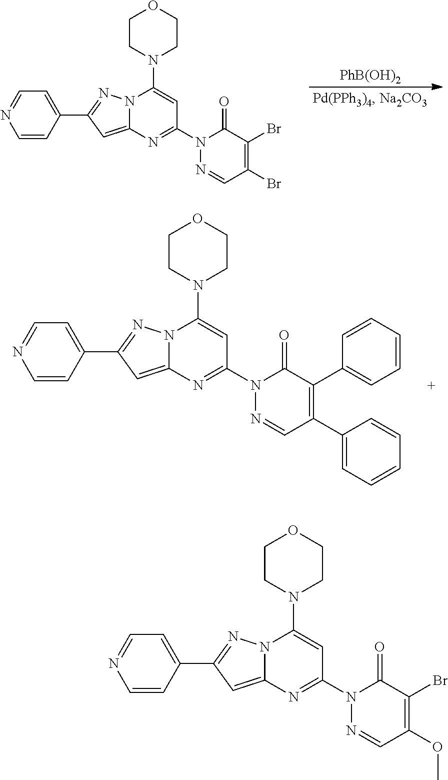

To a solution of 4,5-dibromo-2-(7-morpholino-2-(pyridin-4-yl)pyrazolo[1,5-a]pyrimidin-5-yl)pyridazin-3(2H)-one (150 mg, 0.282 mmol), phenylboronic acid (38 mg, 0.311 mmol), tetrakis(triphenylphosphine) (32 mg, 0.028 mmol) in methanol/toluene (2 mL/5 mL) was added a solution of aqueous sodium carbonate (2N, 0.5 mL) in portions at 20° C. under nitrogen atmosphere. Then the mixture was stirred at 110° C. for 1 h in a microwave reactor. The mixture was concentrated, and the residue was subjected to prep-HPLC (SunFire C18, 4.6*50 mm, 3.5 um column Xbridge C18 3.5 μm 4.6×50 mm column. The elution system used was a gradient of 5%-95% over 1.5 min at 2 ml/min and the solvent was acetonitrile/0.01% aqueous NH₄HCO₃) to obtain 2-(7-morpholino-2-(pyridin-4-yl)pyrazolo[1,5-a]pyrimidin-5-yl)-4,5-diphenylpyridazin-3(2H)-one (21.4 mg, 14%) and 4-bromo-5-methoxy-2-(7-morpholino-2-(pyridin-4-yl)pyrazolo[1,5-a]pyrimidin-5-yl)pyridazin-3(2H)-one (17.1 mg, 12%, byproduct) as white solids.

Compound 6: ¹H NMR (400 MHz, TFA-d) δ 9.00 (d, J=6.5 Hz, 2H), 8.72 (d, J=6.5 Hz, 2H), 8.60 (s, 1H), 7.95 (s, 1H), 7.61 (s, 1H), 7.55-7.34 (m, 6H), 7.27 (t, J=7.4 Hz, 4H), 4.76 (Bs, 4H), 4.39 (s, 4H). LCMS (ESI) m/z: 528.2 [M+H]+.

Compound 7: ¹H NMR (400 MHz, DMSO-d₆) δ 8.72 (d, J=5.9 Hz, 2H), 8.37 (s, 1H), 8.03 (d, J=6.0 Hz, 2H), 7.36 (s, 1H), 6.69 (s, 1H), 4.17 (s, 3H), 3.91 (d. J=5.2 Hz, 4H), 3.88 (d, J=4.4 Hz, 4H); LCMS (ESI) m/z: 484.0/486.0 [M+H]+.

### Synthesis of 2-(7-morpholino-2-(pyridin-4-yl)pyrazolo[1,5-a]pyrimidin-5-yl)-5-phenylpyridazin-3(2H)-one (Compound 8)

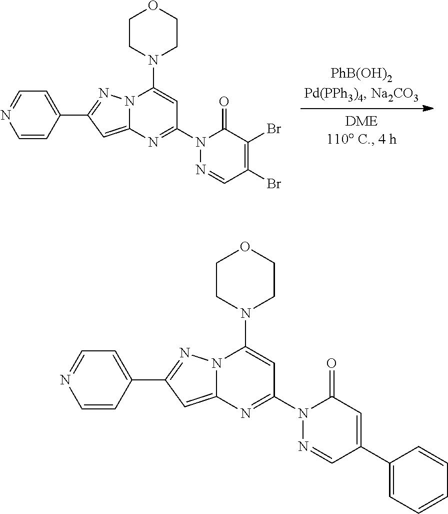 [SMILES: O=c1c(Br)c(Br)cnn1-c1cc(N2CCOCC2)n2nc(-c3ccncc3)cc2n1 ; O=c1cc(-c2ccccc2)cnn1-c1cc(N2CCOCC2)n2nc(-c3ccncc3)cc2n1]

To a solution of 4,5-dibromo-2-(7-morpholino-2-(pyridin-4-yl)pyrazolo[1,5-a]pyrimidin-5-yl)pyridazin-3(2H)-one (150 mg, 0.282 mmol), phenylboronic acid (38 mg, 0.311 mmol), tetrakis(triphenylphosphine) (32 mg, 0.028 mmol) in 1,2-dimethoxyethane (5 mL) was added aqueous sodium carbonate solution (2N, 0.5 mL) in portions at 20° C. under nitrogen atmosphere. Then the mixture was stirred at 110° C. for 6 h and concentrated. The residue was subjected to prep-TLC (dichloromethane: /methanol=20:1) to obtain 2-(7-morpholino-2-(pyridin-4-yl)pyrazolo[1,5-a]pyrimidin-5-yl)-5-phenylpyridazin-3(2H)-one(2.1 mg) as light-yellow solid. ¹H NMR (400 MHz, DMSO-d₆) δ 8.73 (d, J=5.1 Hz, 2H), 8.58 (d, J=2.0 Hz, 1H), 8.04 (d, J=5.8 Hz, 2H), 7.94 (d, J=3.5 Hz, 2H), 7.64-7.54 (m, 3H), 7.43 (d, J=2.0 Hz, 1H), 7.38 (s, 1H), 6.75 (s, 1H), 3.91 (bs, 4H), 3.89 (bs, 4H); LCMS (ESI) m/z: 452.3 [M+H]+.

### Synthesis of 4-(5-(2-phenylpyrimidin-4-yl)-2-(pyridin-4-yl)pyrazolo[1,5-a]pyrimidin-7-yl)morpholine (Compound 9)

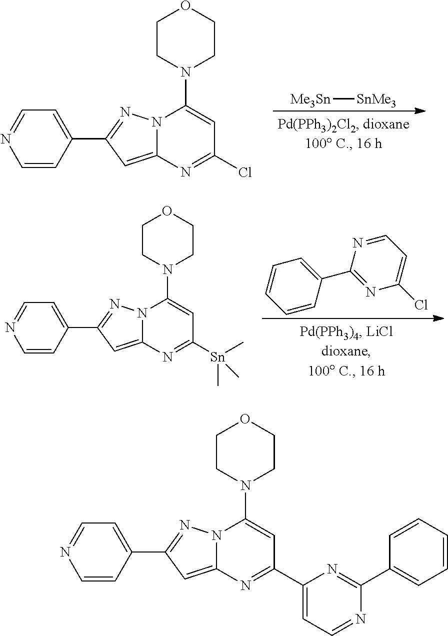

### Step 1: Synthesis of 4-(2-(pyridin-4-yl)-5-(trimethylstannyl)pyrazolo[1,5-a]pyrimidin-7-yl)morpholine

To a solution of 4-(5-chloro-2-(pyridin-4-yl)pyrazolo[1,5-a]pyrimidin-7-yl)morpholine (200 mg, 0.6 mmol) in dioxane (10 mL) were added 1,1,1,2,2,2-hexamethyldistannane (250 mg, 0.76 mmol) and bis(triphenylphosphine)palladium(II) chloride (40 mg, 0.06 mmol) at 25° C. under argon atmosphere. The mixture was stirred at 100° C. for 16 h under argon atmosphere, then cooled to 25° C. and filtered to remove the solids. The filtrate was washed with aqueous potassium fluoride (10 mL) and concentrated. The crude product that was obtained as white solid was used in next step without further purification (70 mg, 25%). LCMS (ESI) m/z: 446.1 [M+H]+.

### Step 2: Synthesis of 4-(5-(2-phenylpyrimidin-4-yl)-2-(pyridin-4-yl)pyrazolo[1,5-a]pyrimidin-7-yl)morpholine

To a solution of 4-(2-(pyridin-4-yl)-5-(trimethylstannyl)pyrazolo[1,5-a]pyrimidin-7-yl)morpholine (40 mg, 0.1 mmol) in dioxane (6 mL) were added 4-chloro-2-phenylpyrimidine (30 mg, 0.2 mmol), lithium chloride (20 mg, 0.5 mmol) and tetrakis(triphenylphosphine)palladium (10 mg, 0.01 mmol) at 25° C. under argon atmosphere. The resultant mixture was stirred at 100° C. for 16 h under argon atmosphere. The reaction mixture was then filtered to remove the solids, the filtrate was concentrated and then subjected to prep-HPLC (Boston C18 21*250 mm 10 μm column. The mobile phase was acetonitrile/10 mM ammonium bicarbonate aqueous solution.) to obtain 4-(5-(2-phenylpyrimidin-4-yl)-2-(pyridin-4-yl)pyrazolo[1,5-a]pyrimidin-7-yl)morpholine (2.7 mg, 7%) as white solid. ¹H NMR (400 MHz, DMSO-d₆) δ 9.12 (d, J=5.1 Hz, 1H), 8.73 (d, J=6.0 Hz, 2H), 8.59 (dd, J=6.7, 3.0 Hz, 2H), 8.34 (d, J=5.1 Hz, 1H), 8.05 (d, J=6.0 Hz, 2H), 7.61 (dd, J=6.2, 3.6 Hz, 4H), 7.50 (s, 1H), 4.00 (d, J=5.6 Hz, 4H), 3.96 (d, J=5.6 Hz, 4H).; LCMS (ESI) m/z: 436.2 [M+H]+.

The following compounds were synthesized as above:

| Name | Structure | NMR, LC-MS | Comp# |
|---|---|---|---|
| 4-(5-(6- phenylpyrazin- 2-yl)-2-(pyridin- 4- yl)pyrazolo[1,5- a]pyrimidin-7- yl)morpholine | 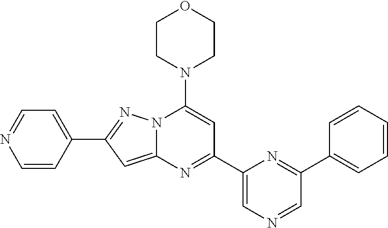 [SMILES: c1ccc(-c2cncc(-c3cc(N4CCOCC4)n4nc(-c5ccncc5)cc4n3)n2)cc1] | ¹H NMR (400 MHz, DMSO-d₆) δ 9.54 (s, 1H), 9.40 (s, 1H), 8.73 (d, J = 5.4 Hz, 2H), 8.33 (d, J = 6.9 Hz, 2H), 8.04 (d, J = 5.6 Hz, 2H), 7.67-7.54 (m, 4H), 7.47 (d, J = 2.3 Hz, 2H), 3.96 (s, 8H). ; LCMS (ESI) m/z: 436.2 [M + H]+. | 10 |
| 4-(5-(6- phenylpyridin-2- yl)-2-(pyridin-4- yl)pyrazolo[1,5- a]pyrimidin-7- yl)morpholine | 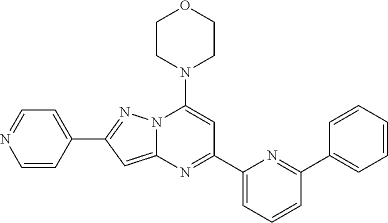 [SMILES: c1ccc(-c2cccc(-c3cc(N4CCOCC4)n4nc(-c5ccncc5)cc4n3)n2)cc1] | ¹H NMR (400 MHz, DMSO-d₆) δ 8.72 (d, J = 6.0 Hz, 2H), 8.42 (dd, J = 7.2, 1.4 Hz, 1H), 8.26 (d, J = 7.2 Hz, 2H), 8.15-8.07 (m, 2H), 8.04 (d, J = 6.0 Hz, 2H), 7.58 (dd, J = 8.8, 6.0 Hz, 3H), 7.51 (t, J = 7.2 Hz, 1H), 7.42 (s, 1H), 3.94 (s, 8H). ; LCMS (ESI) m/z: 435.1 [M + H]+. | 11 |

### Synthesis of 4-(5-(1-(pyridin-3-yl)-1H-pyrazol-5-yl)-2-(pyridin-4-yl)pyrazolo[1,5-a]pyrimidin-7-yl)morpholine (Compound 12) and 4-(5-(1-(pyridin-3-yl)-1H-pyrazol-3-yl)-2-(pyridin-4-yl)pyrazolo[1,5-a]pyrimidin-7-yl)morpholine (Compound 13)

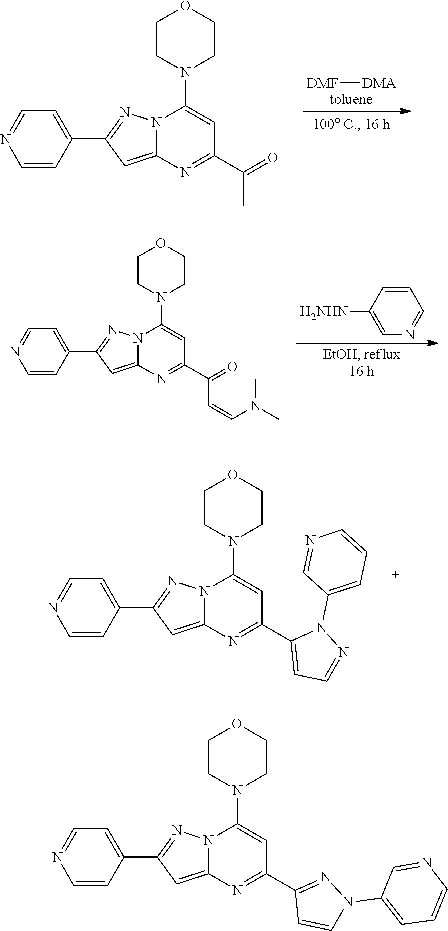

### Step 1: Synthesis of (Z)-3-(dimethylamino)-1-(7-morpholino-2-(pyridin-4-yl)pyrazolo[1,5-a]pyrimidin-5-yl)prop-2-en-1-one

To a solution of 1-(7-morpholino-2-(pyridin-4-yl)pyrazolo[1,5-a]pyrimidin-5-yl)ethan-1-one (323 mg, 1 mmol) in toluene (10 mL) was added N,N-dimethylformamide dimethyl acetal (595 mg, 5 mmol) and the reaction mixture was stirred at 110° C. for 16h. It was then concentrated to obtain (Z)-3-(dimethylamino)-1-(7-morpholino-2-(pyridin-4-yl)pyrazolo[1,5-a]pyrimidin-5-yl)prop-2-en-1-one as yellow solid (196 mg, 52%). LCMS (ESI) m/z: 379.2 [M+H]+.

### Step 2: Synthesis of 4-(5-(1-(pyridin-3-yl)-1H-pyrazol-3-yl)-2-(pyridin-4-yl)pyrazolo[1,5-a]pyrimidin-7-yl)morpholine

A mixture of (Z)-3-(dimethylamino)-1-(7-morpholino-2-(pyridin-4-yl)pyrazolo[1,5-a]pyrimidin-5-yl)prop-2-en-1-one (151.2 mg, 0.4 mmol) and 3-hydrazineylpyridine (145.6 mg, 0.8 mmol) in ethanol (10 mL) was stirred at 80° C. for 16 h and concentrated. The residue was triturated with aqueous sodium bicarbonate solution (10 mL) and the resultant precipitate was collected by filtration, washed with ethanol (10 mL) and dried. The crude product thus obtained was subjected to prep-HPLC conditions to afford 4-(5-(1-(pyridin-3-yl)-1H-pyrazol-5-yl)-2-(pyridin-4-yl)pyrazolo[1,5-a]pyrimidin-7-yl)morpholine (13 mg, 8%) and 4-(5-(1-(pyridin-3-yl)-1H-pyrazol-3-yl)-2-(pyridin-4-yl)pyrazolo[1,5-a]pyrimidin-7-yl)morpholine as white solid (11.2 mg, 7%).

Compound 12: ¹H NMR (400 MHz, DMSO-d₆) δ 8.66 (d, J=6.0 Hz, 2H), 8.59 (dt, J=7.6, 2.8 Hz, 2H), 7.97-7.82 (m, 4H), 7.50 (dd, J=8.0, 4.8 Hz, 1H), 7.24 (d, J=2.0 Hz, 1H), 7.05 (s, 1H), 6.69 (s, 1H), 3.86 (s, 8H); LCMS (ESI) m/z: 425.3 [M+H]+.

Compound 13: ¹H NMR (400 MHz, DMSO-d₆) δ 9.29 (d, J=2.4 Hz, 1H), 8.77 (d, J=2.8 Hz, 1H), 8.71-8.72 (m, 2H), 8.59-8.61 (m, 1H), 8.39-8.42 (m, 1H), 8.01-8.01 (m, 2H), 7.61-7.64 (m, 1H), 7.33 (s, 1H), 7.26 (d, J=2.8 HZ, 1H), 7.12 (s, 1H), 3.91 (d, J=5.2 Hz, 8H); LCMS (ESI) m/z: 425.3 [M+H]+.

### Synthesis of 4-(5-(1-phenyl-1H-pyrazol-3-yl)-2-(pyridin-4-yl)pyrazolo[1,5-a]pyrimidin-7-yl)morpholine (Compound 14)

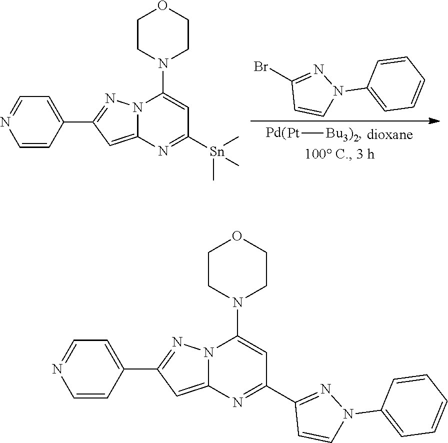

A mixture of 4-(2-(pyridin-4-yl)-5-(trimethylstannyl)pyrazolo[1,5-a]pyrimidin-7-yl)morpholine (0.222 g, 0.5 mmol), 3-bromo-1-phenyl-1H-pyrazole (0.222 g, 1 mmol) and bis(tri-tert-butylphosphine)palladium(0) (25 mg, 0.05 mmol) in dioxane (10 mL) was stirred at 100° C. for 3 h under argon atmosphere. It was filtered to remove the insoluble material and the filtrate was concentrated. The residue was subjected to prep-HPLC (BOSTON pHlex ODS 10 μm 21.2×250 mm120 A. The mobile phase was acetonitrile/0.1% Ammonium bicarbonate) to obtain 4-(5-(1-phenyl-1H-pyrazol-3-yl)-2-(pyridin-4-yl)pyrazolo[1,5-a]pyrimidin-7-yl)morpholine as white solid. (5 mg, 2.4%). ¹H NMR (400 MHz, DMSO-d₆) δ 8.71 (d, J=6.0 Hz, 2H), 8.67 (d, J=2.4 Hz, 1H), 8.04-7.99 (m, 4H), 7.59-7.55 (m, 2H), 7.41-7.37 (m, 1H), 7.32 (s, 1H), 7.21 (d, J=2.4 Hz, 1H), 7.09 (s, 1H), 3.93-3.89 (m, 8H); LCMS (ESI) m/z: 424.2 [M+H]+.

### Synthesis of tert-butyl 3-(1-(7-morpholino-2-(pyridin-4-yl)pyrazolo[1,5-a]pyrimidin-5-yl)-1H-imidazol-4-yl)piperidine-1-carboxylate (Compound 15), 4-(5-(4-(piperidin-3-yl)-1H-imidazol-1-yl)-2-(pyridin-4-yl)pyrazolo[1,5-a]pyrimidin-7-yl)morpholine (Compound 16), 4-(2-(pyridin-4-yl)-5-(4-(1,2,5,6-tetrahydropyridin-3-yl)-1H-imidazol-1-yl)pyrazolo[1,5-a]pyrimidin-7-yl)morpholine (Compound 17) and 4-(5-(4-(1-methylpiperidin-3-yl)-1H-imidazol-1-yl)-2-(pyridin-4-yl)pyrazolo[1,5-a]pyrimidin-7-yl)morpholine (Compound 18)

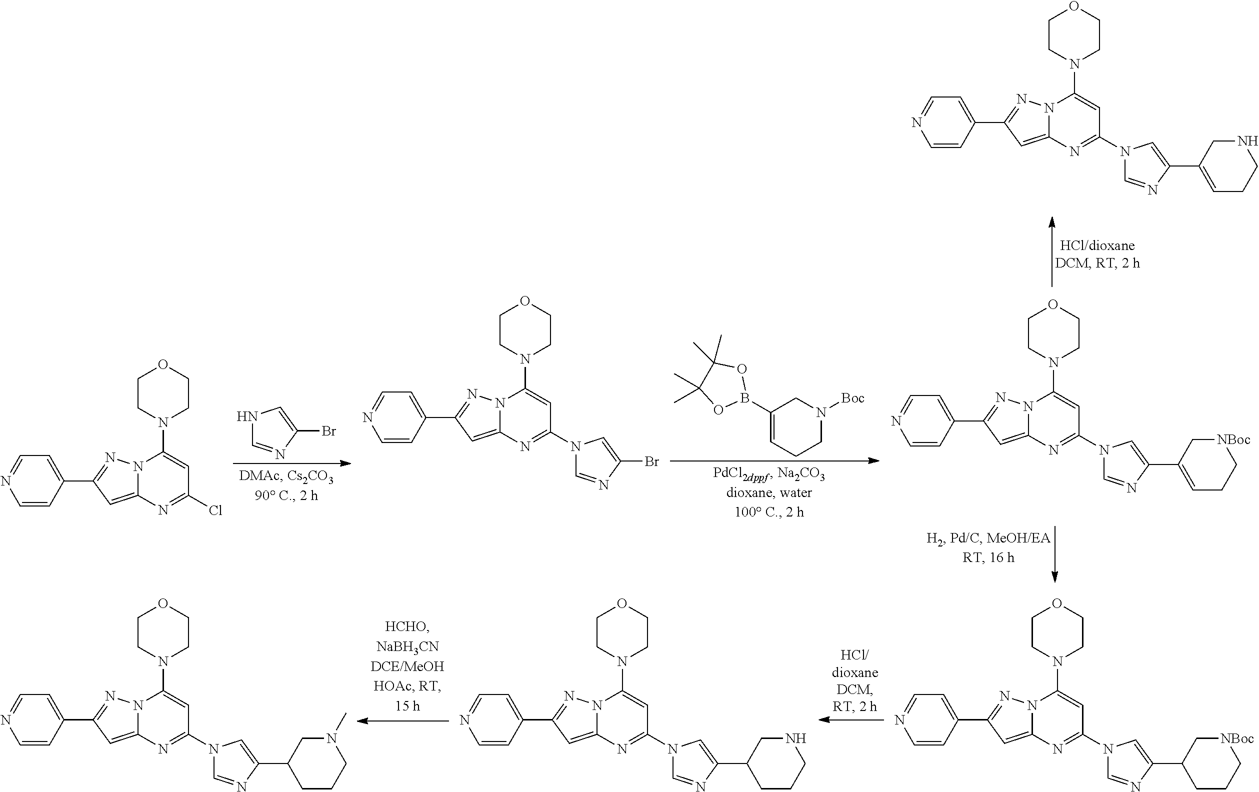

### Step 1: Synthesis of 4-(5-(4-bromo-1H-imidazol-1-yl)-2-(pyridin-4-yl)pyrazolo[1,5-a]pyrimidin-7-yl)morpholine

A mixture of 4-(5-chloro-2-(pyridin-4-yl)pyrazolo[1,5-a]pyrimidin-7-yl)morpholine (32 mg, 0.1 mmol), 4-bromo-1H-imidazole (30 mg, 0.2 mmol) and cesium carbonate (0.3 mmol, 98 mg) in DMAc (5 mL) was stirred at 90° C. for 2 h. The mixture was diluted with water (10 mL), the precipitate formed was collected by filtration, the solids were washed with water (10 mL) and vacuum dried to obtain 4-(5-(4-bromo-1H-imidazol-1-yl)-2-(pyridin-4-yl)pyrazolo[1,5-a]pyrimidin-7-yl)morpholine as yellow solid. LCMS (ESI) m/z: 426.0 [M+H]+.

### Step 2: Synthesis of tert-butyl 5-(1-(7-morpholino-2-(pyridin-4-yl)pyrazolo[1,5-a]pyrimidin-5-yl)-1H-imidazol-4-yl)-3,6-dihydropyridine-1(2H)-carboxylate

To a solution of 4-(5-(4-bromo-1H-imidazol-1-yl)-2-(pyridin-4-yl)pyrazolo[1,5-a]pyrimidin-7-yl)morpholine (425 mg, 1 mmol) in dioxane (10 mL) and water (2 mL) were added tert-butyl 5-(4,4,5,5-tetramethyl-1,3,2-dioxaborolan-2-yl)-3,6-dihydropyridine-1(2H)-carboxylate (618 mg, 2 mmol), [1,1′-bis(diphenylphosphino)ferrocene]dichloropalladium(II) (82 mg, 0.1 mmol) and sodium carbonate (318 mg, 3 mmol) at 25° C. The resultant mixture was heated up and stirred for 2 h at 100° C. under argon protection. The reaction was quenched by the addition with water (20 mL), the resultant precipitates were collected by filtration and vacuum dried to obtain tert-butyl 5-(1-(7-morpholino-2-(pyridin-4-yl)pyrazolo[1,5-a]pyrimidin-5-yl)-1H-imidazol-4-yl)-3,6-dihydropyridine-1(2H)-carboxylate as yellow solid. (528 mg, 99%). LCMS (ESI) m/z: 529.1 [M+H]+.

### Step 3: Synthesis tert-butyl 3-(1-(7-morpholino-2-(pyridin-4-yl)pyrazolo[1,5-a]pyrimidin-5-yl)-1H-imidazol-4-yl)piperidine-1-carboxylate

A mixture of tert-butyl 5-(1-(7-morpholino-2-(pyridin-4-yl)pyrazolo[1,5-a]pyrimidin-5-yl)-1H-imidazol-4-yl)-3,6-dihydropyridine-1(2H)-carboxylate (106 mg, 0.2 mmol) and palladium on charcoal (10%, 25 mg) in methanol (5 mL) and ethyl acetate (5 mL) was stirred at room temperature for 16 h under hydrogen atmosphere. The resultant slurry was filtered to remove the solids and the filtrate was concentrated. The crude product isolated was subjected to prep-HPLC (BOSTON pHlex ODS 10 μm 21.2×250 mm120 A. The mobile phase was acetonitrile/0.1% Ammonium bicarbonate) to obtain tert-butyl 3-(1-(7-morpholino-2-(pyridin-4-yl)pyrazolo[1,5-a]pyrimidin-5-yl)-1H-imidazol-4-yl)piperidine-1-carboxylate as yellow solid (50 mg, 47%). ¹H NMR (400 MHz, DMSO-d₆) δ 8.70 (d, J=6.0 Hz, 2H), 8.62 (s, 1H), 7.99 (dd, J=4.8, 1.2 Hz, 2H), 7.87 (s, 1H), 7.18 (s, 1H), 6.76 (s, 1H), 4.25-3.85 (m, 10H), 3.10-2.73 (m, 2H), 2.65-2.55 (m, 1H), 2.08-2.01 (m, 1H), 1.77-1.43 (m, 3H), 1.41 (s, 9H); LCMS (ESI) m/z: 531.2 [M+H]+.

### Step 4: Synthesis of 4-(5-(4-(piperidin-3-yl)-1H-imidazol-1-yl)-2-(pyridin-4-yl)pyrazolo[1,5-a]pyrimidin-7-yl)morpholine

A mixture of tert-butyl 3-(1-(7-morpholino-2-(pyridin-4-yl)pyrazolo[1,5-a]pyrimidin-5-yl)-1H-imidazol-4-yl)piperidine-1-carboxylate (106 mg, 2 mmol) in dichloromethane (4 mL) and hydrochloric acid/dioxane (4M, 2 mL) was stirred at room temperature for 2 h. The mixture was concentrated, and the residue was subjected to prep-HPLC (BOSTON pHlex ODS 10 μm 21.2×250 mm120 A. The mobile phase was acetonitrile/0.1% Formic acid) to obtain 4-(5-(4-(piperidin-3-yl)-1H-imidazol-1-yl)-2-(pyridin-4-yl)pyrazolo[1,5-a]pyrimidin-7-yl)morpholine as white solid. (50 mg, 58%, isolated as formate salt). ¹H NMR (400 MHz, CD₃OD) δ 9.96 (s, 1H), 8.96 (d, J=6.8 Hz, 2H), 8.75 (d, J=6.8 Hz, 2H), 8.58 (s, 1H), 7.51 (s, 1H), 7.05 (s, 1H), 4.18-4.02 (m, 8H), 3.77-3.74 (m, 1H), 3.52-3.46 (m, 2H), 3.30-3.12 (m, 2H), 2.36-1.96 (m, 3H); LCMS (ESI) m/z: 431.1 [M−HCOOH+H]+.

### Step 5: Synthesis of 4-(2-(pyridin-4-yl)-5-(4-(1,2,5,6-tetrahydropyridin-3-yl)-1H-imidazol-1-yl)pyrazolo[1,5-a]pyrimidin-7-yl)morpholine

A mixture of tert-butyl 5-(1-(7-morpholino-2-(pyridin-4-yl)pyrazolo[1,5-a]pyrimidin-5-yl)-1H-imidazol-4-yl)-3,6-dihydropyridine-1(2H)-carboxylate (53 mg, 1 mmol) in dichloromethane (2 mL) and hydrochloric acid/dioxane (4M, 1 mL) was stirred at room temperature for 2 h. The mixture was concentrated, and the residue was subjected to prep-HPLC (BOSTON pHlex ODS 10 μm 21.2×250 mm120 A. The mobile phase was acetonitrile/0.1% Formic acid) to obtain 4-(2-(pyridin-4-yl)-5-(4-(1,2,5,6-tetrahydropyridin-3-yl)-1H-imidazol-1-yl)pyrazolo[1,5-a]pyrimidin-7-yl)morpholine as white solid. (22 mg, 46%, isolated as formate salt). ¹H NMR (400 MHz, DMSO-d₆) δ 8.70 (d, J=5.6 Hz, 2H), 8.67 (s, 1H), 8.31 (s, 1H), 8.06 (s, 1H), 7.99 (d, J=6.0 Hz, 2H), 7.19 (s, 1H), 6.78 (s, 1H), 6.53 (s, 1H), 3.98-3.89 (m, 8H), 3.72-3.68 (m, 2H), 3.00 (bs, 2H), 2.28 (bs, 2H); LCMS (ESI) m/z: 429.1 [M−HCOOH+H]+.

### Step-6: 4-(5-(4-(1-methylpiperidin-3-yl)-1H-imidazol-1-yl)-2-(pyridin-4-yl)pyrazolo[1,5-a]pyrimidin-7-yl)morpholine

To a solution of 4-(5-(4-(piperidin-3-yl)-1H-imidazol-1-yl)-2-(pyridin-4-yl)pyrazolo[1,5-a]pyrimidin-7-yl)morpholine (40 mg, 0.09 mmol) and 37% formaldehyde (6 drops) in methanol (0.5 mL) and 1,2-dichloroethane (3 mL) was added a drop of acetic acid and the mixture was stirred at room temperature for 1 h. Then sodium cyanoborohydride (29.3 mg, 0.47 mmol) was added to the mixture and the stirring was continued for another 16 h. The reaction was quenched by the addition with water (10 ml) and the mixture was extracted with dichloromethane (10 mL*2). The combined organic phase was concentrated, and the residue was subjected to prep-TLC (Silica, UV254, dichloromethane/methanol=25/1) to obtain 4-(5-(4-(1-methylpiperidin-3-yl)-1H-imidazol-1-yl)-2-(pyridin-4-yl)pyrazolo[1,5-a]pyrimidin-7-yl)morpholine (1.5 mg, 3%) as white solid. ¹H NMR (400 MHz, CDCL3) δ 8.73 (d, J=5.6 Hz, 2H), 8.29 (s, 1H), 7.83 (d, J=5.6 Hz, 2H), 7.46 (s, 1H), 6.89 (s, 1H), 6.13 (s, 1H), 4.07-4.03 (m, 4H), 3.93-3.930 (m, 4H), 3.24-3.21 (m, 1H), 3.10-2.97 (m, 2H), 2.43 (s, 3H), 2.24-2.07 (m, 3H), 1.86 (s, 2H), 1.25 (s, 1H). LCMS(ESI)m/z: 445.3[M+H]*.

### Synthesis of 4-(5-(1-methyl-5-phenylpyrrolidin-3-yl)-2-(pyridin-4-yl)pyrazolo[1,5-a]pyrimidin-7-yl)morpholine (Compound 19)

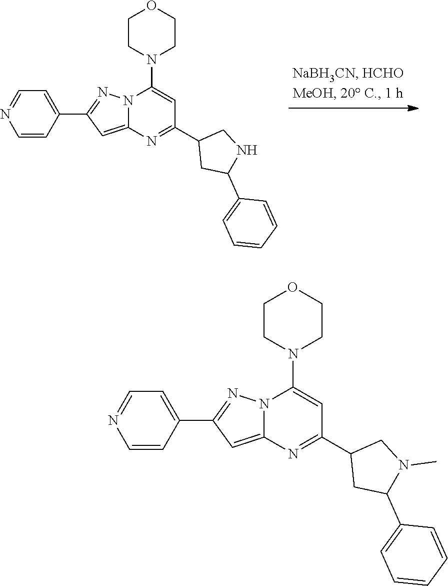 [SMILES: c1ccc(C2CC(c3cc(N4CCOCC4)n4nc(-c5ccncc5)cc4n3)CN2)cc1 ; CN1CC(c2cc(N3CCOCC3)n3nc(-c4ccncc4)cc3n2)CC1c1ccccc1]

To a solution of 4-(5-(5-phenylpyrrolidin-3-yl)-2-(pyridin-4-yl)pyrazolo[1,5-a]pyrimidin-7-yl)morpholine (0.12 g, 0.03 mmol) in methanol (4 mL) were added formaldehyde (0.3 mL), acetic acid (0.05 mL) and sodium cyanoborohydride (0.05 g, 0.8 mmol) at 20° C. The reaction mixture was stirred at 20° C. for 1 h and filtered to remove the solids. The filtrate was concentrated, and the residue was subjected to prep-HPLC (Boston C18 21*250 mm 10 μm column. The mobile phase was acetonitrile/10 mM ammonium bicarbonate aqueous solution.) to obtain 4-(5-(1-methyl-5-phenylpyrrolidin-3-yl)-2-(pyridin-4-yl)pyrazolo[1,5-a]pyrimidin-7-yl)morpholine as white solid.(8.6 mg, 7%). ¹H NMR (400 MHz, DMSO-d6) δ 8.69 (d, J=6.0 Hz, 2H), 7.98 (dd, J=4.6, 1.5 Hz, 2H), 7.42 (d, J=7.4 Hz, 2H), 7.35 (t, J=7.5 Hz, 2H), 7.26 (t, J=7.2 Hz, 1H), 7.19 (s, 1H), 6.52 (s, 1H), 3.95-3.85 (m, 4H), 3.79 (d, J=5.2 Hz, 4H), 3.52 (s, 2H), 3.32-3.25 (m, 1H), 2.74 (s, 1H), 2.62 (s, 1H), 2.12 (s, 3H), 2.06 (s, 1H).; LCMS (ESI) m/z: 441.3 [M+H]+.

### Synthesis of N-hydroxy-7-morpholino-2-(pyridin-4-yl)pyrazolo[1,5-a]pyrimidine-5-carboximidamide (Compound 20) and 4-(5-(5-phenyl-1,2,4-oxadiazol-3-yl)-2-(pyridin-4-yl)pyrazolo[1,5-a]pyrimidin-7-yl)morpholine (Compound 21)

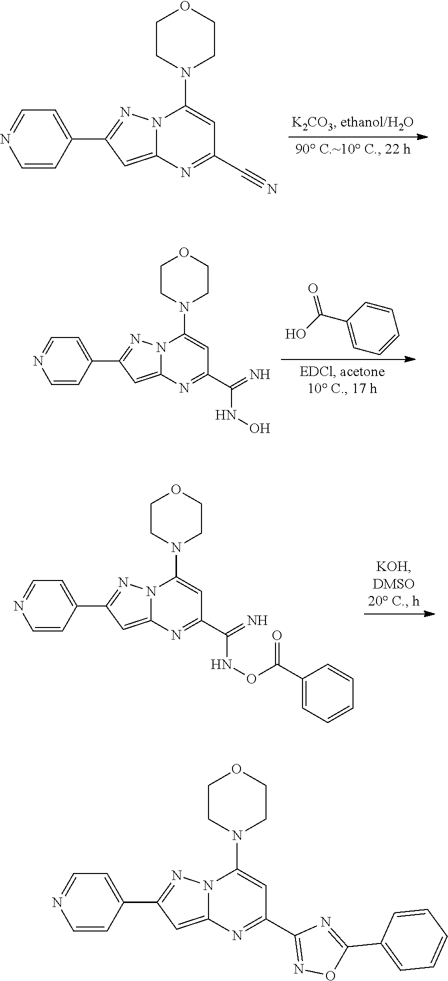 [SMILES: N#Cc1cc(N2CCOCC2)n2nc(-c3ccncc3)cc2n1 ; N=C(NO)c1cc(N2CCOCC2)n2nc(-c3ccncc3)cc2n1 ; O=C(O)c1ccccc1 ; N=C(NOC(=O)c1ccccc1)c1cc(N2CCOCC2)n2nc(-c3ccncc3)cc2n1 ; c1ccc(-c2nc(-c3cc(N4CCOCC4)n4nc(-c5ccncc5)cc4n3)no2)cc1]

### Step 1: Synthesis of N-hydroxy-7-morpholino-2-(pyridin-4-yl)pyrazolo[1,5-a]pyrimidine-5-carboximidamide

To a solution of 7-morpholino-2-(pyridin-4-yl)pyrazolo[1,5-a]pyrimidine-5-carbonitrile (600 mg, 1.96 mmol) in ethanol (14 mL) and water (20 mL) under argon atmosphere were added hydroxylamine hydrochloride (270.5 mg, 3.92 mml) and potassium carbonate (811.4 mg, 5.88 mmol). The mixture was stirred at 90° C. for 5 h and at 10° C. for 17h. It was concentrated and the residue was subjected to prep-HPLC (Boston C18 21*250 mm 10 μm column. The mobile phase was acetonitrile/0.01% aqueous FA.) to obtain N-hydroxy-7-morpholino-2-(pyridin-4-yl)pyrazolo[1,5-a]pyrimidine-5-carboximidamide as yellow solid (400 mg, 60.2%).

### Step 2: Synthesis of N-(benzoyloxy)-7-morpholino-2-(pyridin-4-yl)pyrazolo[1,5-a]pyrimidine-5-carboximidamide

To a solution of benzoic acid (235.5 mg, 1.93 mmol) in acetone (20 mL) was added 1-(3-dimethylaminopropyl)-3-ethylcarbodiimide hydrochloride (741.9 mg, 3.87 mmol) and the mixture was stirred at 10° C. for 30 min. Then N-hydroxy-7-morpholino-2-(pyridin-4-yl)pyrazolo[1,5-a]pyrimidine-5-carboximidamide (440 mg, 1.29 mmol) was added to the mixture and stirring was continued for another 17 h. The mixture was then filtered, the filtrate was concentrated, and the residue was subjected to prep-TLC (DCM: MeOH=15:1) to afford N-(benzoyloxy)-7-morpholino-2-(pyridin-4-yl)pyrazolo[1,5-a]pyrimidine-5-carboximidamide as a yellow solid (200 mg, 34.9%).

### Step 3: Synthesis of 4-(5-(5-phenyl-1,2,4-oxadiazol-3-yl)-2-(pyridin-4-yl)pyrazolo[1,5-a]pyrimidin-7-yl)morpholine

To a mixture of N-(benzoyloxy)-7-morpholino-2-(pyridin-4-yl)pyrazolo[1,5-a]pyrimidine-5-carboximidamide (200 mg, 0.45 mmol) in DMSO (6 mL) was added potassium hydroxide (38.2 mg, 0.67 mmol) and the mixture was stirred at 10° C. for 0.5h. The resultant mixture was then subjected to prep-HPLC (Boston C18 21*250 mm 10 μm column. The mobile phase was acetonitrile/0.01% aqueous formic acid) to obtain 4-(5-(5-phenyl-1,2,4-oxadiazol-3-yl)-2-(pyridin-4-yl)pyrazolo[1,5-a]pyrimidin-7-yl)morpholine (19.9 mg, 10.4%) as white solid. ¹H NMR (400 MHz, DMSO) δ 8.74 (d, J=5.7 Hz, 2H), 8.26 (d, J=7.5 Hz, 2H), 8.05 (d, J=5.8 Hz, 2H), 7.73 (dt, J=15.3, 7.5 Hz, 3H), 7.51 (s, 1H), 7.06 (s, 1H), 3.96 (bs, 4H), 3.93 (bs, 4H); LCMS (ESI) m/z: 426.1 [M+H]+.

### Synthesis of 4-(5-(5-phenyloxazol-2-yl)-2-(pyridin-4-yl)pyrazolo[1,5-a]pyrimidin-7-yl)morpholine (Compound 22)

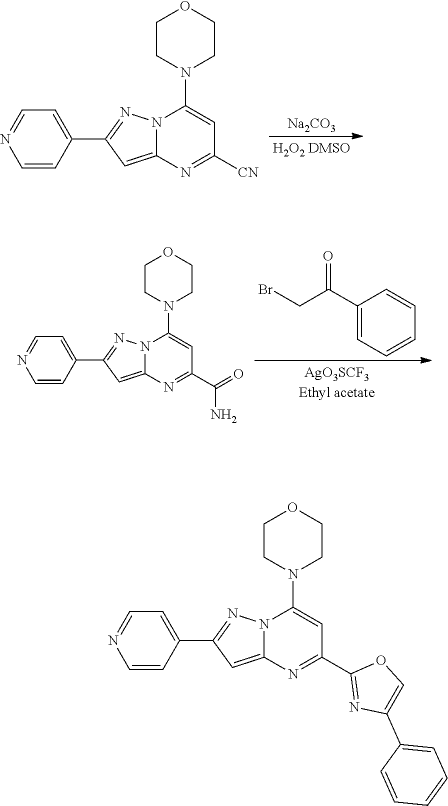

### Step 1: Synthesis of 7-morpholino-2-(pyridin-4-yl)pyrazolo[1,5-a]pyrimidine-5-carboxamide

A mixture of 7-morpholino-2-(pyridin-4-yl)pyrazolo[1,5-a]pyrimidine-5-carbonitrile (1 g, 3.26 mmol), sodium carbonate (3.46 g, 32.6 mmol) and hydrogen peroxide (6 mL) in dimethyl sulfoxide (10 mL) was stirred at 25° C. for 5 h. The resultant mixture was filtered, and the filtrate was concentrated to obtain 7-morpholino-2-(pyridin-4-yl)pyrazolo[1,5-a]pyrimidine-5-carboxamide as off-white solid (350 mg, 33%) LCMS (ESI) m/z: 325.2 [M+H]+.

### Step 2: Synthesis of 4-(5-(5-phenyloxazol-2-yl)-2-(pyridin-4-yl)pyrazolo[1,5-a]pyrimidin-7-yl)morpholine

To a solution of 7-morpholino-2-(pyridin-4-yl)pyrazolo[1,5-a]pyrimidine-5-carboxamide (0.15 g, 0.45 mmol) in ethyl acetate (1.8 mL) were added 2-bromo-1-phenylethan-1-one (0.09 g, 0.42 mmol) and silver trifluoromethanesulfonate (0.12 g, 0.45 mmol) and the resultant mixture was stirred at 60° C. for 4 h under argon atmosphere. The mixture was filtered, the filtrate was concentrated, and the residue was subjected to prep-HPLC (Boston C18 21*250 mm 10 μm column. The mobile phase was acetonitrile/10 mM formic acid aqueous solution.) to obtain 4-(5-(5-phenyloxazol-2-yl)-2-(pyridin-4-yl)pyrazolo[1,5-a]pyrimidin-7-yl)morpholine (11.9 mg, 10%) as white solid. ¹H NMR (400 MHz, DMSO-d6) δ 8.97 (d, J=6.9 Hz, 2H), 8.81 (d, J=6.9 Hz, 2H), 8.54 (s, 1H), 8.20-8.12 (m, 2H), 7.80 (t, J=7.5 Hz, 1H), 7.67 (t, J=7.8 Hz, 2H), 7.64 (s, 1H), 6.97 (s, 1H), 4.10-4.05 (m, 4H), 4.02-3.97 (m, 4H).; LCMS (ESI) m/z: 425.1 [M+H]+.

### Synthesis of 1-(7-morpholino-2-(pyridin-4-yl)pyrazolo[1,5-a]pyrimidin-5-yl)-3-phenylpyrrolidin-2-one (Compound 23)

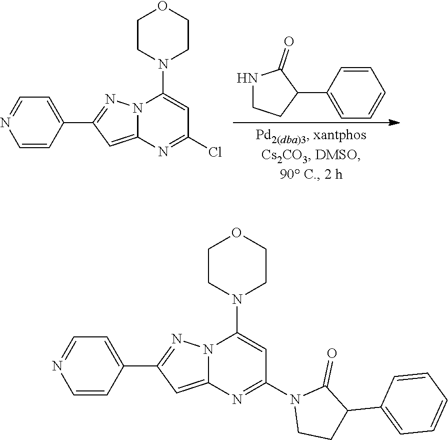 [SMILES: Clc1cc(N2CCOCC2)n2nc(-c3ccncc3)cc2n1 ; O=C1NCCC1c1ccccc1 ; O=C1C(c2ccccc2)CCN1c1cc(N2CCOCC2)n2nc(-c3ccncc3)cc2n1]

To a solution of 4-(5-chloro-2-(pyridin-4-yl)pyrazolo[1,5-a]pyrimidin-7-yl)morpholine (350 mg, 1.1 mmol) in dimethyl sulfoxide (35 mL) were added 3-phenylpyrrolidin-2-one (195 mg, 1.21 mmol), cesium carbonate (717 mg, 2.2 mmol), tris(dibenzylideneacetone)dipalladium(0) (101 mg, 0.11 mmol) and 4,5-bis(diphenylphosphino)-9,9-dimethylxanthene (64 mg, 0.11 mmol) at 90° C. and the resultant mixture was stirred at that temperature for 2 h. The mixture was cooled and extracted with ethyl acetate (80 mL*2). The combined organic phase was washed with water (60 mL) and brine (60 mL), dried over sodium sulfate and concentrated. The residue was subjected to prep-HPLC (BOSTON pHlex ODS 10 μm 21.2×250 mm 120 A. The mobile phase was acetonitrile/0.1% aqueous formic acid) to obtain 1-(7-morpholino-2-(pyridin-4-yl)pyrazolo[1,5-a]pyrimidin-5-yl)-3-phenylpyrrolidin-2-one (85.9 mg, 17.7%) as white solid. ¹H NMR (400 MHz, DMSO-d6) δ 8.69 (d, J=5.0 Hz, 2H), 7.97 (d, J=5.0 Hz, 2H), 7.57 (s, 1H), 7.46-7.24 (m, 5H), 7.15 (s, 1H), 4.25 (t, J=8.8 Hz, 1H), 4.11 (t, J=9.5 Hz, 1H), 3.98 (dd, J=18.6, 8.6 Hz, 1H), 3.86 (d, J=4.1 Hz, 4H), 3.79-3.69 (m, 4H), 2.59-2.55 (m, 1H), 2.26-2.21 (m, 1H); LCMS (ESI) m/z: 441.1 [M+H]+.

The following compounds were synthesized according to the protocol described above:

| Name | Structure | NMR, LC-MS | Comp # |
|---|---|---|---|
| 1-(7-morpholino-2- (pyridin-4- yl)pyrazolo[1,5-a] pyrimidin-5- yl)-3- phenylpiperidin- 2-one | 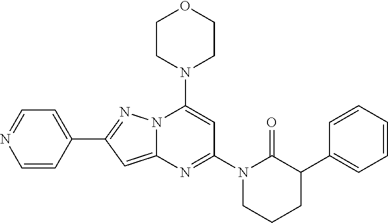 [SMILES: O=C1C(c2ccccc2)CCCN1c1cc(N2CCOCC2)n2nc(-c3ccncc3)cc2n1] | 1H NMR (400 MHz, DMSO-d6) δ 8.69 (d, J = 6.0 Hz, 2H), 7.98 (dd, J = 4.4, 1.6 Hz, 2H), 7.36-7.24 (m, 5H), 7.20 (s, 1H), 6.98 (s, 1H), 4.20 − 4.15 (m, 1H), 4.04-3.95 (m, 2H), 3.85 (t, J = 4.6 Hz, 4H), 3.75 − 3.66 (m, 4H), 2.27 − 2.13 (m, 1H), 2.11 − 1.95 (m, 3H); LCMS (ESI) m/z: 455.3 [M + H]+. | 24 |
| 1-(7-morpholino- 2-(pyridin-4- yl)pyrazolo[1,5- a]pyrimidin- 5-yl)-4- phenylpyrrolidin- 2-one | 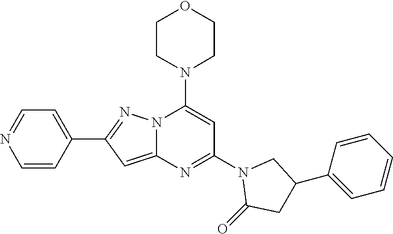 [SMILES: O=C1CC(c2ccccc2)CN1c1cc(N2CCOCC2)n2nc(-c3ccncc3)cc2n1] | 1H NMR (400 MHz, DMSO-d6) δ 8.68 (d, J = 5.8 Hz, 2H), 7.96 (dd, J = 4.6, 1.4 Hz, 2H), 7.58 (s, 1H), 7.46 − 7.34 (m, 4H), 7.31-7.27 (m, 1H), 7.09 (s, 1H), 4.54-4.49 (m, 1H), 4.02 − 3.86 (m, 5H), 3.83 − 3.65 (m, 5H), 3.07- 3.01 (m, 1H), 2.92-2.86 (m, 1H); LCMS (ESI) m/z: 441.1[M + H]+. | 25 |
| 1-(7- morpholino- 2-(pyridin-4- yl)pyrazolo[1, 5- a]pyrimidin- 5-yl)-5- phenylpiperidin- 2-one | 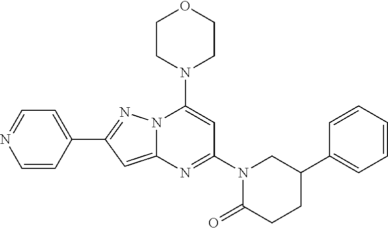 [SMILES: O=C1CCC(c2ccccc2)CN1c1cc(N2CCOCC2)n2nc(-c3ccncc3)cc2n1] | 1H NMR (400 MHz, DMSO-d6) 1H NMR (400 MHz, DMSO) δ 8.68 (dd, J = 4.6, 1.4 Hz, 2H), 7.94 (dd, J = 4.4, 1.6 Hz, 2H), 7.48 − 7.33 (m, 4H), 7.32 − 7.23 (m, 1H), 7.13 (s, 1H), 7.06 (s, 1H), 4.23-4.19 (m, 1H), 3.89-3.81 (m, 5H), 3.80-3.79 (m, 2H), 3.71 − 3.65 (m, 2H), 3.28-3.23 (m, 1H), 2.81 − 2.66 (m, 2H), 2.24-2.17 (m, 1H), 2.08- 2.05 (m, 1H); LCMS (ESI) m/z: 455.3[M + H]+. | 26 |

### Synthesis of 4-methyl-1-(7-morpholino-2-(pyridin-4-yl)pyrazolo[1,5-a]pyrimidin-5-yI)-3-phenylpiperazin-2-one (Compound 27)

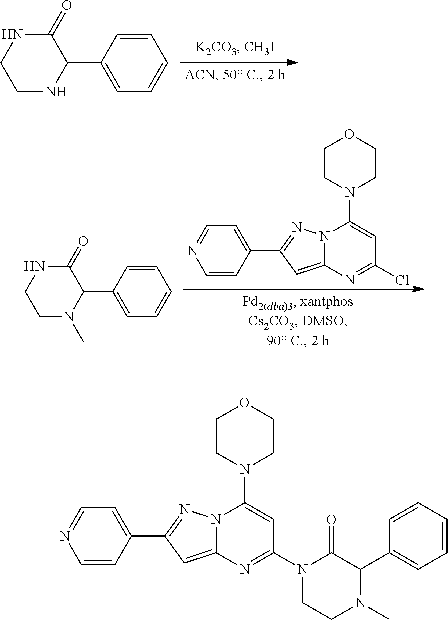

### Step 1: Synthesis of 4-methyl-3-phenylpiperazin-2-one

To a solution of 3-phenylpiperazin-2-one (650 mg, 3.69 mmol) in acetonitrile (50 mL) were added potassium carbonate (1.02 g, 7.38 mmol) and iodomethane (551 mg, 3.88 mmol) and the mixture was stirred at 50° C. for 2h. It was cooled, filtered to remove the solids and the filtrate was concentrated. The residue was subjected to flash column chromatography [(dichloromethane: ammonia in methanol (7N)=30:1)] to obtain 4-methyl-3-phenylpiperazin-2-one as a white solid (0.4 g, 57%). LCMS (ESI) m/z: 191.3 [M+H]+.

### Step 2: Synthesis of 4-methyl-1-(7-morpholino-2-(pyridin-4-yl)pyrazolo[1,5-a]pyrimidin-5-yl)-3-phenylpiperazin-2-one

To a solution of 4-(5-chloro-2-(pyridin-4-yl)pyrazolo[1,5-a]pyrimidin-7-yl)morpholine (315 mg, 0.63 mmol) in dimethyl sulfoxide (30 mL) were added 4-methyl-3-phenylpiperazin-2-one (247 mg, 1.3 mmol), cesium carbonate (652 mg, 2 mmol), tris(dibenzylideneacetone)dipalladium(0) (92 mg, 0.1 mmol) and 4,5-bis(diphenylphosphino)-9,9-dimethylxanthene (58 mg, 0.1 mmol) at 90° C. The resulting mixture was stirred at 90° C. for 2 h, then cooled and the mixture was extracted with ethyl acetate (60 mL*2). The combined organic layer was washed with water (60 mL), brine (60 mL), dried over sodium sulfate and concentrated. The residue was subjected to prep-HPLC (BOSTON pHlex ODS 10 μm 21.2×250 mm 120 A. The mobile phase was acetonitrile/0.1% Ammonium bicarbonate) to obtain 4-methyl-1-(7-morpholino-2-(pyridin-4-yl)pyrazolo[1,5-a]pyrimidin-5-yl)-3-phenylpiperazin-2-one as white solid (31.5 mg, 6.7%). 1H NMR (400 MHz, DMSO-d6) δ 8.69 (dd, J=4.6, 1.6 Hz, 2H), 7.97 (dd, J=4.6, 1.6 Hz, 2H), 7.41 (dd, J=8.2, 1.4 Hz, 2H), 7.38-7.29 (m, 3H), 7.20 (s, 1H), 7.15 (s, 1H), 4.32-4.17 (m, 1H), 4.08-3.94 (m, 2H), 3.84 (t, J=4.6 Hz, 4H), 3.76-3.59 (m, 4H), 3.22 (dd, J=9.0, 3.0 Hz, 1H), 2.73-2.50 (m, 1H), 2.13 (s, 3H); LCMS (ESI) m/z: 470.3 [M+H]+.

### Synthesis of 3-benzyl-1-methyl-4-(7-morpholino-2-(pyridin-4-yl)pyrazolo[1,5-a]pyrimidin-5-yl)piperazin-2-one (Compound 28) and 4-(5-fluoro-2-(pyridin-4-yl)pyrazolo[1,5-a]pyrimidin-7-yl)morpholine (Compound 29)

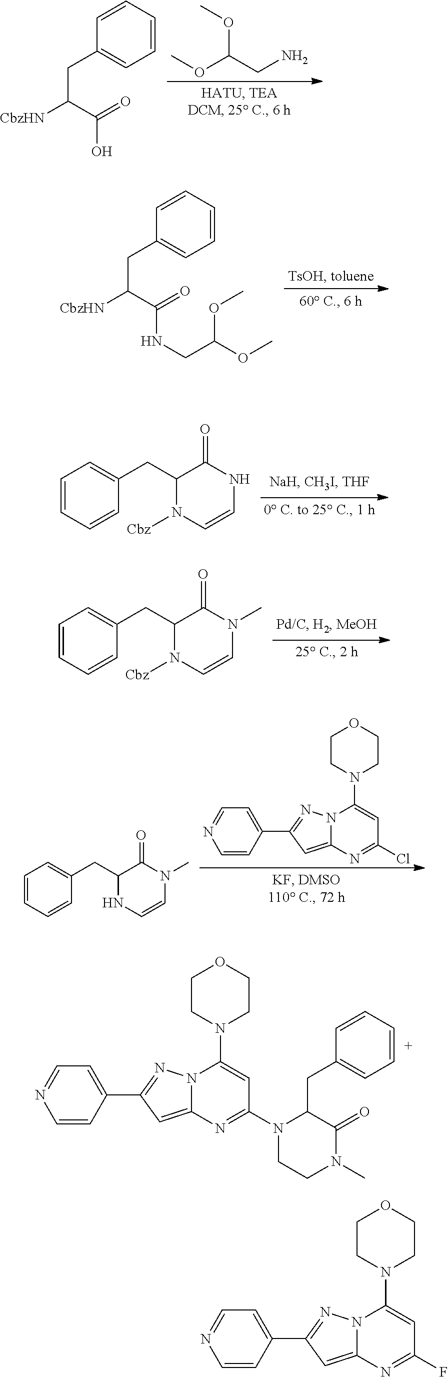

### Step 1: Synthesis of benzyl (1-((2,2-dimethoxyethyl)amino)-1-oxo-3-phenylpropan-2-yl)carbamate

To a solution of ((benzyloxy)carbonyl)phenylalanine (2.99 g, 10.0 mmol) in dichloromethane (100 mL) were added triethylamine (3.03 g, 30.0 mmol), 1-[Bis(dimethylamino)methylene]-1H-1,2,3-triazolo[4,5-b]pyridinium 3-oxid hexafluorophosphate (3.80 g, 10.0 mmol) and 2,2-dimethoxyethan-1-amine (1.16 g, 11.0 mmol) at 25° C. under argon atmosphere. The mixture was stirred at that temperature for 6h and then diluted with water (50 mL). The resultant heterogeneous mixture was washed with 1N hydrochloric acid (50 mL*2) and sodium bicarbonate aqueous(50 mL*2). The organic layer was dried over sodium sulfate, filtered and concentrated. The title compound was obtained as colorless oil (5.1 g) which was used in the next step without further purification. LCMS (ESI) m/z: 355.2 [M+H]+.

### Step 2: Synthesis of benzyl 2-benzyl-3-oxo-3,4-dihydropyrazine-1(2H)-carboxylate

To a solution of benzyl (1-((2,2-dimethoxyethyl)amino)-1-oxo-3-phenylpropan-2-yl)carbamate (4.7 g, 9.0 mmol) in toluene (50 mL) was added 4-methylbenzenesulfonic acid (155 mg, 0.9 mmol) at 25° C. under argon. The mixture was stirred at 60° C. for 16 h, then cooled and diluted with ethyl acetate (150 mL). The organic layer was separated, washed with water (80 mL), sodium bicarbonate aqueous (80 mL), dried over sodium sulfate, filtered and concentrated. The residue was subjected to silica gel column chromatography (dichloromethane:ethyl acetate=8:1 to 4:1) to obtain benzyl 2-benzyl-3-oxo-3,4-dihydropyrazine-1(2H)-carboxylate as a colorless oil (2.27 g, 78%). LCMS (ESI) m/z: 323.1 [M+H]+.

### Step 3: Synthesis of benzyl 2-benzyl-3-oxo-3,4-dihydropyrazine-1(2H)-carboxylate

To a solution of benzyl 2-benzyl-3-oxo-3,4-dihydropyrazine-1(2H)-carboxylate (967 mg, 3.0 mmol) in tetrahydrofuran (50 mL) was added sodium hydride (132 mg, 3.3 mmol) at 0° C. under argon atmosphere. The mixture was warmed up and stirred at 25° C. for 1 h. Then iodomethane (468 mg, 3.3 mmol) was added and the mixture was stirred at 25° C. for another 16 h. The reaction was quenched with water, the mixture was extracted with dichloromethane (80 mL*2), the combined organic layer was dried over sodium sulfate, filtered and concentrated. The residue was subjected to silica gel column chromatography (dichloromethane:ethyl acetate=16:1 to 8:1) to obtain benzyl 2-benzyl-4-methyl-3-oxo-3,4-dihydropyrazine-1(2H)-carboxylate as a white solid (910 mg, 90%). LCMS (ESI) m/z: 337.2 [M+H]+.

### Step 4: Synthesis of 3-benzyl-1-methylpiperazin-2-one

To a solution of benzyl 2-benzyl-4-methyl-3-oxo-3,4-dihydropyrazine-1(2H)-carboxylate (960 mg, 2.85 mmol) in methanol (50 mL) was added palladium on activated carbon [10% Pd (100 mg)] at 25° C. The resultant slurry was stirred under hydrogen atmosphere at 25° C. for 2 h. The mixture was then filtered through a bed of celite and washed with methanol. The filtrates were collected and concentrated. The residue was subjected to silica gel column chromatography (dichloromethane: ammonia in methanol (7N)=10:1) to obtain 3-benzyl-1-methylpiperazin-2-one as a pink-colored oil (600 mg, 93%). LCMS (ESI) m/z: 205.1 [M+H]+.

### Step 5: Synthesis of 3-benzyl-1-methyl-4-(7-morpholino-2-(pyridin-4-yl)pyrazolo[1,5-a]pyrimidin-5-yl)piperazin-2-one and 4-(5-fluoro-2-(pyridin-4-yl)pyrazolo[1,5-a]pyrimidin-7-yl)morpholine

To a solution of 4-(5-chloro-2-(pyridin-4-yl)pyrazolo[1,5-a]pyrimidin-7-yl)morpholine (316 mg, 1.0 mmol) and 3-benzyl-1-methylpiperazin-2-one (204 mg, 1.0 mmol) in dimethyl sulfoxide (10 mL) was added potassium fluoride (175 mg, 3.0 mmol) at 25° C. under nitrogen atmosphere. The mixture was stirred at 110° C. for 72h and then diluted with ethyl acetate (100 mL). The resultant mixture was washed with water (80 mL*3), the organic layer was dried over sodium sulfate, filtered and concentrated. The residue was subjected to prep-HPLC (Boston C18 21*250 mm 10 μm column. The mobile phase was acetonitrile/10 mM ammonium acetate aqueous solution.) to obtain 3-benzyl-1-methyl-4-(7-morpholino-2-(pyridin-4-yl)pyrazolo[1,5-a]pyrimidin-5-yl)piperazin-2-one as yellow solid (24.0 mg, 5%). 1H NMR (400 MHz, DMSO-d6) δ 8.63 (dd, J=4.4, 1.6 Hz 2H), 7.85 (dd, J=4.4, 1.6 Hz, 2H), 7.20-7.25 (m, 4H), 7.13-7.18 (m, 1H), 6.66 (s, 1H), 5.40 (bs,1H), 5.04 (bs,1H), 4.52 (bs,1H), 3.78-3.84 (m, 4H), 3.41-3.50 (m, 5H), 3.14-3.34 (m, 4H), 2.93 (s, 3H); LCMS (ESI) m/z: 484.2 [M+H]+. And the by-product 4-(5-fluoro-2-(pyridin-4-yl)pyrazolo[1,5-a]pyrimidin-7-yl)morpholine as yellow solid (37.6 mg, 13%). 1H NMR (400 MHz, DMSO-d6) δ 8.70 (dd, J₁=4.4, 1.2 Hz, 2H), 7.97 (dd, J₁=4.8, 1.6 Hz, 2H), 7.27 (s, 1H), 6.31 (s, 1H), 3.91-3.93 (m, 4H), 3.84-3.87 (m, 4H); LCMS (ESI) m/z: 300.1 [M+H]+.

### Synthesis of 4-(5-(2-benzylpiperidin-1-yl)-2-(pyridin-4-yl)pyrazolo[1,5-a]pyrimidin-7-yl)morpholine (Compound 30) and N,N-dimethyl-7-morpholino-2-(pyridin-4-yl)pyrazolo[1,5-a]pyrimidin-5-amine(Compound 31)

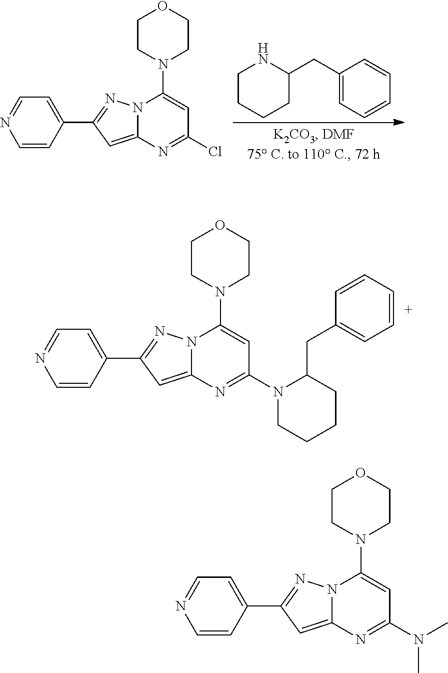 [SMILES: Clc1cc(N2CCOCC2)n2nc(-c3ccncc3)cc2n1 ; c1ccc(CC2CCCCN2)cc1 ; c1ccc(CC2CCCCN2c2cc(N3CCOCC3)n3nc(-c4ccncc4)cc3n2)cc1 ; CN(C)c1cc(N2CCOCC2)n2nc(-c3ccncc3)cc2n1]

To a solution of 4-(5-chloro-2-(pyridin-4-yl)pyrazolo[1,5-a]pyrimidin-7-yl)morpholine (158 mg, 0.5 mmol) and 2-benzylpiperidine (130 mg, 0.75 mmol) in N,N-dimethylformamide (5 mL) was added potassium carbonate (173 mg, 1.25 mmol) at 25° C. under nitrogen atmosphere. The resulting mixture was heated to 110° C. and stirred for 72h. It was diluted with ethyl acetate (50 mL), washed with brine (30 mL*3) and the organic layer was dried over sodium sulfate, filtered and concentrated. The residue was subjected to prep-HPLC (Boston C18 21*250 mm 10 μm column. The mobile phase was acetonitrile/10 mM ammonium acetate aqueous solution.) to obtain 4-(5-(2-benzylpiperidin-1-yl)-2-(pyridin-4-yl)pyrazolo[1,5-a]pyrimidin-7-yl)morpholine as yellow solid (10.2 mg, 4%). 1H NMR (400 MHz, DMSO-d6) δ 8.63 (dd, J=4.4, 1.2 Hz, 2H), 7.87 (dd, J=4.8, 1.6 Hz, 2H), 7.23-7.30 (m, 4H), 7.15 (t, J=6.8 Hz, 1H), 6.63 (s, 1H), 5.68 (s, 1H), 4.78-4.80 (m, 1H), 4.42-4.46 (m, 1H), 3.82-3.86 (m, 4H), 3.52-3.59 (m, 4H), 3.08-3.14 (m, 1H), 2.89-2.98 (m, 2H), 1.77-1.82 (m, 2H), 1.42-1.62 (m, 4H); LCMS (ESI) m/z: 455.3 [M+H]+. And the byproduct N,N-dimethyl-7-morpholino-2-(pyridin-4-yl)pyrazolo[1,5-a]pyrimidin-5-amine (43.9 mg, 27%). as yellow solid. 1H NMR (400 MHz, DMSO-d6) δ 8.64 (dd, J=4.4, 1.6 Hz, 2H), 7.87 (dd, J=4.8, 1.6 Hz, 2H), 6.64 (s, 1H), 5.77 (s, 1H), 3.85-3.87 (m, 4H), 3.63-3.66 (m, 4H), 3.82-3.12 (s, 6H); LCMS (ESI) m/z: 325.2 [M+H]+. The byproduct is likely formed from the dimethylamine impurity present in the solvent.

### Synthesis of 4-(5-(3-phenylazetidin-1-yl)-2-(pyridin-4-yl)pyrazolo[1,5-a]pyrimidin-7-yl)morpholine (Compound 32)

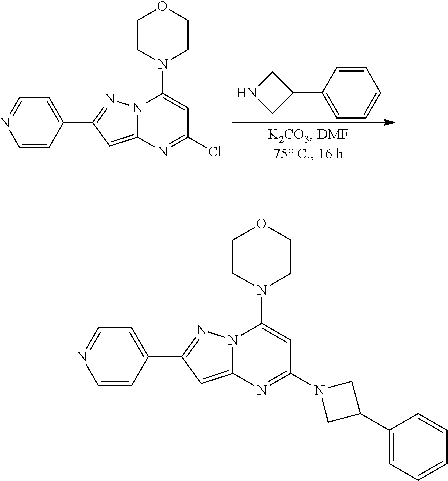 [SMILES: Clc1cc(N2CCOCC2)n2nc(-c3ccncc3)cc2n1 ; c1ccc(C2CNC2)cc1 ; c1ccc(C2CN(c3cc(N4CCOCC4)n4nc(-c5ccncc5)cc4n3)C2)cc1]

To a solution of 4-(5-chloro-2-(pyridin-4-yl)pyrazolo[1,5-a]pyrimidin-7-yl)morpholine (150 mg, 0.5 mmol) in DMF (3 mL) were added potassium carbonate (160 mg, 1.2 mmol) and 3-phenylazetidine (95 mg, 0.7 mmol). The mixture was stirred at 75° C. for 16 h, then filtered to remove the solids and the filtrate was subjected to prep-HPLC (Boston 018 21*250 mm 10 μm column. The mobile phase was acetonitrile/10 mM ammonium bicarbonate aqueous solution.) to obtain 4-(5-(3-phenylazetidin-1-yl)-2-(pyridin-4-yl)pyrazolo[1,5-a]pyrimidin-7-yl)morpholine as a white solid (139.8 mg, 71%). ¹H NMR (400 MHz, DMSO-d₆) δ 8.65 (dd, J=4.5, 1.6 Hz, 2H), 7.88 (dd, J=4.5, 1.6 Hz, 2H), 7.43-7.35 (m, 4H), 7.31-7.22 (i, 1H), 6.70 (s, 1H), 5.56 (s, 11H), 4.50 (t, J=8.4 Hz, 2H), 4.11-4.04 (i, 2H), 3.99 (dt, J=14.3, 7.0 Hz, 1H), 3.89-3.82 (m, 4H), 3.71-3.60 (s, 4H).; LCMS (ESI) m/z: 413.2 [M+H]+.

The following compounds were synthesized according to the protocol described above:

| Name | Structure | NMR, MS | Compd # |
|---|---|---|---|
| 4-(5-(3- phenylpyrrolidin- 1-yl)-2- (pyridin-4- yl)pyrazolo[1,5- a]pyrimidin-7- yl)morpholine | 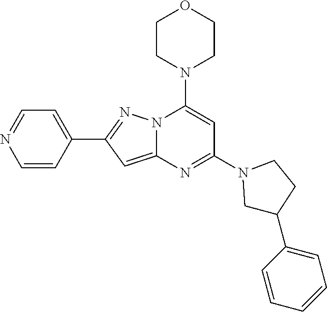 [SMILES: c1ccc(C2CCN(c3cc(N4CCOCC4)n4nc(-c5ccncc5)cc4n3)C2)cc1] | ¹H NMR (400 MHz, DMSO-d₆) δ 8.64 (d, J = 4.8 Hz, 2H), 7.88 (d, J = 4.8 Hz, 2H), 7.43 − 7.31 (m, 4H), 7.26 (s, 1H), 6.66 (s, 1H), 5.68 (s, 1H), 4.03 (s, 1H), 3.85 (s, 4H), 3.81 − 3.73 (m, 1H), 3.65 (d, J = 5.1 Hz, 4H), 3.56 (d, J = 7.5 Hz, 2H), 3.45 (t, J = 9.5 Hz, 1H), 2.37 (s, 1H), 2.11 (d, J = 10.2 Hz, 1H).; LCMS (ESI) m/z: 427.2 [M + H]+. | 33 |
| 4-(5-(3- phenylpiperidin- 1-yl)-2- (pyridin-4- yl)pyrazolo[1,5- a]pyrimidin-7- yl)morpholine | 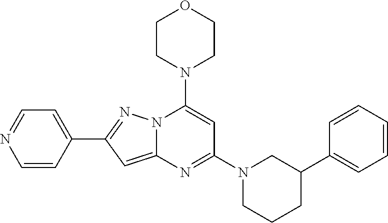 [SMILES: c1ccc(C2CCCN(c3cc(N4CCOCC4)n4nc(-c5ccncc5)cc4n3)C2)cc1] | ¹H NMR (400 MHz, DMSO-d₆) δ 8.64 (dd, J = 4.5, 1.5 Hz, 2H), 7.87 (dd, J = 4.5, 1.5 Hz, 2H), 7.39 − 7.31 (m, 4H), 7.30 − 7.19 (m, 1H), 6.66 (s, 1H), 5.96 (s, 1H), 4.52 (d, J = 12.4 Hz, 2H), 3.94 − 3.77 (m, 4H), 3.65 (d, J = 4.7 Hz, 4H), 3.08 − 2.90 (m, 2H), 2.78 − 2.65 (m, 1H), 1.95 (d, J = 10.8 Hz, 1H), 1.77 (dt, J = 12.3, 9.2 Hz, 2H), 1.61 (t, J = 12.4 Hz, 1H).; LCMS (ESI) m/z: 441.3 [M + H]+. | 34 |
| 4-(5-(3- phenylpiperazin- 1-yl)-2- (pyridin-4- yl)pyrazolo[1,5- a]pyrimidin-7- yl)morpholine | 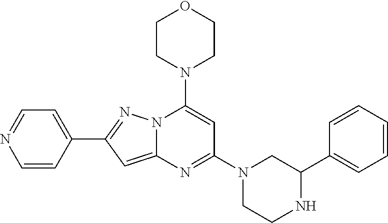 [SMILES: c1ccc(C2CN(c3cc(N4CCOCC4)n4nc(-c5ccncc5)cc4n3)CCN2)cc1] | ¹H NMR (400 MHz, DMSO-d₆) δ 8.64 (dd, J = 4.5, 1.6 Hz, 2H), 7.87 (dd, J = 4.5, 1.6 Hz, 2H), 7.49 (d, J = 7.1 Hz, 2H), 7.38 (t, J = 7.3 Hz, 2H), 7.30 (t, J = 7.3 Hz, 1H), 6.66 (s, 1H), 5.95 (s, 1H), 4.37 (s, 2H), 3.90 − 3.80 (m, 4H), 3.72 (d, J = 8.1 Hz, 1H), 3.65 (d, J = 4.5 Hz, 4H), 3.09 (d, J = 11.1 Hz, 1H), 2.96 (t, J = 12.1 Hz, 1H), 2.87 − 2.70 (m, 3H).; LCMS (ESI) m/z: 442.2 [M + H]+. | 35 |
| 3-benzyl-4-(7- morpholino-2- (pyridin-4- yl)pyrazolo[1,5- a]pyrimidin-5- yl)morpholine | 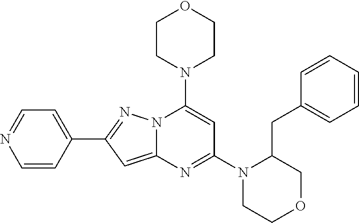 [SMILES: c1ccc(CC2COCCN2c2cc(N3CCOCC3)n3nc(-c4ccncc4)cc3n2)cc1] | ¹H NMR (400 MHz, DMSO-d₆) δ 8.64 (dd, J = 4.6, 1.4 Hz, 2H), 7.88 (dd, J = 4.6, 1.4 Hz, 2H), 7.35 − 7.22 (m, 4H), 7.16 (t, J = 6.9 Hz, 1H), 6.69 (s, 1H), 5.70 (s, 1H), 4.52 (s, 1H), 4.23 (d, J = 11.2 Hz, 1H), 4.01 (dd, J = 11.2, 2.9 Hz, 1H), 3.84 (t, J = 4.6 Hz, 4H), 3.74 (d, J = 11.5 Hz, 1H), 3.64 − 3.43 (m, 6H), 3.38 −3.31 (m, 1H), 3.08 − 2.83 (m, 2H); LCMS (ESI) m/z: 457.2 [M + H]+. | 36 |

### Synthesis of 1-methyl-4-(7-morpholino-2-(pyridin-4-yl)pyrazolo[1,5-a]pyrimidin-5-yl)-6-phenylpiperazin-2-one (Compound 37)

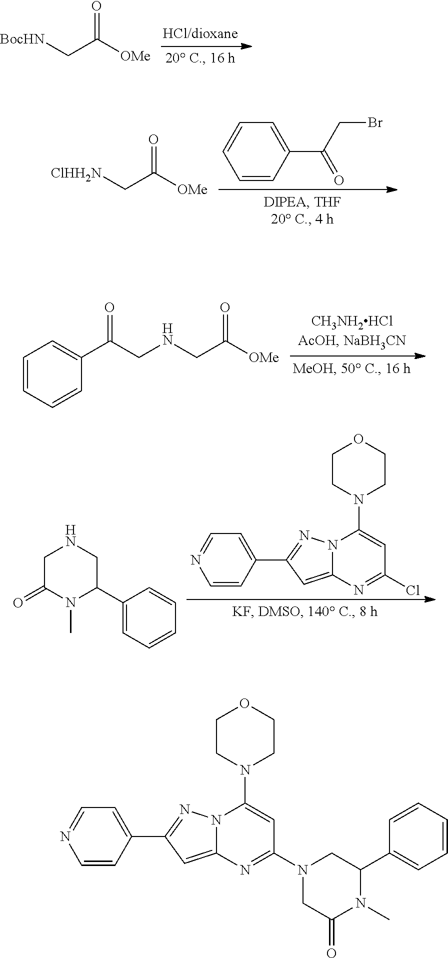

### Step 1: Synthesis of methyl 2-aminoacetate hydrochloride

A solution of methyl 2-(tert-butoxycarbonylamino)acetate (5.0 g, 26.44 mmol) in hydrochloric acid/dioxane (40 mL) was stirred at 20° C. for 16 h and concentrated to obtain methyl 2-amninoacetate hydrochloride as white solid (3.0 g). ¹H NMR (400 MHz, DMSO) δ 8.69 (s, 3H), 3.77 (s, 2H), 3.73 (s, 3H).

### Step 2: Synthesis of methyl 2-(2-oxo-2-phenylethylamino)acetate

A solution of methyl 2-aminoacetate hydrochloride (2.0 g, 16.00 mmol) and DIPEA (5.17 g, 40.00 mmol) in tetrahydrofuran (50 mL) was stirred at 20° C. for 4 h. Then a solution of 2-bromo-1-phenylethanone (3.17 g, 16.00 mmol) in tetrahydrofuran (5 mL) was added to the mixture and stirring was continued for another 4 h. The resultant mixture was filtered to remove the solids and the filtrate was concentrated. The residue obtained was subjected to silica gel column chromatography (petroleum ether/acetic ester=10:1) to obtain methyl 2-(2-oxo-2-phenylethylamino)acetate as a yellow oil (1.21 g). LCMS (ESI) m/z: 208.1 [M+H]+.

### Step 3: Synthesis of 1-methyl-6-phenylpiperazin-2-one

To a solution of methyl 2-(2-oxo-2-phenylethylamino)acetate (1.2 g, 5.79 mmol) and methanamine hydrochloride (582 mg, 8.691 mmol) in methanol (6 mL) was added a drop of acetic acid. The mixture was then stirred at 20° C. for 30 min and then sodium cyanoborohydride (548 mg, 8.691 mmol) was added the mixture and stirring was continued at 50° C. for another 16 h. The mixture was then concentrated, and the residue was subjected to silica gel column chromatography (dichloromethane:/methanol=20:1) to obtain 1-methyl-6-phenylpiperazin-2-one(610 mg) as light-yellow oil. LCMS (ESI) m/z: 191.1 [M+H]+.

### Step 4: Synthesis of 1-methyl-4-(7-morpholino-2-(pyridin-4-yl)pyrazolo[1,5-a]pyrimidin-5-yl)-6-phenylpiperazin-2-one

To a solution of 4-(5-chloro-2-(pyridin-4-yl)pyrazolo[1,5-a]pyrimidin-7-yl)morpholine (182 mg, 0.579 mmol) and 1-methyl-6-phenylpiperazin-2-one (110 mg, 0.579 mmol) in DMSO (5 mL) was added potassium fluoride (117 mg, 2.027 mmol). The mixture was stirred at 140° C. for 8 h and the desired product formed was isolated from the crude mixture by prep-HPLC (SunFire C18, 4.6*50 mm, 3.5 um column Xbridge C18 3.5 μm 4.6×50 mm column. The elution system used was a gradient of 5%-95% over 1.5 min at 2 ml/min and the solvent was acetonitrile/0.01% aqueous NH₄HCO₃). The compound 1-methyl-4-(7-morpholino-2-(pyridin-4-yl)pyrazolo[1,5-a]pyrimidin-5-yl)-6-phenylpiperazin-2-one (15.7 mg, 6%) was obtained as white solid. ¹H NMR (400 MHz, DMSO) δ 8.63 (d, J=5.9 Hz, 2H), 7.84 (dd, J=4.5, 1.5 Hz, 2H), 7.35-7.23 (m, 5H), 6.65 (s, 1H), 5.61 (s, 1H), 4.79 (t, J=3.5 Hz, 1H), 4.70 (d, J=17.8 Hz, 1H), 4.40-4.29 (m, 1H), 4.14 (d, J=17.7 Hz, 1H), 3.93 (dd, J=13.8, 3.9 Hz, 1H), 3.79 (t, J=4.6 Hz, 4H), 3.66-3.46 (m, 4H), 2.78 (s, 3H); LCMS (ESI) m/z: 470.1 [M+H]+.

### Synthesis of 4-(5-(4-methyl-3-phenylpiperazin-1-yl)-2-(pyridin-4-yl)pyrazolo[1,5-a]pyrimidin-7-yl)morpholine (Compound 38)

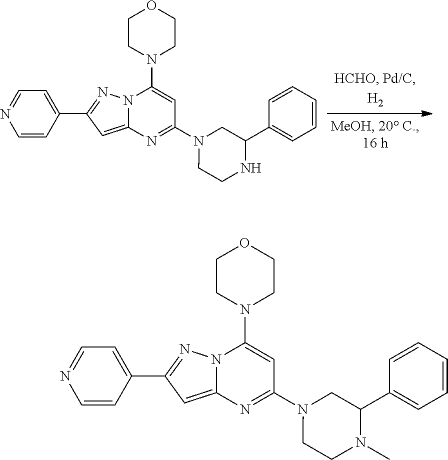 [SMILES: c1ccc(C2CN(c3cc(N4CCOCC4)n4nc(-c5ccncc5)cc4n3)CCN2)cc1 ; CN1CCN(c2cc(N3CCOCC3)n3nc(-c4ccncc4)cc3n2)CC1c1ccccc1]

To a solution of 4-(5-(3-phenylpiperazin-1-yl)-2-(pyridin-4-yl)pyrazolo[1,5-a]pyrimidin-7-yl)morpholine (75 mg, 0.3 mmol) in methanol (4 mL) were added formalin (0.6 mL) and palladium on activated carbon 10% (200 mg). The resultant mixture was stirred at 20° C. for 16 h under hydrogen atmosphere. It was then filtered to remove the solids and the filtrate was concentrated. The residue was then subjected to prep-HPLC (Boston C18 21*250 mm 10 μm column. The mobile phase was acetonitrile/10 mM ammonium bicarbonate aqueous solution.) to obtain 4-(5-(4-methyl-3-phenylpiperazin-1-yl)-2-(pyridin-4-yl)pyrazolo[1,5-a]pyrimidin-7-yl)morpholine as off-white solid (2.8 mg, 3.6%). ¹H NMR (400 MHz, DMSO-d6) δ 8.63 (d, J=6.0 Hz, 2H), 7.86 (dd, J=4.6, 1.5 Hz, 2H), 7.48-7.37 (m, 4H), 7.33 (t, J=6.9 Hz, 1H), 6.65 (s, 1H), 5.96 (s, 1H), 4.46 (d, J=12.8 Hz, 1H), 4.30 (d, J=12.6 Hz, 1H), 3.94-3.75 (m, 4H), 3.65 (d, J=4.4 Hz, 4H), 3.13 (t, J=11.2 Hz, 1H), 3.01 (dd, J=13.8, 6.1 Hz, 2H), 2.91-2.79 (m, 1H), 2.25 (dd, J=11.9, 9.1 Hz, 1H), 1.97 (s, 3H).; LCMS (ESI) m/z: 456.3 [M+H]+.

### Synthesis of 4-(5-(2-benzylpyridin-3-yl)-2-(pyridin-4-yl)pyrazolo[1,5-a]pyrimidin-7-yl)morpholine (Compound 39)

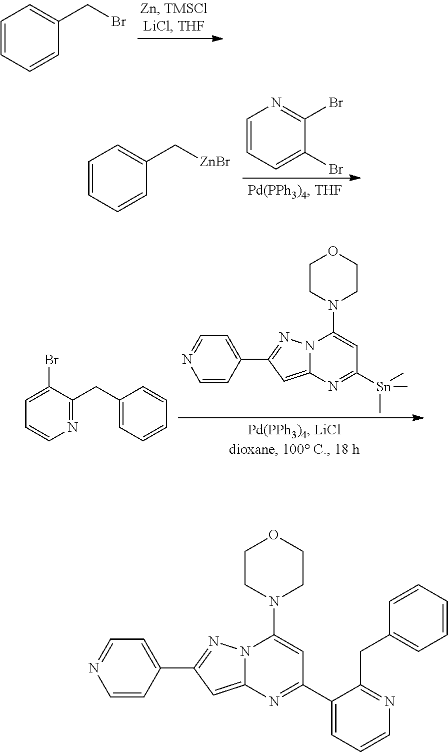

### Step 1: Synthesis of benzylzinc (II) bromide

A 2-necked flask equipped with a magnetic stirring bar and a condenser was charged with lithium chloride (2.67 g, 63 mmol) and the flask was heated with a heat-gun (400° C.) for 10 min under high vacuum. After cooling the flask to 25° C., the flask was flushed with argon (3 times). Activated zinc dust (9.2 g, 141 mmol) was then added to the flask followed by THE (50 mL). A solution of 1,2-dibromethane (1.46 g, 7.76 mmol) in THE (5 mL) was added dropwise over a period of 5 min and the reaction mixture was heated (60° C.) until ebullition occurs (5 min). After cooling to 25° C., a solution of trimethylsilyl chloride (1.27 g, 11.65 mmol) in THE (5 mL) was added dropwise over a period of 5 min and the mixture was heated again until ebullition occurs (30 min). The reaction mixture was used directly in next step without further purification LCMS (ESI) m/z: 250.0 [M+H]+.

### Step 2: Synthesis of 2-benzyl-3-bromopyridine

A solution of benzylzinc (II) bromide (10.4 mL, 6.75 mmol) was added dropwise to a mixture of 2,3-dibromopyridine (1 g, 4.2 mmol) and (0) (0.15 g, 0.13 mmol) in THE (10 mL) over a period of 5 min. The resultant mixture was stirred at 25° C. for 18 h, then quenched with aqueous saturated ammonium chloride solution (25 mL) and the mixture was extracted with ethyl acetate (25 mL×3). The combined organic layers were washed (brine, 25 mL), dried over anhydrous sodium sulfate, filtered and concentrated. The residue was subjected to column chromatography (ethyl acetate/petroleum ether=10:1) to obtain 2-benzyl-3-bromopyridine as yellow solid (240 mg, 23%) LCMS (ESI) m/z: 248.0 [M+H]+.

### Step 3: Synthesis of 4-(5-(2-benzylpyridin-3-yl)-2-(pyridin-4-yl)pyrazolo[1,5-a]pyrimidin-7-yl)morpholine

To a solution of 4-(2-(pyridin-4-yl)-5-(trimethylstannyl)pyrazolo[1,5-a]pyrimidin-7-yl)morpholine (0.15 g, 0.33 mmol) in dioxane (5 mL) were added 2-benzyl-3-bromopyridine (0.13 g, 0.51 mmol), lithium chloride (0.04 g, 0.84 mmol) and tetrakis(triphenylphosphine)palladium (0.04 g, 0.03 mmol) at 25° C. and the resultant mixture was stirred at 100° C. for 18 h under argon atmosphere. The reaction mixture was then filtered, and the filtrate was concentrated. The residue was then subjected to prep-HPLC (Boston C18 21*250 mm 10 μm column. The mobile phase was acetonitrile/10 mM Formic acid aqueous solution.) to obtain 4-(5-(2-benzylpyridin-3-yl)-2-(pyridin-4-yl)pyrazolo[1,5-a]pyrimidin-7-yl)morpholine as a white solid.(38 mg, 25%)¹H NMR (400 MHz, DMSO-d6) δ 8.74-8.68 (m, 2H), 8.63 (dd, J=4.8, 1.7 Hz, 1H), 8.03 (dd, J=4.5, 1.6 Hz, 2H), 7.95 (dd, J=7.7, 1.7 Hz, 1H), 7.43 (dd, J=7.7, 4.8 Hz, 1H), 7.34 (s, 1H), 7.14 (dd, J=14.4, 7.0 Hz, 3H), 7.06 (d, J=6.8 Hz, 2H), 6.40 (s, 1H), 4.34 (s, 2H), 3.81 (d, J=5.2 Hz, 2H), 3.77 (d, J=5.2 Hz, 2H); LCMS (ESI) m/z: 449.1 [M+H]+.

### Synthesis of 4-(5-(3-benzylpyridin-2-yl)-2-(pyridin-4-yl)pyrazolo[1,5-a]pyrimidin-7-yl)morpholine (Compound 40)

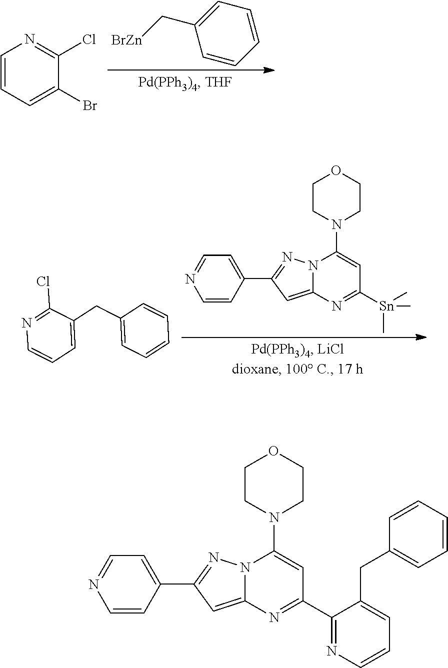

### Step 1: Synthesis of 3-benzyl-2-chloropyridine

A solution of benzylzinc(II) bromide (15.6 mL, 10.1 mmol) was added dropwise to a solution of 3-bromo-2-chloropyridine (1.5 g, 7.8 mmol) and tetrakis)triphenypsphosdium)palladium(0)(0.27 g, 0.23 mmol) in THE (15 mL) over a period of 5 min. Then the mixture was stirred at 25° C. for 18 h and then quenched with aqueous saturated ammonium chloride solution (25 mL). The resultant mixture was extracted with ethyl acetate (25 mL×3), the combined organic layers were washed (brine, 25 mL×1), dried over anhydrous sodium sulfate, filtered and concentrated. The residue was subjected to column chromatography (ethyl acetate/petroleum ether=10:1) to obtain 3-benzyl-2-chloropyridine as yellow solid (600 mg, 38%) LCMS (ESI) m/z: 204.1 [M+H]+.

### Step 2: Synthesis of 4-(5-(3-benzylpyridin-2-yl)-2-(pyridin-4-yl)pyrazolo[1,5-a]pyrimidin-7-yl)morpholine

To a solution of 4-(2-(pyridin-4-yl)-5-(trimethylstannyl)pyrazolo[1,5-a]pyrimidin-7-yl)morpholine (0.15 g, 0.33 mmol) in dioxane (6 mL) were added 3-benzyl-2-chloropyridine (0.1 g, 0.51 mmol), lithium chloride (0.03 g, 0.84 mmol) and tetrakis(triphenylphosphine)palladium (0.04 g, 0.03 mmol) at 25° C. under argon atmosphere. The resulting mixture was stirred at 100° C. for 17 h under argon atmosphere and concentrated. The residue was subjected to prep-HPLC (Boston C18 21*250 mm 10 μm column. The mobile phase was acetonitrile/10 mM Formic acid aqueous solution.) to obtain 4-(5-(3-benzylpyridin-2-yl)-2-(pyridin-4-yl)pyrazolo[1,5-a]pyrimidin-7-yl)morpholine as white solid.(42.8 mg, 28%)¹H NMR (400 MHz, DMSO-d₆) δ 8.71 (s, 2H), 8.59 (s, 1H), 8.04 (d, J=3.2 Hz, 2H), 7.77 (d, J=7.7 Hz, 1H), 7.48 (s, 1H), 7.38 (s, 1H), 7.19 (d, J=7.1 Hz, 2H), 7.11 (d, J=7.2 Hz, 3H), 6.81 (s, 1H), 4.48 (s, 2H), 3.86 (s, 4H), 3.79 (s, 4H); LCMS (ESI) m/z: 449.2 [M+H]+.

### Synthesis of 4-(5-(3-phenoxypyridin-2-yl)-2-(pyridin-4-yl)pyrazolo[1,5-a]pyrimidin-7-yl)morpholine (Compound 41)

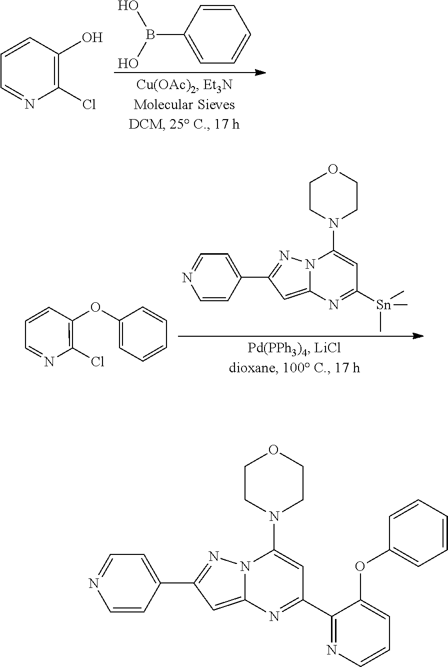

### Step 1: Synthesis of 2-chloro-3-phenoxypyridine

To a solution of 2-chloropyridin-3-ol (1 g, 7.72 mmol) in dichloromethane (80 mL) was added phenylboronic acid (1.88 g, 15.44 mmol), cupric acetate (1.4 g, 7.72 mmol), triethylamine (3.91 g, 38.6 mmol) and 4 Å molecular sieves (5 g) at 25° C. under argon atmosphere. The mixture was then stirred at 100° C. for 17 h and cooled. The mixture was filtered to remove the solids and the filtrate was concentrated. The residue was then subjected to silica gel column chromatography (petroleum ether: ethyl acetate=5:1) to obtain 2-chloro-3-phenoxypyridine as yellow oil. (0.5 g, 32%) LCMS (ESI) m/z: 206.1 [M+H]+.

### Step 2: Synthesis of 4-(5-(3-phenoxypyridin-2-yl)-2-(pyridin-4-yl)pyrazolo[1,5-a]pyrimidin-7-yl)morpholine

To a solution of 4-(2-(pyridin-4-yl)-5-(trimethylstannyl)pyrazolo[1,5-a]pyrimidin-7-yl)morpholine (0.15 g, 0.33 mmol) in dioxane (5 mL) were added 2-chloro-3-phenoxypyridine (0.1 g, 0.51 mmol), lithium chloride (0.03 g, 0.68 mmol) and tetrakis(triphenylphosphine)palladium (0.04 g, 0.03 mmol) at 25° C. under nitrogen atmosphere. The mixture was then stirred at 100° C. for 17 h and cooled. It was filtered to remove the solids; the filtrate was concentrated, and the residue was subjected to prep-HPLC (Boston C18 21*250 mm 10 μm column. The mobile phase was acetonitrile/10 mM formic acid aqueous solution.) to obtain 4-(5-(3-phenoxypyridin-2-yl)-2-(pyridin-4-yl)pyrazolo[1,5-a]pyrimidin-7-yl)morpholine as a white solid.(17.2 mg, 11%)¹H NMR (400 MHz, DMSO-d₆) δ 8.69 (d, J=5.8 Hz, 2H), 8.56 (t, J=2.8 Hz, 1H), 8.39 (s, 1H), 8.00 (d, J=5.9 Hz, 2H), 7.58 (d, J=2.8 Hz, 2H), 7.35 (t, J=8.0 Hz, 2H), 7.26 (s, 1H), 7.10 (t, J=7.4 Hz, 1H), 7.01 (d, J=7.8 Hz, 2H), 6.86 (s, 1H), 3.85 (d, J=4.4 Hz, 4H), 3.79 (d, J=3.6 Hz, 4H); LCMS (ESI) m/z: 451.2 [M+H]+.

### Synthesis of 4-(5-(2-phenoxypyridin-3-yl)-2-(pyridin-4-yl)pyrazolo[1,5-a]pyrimidin-7-yl)morpholine (Compound 42)

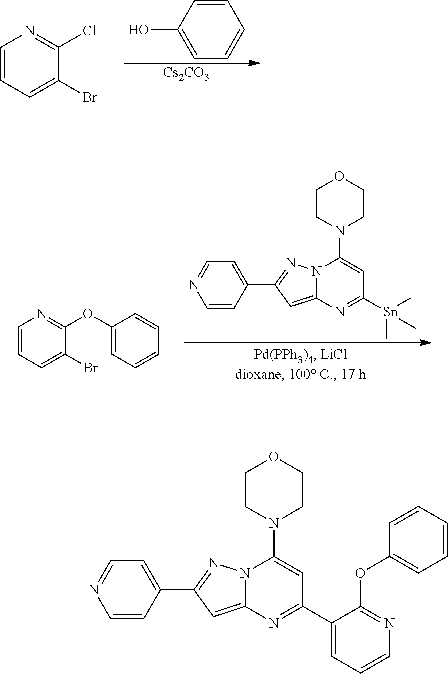

### Step 1: Synthesis of 3-bromo-2-phenoxypyridine

A mixture of 3-bromo-2-chloropyridine (1 g, 5.2 mmol), cesium carbonate (3.39 g, 10.4 mmol) and phenol (0.73 g, 7.8 mmol) in dimethyl sulfoxide (15 mL) was stirred at 120° C. for 5 h. The mixture was poured into water (50 mL), extracted with ethyl acetate (15 mL*3), the combined organic phase was dried over sodium sulfate, filtered and concentrated. The residue was subjected to silica gel chromatography (petroleum ether: ethyl acetate=5:1) to obtain 3-bromo-2-phenoxypyridine as off-white solid (0.7 g, 54%); LCMS (ESI) m/z: 250.0 [M+H]+.

### Step 2: Synthesis of 4-(5-(2-phenoxypyridin-3-yl)-2-(pyridin-4-yl)pyrazolo[1,5-a]pyrimidin-7-yl)morpholine

To a solution of 4-(2-(pyridin-4-yl)-5-(trimethylstannyl)pyrazolo[1,5-a]pyrimidin-7-yl)morpholine (0.15 g, 0.34 mmol) in dioxane (5 mL) were added 3-bromo-2-phenoxypyridine (0.13 g, 0.51 mmol), lithium chloride (0.03 g, 0.68 mmol) and tetrakis(triphenylphosphine)palladium (0.04 g, 0.03 mmol) at 25° C. under argon atmosphere. The reaction mixture was stirred at 100° C. for 7 h under argon atmosphere. The resultant mixture was filtered to remove the solids and the filtrate was concentrated. The residue was subjected to prep-HPLC (Boston C18 21*250 mm 10 μm column. The mobile phase was acetonitrile/10 mM ammonium bicarbonate aqueous solution.) to obtain 4-(5-(2-phenoxypyridin-3-yl)-2-(pyridin-4-yl)pyrazolo[1,5-a]pyrimidin-7-yl)morpholine as a white solid.(38 mg, 25%)¹H NMR (400 MHz, DMSO-d₆) δ 8.71 (dd, J=4.6, 1.5 Hz, 2H), 8.38 (dd, J=7.5, 1.9 Hz, 1H), 8.26 (dd, J=4.8, 1.9 Hz, 1H), 8.02 (dd, J=4.5, 1.5 Hz, 2H), 7.48-7.39 (m, 2H), 7.36 (s, 1H), 7.34 (dd, J=7.5, 4.8 Hz, 1H), 7.24 (s, 1H), 7.22 (dt, J=4.8, 2.1 Hz, 2H), 7.09 (s, 1H), 3.85 (bs, 4H), 3.82 (bs, 4H); LCMS (ESI) m/z: 451.2 [M+H]+.

### Synthesis of 3-(3-fluorophenyl)-1-(7-morpholino-2-(pyridin-4-yl)pyrazolo[1,5-a]pyrimidin-5-yl)-1H-pyrazol-5-ol (Compound 43)

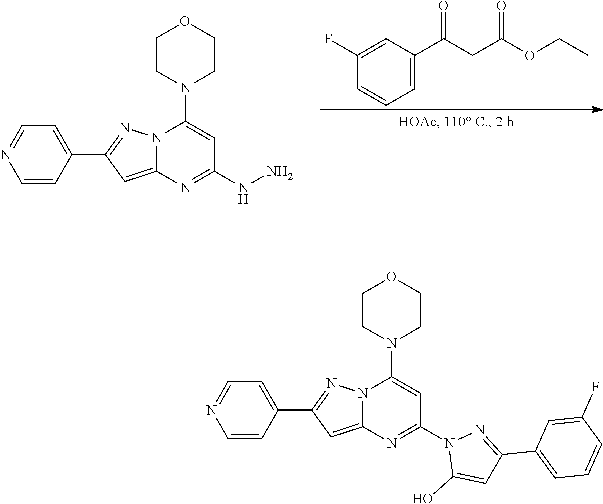 [SMILES: NNc1cc(N2CCOCC2)n2nc(-c3ccncc3)cc2n1 ; CCOC(=O)CC(=O)c1cccc(F)c1 ; Oc1cc(-c2cccc(F)c2)nn1-c1cc(N2CCOCC2)n2nc(-c3ccncc3)cc2n1]

A Mixture of 4-(5-hydrazineyl-2-(pyridin-4-yl)pyrazolo[1,5-a]pyrimidin-7-yl)morpholine (200 mg, 0.64 mmol) and ethyl 3-(3-fluorophenyl)-3-oxopropanoate (124 mg, 0.64 mmol) in acetic acid (5 mL) was stirred at 110° C. for 2 h under argon atmosphere. The mixture was concentrated, the residue was dissolved in ethyl acetate (50 mL) and washed with saturated aqueous sodium bicarbonate (10 mL). The organic layer was concentrated, and residue was slurred with ethanol (20 mL), the precipitate formed was collected by filtration and dried to obtain 3-(3-fluorophenyl)-1-(7-morpholino-2-(pyridin-4-yl)pyrazolo[1,5-a]pyrimidin-5-yl)-1H-pyrazol-5-ol as white solid (106 mg, 36%). ¹H NMR (400 MHz, DMSO-d₆) δ 8.79 (d, J=6.0 Hz, 2H), 8.14 (d, J=5.0 Hz, 2H), 7.80-7.72 (m, 2H), 7.52 (dd, J=14.1, 7.9 Hz, 1H), 7.33 (s, 1H), 7.26 (s, 1H), 6.96 (s, 1H), 6.29 (s, 1H), 3.93 (d, J=5.9 Hz, 4H), 3.87 (bs, 4H); LCMS (ESI) m/z: 458.1 [M+H]+.

### Synthesis of 1-(7-morpholino-2-(pyridin-4-yl)pyrazolo[1,5-a]pyrimidin-5-yl)-3-(pyrimidin-5-yl)-1H-pyrazol-5-ol (Compound 44)

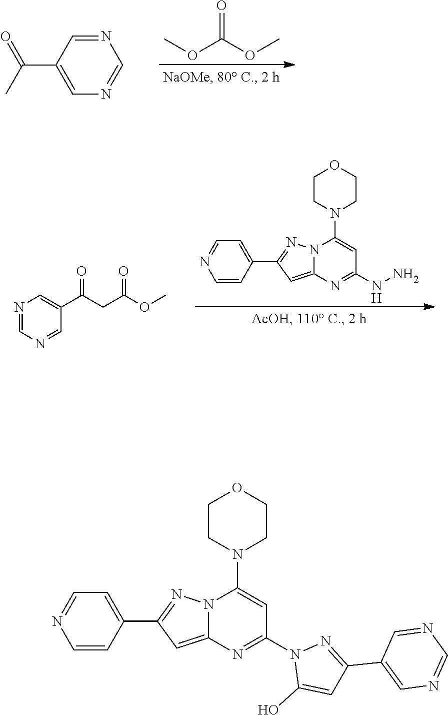

### Step 1: Synthesis of methyl 3-oxo-3-(pyrimidin-5-yl)propanoate

To a mixture of 1-(pyrimidin-5-yl)ethanone (120 mg, 1 mmol) and dimethyl carbonate (4 mL) was added sodium methanolate (540 mg, 10 mmol) and the mixture was stirred at 80° C. for 2h. It was cooled and the mixture was diluted with ethyl acetate (50 mL) followed by the addition of 6N HCl until the pH-6-7 was reached. The resultant precipitate was removed by filtration and the filtrate was extracted with ethyl acetate (100 mL*2), the combined organic phase was washed with brine (100 mL), dried and concentrated to obtain methyl 3-oxo-3-(pyrimidin-5-yl)propanoate as yellow oil (100 mg, 55%). LCMS (ESI) m/z: 181.1 [M+H]+.

### Step 2: Synthesis of 1-(7-morpholino-2-(pyridin-4-yl)pyrazolo[1,5-a]pyrimidin-5-yl)-3-(pyrimidin-5-yl)-1H-pyrazol-5-ol

A mixture of 4-(5-hydrazinyl-2-(pyridin-4-yl)pyrazolo[1,5-a]pyrimidin-7-yl)morpholine (93 mg, 0.3 mmol) and methyl 3-oxo-3-(pyrimidin-5-yl)propanoate (108 mg, 0.6 mmol) in acetic acid (5 mL) was stirred at 110° C. for 2 h and concentrated. The resultant residue was subjected to prep-HPLC (BOSTON pHlex ODS 10 μm 21.2×250 mm120 A. The mobile phase was acetonitrile/0.1% Formic acid) to obtain 1-(7-morpholino-2-(pyridin-4-yl)pyrazolo[1,5-a]pyrimidin-5-yl)-3-(pyrimidin-5-yl)-1H-pyrazol-5-ol (5 mg, 4%) as yellow solid. ¹H NMR (400 MHz, CDCl₃+CD₃OD) δ 9.25 (s, 2H), 9.18 (s, 1H), 8.74 (d, J=6.4 Hz, 2H), 8.21 (s, 2H), 7.61-7.61 (m, 1H), 7.10 (s, 1H), 7.05 (s, 1H), 4.07 (s, 8H); LCMS (ESI) m/z: 442.0 [M+H]+.

The following compounds were synthesized according to the protocols described above:

| Name | Structure | NMR, MS | Compd # |
|---|---|---|---|
| 3-(4-fluorophenyl)-1- (7-morpholino-2- (pyridin-4- yl)pyrazolo[1,5- a]pyrimidin-5-yl)-1H- pyrazol-5-ol | 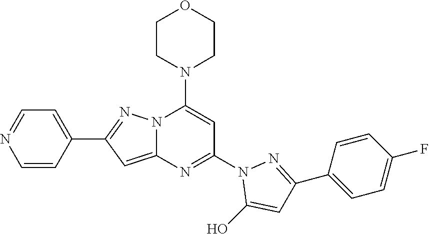 [SMILES: Oc1cc(-c2ccc(F)cc2)nn1-c1cc(N2CCOCC2)n2nc(-c3ccncc3)cc2n1] | ¹H NMR (400 MHz, DMSO-d₆) δ 8.79 (d, J = 5.3 Hz, 2H), 8.15 (s, 2H), 8.04 − 7.89 (m, 2H), 7.34 (s, 1H), 7.31 (d, J = 4.2 Hz, 2H), 7.16 − 6.72 (m, 1H), 6.19 (s, 1H), 3.92 (s, 8H).; LCMS (ESI) m/z: 458.1 [M + H]+. | 45 |
| 1-(7-morpholino-2- (pyridin-4- yl)pyrazolo[1,5- a]pyrimidin-5-yl)-3- (o-tolyl)-1H-pyrazol- 5-ol | 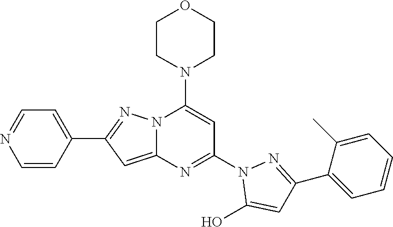 [SMILES: Cc1ccccc1-c1cc(O)n(-c2cc(N3CCOCC3)n3nc(-c4ccncc4)cc3n2)n1] | ¹H NMR (400 MHz, MeOD) δ 8.65 (d, J = 5.8 Hz, 2H), 8.30 (s, 4H), 8.04 (d, J = 5.3 Hz, 2H), 7.56 (s, 1H), 7.40-7.25 (m, 4H), 7.06 (s, 1H), 4.01 (s, 8H), 2.53 (s, 3H).; LCMS (ESI) m/z: 454.2 [M + H]+. | 46 |
| 1-(7-morpholino-2- (pyridin-4- yl)pyrazolo[1,5- a]pyrimidin-5-yl)-3- (pyridin-3-yl)-1H- pyrazol-5-ol | 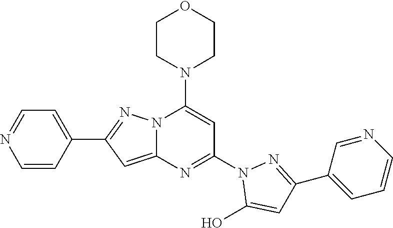 [SMILES: Oc1cc(-c2cccnc2)nn1-c1cc(N2CCOCC2)n2nc(-c3ccncc3)cc2n1] | ¹H NMR (400 MHz, TFA) δ 9.67-9.58 (m, 1H), 9.35 − 9.14 (m, 2H), 9.10 (d, J = 6.3 Hz, 2H), 9.02 (d, J = 14.2 Hz, 1H), 8.82 (s, 2H), 8.47-8.38 (m, 1H), 7.63 − 7.56 (m, 2H), 4.81 (s, 4H), 4.49 (s, 4H).; LCMS (ESI) m/z: 441.1 [M + H]+. | 47 |
| 3-ethyl-1-(7- morpholino-2- (pyridin-4- yl)pyrazolo[1,5- a]pyrimidin-5-yl)-1H- pyrazol-5-ol | 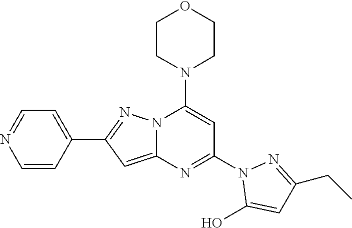 [SMILES: CCc1cc(O)n(-c2cc(N3CCOCC3)n3nc(-c4ccncc4)cc3n2)n1] | ¹H NMR (400 MHz, DMSO-d₆) δ 8.68 (d, J = 5.9 Hz, 2H), 7.95 (d, J = 5.1 Hz, 2H), 7.87 (s, 1H), 6.98 (s, 1H), 4.85 (s, 1H), 3.88 (d, J = 4.9 Hz, 4H), 3.73 (s, 4H), 2.44 (q, J = 7.1 Hz, 2H), 1.15 (t, J = 7.6 Hz, 3H).; LCMS (ESI) m/z: 392.1 [M + H]+. | 48 |
| 3-cyclohexyl-1-(7- morpholino-2- (pyridin-4- yl)pyrazolo[1,5- a]pyrimidin-5-yl)- 1H-pyrazol-5-ol | 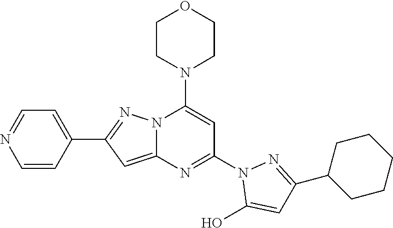 [SMILES: Oc1cc(C2CCCCC2)nn1-c1cc(N2CCOCC2)n2nc(-c3ccncc3)cc2n1] | ¹H NMR (400 MHz, DMSO-d₆) δ 8.70 (d, J = 6.0 Hz, 2H), 7.98 (d, J = 6.0 Hz, 2H), 7.65 − 7.38 (m, 1H), 7.07 (s, 1H), 5.20 (s, 1H), 3.89 (d, J = 5.1 Hz, 4H), 3.80 (s, 4H), 1.88 (s, 2H), 1.75 (s, 2H), 1.67 (s, 1H), 1.46 − 1.17 (m, 6H).; LCMS (ESI) m/z: 446.3 [M + H]+. | 49 |
| 1-(7-morpholino-2- (pyridin-4- yl)pyrazolo[1,5- a]pyrimidin-5-yl)-3- (tetrahydro-2H- pyran-4-yl)-1H- pyrazol-5-ol | 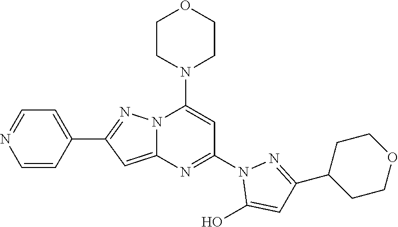 [SMILES: Oc1cc(C2CCOCC2)nn1-c1cc(N2CCOCC2)n2nc(-c3ccncc3)cc2n1] | ¹H NMR (400 MHz, DMSO-d₆) δ 8.69 (d, J = 6.0 Hz, 3H), 8.27 (s, 1H), 7.96 (d, J = 4.9 Hz, 2H), 7.47 (s, 1H), 7.07 (s, 1H), 5.21 (s, 1H), 3.93 (s, 2H), 3.90 − 3.87 (m, 4H), 3.79 (s, 4H), 3.39 (d, J = 11.6 Hz, 3H), 2.79 (s, 1H), 1.80 (d, J = 11.9 Hz, 2H), 1.65 (dt, J = 11.7, 8.0 Hz, 2H).; LCMS (ESI) m/z: 448.2 [M + H]+. | 50 |
| 1-(7-morpholino-2- (pyridin-4- yl)pyrazolo[1,5- a]pyrimidin-5-yl)-3- phenyl-1H-pyrazol- 5-ol | 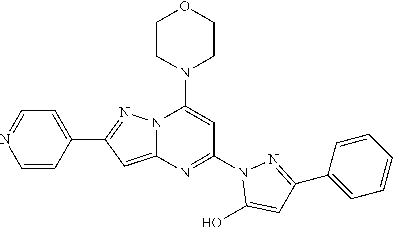 [SMILES: Oc1cc(-c2ccccc2)nn1-c1cc(N2CCOCC2)n2nc(-c3ccncc3)cc2n1] | ¹H NMR (400 MHz, TFA) δ 8.98 (d, J = 6.4 Hz, 2H), 8.69 (d, J = 6.2 Hz, 2H), 7.87 (d, J = 7.6 Hz, 2H), 7.58 (d, J = 7.6 Hz, 3H), 7.44 (d, J = 19.1 Hz, 2H), 6.80 (s, 1H), 4.65 (s, 4H), 4.37 (s, 4H); LCMS (ESI) m/z: 440.3 [M + H]+. | 51 |
| 2-(7-morpholino-2- (pyridin-4- yl)pyrazolo[1,5- a]pyrimidin-5-yl)-5- (m-tolyl)-2,4- dihydro-3H- pyrazol-3-one | 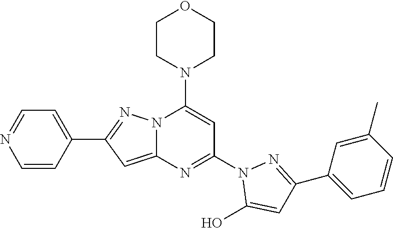 [SMILES: Cc1cccc(-c2cc(O)n(-c3cc(N4CCOCC4)n4nc(-c5ccncc5)cc4n3)n2)c1] | ¹H NMR (400 MHz, TFA) δ 12.35 (s, 1H), 8.72 (d, J = 5.6 Hz, 2H), 7.99 (d, J = 5.6 Hz, 2H), 7.77 − 7.65 (m, 2H), 7.37 (t, J = 7.6 Hz, 1H), 7.24 (d, J = 12.4 Hz, 2H), 7.16 − 6.90 (m, 1H), 6.13 (s, 1H), 3.92 (s, 8H), 2.39 (s, 3H).; LCMS (ESI) m/z: 454.2 [M + H]+. | 52 |
| 3-(2-fluorophenyl)- 1-(7-morpholino-2- (pyridin-4- yl)pyrazolo[1,5- a]pyrimidin-5-yl)- 1H-pyrazol-5-ol | 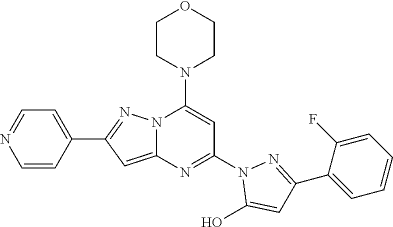 [SMILES: Oc1cc(-c2ccccc2F)nn1-c1cc(N2CCOCC2)n2nc(-c3ccncc3)cc2n1] | ¹H NMR (400 MHz, TFA) δ 8.97 (d, J = 6.3 Hz, 2H), 8.68 (d, J = 6.3 Hz, 2H), 8.09 (t, J = 7.0 Hz, 1H), 7.67 (dd, J = 13.6, 6.4 Hz, 1H), 7.48 (s, 1H), 7.43 (s, 1H), 7.38 (d, J = 7.6 Hz, 1H), 7.28 (dd, J = 11.5, 8.6 Hz, 1H), 4.66 (bs, 4H), 4.38 (t, J = 4.8 Hz, 4H).; LCMS (ESI) m/z: 458.1 [M + H]+. | 53 |
| 3-isopropyl-1-(7- morpholino-2- (pyridin-4- yl)pyrazolo[1,5- a]pyrimidin-5-yl)- 1H-pyrazol-5-ol | 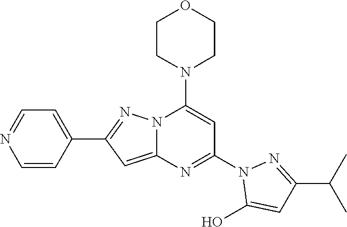 [SMILES: CC(C)c1cc(O)n(-c2cc(N3CCOCC3)n3nc(-c4ccncc4)cc3n2)n1] | ¹H NMR (400 MHz, DMSO-d₆) δ 8.70 (d, J = 5.8 Hz, 2H), 7.98 (d, J = 5.7 Hz, 2H), 7.53 (s, 1H), 7.08 (s, 1H), 5.25 (s, 1H), 3.88 (d, J = 4.4 Hz, 4H), 3.81 (d, J = 4.4 Hz, 4H), 2.87 (hept, J = 6.9 Hz, 1H), 1.22 (d, J = 6.9 Hz, 6H).; LCMS (ESI) m/z: 406.2 [M + H]+. | 54 |
| 2-(7-morpholino-2- (pyridin-4- yl)pyrazolo[1,5- a]pyrimidin-5-yl)- 4,5,6,7-tetrahydro- 2H-indazol-3-ol | 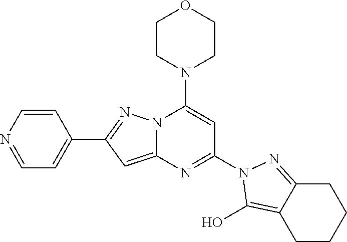 [SMILES: Oc1c2c(nn1-c1cc(N3CCOCC3)n3nc(-c4ccncc4)cc3n1)CCCC2] | ¹H NMR (400 MHz, DMSO-d₆) δ 11.55 (s, 1H), 8.69 (d, J = 6.0 Hz, 2H), 7.99 (d, J = 6.0 Hz, 2H), 7.71 (s, 1H), 7.03 (s, 1H), 3.88 (d, J = 4.8 Hz, 4H), 3.80 (d, J = 4.8 Hz, 4H), 2.50 (d, J = 1.6 Hz, 2H), 2.17 (s, 2H), 1.86 − 1.53 (m, 4H); LCMS (ESI) m/z: 418.2 [M + H]+. | 55 |
| 3-(1- methylpiperidin-4- yl)-1-(7- morpholino-2- (pyridin-4- yl)pyrazolo[1,5- a]pyrimidin-5-yl)- 1H-pyrazol-5-ol | 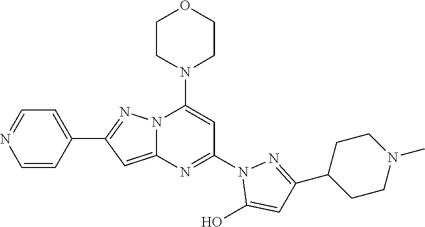 [SMILES: CN1CCC(c2cc(O)n(-c3cc(N4CCOCC4)n4nc(-c5ccncc5)cc4n3)n2)CC1] | ¹H NMR (400 MHz, DMSO-d₆) δ 8.70 (dd, J = 4.8, 1.6 Hz, 2H), 7.98 (dd, J = 4.4, 1.2 Hz, 2H), 7.45 (bs, 1H), 7.08 (s, 1H), 5.28 (s, 1H), 3.90-3.81 (m, 8H), 2.92-2.89 (m, 2H), 2.51-2.45 (m, 1H), 2.26 (s, 3H), 2.11-2.05 (m, 2H), 1.89-1.87 (m, 2H), 1.71-1.61 (m, 2H); LCMS (ESI) m/z: 461.3 [M + H]+. | 56 |

### Synthesis of 3-(7-morpholino-2-(pyridin-4-yl)pyrazolo[1,5-a]pyrimidin-5-yl)-1-phenyl-1H-pyrazol-5-ol (Compound 57)

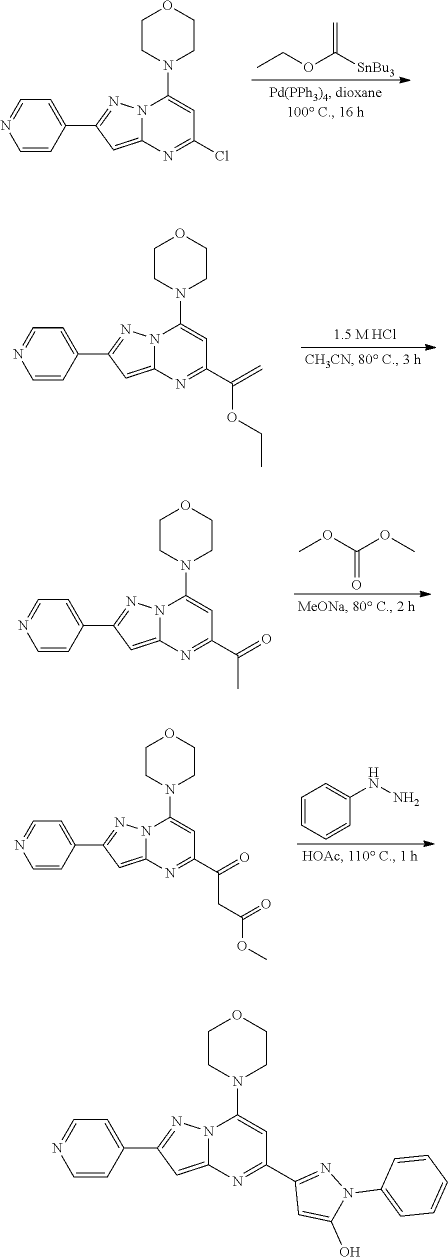

### Step 1: Synthesis of 4-(5-(1-ethoxyvinyl)-2-(pyridin-4-yl)pyrazolo[1,5-a]pyrimidin-7-yl)morpholin

A mixture of 4-(5-chloro-2-(pyridin-4-yl)pyrazolo[1,5-a]pyrimidin-7-yl)morpholine (3.47 g, 11 mmol), tributyl(1-ethoxyvinyl)stannane (5.92 mg, 16.5 mmol) and tetrakis(triphenylphosphine)palladium (127 mg, 0.11 mmol) in dioxane (220 mL) was stirred at 100° C. for 16 h under argon atmosphere. The reaction mixture was filtered, and the filtrate was concentrated. The residue was triturated with ethyl acetate (50 mL), the resultant precipitate was collected by filtration and vacuum dried to obtain 4-(5-(1-ethoxyvinyl)-2-(pyridin-4-yl)pyrazolo[1,5-a]pyrimidin-7-yl)morpholine (2.6 g, 67.2%) as brown solid. LCMS (ESI) m/z: 352.2[M+H]+.

### Step 2: Synthesis of 1-(7-morpholino-2-(pyridin-4-yl)pyrazolo[1,5-a]pyrimidin-5-yl)ethan-1-on

To a solution of 4-(5-(1-ethoxyvinyl)-2-(pyridin-4-yl)pyrazolo[1,5-a]pyrimidin-7-yl)morpholine (2.58 g, 7.34 mmol) in acetonitrile (250 mL) was added hydrochloric acid (1.5M, 32.2 mL) and the mixture was stirred at 80° C. for 3 h. It was concentrated and the residue was triturated with aqueous sodium bicarbonate solution and extracted with ethyl acetate (25 mL×3). The combined organic phase was evaporated to obtain 1-(7-morpholino-2-(pyridin-4-yl)pyrazolo[1,5-a]pyrimidin-5-yl)ethan-1-one as yellow solid (1.6 g, 67.5%). LCMS (ESI) m/z: 324.1[M+H]+.

### Step 3: Synthesis of methyl 3-(7-morpholino-2-(pyridin-4-yl)pyrazolo[1,5-a]pyrimidin-5-yl)-3-oxopropanoat

To a mixture of 1-(7-morpholino-2-(pyridin-4-yl)pyrazolo[1,5-a]pyrimidin-5-yl)ethan-1-one (400 mg, 1.24 mmol) and dimethyl carbonate (25 mL) was added sodium methanolate (670 mg, 12.4 mmol) and the mixture was stirred at 80° C. for 2 h. It was then cooled to 5° C.-10° C., diluted with ethyl acetate (50 mL) and the mixture was treated with 6N hydrochloric acid until pH-6-7. The resultant precipitate was removed by filtration and the filtrate was extracted with ethyl acetate (100 mL*2). The combined organic phase was washed with brine (100 mL), dried over sodium sulfate and concentrated to obtain methyl 3-(7-morpholino-2-(pyridin-4-yl)pyrazolo[1,5-a]pyrimidin-5-yl)-3-oxopropanoate (400 mg, crude) as thick brown liquid. LCMS (ESI) m/z: 382.2[M+H]+.

### Step 4: Synthesis of 3-(7-morpholino-2-(pyridin-4-yl)pyrazolo[1,5-a]pyrimidin-5-yl)-1-phenyl-1H-pyrazol-5-ol

A solution of methyl 3-(7-morpholino-2-(pyridin-4-yl)pyrazolo[1,5-a]pyrimidin-5-yl)-3-oxopropanoate (229 mg, 0.6 mmol) and phenylhydrazine (78 mg, 0.72 mmol) in acetic acid (6 mL) was stirred at 110° C. for 1 h. It was concentrated, the residue was dissolved in ethyl acetate (160 mL), washed with water (60 mL), brine (60 mL), dried over sodium sulfate and concentrated. The residue was subjected to prep-HPLC (BOSTON pHlex ODS 10 μm 21.2×250 mm 120 A. The mobile phase was acetonitrile/0.1% aqueous formic acid) to obtain 3-(7-morpholino-2-(pyridin-4-yl)pyrazolo[1,5-a]pyrimidin-5-yl)-1-phenyl-1H-pyrazol-5-ol as yellow solid (21.2 mg, 8.1%). ¹H NMR (400 MHz, DMSO-d6) δ 8.70 (d, J=6.0 Hz, 2H), 8.00 (d, J=6.0 Hz, 2H), 7.91 (d, J=7.8 Hz, 2H), 7.52 (t, J=7.9 Hz, 2H), 7.33 (t, J=7.4 Hz, 1H), 7.29 (s, 1H), 7.00 (s, 1H), 6.12 (s, 1H), 3.90 (d, J=4.8 Hz, 4H), 3.83 (s, 4H); LCMS (ESI) m/z: 440.2 [M+H]+.

The following compounds were synthesized according to the protocol described above:

| Name | Structure | NMR, MS | Compd # |
|---|---|---|---|
| 3-(7-morpholino- 2-(pyridin-4- yl)pyrazolo[1,5- a]pyrimidin-5-yl)- 1-(pyridin-3-yl)- 1H-pyrazol-5-ol | 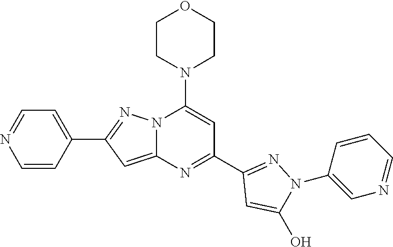 [SMILES: Oc1cc(-c2cc(N3CCOCC3)n3nc(-c4ccncc4)cc3n2)nn1-c1cccnc1] | ¹H NMR (400 MHz, DMSO-d₆) δ 9.23 (s, 1H), 8.70 (dd, J = 4.8 Hz, 2H), 8.49 (d, J = 4.0 Hz, 1H), 8.36 (d, J = 8.4 Hz, 1H), 7.99 (dd, J = 4.4 Hz, 2H), 7.52-7.55 (m, 1H), 7.28 (s, 1H), 7.03 (s, 1H), 6.02 (s, 1H), 3.82-3.91 (m, 8H); LCMS (ESI) m/z: 441.1 [M + H]+. | 58 |
| 1-(3- fluorophenyl)-3- (7-morpholino-2- (pyridin-4- yl)pyrazolo[1,5- a]pyrimidin-5-yl)- 1H-pyrazol-5-ol | 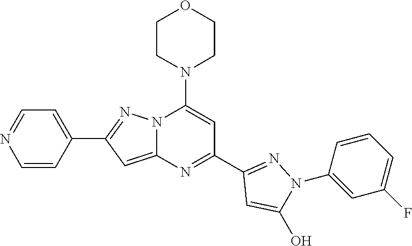 [SMILES: Oc1cc(-c2cc(N3CCOCC3)n3nc(-c4ccncc4)cc3n2)nn1-c1cccc(F)c1] | ¹H NMR (400 MHz, DMSO-d₆) δ 12.49 (bs, 1H), 8.95 (d, J = 6.0 Hz, 2H), 8.50 (d, J = 6.4 Hz, 2H), 7.73- 7.80 (m, 2H), 7.55-7.61 (m, 2H), 7.18- 7.23 (m, 1H), 7.08 (s, 1H), 6.25 (s, 1H), 3.86-3.92 (m, 8H); LCMS (ESI) m/z: 458.1 [M + H]+. | 59 |

### Synthesis of 4-(5-(3-(5-fluoropyridin-3-yl)-1H-pyrazol-1-yl)-2-(pyridin-4-yl)pyrazolo[1,5-a]pyrimidin-7-yl)morpholine (Compound 60)

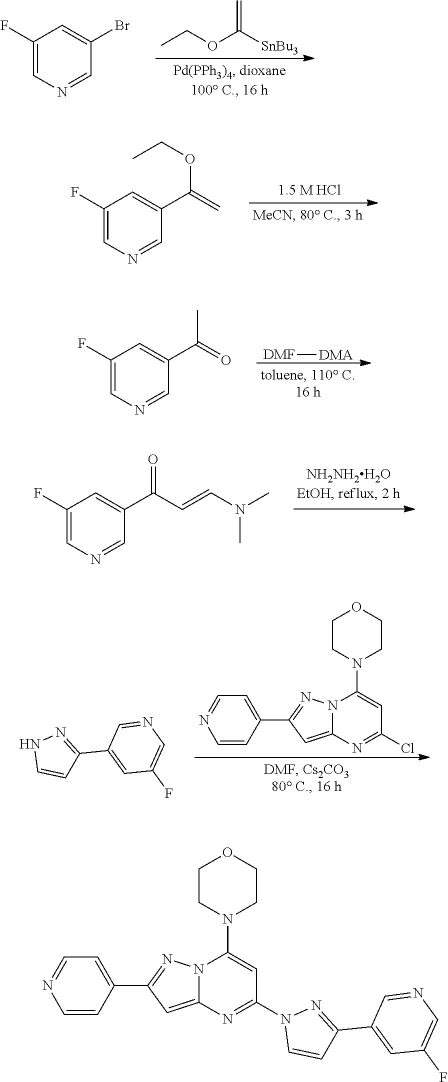

### Step 1: Synthesis of 3-(1-ethoxyvinyl)-5-fluoropyridin

To a solution of 3-bromo-5-fluoropyridine (0.7 g, 4 mmol) in dioxane (20 mL) was added tributyl(1-ethoxyvinyl)stannane (1.74 g, 4.8 mmol) and tetrakis(triphenylphosphine)palladium (0.4 mmol, 462 mg). The mixture was stirred at 100° C. for 16 h, then filtered to remove the solids and the filtrate was concentrated to afford 3-(1-ethoxyvinyl)-5-fluoropyridine as a yellow oil (668 mg, 99% yield). LCMS (ESI) m/z: 168.1 [M+H]+. It was used in the next step without further purification.

### Step 2: Synthesis of 1-(5-fluoropyridin-3-yl)ethan-1-on

To a solution of 3-(1-ethoxyvinyl)-5-fluoropyridine (668 mg, 1 mmol) in acetonitrile (20 mL) was added hydrochloric acid (8 mL, 1.5M, 12 mmol) and the mixture was stirred at 80° C. for 3 h. Then the solution was concentrated and to the residue was added sodium bicarbonate (50 mL) and stirred. The resultant precipitate was collected by filtration, the solids were washed with a mixture of ethyl acetate/petroleum ether (50 mL, 1/1) and dried to obtain 1-(5-fluoropyridin-3-yl)ethan-1-one as yellow solid (300 mg, 54%). LCMS (ESI) m/z: 140.1 [M+H]+.

### Step 3: Synthesis of (E)-3-(dimethylamino)-1-(5-fluoropyridin-3-yl)prop-2-en-1-on

To a solution of 1-(5-fluoropyridin-3-yl)ethan-1-one (238 mg, 1.71 mmol) was added DMF-DMA (1.22 g, 10.27 mmol and the reaction mixture was stirred at 110° C. for 16 h. It was then cooled and concentrated to obtain (E)-3-(dimethylamino)-1-(5-fluoropyridin-3-yl)prop-2-en-1-one as a yellow solid (331 mg, 99%) LCMS (ESI) m/z: 195.1 [M+H]+.

### Step 4: Synthesis of 3-fluoro-5-(1H-pyrazol-3-yl)pyridin

A mixture of (E)-3-(dimethylamino)-1-(5-fluoropyridin-3-yl)prop-2-en-1-one (331 mg, 1.71 mmol) and hydrazine hydrate (256 mg, 5.13 mmol) in ethanol (5 mL) was stirred at 90° C. for 2 h. It was then concentrated to obtain 3-fluoro-5-(1H-pyrazol-3-yl)pyridine as red solid. (278 mg, 99%). LCMS (ESI) m/z: 164.2 [M+H]+.

### Step 5: Synthesis of 4-(5-(3-(5-fluoropyridin-3-yl)-1H-pyrazol-1-yl)-2-(pyridin-4-yl)pyrazolo[1,5-a]pyrimidin-7-yl)morpholin

A mixture of 4-(5-chloro-2-(pyridin-4-yl)pyrazolo[1,5-a]pyrimidin-7-yl)morpholine (158 mg, 0.5 mmol), 3-fluoro-5-(1H-pyrazol-3-yl)pyridine (163 mg, 1 mmol) and cesium carbonate (1.5 mmol, 488 mg) in DMF (10 mL) was stirred at 80° C. for 16 h. The mixture was then diluted with water (10 mL), the resultant precipitate was collected by filtration and the solid was washed successively with water (10 mL) and ethanol (10 mL). The solid thus obtained was then vacuum dried to isolate 4-(5-(3-(5-fluoropyridin-3-yl)-1H-pyrazol-1-yl)-2-(pyridin-4-yl)pyrazolo[1,5-a]pyrimidin-7-yl)morpholine as yellow solid. (62.5 mg, 28%).

¹H NMR (400 MHz, CDCL3) δ 8.96 (s, 1H), 8.70-8.74 (m, 3H), 8.49 (d, J=2.8 Hz, 1H), 7.96-7.99 (m, 1H), 7.84-7.85 (m, 2H), 7.07 (s, 1H), 6.88 (d, J=3.2 Hz, 2H), 4.06-4.08 (m, 4H), 3.96-3.98 (m, 4H); LCMS (ESI) m/z: 443.3 [M+H]+.

### Synthesis of 4-(2-(pyridin-4-yl)-5-(3-(tetrahydro-2H-pyran-3-yl)-1H-pyrazol-1-yl)pyrazolo[1,5-a]pyrimidin-7-yl)morpholine (Compound 61)

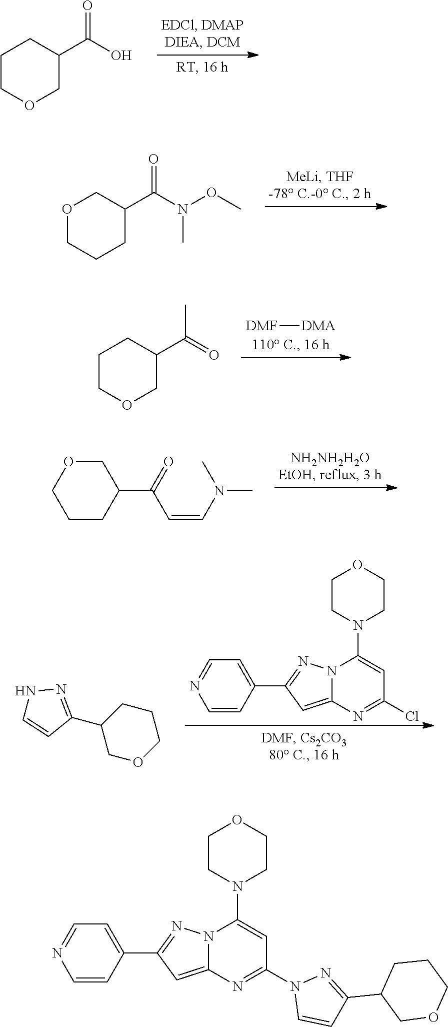

### Step 1: Synthesis of tetrahydro-2H-pyran-3-carboxylic aci

To a solution of N,O-dimethylhydroxylamine hydrochloride (1.46 g, 15 mmol) in DCM (10 mL) were added tetrahydro-2H-pyran-3-carboxylic acid (1.30 g, 10 mmol) and N-(3-dimethylaminopropyl)-N′-ethylcarbodiimide hydrochloride (2.87 g, 15 mmol), N,N-diisopropylethylamine (2.58 g, 20 mmol) and 4-dimethylaminepyridine (0.123 g, 1 mmol) at 25° C. The resultant reaction mixture was stirred at room temperature for 16 h, then diluted with water (20 mL) and extracted with dichloromethane (20 mL*2). The organic layer was dried and concentrated to obtain N-methoxy-N-methyltetrahydro-2H-pyran-3-carboxamide as yellow oil. (1.73 g, 99%). LCMS (ESI) m/z: 174.1 [M+H]+. The crude material was used in the next step without further purification.

### Step 2: Synthesis of 1-(tetrahydro-2H-pyran-3-yl)ethan-1-on

To a solution of N-methoxy-N-methyltetrahydro-2H-pyran-3-carboxamide (1.73 g, 10 mmol) in dry tetrahydrofuran (10 mL) was added methyllithium (1.6M, 10 mmol, 6.25 mL) at −78° C. and the reaction was warmed up to 0° C. and stirred for 1.5 h. The mixture was then quenched with 0.5M hydrochloric acid (8.3 mL) and extracted with ethyl acetate (20 mL*2). The combined organic phase was washed with water (10 mL) and concentrated to obtain 1-(tetrahydro-2H-pyran-3-yl)ethan-1-one as yellow oil. (0.911 g, 71.2%).

### Step 3: Synthesis of (Z)-3-(dimethylamino)-1-(tetrahydro-2H-pyran-3-yl)prop-2-en-1-one

A solution of 1-(tetrahydro-2H-pyran-3-yl)ethan-1-one (1.28 g, 10 mmol) in N,N-dimethylformamide dimethyl acetal (7.14 g, 60 mmol) was stirred at 110° C. for 16 h and concentrated to obtain (Z)-3-(dimethylamino)-1-(tetrahydro-2H-pyran-3-yl)prop-2-en-1-one as yellow solid. (1.41 g, 77%). LCMS (ESI) m/z: 184.2[M+H]+.

### Step 4: Synthesis of 3-(tetrahydro-2H-pyran-3-yl)-1H-pyrazol

A mixture of (Z)-3-(dimethylamino)-1-(tetrahydro-2H-pyran-3-yl)prop-2-en-1-one (1.83 g, 1 mmol) and hydrazine hydrate (1.5 g, 3 mmol) in ethanol (10 mL) was stirred at 90° C. for 2 h. It was then concentrated to give 3-(tetrahydro-2H-pyran-3-yl)-1H-pyrazole as red solid. (1.04 g, 68%). LCMS (ESI) m/z: 153.3 [M+H]+.

### Step 5: Synthesis of 4-(2-(pyridin-4-yl)-5-(3-(tetrahydro-2H-pyran-3-yl)-1H-pyrazol-1-yl)pyrazolo[1,5-a]pyrimidin-7-yl)morpholin

A mixture of 4-(5-chloro-2-(pyridin-4-yl)pyrazolo[1,5-a]pyrimidin-7-yl)morpholine (158 mg, 0.5 mmol), 3-(tetrahydro-2H-pyran-3-yl)-1H-pyrazole (152 mg, 1 mmol) and cesium carbonate (1.5 mmol, 488 mg) in N,N-dimethylformamide (10 mL) was stirred at 80° C. for 16 h. The mixture was diluted with water (10 mL) and the precipitate formed was collected by filtration. The solid was washed successively with water (10 mL), ethanol (10 mL) and vacuum dried to obtain 4-(2-(pyridin-4-yl)-5-(3-(tetrahydro-2H-pyran-3-yl)-1H-pyrazol-1-yl)pyrazolo[1,5-a]pyrimidin-7-yl)morpholine as yellow solid. (55.3 mg, 26%). ¹H NMR (400 MHz, CDCL3) δ 8.71 (d, J=6.4 Hz, 2H), 8.51 (d, J=2.8 Hz, 1H), 7.83 (d, J=6.0 Hz, 2H), 6.97 (s, 1H), 6.83 (s, 1H), 6.36 (d, J=2.8 Hz, 1H), 4.13-4.17 (m, 1H), 4.03-4.06 (m, 4H), 3.99 (d, J=11.6 Hz, 1H), 3.90-3.92 (m, 4H), 3.49-3.58 (m, 2H), 3.06-3.11 (m, 1H), 2.18-2.19 (m, 1H), 1.72-1.83 (m, 3H); LCMS (ESI) m/z: 432.3 [M+H]+.

### Syntheses of 4-(5-(3-(piperidin-4-yl)-1H-pyrazol-1-yl)-2-(pyridin-4-yl)pyrazolo[1,5-a]pyrimidin-7-yl)morpholine (Compound 62) and 4-(5-(3-(1-methylpiperidin-4-yl)-1H-pyrazol-1-yl)-2-(pyridin-4-yl)pyrazolo[1,5-a]pyrimidin-7-yl)morpholine (Compound 63)

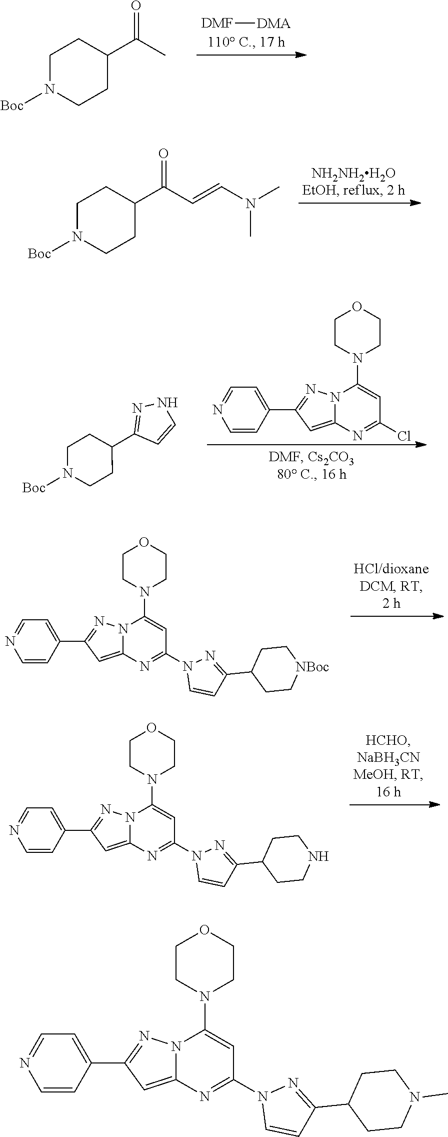

### Step 1: Synthesis of tert-butyl 4-(3-(dimethylamino)acryloyl)piperidine-1-carboxylate

A solution of tert-butyl 4-acetylpiperidine-1-carboxylate (908 mg, 4 mmol) in N,N-dimethylformamide dimethyl acetal (5 mL) was stirred at 110° C. for 17 h and concentrated to obtain tert-butyl 4-(3-(dimethylamino)acryloyl)piperidine-1-carboxylateas as yellow solid. (1.13 g, 99%). LCMS (ESI) m/z: 283.2 [M+H]+.

### Step 2: Synthesis of tert-butyl 4-(1H-pyrazol-3-yl)piperidine-1-carboxylate

A mixture of tert-butyl 4-(3-(dimethylamino)acryloyl)piperidine-1-carboxylate (1.13 g, 4 mmol) and hydrazine hydrate (600 mg, 12 mmol) in ethanol (10 mL) was stirred at reflux for 2 h. The reaction mixture was concentrated to obtain tert-butyl 4-(1H-pyrazol-3-yl)piperidine-1-carboxylate as yellow solid. (1 g, 99%). LCMS (ESI) m/z: 196.2 [M−56+H]+.

### Step 3: Synthesis of tert-butyl 4-(1-(7-morpholino-2-(pyridin-4-yl)pyrazolo[1,5-a]pyrimidin-5-yl)-1H-pyrazol-3-yl)piperidine-1-carboxylate

A mixture of 4-(5-chloro-2-(pyridin-4-yl)pyrazolo[1,5-a]pyrimidin-7-yl)morpholine (158 mg, 0.5 mmol), tert-butyl 4-(1H-pyrazol-3-yl)piperidine-1-carboxylate (251 mg, 1 mmol) and cesium carbonate (1.5 mmol, 488 mg) in DMF (10 mL) was stirred at 80° C. for 16 h. The mixture was diluted with water (10 mL) and the resultant precipitate was collected by filtration. The solid was washed with water (10 mL), EtOH (5 mL) and then dried to obtain tert-butyl 4-(1-(7-morpholino-2-(pyridin-4-yl)pyrazolo[1,5-a]pyrimidin-5-yl)-1H-pyrazol-3-yl)piperidine-1-carboxylate as yellow solid. (265 mg, 99%). LCMS (ESI) m/z: 531.3 [M+H]+.

### Step 4: Synthesis of 4-(5-(3-(piperidin-4-yl)-1H-pyrazol-1-yl)-2-(pyridin-4-yl)pyrazolo[1,5-a]pyrimidin-7-yl)morpholin

A mixture of tert-butyl 4-(1-(7-morpholino-2-(pyridin-4-yl)pyrazolo[1,5-a]pyrimidin-5-yl)-1H-pyrazol-3-yl)piperidine-1-carboxylate (265 mg, 0.5 mmol) in dichloromethane (2 mL) and HCl (4M in dioxane, 2 mL) was stirred at 25° C. for 2 h. The mixture was concentrated, and the residue was subjected to prep-HPLC (BOSTON pHlex ODS 10 μm 21.2×250 mm120 A. The mobile phase was acetonitrile/0.1% Formic acid) to obtain 4-(5-(3-(piperidin-4-yl)-1H-pyrazol-1-yl)-2-(pyridin-4-yl)pyrazolo[1,5-a]pyrimidin-7-yl)morpholine (100 mg, 42%) as yellow solid. ¹H NMR (400 MHz, CD₃OD) δ 8.63 (d, J=5.6 Hz, 2H), 8.58 (d, J=2.8 Hz, 1H), 8.01 (d, J=6.0 Hz, 2H), 6.98 (s, 1H), 6.95 (s, 1H), 6.54 (d, J=2.4 Hz, 1H), 4.01 (dd, J=6.4, 4.0 Hz, 4H), 3.93 (dd, J=4.8, 2.4 Hz, 4H), 3.51-3.54 (m, 2H), 3.16-3.24 (m, 3H), 2.29-2.34 (m, 2H), 2.02-2.05 (m, 2H); LCMS (ESI) m/z: 431.1 [M−HCOOH+H]+.

### Step 5: Synthesis of 4-(5-(3-(1-methylpiperidin-4-yl)-1H-pyrazol-1-yl)-2-(pyridin-4-yl)pyrazolo[1,5-a]pyrimidin-7-yl)morpholine

A solution of 4-(5-(3-(piperidin-4-yl)-1H-pyrazol-1-yl)-2-(pyridin-4-yl)pyrazolo[1,5-a]pyrimidin-7-yl)morpholine (100 mg, 0.23 mmol), 37% formaldehyde (5 drops) in methanol (5 mL) was stirred at room temperature for 1 h. Then sodium cyanoborohydride (72 mg, 1.15 mmol) was added and the reaction mixture was stirred for 16 h. The reaction mixture was concentrated, and the residue was subjected to prep-HPLC (BOSTON pHlex ODS 10 μm 21.2×250 mm120 A. The mobile phase was acetonitrile/0.1% Ammonium bicarbonate) to obtain 4-(5-(3-(1-methylpiperidin-4-yl)-1H-pyrazol-1-yl)-2-(pyridin-4-yl)pyrazolo[1,5-a]pyrimidin-7-yl)morpholine (40 mg, 38%) as white solid. ¹H NMR (400 MHz, CDCl₃) δ 8.70-8.72 (m, 2H), 8.50 (d, J=2.4 Hz, 1H), 7.83 (dd, J=4.4, 1.2 Hz, 2H), 6.97 (s, 1H), 6.83 (s, 1H), 6.36 (d, J=2.4 Hz, 1H), 3.89-4.05 (m, 8H), 3.01-3.04 (m, 2H), 2.74-2.76 (m, 1H), 2.38 (s, 3H), 1.89-2.19 (m, 6H); LCMS (ESI) m/z: 445.4 [M+H]+.

The following compounds were synthesized according to the protocols described above:

| Name | Structure | NMR, MS | Compd # |
|---|---|---|---|
| 4-(5-(3-(3- fluorophenyl)- 1H-pyrazol-1- yl)-2-(pyridin-4- yl)pyrazolo[1,5- a]pyrimidin-7- yl)morpholine | 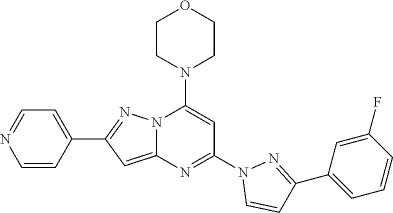 [SMILES: Fc1cccc(-c2ccn(-c3cc(N4CCOCC4)n4nc(-c5ccncc5)cc4n3)n2)c1] | ¹H NMR (400 MHz, DMSO-d₆) δ 8.76 (d, J = 3.2 Hz, 1H), 8.71 (dd, J = 4.8 1.2 Hz, 2H), 8.01 (dd, J = 4.8, 1.2 Hz, 2H), 7.85-7.89 (m, 2H), 7.54-7.58 (m, 1H), 7.24-7.26 (m, 3H), 7.07 (s, 1H), 3.93-3.95 (m, 8H); LCMS (ESI) m/z: 442.2 [M + H]⁺. | 64 |
| 4-(2-(pyridin-4- yl)-5-(3-(m- tolyl)-1H- pyrazol-1- yl)pyrazolo[1,5- a]pyrimidin-7- yl)morpholine | 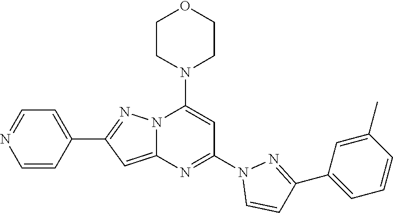 [SMILES: Cc1cccc(-c2ccn(-c3cc(N4CCOCC4)n4nc(-c5ccncc5)cc4n3)n2)c1] | ¹H NMR (400 MHz, DMSO-d₆) δ 8.72 (t, J = 4.4 Hz, 3H), 8.00 (d, J = 5.9 Hz, 2H), 7.82 (d, J = 11.8 Hz, 2H), 7.38 (t, J = 7.6 Hz, 1H), 7.24 (d, J = 7.5 Hz, 1H), 7.22 (s, 1H), 7.15 (d, J = 2.6 Hz, 1H), 7.04 (s, 1H), 3.93 (s, 8H), 2.41 (s, 3H). LCMS (ESI) m/z: 438.2 [M + H]⁺. | 65 |
| 4-(5-(3- methoxy-4- phenyl-1H- pyrazol-1-yl)-2- (pyridin-4- yl)pyrazolo[1,5- a]pyrimidin-7- yl)morpholine | 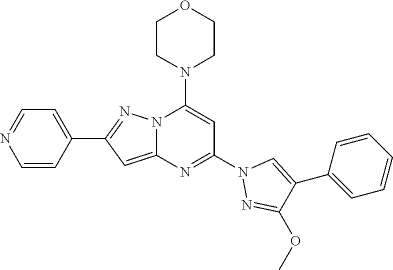 [SMILES: COc1nn(-c2cc(N3CCOCC3)n3nc(-c4ccncc4)cc3n2)cc1-c1ccccc1] | ¹H NMR (400 MHz, DMSO-d₆) δ 8.97 (s, 1H), 8.73 (d, J = 5.7 Hz, 2H), 8.04 (d, J = 5.8 Hz, 2H), 7.84 (d, J = 7.3 Hz, 2H), 7.41 (t, J = 7.7 Hz, 2H), 7.29 (d, J = 7.5 Hz, 1H), 7.16 (s, 1H), 6.84 (s, 1H), 4.12 (s, 3H), 3.91 (s, 8H). LCMS (ESI) m/z: 454.2 [M + H]⁺. | 66 |
| 4-(5-(3- (piperidin-3-yl)- 1H-pyrazol-1- yl)-2-(pyridin-4- yl)pyrazolo[1,5- a]pyrimidin-7- yl)morpholine | 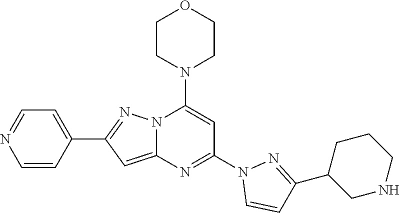 [SMILES: c1cc(-c2cc3nc(-n4ccc(C5CCCNC5)n4)cc(N4CCOCC4)n3n2)ccn1] | ¹H NMR (400 MHz, CD₃OD) δ 8.61 (d, J = 6.0 Hz, 2H), 8.51 (d, J = 2.4 Hz, 1H), 7.98 (d, J = 6.0 Hz, 2H), 6.93 (s, 1H), 6.92 (s, 1H), 6.46 (d, J = 2.8 Hz, 1H), 4.00 (d, J = 4.8 Hz, 4H), 3.91 (d, J = 4.4 Hz, 4H), 2.68- 3.29 (m, 5H), 1.69-2.13 (m, 4H); LCMS (ESI) m/z: 431.1 [M + H]⁺. | 67 |
| 4-(5-(3-(1- methylpiperidin- 3-yl)-1H- pyrazol-1-yl)-2- (pyridin-4- yl)pyrazolo[1,5- a]pyrimidin-7- yl)morpholine | 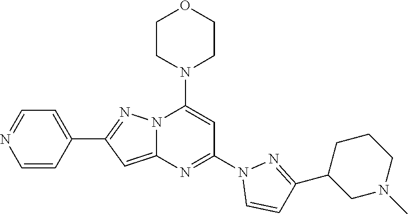 [SMILES: CN1CCCC(c2ccn(-c3cc(N4CCOCC4)n4nc(-c5ccncc5)cc4n3)n2)C1] | ¹H NMR (400 MHz, CDCL3) δ 8.71 (dd, J = 4.8, 1.6 Hz, 2H), 8.50 (d, J = 2.8 Hz, 1H), 7.82-7.84 (m, 2H), 6.98 (s, 1H), 6.83 (s, 1H), 6.35 (d, J = 2.4 Hz, 1H), 4.03-4.06 (m, 4H), 3.90- 3.92 (m, 4H), 3.04-3.13 (m, 2H), 2.90 (d, J = 11.2 Hz, 1H), 2.35 (s, 3H), 1.98-2.13 (m, 3H), 1.75-1.82 (m, 2H), 1.46-1.50 (m, 1H); LCMS (ESI) m/z: 445.3 [M + H]⁺. | 68 |
| 4-(5-(3- (pyridazin-4-yl)- 1H-pyrazol-1- yl)-2-(pyridin-4- yl)pyrazolo[1,5- a]pyrimidin-7- yl)morpholine | 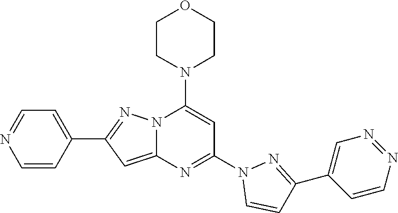 [SMILES: c1cc(-c2cc3nc(-n4ccc(-c5ccnnc5)n4)cc(N4CCOCC4)n3n2)ccn1] | ¹H NMR (400 MHz, CDCL3) δ 9.76 (s, 1H), 9.28 (d, J = 5.2 Hz, 1H), 8.76 − 8.73 (m, 3H), 7.92 − 7.90 (m, 1H), 7.86 − 7.84 (m, 2H), 7.08 (s, 1H), 6.99 (d, J = 2.4 Hz, 1H), 6.91 (s, 1H), 4.09 − 4.06 (m, 4H), 4.00-3.98 (m, 4H); LCMS (ESI) m/z: 426.1 [M + H]⁺. | 69 |
| 4-(5-(3-(pyridin- 2-yl)-1H- pyrazol-1-yl)-2- (pyridin-4- yl)pyrazolo[1,5- a]pyrimidin-7- yl)morpholine | 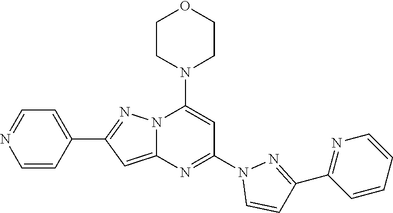 [SMILES: c1ccc(-c2ccn(-c3cc(N4CCOCC4)n4nc(-c5ccncc5)cc4n3)n2)nc1] | ¹H NMR (400 MHz, CDCL3) δ 8.73- 8.67 (m, 4H), 8.13 (d, J = 8 Hz, 1H), 7.85-7.78 (m, 3H), 7.31-7.26 (m, 1H), 7.17 (d, J = 2.8 HZ, 1H), 7.12 (s, 1H), 6.88 (s,1H), 4.07-4.05(m,4H), 3.96-3.94 (m, 4H); LCMS (ESI) m/z: 425.3 [M + H]⁺. | 70 |
| 4-(5-(3-(3- methoxyphenyl)- 1H-pyrazol-1- yl)-2-(pyridin-4- yl)pyrazolo[1,5- a]pyrimidin-7- yl)morpholine | 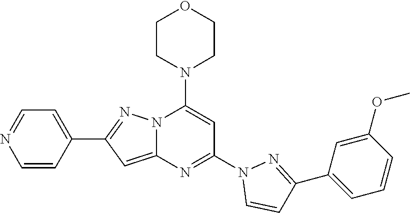 [SMILES: COc1cccc(-c2ccn(-c3cc(N4CCOCC4)n4nc(-c5ccncc5)cc4n3)n2)c1] | ¹H NMR (400 MHz, CDCL3) δ 8.72 (dd, J = 4.0, 1.2 Hz, 2H), 8.65 (d, J = 2.4 Hz, 1H), 7.84 (dd, J = 4.0, 1.6 Hz, 2H), 7.52-7.51 (m, 2H), 7.38 (t, J = 6.4 HZ, 1H), 7.11 (s, 1H), 6.96-6.94 (m, 1H), 6.86 (s, 1H), 6.83 (d, J = 2.0HZ, 1H), 4.07-4.05 (m, 4H), 3.95- 3.93 (m, 4H), 3.91 (s, 3H); LCMS (ESI) m/z: 454.2 [M + H]⁺. | 71 |
| 4-(2-(pyridin-4- yl)-5-(3- (pyrimidin-5- yl)-1H-pyrazol- 1- yl)pyrazolo[1,5- a]pyrimidin-7- yl)morpholine | 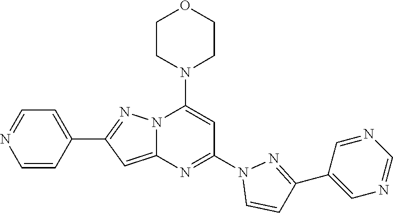 [SMILES: c1cc(-c2cc3nc(-n4ccc(-c5cncnc5)n4)cc(N4CCOCC4)n3n2)ccn1] | ¹H NMR (400 MHz, CDCL3) δ 9.26 (s, 2H), 9.23 (s, 1H), 8.74-8.72 (m, 3H), 7.85 (dd, J = 4.4, 1.4 Hz, 2H), 7.08 (s, 1H), 6.91-6.90 (m, 2H), 4.08- 4.06 (m, 4H), 3.99-3.96 (m, 4H); LCMS (ESI) m/z: 426.1 [M + H]⁺. | 72 |
| 4-(5-(3- (Azetidin-3-yl)- 1H-pyrazol-1- yl)-2-(pyridin-4- yl)pyrazolo[1,5- a]pyrimidin-7- yl)morpholine | 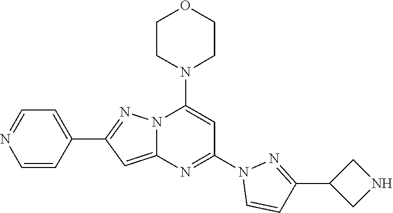 [SMILES: c1cc(-c2cc3nc(-n4ccc(C5CNC5)n4)cc(N4CCOCC4)n3n2)ccn1] | ¹H NMR (400 MHz, DMSO-d₆) δ 8.71-8.70 (m, 2H), 8.60 (d, J = 1.6 Hz, 1H), 7.99-7.98 (m, 2H), 7.19 (s, 1H), 6.89 (s, 1H), 6.70 (d, J = 2.4 Hz, 1H), 3.97-3.95 (m, 1H), 3.89 (s, 8 H), 3.80-3.77 (m, 4H); LCMS (ESI) m/z: 403.2 [M + H]⁺. | 73 |
| 4-(5-(3-(5,5- difluoropiperidin- 3-yl)-1H- pyrazol-1-yl)-2- (pyridin-4- yl)pyrazolo[1,5- a]pyrimidin-7- yl)morpholine | 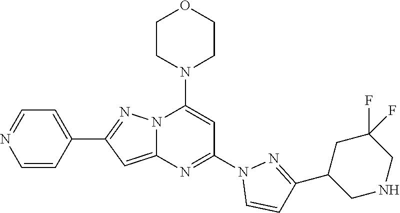 [SMILES: FC1(F)CNCC(c2ccn(-c3cc(N4CCOCC4)n4nc(-c5ccncc5)cc4n3)n2)C1] | ¹H NMR (400 MHz, DMSO-d₆) δ 8.71-8.69 (m, 2H), 8.60 (d, J = 2.8 Hz, 1H), 7.99-7.98 (m, 2H), 7.19 (s, 1H), 6.89 (s, 1H), 6.62 (d, J = 2.8 Hz, 1H), 3.89 (s, 8H), 3.14-3.06 (m, 3H), 2.86-2.66 (m, 1H), 2.39-2.17 (m, 3H); LCMS (ESI) m/z: 467.3 [M + H]⁺. | 74 |

### Synthesis of enantiomer 1 (Compound 75) and enantiomer 2 (Compound 76) of 4-(5-(3-(piperidin-3-yl)-1H-pyrazol-1-yl)-2-(pyridin-4-yl)pyrazolo[1,5-a]pyrimidin-7-yl)morpholine

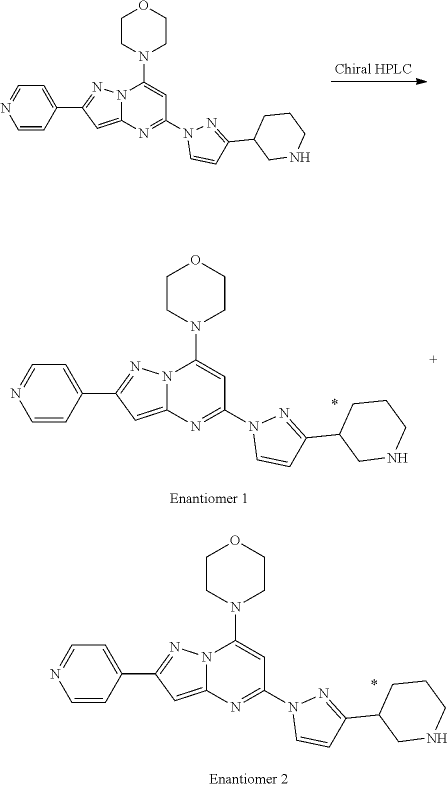 [SMILES: c1cc(-c2cc3nc(-n4ccc(C5CCCNC5)n4)cc(N4CCOCC4)n3n2)ccn1 ; c1cc(-c2cc3nc(-n4ccc(C5CCCNC5)n4)cc(N4CCOCC4)n3n2)ccn1 ; c1cc(-c2cc3nc(-n4ccc(C5CCCNC5)n4)cc(N4CCOCC4)n3n2)ccn1]

The racemic 4-(5-(3-(piperidin-3-yl)-1H-pyrazol-1-yl)-2-(pyridin-4-yl)pyrazolo[1,5-a]pyrimidin-7-yl)morpholine (100 mg, 0.23 mmol) was subjected to chiral-HPLC conditions to obtain enantiomer 1 (15 mg, 15%) and enantiomer 2 (7 mg, 7%) as white solids.

Compound 75: ¹H NMR (400 MHz, CD₃OD) δ 8.63 (dd, J=4.8, 1.6 Hz, 2H), 8.54 (d, J=2.4 Hz, 1H), 8.01 (dd, J=4.8, 1.6 Hz, 2H), 6.98 (s, 1H), 6.97 (s, 1H), 6.48 (d, J=2.8 Hz, 1H), 4.03-3.94 (m, 8H), 3.30-2.68 (m, 5H), 2.18-1.70 (m, 4H); LCMS (ESI) m/z: 431.3 [M+H]+; (Rt: 11.18 min).

Compound 76: ¹H NMR (400 MHz, CD₃OD) δ 8.64 (d, J=6.0 Hz, 2H), 8.55 (d, J=2.4 Hz, 1H), 8.02 (d, J=5.6 Hz, 2H), 6.99 (s, 1H), 6.98 (s, 1H), 6.49 (d, J=2.8 Hz, 1H), 4.03-3.94 (m, 8H), 3.30-2.69 (m, 5H), 2.18-1.68 (m, 4H); LCMS (ESI) m/z: 431.3 [M+H]+; (Rt: 13.28 min).

### Synthesis of 4-(5-(3-methoxy-4-phenyl-1H-pyrazol-1-yl)pyrazolo[1,5-a]pyrimidin-7-yl)morpholine (Compound 77)

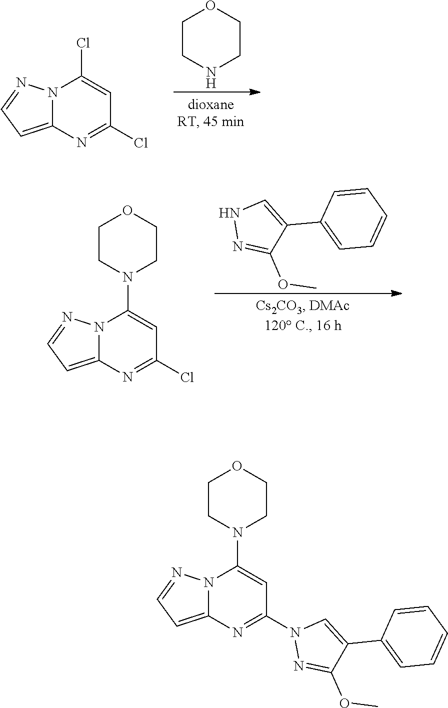 [SMILES: Clc1cc(Cl)n2nccc2n1 ; C1COCCN1 ; Clc1cc(N2CCOCC2)n2nccc2n1 ; COc1n[nH]cc1-c1ccccc1 ; COc1nn(-c2cc(N3CCOCC3)n3nccc3n2)cc1-c1ccccc1]

### Step 1: Synthesis of 4-(5-chloropyrazolo[1,5-a]pyrimidin-7-yl)morpholin

To a suspension of 5,7-dichloropyrazolo[1,5-a]pyrimidine (0.4 g, 2.13 mmol) in 1,4-dioxane (10 mL) was added morpholine (0.37 g, 4.25 mmol) and the resulting mixture was stirred at room temperature for 45 min. The mixture was then concentrated, and the residue was subjected to silica gel column chromatography (eluted with ethyl acetate in petroleum ether from 20% to 40%) to afford 4-(5-chloropyrazolo[1,5-a]pyrimidin-7-yl)morpholine (0.48 g, 94.7%) as pale-yellow solid. LCMS (ESI) m/z: 239.1 [M+H]⁺.

### Step 2: Synthesis of 4-(5-(3-methoxy-4-phenyl-1H-pyrazol-1-yl)pyrazolo[1,5-a]pyrimidin-7-yl)morpholin

A mixture of 4-(5-chloropyrazolo[1,5-a]pyrimidin-7-yl)morpholine (0.1 g, 0.42 mmol), 3-methoxy-4-phenyl-1H-pyrazole (73 mg, 0.42 mmol) and cesium carbonate (0.27 g, 0.84 mmol) in N,N-dimethylacetamide (10 mL) was stirred at 120° C. for 16 h. The reaction was cooled, the mixture was filtered to remove the solids and the filtrate was concentrated. The residue was subjected to prep-HPLC (BOSTON pHlex ODS 10 μm 21.2×250 mm 120 A. The mobile phase was acetonitrile/0.1% ammonium bicarbonate) to obtain 4-(5-(3-methoxy-4-phenyl-1H-pyrazol-1-yl)pyrazolo[1,5-a]pyrimidin-7-yl)morpholine (62.6 mg, 39.6%) as white solid. ¹H NMR (400 MHz, CDCl₃) δ 8.70 (s, 1H), 8.02 (d, J=2.2 Hz, 1H), 7.80-7.70 (m, 2H), 7.43-7.35 (m, 2H), 7.28-7.23 (m, 1H), 6.81 (s, 1H), 6.43 (d, J=2.3 Hz, 1H), 4.14 (s, 3H), 4.04-3.96 (m, 4H), 3.83 (dd, J=5.7, 3.7 Hz, 4H). LCMS (ESI) m/z: 377.1 [M+H]⁺.

The following compound was synthesized according to the protocol described above:

| Name | Structure | NMR, MS | Compd # |
|---|---|---|---|
| 4-(5-(3-(m- tolyl)-1H- pyrazol-1- yl)pyrazolo[1,5- a]pyrimidin-7- yl)morpholine | 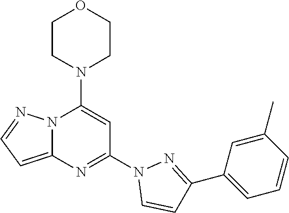 [SMILES: Cc1cccc(-c2ccn(-c3cc(N4CCOCC4)n4nccc4n3)n2)c1] | ¹H NMR (400 MHz, DMSO-d₆) δ 8.70 (d, J = 2.7 Hz, 1H), 8.17 (d, J = 2.3 Hz, 1H), 7.81 (d, J = 11.6 Hz, 2H), 7.38 (t, J = 7.6 Hz, 1H), 7.23 (d, J = 7.6 Hz, 1H), 7.12 (d, J = 2.7 Hz, 1H), 6.99 (s, 1H), 6.54 (d, J = 2.3 Hz, 1H), 3.87 (s, 8H), 2.40 (s, 3H). LCMS (ESI) m/z: 361.2 [M + H]⁺. | 78 |

### Synthesis of 2-(1-(7-morpholino-2-(pyridin-4-yl)pyrazolo[1,5-a]pyrimidin-5-yl)-1H-pyrazol-3-yl)ethan-1-ol (Compound 80) and 4-(5-(3-(2-methoxyethyl)-1H-pyrazol-1-yl)-2-(pyridin-4-yl)pyrazolo[1,5-a]pyrimidin-7-yl)morpholine (Compound 81)

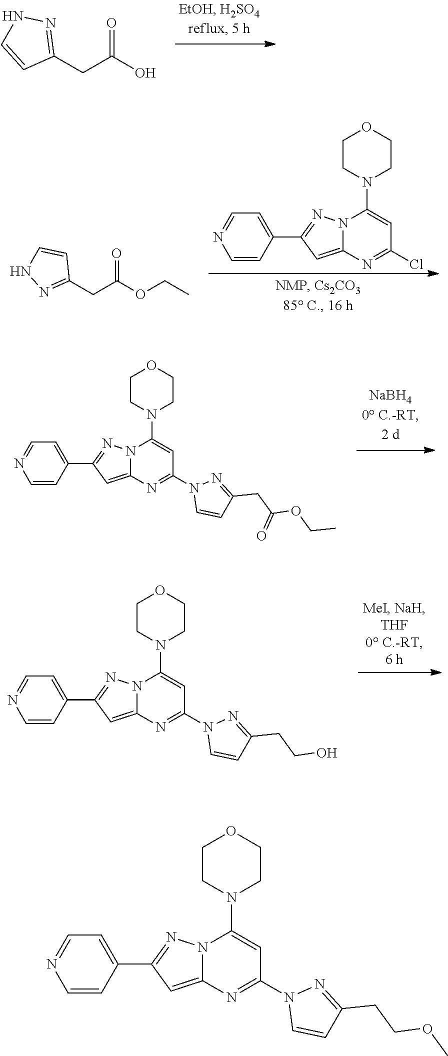 [SMILES: O=C(O)Cc1cc[nH]n1 ; CCOC(=O)Cc1cc[nH]n1 ; Clc1cc(N2CCOCC2)n2nc(-c3ccncc3)cc2n1 ; CCOC(=O)Cc1ccn(-c2cc(N3CCOCC3)n3nc(-c4ccncc4)cc3n2)n1 ; OCCc1ccn(-c2cc(N3CCOCC3)n3nc(-c4ccncc4)cc3n2)n1 ; COCCc1ccn(-c2cc(N3CCOCC3)n3nc(-c4ccncc4)cc3n2)n1]

### Step 1: Synthesis of ethyl 2-(1H-pyrazol-3-yl)acetate

To a solution of 2-(1H-pyrazol-3-yl)acetic acid (126 mg, 1 mmol) in ethanol (5 mL) was added thionyl chloride (590 mg, 5 mmol) dropwise and the mixture was heated to reflux for 5 h. It was then cooled and concentrated. To the resultant residue ammonia/methanol (7M, 5 mL) was added and then it was concentrated again. The residue was dissolved in dichloromethane (10 mL), the insoluble were filtered-off and the filtrate was concentrated to obtain ethyl 2-(1H-pyrazol-3-yl)acetate (154 mg, 99%) as yellow solid.

LCMS (ESI) m/z: 155.2 [M+H]+. This crude product was taken to the next step without further purification.

### Step 2: Synthesis of ethyl 2-(1-(7-morpholino-2-(pyridin-4-yl)pyrazolo[1,5-a]pyrimidin-5-yl)-1H-pyrazol-3-yl)acetate

A mixture of 4-(5-chloro-2-(pyridin-4-yl)pyrazolo[1,5-a]pyrimidin-7-yl)morpholine (158 mg, 0.5 mmol), ethyl 2-(1H-pyrazol-3-yl)acetate (154 mg, 1 mmol) and cesium carbonate (1.5 mmol, 488 mg) in N,N-dimethylformamide (10 mL) was stirred at 85° C. for 16 h. The reaction was quenched by the addition with water (20 mL) and the resultant mixture was extracted with dichloromethane (20 mL*3). The combined organic layer was washed with water (20 mL), dried over sodium sulfate and concentrated to obtain ethyl 2-(1-(7-morpholino-2-(pyridin-4-yl)pyrazolo[1,5-a]pyrimidin-5-yl)-1H-pyrazol-3-yl)acetate as yellow solid. (100 mg, 48%). LCMS (ESI) m/z: 434.3 [M+H]+.

### Step 3: Synthesis 2-(1-(7-morpholino-2-(pyridin-4-yl)pyrazolo[1,5-a]pyrimidin-5-yl)-1H-pyrazol-3-yl)ethan-1-ol

To a mixture of ethyl 2-(1-(7-morpholino-2-(pyridin-4-yl)pyrazolo[1,5-a]pyrimidin-5-yl)-1H-pyrazol-3-yl)acetate (43 mg, 0.1 mmol) in ether (5 mL) was added sodium borohydride (18 mg, 0.5 mmol) slowly at 0° C. and then the mixture was stirred at room temperature for 2 days. The reaction mixture was concentrated, and the residue was subjected to prep-HPLC (BOSTON pHlex ODS 10 μm 21.2×250 mm 120 A. The mobile phase was acetonitrile/0.1% Ammonium bicarbonate) to obtain 2-(1-(7-morpholino-2-(pyridin-4-yl)pyrazolo[1,5-a]pyrimidin-5-yl)-1H-pyrazol-3-yl)ethan-1-ol as white solid. (10 mg, 26%). ¹H NMR (400 MHz, CDCl₃) δ 8.71 (dd, J=4.4, 1.6 Hz, 2H), 8.54 (d, J=2.4 Hz, 1H), 7.83 (dd, J=4.4, 1.6 Hz, 2H), 6.92 (s, 1H), 6.84 (s, 1H), 6.39 (d, J=2.4 Hz, 1H), 4.05-3.89 (m, 10H), 3.00 (t, J=6.0 Hz, 2H), 2.51 (bs, 1H); LCMS (ESI) m/z: 392.3 [M+H]+.

### Step 4: Preparation of 4-(5-(3-(2-methoxyethyl)-1H-pyrazol-1-yl)-2-(pyridin-4-yl)pyrazolo[1,5-a]pyrimidin-7-yl)morpholin

To a mixture of 2-(1-(7-morpholino-2-(pyridin-4-yl)pyrazolo[1,5-a]pyrimidin-5-yl)-1H-pyrazol-3-yl)ethan-1-ol (10 mg, 0.025 mmol) in dry tetrahydrofuran (5 mL) was added sodium hydride (6 mg, 0.15 mmol, 60% suspension) portion-wise at 0° C. After the addition, to the resultant mixture was added iodomethane (14 mg, 0.1 mmol) and the mixture was stirred at room temperature for 6 h. The reaction was quenched by the addition with water (10 mL) and the mixture was extracted with ethyl acetate (10 mL*3). The organic layer was dried over sodium sulfate and concentrated. The residue was subjected to prep-HPLC (BOSTON pHlex ODS 10 μm 21.2×250 mm 120 A. The mobile phase was acetonitrile/0.1% Ammonium bicarbonate) to obtain 4-(5-(3-(2-methoxyethyl)-1H-pyrazol-1-yl)-2-(pyridin-4-yl)pyrazolo[1,5-a]pyrimidin-7-yl)morpholine as yellow solid. (1 mg, 10%). ¹H NMR (400 MHz, CDCl₃) δ 8.73-8.70 (m, 2H), 8.52 (d, J=2.8 Hz, 1H), 7.84-7.83 (m, 2H), 6.98 (s, 1H), 6.83 (s, 1H), 6.40 (d, J=2.8 Hz, 1H), 4.05-4.01 (m, 4H), 3.92-3.89 (m, 4H), 3.74 (t, J=6.8 Hz, 2H), 3.41 (s, 3H), 3.02 (t, J=6.8 Hz, 2H); LCMS (ESI) m/z: 406.2 [M+H]+.

### Synthesis of (1-(1-(7-morpholino-2-(pyridin-4-yl)pyrazolo[1,5-a]pyrimidin-5-yl)-1H-pyrazol-3-yl)cyclopropyl)methanol (Compound 82)

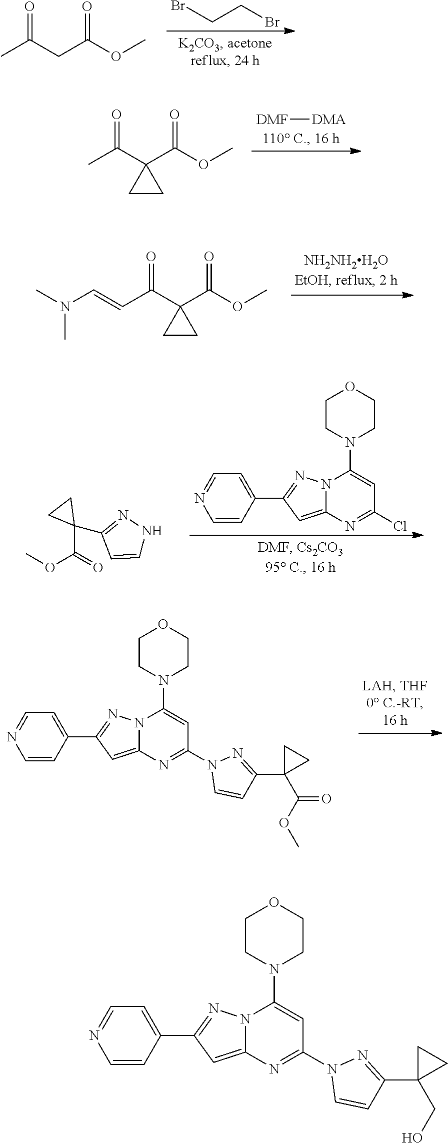

### Step 1: Synthesis of methyl 1-acetylcyclopropane-1-carboxylate

To a mixture of 1,2-dibromoethane (1.86 g, 10 mmol) and methyl 3-oxobutanoate (1.16 g, 10 mmol) in dry acetone (25 mL) was added potassium carbonate (2.07 g, 15 mmol). The reaction mixture was refluxed for 24 h and then cooled to room temperature. It was then diluted with water (50 mL) and the mixture was extracted with ethyl acetate (30 mL*3). The combined organic phase was dried over sodium sulfate and concentrated to obtain methyl 1-acetylcyclopropane-1-carboxylate as yellow oil. (1.42 g, 99%).

LCMS (ESI) m/z: 143.1 [M+H]+.

### Step 2: Synthesis of methyl (E)-1-(3-(dimethylamino)acryloyl)cyclopropane-1-carboxylate

A solution of methyl 1-acetylcyclopropane-1-carboxylate (1.42 g, 10 mmol) in N,N-dimethylformamide dimethyl acetal (10 mL) was stirred at 110° C. for 16 h. It was concentrated to give methyl (E)-1-(3-(dimethylamino)acryloyl)cyclopropane-1-carboxylate as yellow oil.(1.97 g, 99%). LCMS (ESI) m/z: 198.1 [M+H]+.

### Step 3: Synthesis methyl 1-(1H-pyrazol-3-yl)cyclopropane-1-carboxylate

A mixture of methyl (E)-1-(3-(dimethylamino)acryloyl)cyclopropane-1-carboxylate (1.97 g, mmol) and hydrazine hydrate (1.50 g, 30 mol) in ethanol (50 mL) was stirred at reflux temperature for 2 h. The reaction mixture was concentrated, and the residue was subjected to flash chromatography eluting with 0-100% methanol in aq. Ammonium bicarbonate (0.1%) to obtain methyl 1-(1H-pyrazol-3-yl)cyclopropane-1-carboxylate as yellow oil.(0.6 g, 36%). LCMS (ESI) m/z: 167.2 [M+H]+.

### Step 4: Synthesis methyl 1-(1-(7-morpholino-2-(pyridin-4-yl)pyrazolo[1,5-a]pyrimidin-5-yl)-1H-pyrazol-3-yl)cyclopropane-1-carboxylate

A mixture of 4-(5-chloro-2-(pyridin-4-yl)pyrazolo[1,5-a]pyrimidin-7-yl)morpholine (32 mg, 0.1 mmol), methyl 1-(1H-pyrazol-3-yl)cyclopropane-1-carboxylate (33 mg, 0.2 mmol) and cesium carbonate (0.3 mmol, 98 mg) in N,N-dimethylformamide (2 mL) was stirred at 95° C. for 16 h. It was diluted with water (10 mL) and precipitate formed was collected by filtration to obtain methyl 1-(1-(7-morpholino-2-(pyridin-4-yl)pyrazolo[1,5-a]pyrimidin-5-yl)-1H-pyrazol-3-yl)cyclopropane-1-carboxylate as a white solid. (45 mg, 99%). LCMS (ESI) m/z: 446.3 [M+H]+.

### Step 5: Synthesis (1-(1-(7-morpholino-2-(pyridin-4-yl)pyrazolo[1,5-a]pyrimidin-5-yl)-1H-pyrazol-3-yl)cyclopropyl)methano

To a mixture of methyl 1-(1-(7-morpholino-2-(pyridin-4-yl)pyrazolo[1,5-a]pyrimidin-5-yl)-1H-pyrazol-3-yl)cyclopropane-1-carboxylate (45 mg, 0.1 mmol) in ether (5 mL) was added sodium borohydride (18 mg, 0.5 mmol) portion-wise at 0° C. and then the mixture was stirred at room temperature for 16 h. The mixture was then quenched by the addition with water (10 mL) and the mixture was extracted with dichloromethane (10 mL*3). The combined organic phase was dried over sodium sulfate and concentrated. The resultant residue was subjected to prep-HPLC (BOSTON pHlex ODS 10 μm 21.2×250 mm 120 A. The mobile phase was acetonitrile/0.1% Ammonium bicarbonate) to obtain (1-(1-(7-morpholino-2-(pyridin-4-yl)pyrazolo[1,5-a]pyrimidin-5-yl)-1H-pyrazol-3-yl)cyclopropyl)methanol as white solid. (10 mg, 24%). ¹H NMR (400 MHz, CD₃OD) δ 8.64 (dd, J=4.8, 1.2 Hz, 2H), 8.52 (d, J=2.4 Hz, 1H), 8.03 (dd, J=4.8, 1.2 Hz, 2H), 7.02 (s, 1H), 6.98 (s, 1H), 6.49 (d, J=2.8 Hz, 1H), 4.03-4.01 (m, 4H), 3.95-0.3.93 (m, 4H), 3.87 (s, 2H), 1.11-1.10 (m, 4H); LCMS (ESI) m/z: 418.2 [M+H]+.

### Synthesis of 4-(5-(3-(1-(methoxymethyl)cyclopropyl)-1H-pyrazol-1-yl)-2-(pyridin-4-yl)pyrazolo[1,5-a]pyrimidin-7-yl)morpholine (Compound 83)

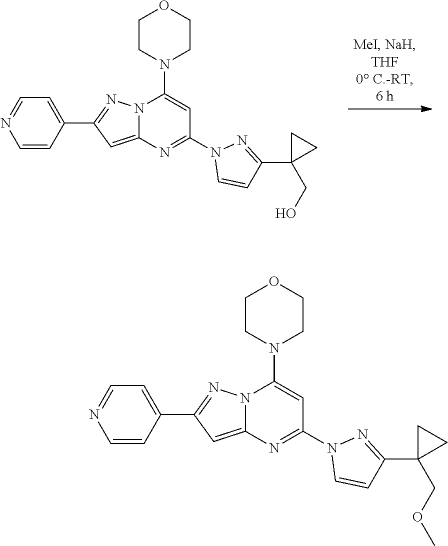 [SMILES: OCC1(c2ccn(-c3cc(N4CCOCC4)n4nc(-c5ccncc5)cc4n3)n2)CC1 ; COCC1(c2ccn(-c3cc(N4CCOCC4)n4nc(-c5ccncc5)cc4n3)n2)CC1]

To a stirred mixture of (1-(1-(7-morpholino-2-(pyridin-4-yl)pyrazolo[1,5-a]pyrimidin-5-yl)-1H-pyrazol-3-yl)cyclopropyl)methanol (42 mg, 0.1 mmol) in dry tetrahydrofuran (5 mL) was added sodium hydride (12 mg, 0.3 mmol, 60% suspension) portion-wise at 0° C. To the resultant mixture was iodomethane (28 mg, 0.2 mmol) and it was stirred at room temperature for 6 h. The reaction was quenched by the addition with ice-water (10 mL) and then the mixture was extracted with dichloromethane (10 mL*3). The combined organic layer was dried, concentrated and the residue was subjected to prep-HPLC (BOSTON pHlex ODS 10 μm 21.2×250 mm 120 A. The mobile phase was acetonitrile/0.1% Ammonium bicarbonate) to obtain 4-(5-(3-(1-(methoxymethyl)cyclopropyl)-1H-pyrazol-1-yl)-2-(pyridin-4-yl)pyrazolo[1,5-a]pyrimidin-7-yl)morpholine as white solid. (18 mg, 42%). ¹H NMR (400 MHz, CD₃OD) δ 8.65 (d, J=6.0 Hz, 2H), 8.53 (d, J=2.8 Hz, 1H), 8.04 (d, J=6.0 Hz, 2H), 7.00 (s, 1H), 6.99 (s, 1H), 6.53 (d, J=2.4 Hz, 1H), 4.03-4.01 (m, 4H), 3.95 (d, J=5.2 Hz, 4H), 3.72 (s, 2H), 3.43 (s, 3H), 1.17 (d, J=2.4 Hz, 2H),; LCMS (ESI) m/z: 432.3 M+H]+.

### Synthesis of 4-(5-(3-(2-(1-methylcyclopropyl)ethyl)-1H-pyrazol-1-yl)-2-(pyridin-4-yl)pyrazolo[1,5-a]pyrimidin-7-yl)morpholine (Compound 84)

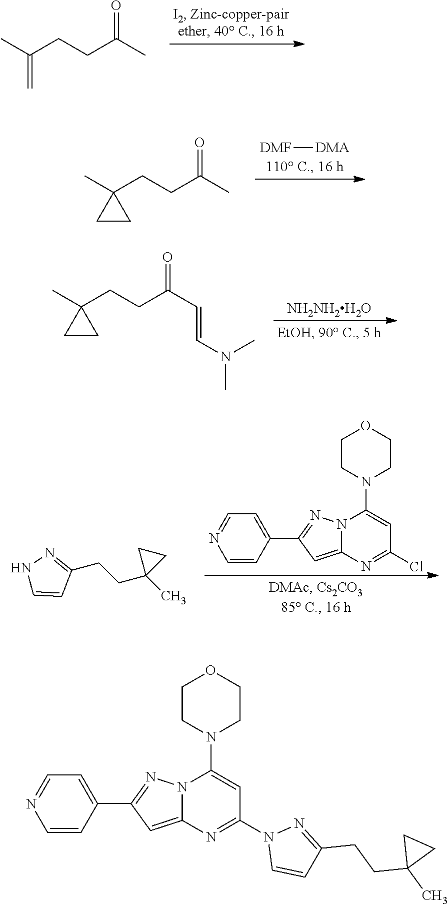

### Step 1: Synthesis of 4-(1-methylcyclopropyl)butan-2-on

To a suspension of zinc-copper-couple (1.29 g, 10 mmol) in 30 mL of ether was added iodine (2.54 g, mmol) at room temperature. And then a solution of 5-methylhex-5-en-2-one (560 mg, 5 mmol) in 2 mL of diethyl ether was added dropwise. After the addition, the reaction mixture was heated to 40° C. and stirred overnight at this temperature. After the cooling, the mixture was treated with diatomaceous earth, and it was washed several times with diethyl ether. The combined filtrates were washed with saturated aqueous sodium bicarbonate solution and water, dried over magnesium sulfate and then concentrated to obtain 4-(1-methylcyclopropyl)butan-2-one (630 mg, 99) as yellow oil. ¹H NMR (400 MHz, CDCl₃) δ 2.52 (t, J=8.0 Hz, 2H), 2.28 (s, 3H), 1.50 (t, J=8.0 Hz, 2H), 1.01 (s, 3H), 0.26-0.25 (m, 4H).

### Step 2: Synthesis of (E)-1-(dimethylamino)-5-(1-methylcyclopropyl)pent-1-en-3-one

A solution of 4-(1-methylcyclopropyl)butan-2-one (630 mg, 5.0 mmol) in N,N-dimethylformamide dimethyl acetal (10 mL) was stirred at 110° C. for 16 h. The mixture was then concentrated to obtain (E)-1-(dimethylamino)-5-(1-methylcyclopropyl)pent-1-en-3-one as yellow oil (905 mg, 99%). LCMS (ESI) m/z: 182.3 [M+H]+. This product was used in the next step without further purification.

### Step 3: Synthesis 3-(2-(1-methylcyclopropyl)ethyl)-1H-pyrazol

A mixture of (E)-1-(dimethylamino)-5-(1-methylcyclopropyl)pent-1-en-3-one (905 mg, 5 mmol) and hydrazine hydrate (750 mg, 15 mol) in ethanol (15 mL) was refluxed for 5 h. It was concentrated and the residue was subjected to column chromatography eluting with 0-100% methanol in aqueous ammonium bicarbonate (0.1%) to obtain 3-(2-(1-methylcyclopropyl)ethyl)-1H-pyrazole as yellow oil.(0.1 g, 13%).

LCMS (ESI) m/z: 151.2 [M+H]+.

### Step 4: Synthesis 4-(5-(3-(2-(1-methylcyclopropyl)ethyl)-1H-pyrazol-1-yl)-2-(pyridin-4-yl)pyrazolo[1,5-a]pyrimidin-7-yl)morpholin

A mixture of 4-(5-chloro-2-(pyridin-4-yl)pyrazolo[1,5-a]pyrimidin-7-yl)morpholine (32 mg, 0.1 mmol), 3-(2-(1-methylcyclopropyl)ethyl)-1H-pyrazole (30 mg, 0.2 mmol) and cesium carbonate (0.3 mmol, 98 mg) in N,N-dimethylformamide (2 mL) was stirred at 85° C. for 16 h. After cooling, the mixture was diluted with water (10 mL) and the resultant precipitate was filtered and dried. This solid was then subjected to silica gel column chromatography to obtain 4-(5-(3-(2-(1-methylcyclopropyl)ethyl)-1H-pyrazol-1-yl)-2-(pyridin-4-yl)pyrazolo[1,5-a]pyrimidin-7-yl)morpholine (18 mg, 41%) as white solid. ¹H NMR (400 MHz, CDCl₃) δ 8.71 (dd, J=4.4, 1.6 Hz, 2H), 8.48 (d, J=2.8 Hz, 1H), 7.83 (dd, J=4.4, 1.6 Hz, 2H), 6.96 (s, 1H), 6.82 (s, 1H), 6.31 (d, J=2.4 Hz, 1H), 4.04-4.02 (m, 4H), 3.91-3.89 (m, 4H), 2.82-2.78 (m, 2H), 1.67-1.63 (m, 2H), 1.13 (s, 3H), 0.33 (d, J=3.2 Hz, 2H), 0.30 (d, J=3.2 Hz, 2H); LCMS (ESI) m/z: 430.2 [M+H]+.

### Syntheses of 4-(2-(pyridin-4-yl)-5-(4,5,6,7-tetrahydro-2H-indazol-2-yl)pyrazolo[1,5-a]pyrimidin-7-yl)morpholine (Compound 85) and 4-(2-(pyridin-4-yl)-5-(4,5,6,7-tetrahydro-1H-indazol-1-yl)pyrazolo[1,5-a]pyrimidin-7-yl)morpholine (Compound 86)

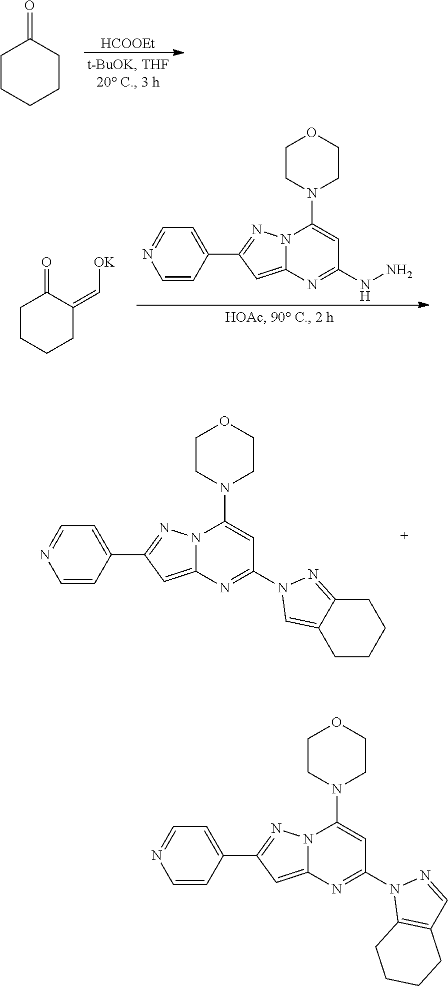

### Step 1: Synthesis of potassium (Z)-(2-oxocyclohexylidene)methanolat

To a solution of cyclohexanone (1.0 g, 10.2 mmol) and ethyl formate (1.21 g, 16.31 mmol) in tetrahydrofuran (50 mL) was added potassium tert-butoxide (10.2 mL, 10.2 mmol) at 0° C. Then the mixture was stirred at 20° C. for 2 h and concentrated. The crude product (Z)-2-(hydroxymethylene)cyclohexanone (1.4 g) thus obtained as yellow solid was used in the next step without further purification.

### Step 2: Synthesis of 4-(2-(pyridin-4-yl)-5-(4,5,6,7-tetrahydro-2H-indazol-2-yl)pyrazolo[1,5-a]pyrimidin-7-yl)morpholine formate

A solution of potassium (Z)-(2-oxocyclohexylidene)methanolate (162 mg, 1.286 mmol), and 4-(5-hydrazinyl-2-(pyridin-4-yl)pyrazolo[1,5-a]pyrimidin-7-yl)morpholine (200 mg, 0.643 mmol) in acetic acid (5 mL) was heated to 90° C. and stirred for 2 h. It was concentrated and the residue was subjected to prep-HPLC (SunFire C18, 4.6*50 mm, 3.5 um column Xbridge C18 3.5 μm 4.6×50 mm column. The elution system used was a gradient of 5%-95% over 1.5 min at 2 ml/min and the solvent was acetonitrile/0.01% aqueous HCOOH) to obtain 4-(2-(pyridin-4-yl)-5-(4,5,6,7-tetrahydro-2H-indazol-2-yl)pyrazolo[1,5-a]pyrimidin-7-yl)morpholine (65 mg, 25%) and 4-(2-(pyridin-4-yl)-5-(4,5,6,7-tetrahydro-1H-indazol-1-yl)pyrazolo[1,5-a]pyrimidin-7-yl)morpholine (34.2 mg, 13%) as white solids.

Compound 85: ¹H NMR (400 MHz, DMSO-d6) δ 8.70 (dd, J=4.5, 1.5 Hz, 2H), 8.49 (s, 1H), 7.99 (dd, J=4.5, 1.6 Hz, 2H), 7.63 (s, 1H), 7.21 (s, 1H), 6.93 (s, 1H), 3.88 (d, J=5.2 Hz, 4H), 3.84 (d, J=4.8 Hz, 4H), 3.20 (t, J=5.9 Hz, 2H), 2.52 (s, 2H), 1.82-1.70 (m, 4H); LCMS (ESI) m/z: 402.1 [M+H]+.

Compound 86: ¹H NMR (400 MHz, DMSO-d₆) δ 8.70 (dd, J=4.5, 1.5 Hz, 2H), 8.34 (s, 1H), 7.98 (dd, J=4.5, 1.6 Hz, 2H), 7.15 (s, 1H), 6.86 (s, 1H), 3.87 (t, J=5.5 Hz, 8H), 2.69 (t, J=6.2 Hz, 2H), 2.60 (t, J=5.9 Hz, 2H), 1.80-1.71 (m, 4H); LCMS (ESI) m/z: 402.1 [M+H]+.

### Synthesis of 2-(7-morpholino-2-(pyridin-4-yl)pyrazolo[1,5-a]pyrimidin-5-yl)-2,4,5,7-tetrahydropyrano[3,4-c]pyrazole (Compound 87) and 1-(7-morpholino-2-(pyridin-4-yl)pyrazolo[1,5-a]pyrimidin-5-yl)-1,4,5,7-tetrahydropyrano[3,4-c]pyrazole (Compound 88)

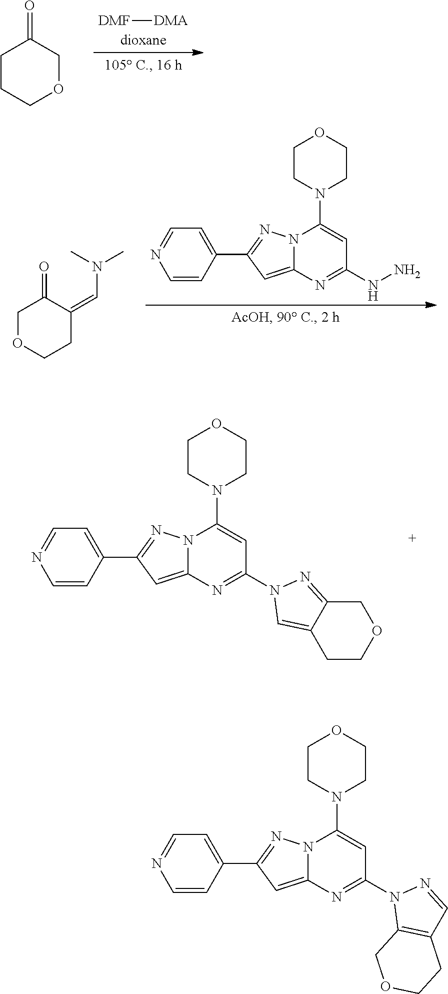

### Step 1: Synthesis of (Z)-4-((dimethylamino)methylene)dihydro-2H-pyran-3(4H)-one

To a solution of dihydro-2H-pyran-3(4H)-one (1.0 g, 9.99 mmol) in dioxane (50 mL) was added N,N-dimethylformamide dimethyl acetal (4.76 g, 39.96 mmol) at 20° C. The mixture was stirred at 105° C. for 16 h and concentrated. The crude product (Z)-4-((dimethylamino)methylene)dihydro-2H-pyran-3(4H)-one (1.4 g) thus obtained as yellow oil was used in the next step without further purification. LCMS (ESI) m/z: 156.1 [M+H]+.

### Step 2: Synthesis of 2-(7-morpholino-2-(pyridin-4-yl)pyrazolo[1,5-a]pyrimidin-5-yl)-2,4,5,7-tetrahydropyrano[3,4-c]pyrazole

A solution of (Z)-4-((dimethylamino)methylene)dihydro-2H-pyran-3(4H)-one (374 mg, 2.410 mmol) and 4-(5-hydrazinyl-2-(pyridin-4-yl)pyrazolo[1,5-a]pyrimidin-7-yl)morpholine (250 mg, 0.803 mmol) in acetic acid (5 mL) was heated to 90° C. and stirred for 2 h. It was then concentrated, and the residue was subjected to prep-HPLC (SunFire C18, 4.6*50 mm, 3.5 um column Xbridge C18 3.5 μm 4.6×50 mm column. The elution system used was a gradient of 5%-95% over 1.5 min at 2 ml/min and the solvent was acetonitrile/0.01% aqueous HCOOH) to obtain 2-(7-morpholino-2-(pyridin-4-yl)pyrazolo[1,5-a]pyrimidin-5-yl)-2,4,5,7-tetrahydropyrano[3,4-c]pyrazole (23.2 mg, 7.2%) and 1-(7-morpholino-2-(pyridin-4-yl)pyrazolo[1,5-a]pyrimidin-5-yl)-1,4,5,7-tetrahydropyrano[3,4-c]pyrazole (7.5 mg, 2.3%) as white solids.

Compound 87: ¹H NMR (400 MHz, DMSO-d₆) δ 8.69 (d, J=6.0 Hz, 2H), 7.97 (d, J=6.0 Hz, 2H), 7.72 (s, 1H), 7.17 (s, 1H), 6.90 (s, 1H), 5.15 (s, 2H), 4.22-3.67 (m, 10H), 2.64 (t, J=4.9 Hz, 2H); LCMS (ESI) m/z: 404.1 [M+H]+.

Compound 88: ¹H NMR (400 MHz, DMSO-d₆) δ 8.70 (s, 2H), 8.47 (s, 1H), 7.99 (s, 2H), 7.19 (s, 1H), 6.86 (s, 1H), 4.75 (s, 2H), 3.90-3.75 (m, 10H), 2.72 (s, 2H); LCMS (ESI) m/z: 404.1 [M+H]+.

The following compound were synthesized according to the protocols described above:

| Name | Structure | NMR, MS | Compd # |
|---|---|---|---|
| 2-(7-morpholino-2-(pyridin- 4-yl)pyrazolo[1,5- a]pyrimidin-5-yl)-2,4,6,7- tetrahydropyrano[4,3- c]pyrazole | 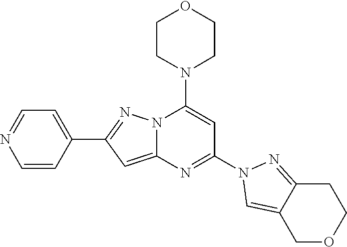 [SMILES: c1cc(-c2cc3nc(-n4cc5c(n4)CCOC5)cc(N4CCOCC4)n3n2)ccn1] | ¹H NMR (400 MHz, DMSO- d₆) δ 8.70 (d, J = 5.7 Hz, 2H), 7.99 (d, J = 5.8 Hz, 2H), 7.68 (s, 1H), 7.21 (s, 1H), 6.93 (s, 1H), 4.64 (s, 2H), 3.90-3.75 (m, 10H), 3.29 (s, 2H); LCMS (ESI) m/z: 404.1[M + H]⁺. | 89 |

### Synthesis of 4-(5-(3-phenyl-1H-pyrazol-1-yl)-2-(tetrahydrofuran-2-yl)pyrazolo[1,5-a]pyrimidin-7-yl)morpholine (Compound 90)

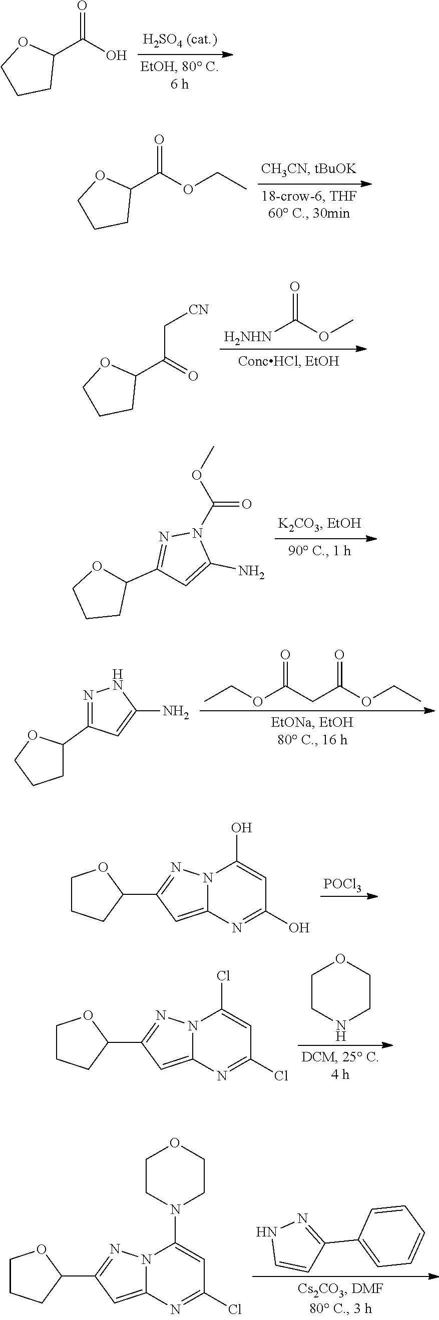

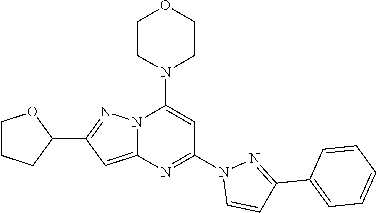 [SMILES: c1ccc(-c2ccn(-c3cc(N4CCOCC4)n4nc(C5CCCO5)cc4n3)n2)cc1]

### Step 1: Synthesis of ethyl tetrahydrofuran-2-carboxylate

To a solution of tetrahydrofuran-2-carboxylic acid (10 g, 86.2 mmol) in anhydrous ethanol (150 mL) was added concentrated sulfuric acid(10 mL). The resulting mixture was stirred at 80° C. for 6 h and concentrated. The residue was diluted with dichloromethane/water (40 mL/40 mL) and neutralized with saturated aqueous sodium bicarbonate solution. The organic layer was separated, and the aqueous layer was extracted with dichloromethane (40 mL) twice. The combined organic phase was dried over sodium sulfate and concentrated. The residue was subjected to flash chromatography (Biotage, 80 g silica gel, eluted with ethyl acetate in petroleum ether from 2% to 6%) to obtain ethyl tetrahydrofuran-2-carboxylate (8.1 g, 65.3%) as light-yellow liquid. LCMS (ESI) m/z: 145.2 [M+H]⁺.

### Step 2: Synthesis of 3-oxo-3-(tetrahydrofuran-2-yl)propanenitril

To a solution of 18-crown-6 (1.2 g, 4.5 mmol) and potassium tert-butoxide (1 M tetrahydrofuran solution, 54 mL, 54 mmol) in dry tetrahydrofuran (30 mL) was added ethyl tetrahydrofuran-2-carboxylate (6.5 g, 45.1 mmol) and the mixture was heated to 60° C. Then acetonitrile (2.2 g, 54.2 mmol) was added under nitrogen atmosphere and the reaction was stirred at 60° C. for an additional 30 min and cooled. It was concentrated to about ⅛^{th} of the volume (yellow oil) and used directly in next step without further purification. LCMS (ESI) m/z: 140.2 [M+H]⁺.

### Step 3: Synthesis of methyl 5-amino-3-(tetrahydrofuran-2-yl)-1H-pyrazole-1-carboxylate

To suspension of the crude 3-oxo-3-(tetrahydrofuran-2-yl)propanenitrile from the above step in ethanol (60 mL) at 0° C., were added concentrated hydrochloric acid (4 mL) and methyl carbazinate (4.9 g, 54.3 mmol). The mixture was stirred at 25° C. for 20 h, the resultant suspension was filtered, and the filtrate was concentrated. The residue was subjected to flash chromatography (Biotage, 40 g silica gel, eluted with ethyl acetate in petroleum ether from 40% to 60%) to obtain methyl 5-amino-3-(tetrahydrofuran-2-yl)-1H-pyrazole-1-carboxylate (2.92 g, 30% yield for two steps) as yellow solid. LCMS (ESI) m/z: 212.1 [M+H]⁺.

### Step 4: Synthesis of 3-(tetrahydrofuran-2-yl)-1H-pyrazol-5-amin

To a suspension of methyl 5-amino-3-(tetrahydrofuran-2-yl)-1H-pyrazole-1-carboxylate (2.92 g, 13.8 mmol) in ethanol (40 mL) was added potassium carbonate (1.4 g, 9.96 mmol) and the mixture was stirred at 90° C. for 2 h. It was concentrated, the residue was diluted with water (50 mL) and extracted with ethyl acetate (50 mL) twice. The combined organic phase was dried over sodium sulfate and concentrated to afford 3-(tetrahydrofuran-2-yl)-1H-pyrazol-5-amine (2.1 g, 99%) as yellow oil. LCMS (ESI) m/z: 154.2 [M+H]⁺.

### Step 5: Synthesis of 2-(tetrahydrofuran-2-yl)pyrazolo[1,5-a]pyrimidine-5,7-dio

To a solution of 3-(tetrahydrofuran-2-yl)-1H-pyrazol-5-amine (2.1 g, 13.7 mmol) in ethanol (35 mL) was added sodium ethoxide (20% in EtOH, 12.6 mL, 30.2 mmol) and diethyl malonate (2.3 mL, 15.1 mmol) and the mixture was stirred for 16 h at reflux temperature. The reaction mixture was cooled, the resultant precipitate was collected by filtration. The solid was slurred in a mixture of ethyl acetate and ethanol (30 mL/5 mL) and filtered to collect the precipitates. The product 2-(tetrahydrofuran-2-yl)pyrazolo[1,5-a]pyrimidine-5,7-diol (2.4 g, 79.1%) was isolated as yellow solid. LCMS (ESI) m/z: 222.1 [M+H]⁺.

### Step 6: Synthesis of 5,7-dichloro-2-(tetrahydrofuran-2-yl)pyrazolo[1,5-a]pyrimidin

A mixture of 2-(tetrahydrofuran-2-yl)pyrazolo[1,5-a]pyrimidine-5,7-diol (1 g, 4.5 mmol) and N,N-dimethylaniline (0.54 g, 4.5 mmol) in phosphorus oxychloride (20 mL) was stirred at 80° C. for 2 h. It was concentrated and the residue was diluted with dichloromethane (20 mL), neutralized with aqueous sodium bicarbonate solution and extracted with dichloromethane (15 mL) twice. The combined organic phase was dried over sodium sulfate and concentrated to obtain 5,7-dichloro-2-(tetrahydrofuran-2-yl)pyrazolo[1,5-a]pyrimidine (0.68 g, 58.6%) as brown oil. LCMS (ESI) m/z: 258.2 [M+H]⁺.

### Step 7: Synthesis of 4-(5-chloro-2-(tetrahydrofuran-2-yl)pyrazolo[1,5-a]pyrimidin-7-yl)morpholin

To a solution of 5,7-dichloro-2-(tetrahydrofuran-2-yl)pyrazolo[1,5-a]pyrimidine (0.65 g, 2.53 mmol) in dichloromethane (20 mL) at 0° C., was added morpholine (0.55 g, 6.32 mmol) dropwise under nitrogen atmosphere. After the addition, the reaction was stirred at 25° C. for 16 h and then diluted with dichloromethane/water (20 mL/20 mL). The organic layer was collected by separation and the aqueous layer was extracted with dichloromethane (20 mL) twice. The combined organic phase was washed with brine (40 mL), dried over sodium sulfate, filtered and concentrated. The residue was subjected to flash chromatography (Biotage, 40 g silica gel, eluted with ethyl acetate in petroleum ether from 30% to 50%) to obtain 4-(5-chloro-2-(tetrahydrofuran-2-yl)pyrazolo[1,5-a]pyrimidin-7-yl)morpholine (0.41 g, 52.6%) as white solid. LCMS (ESI) m/z: 309.3 [M+H]⁺.

### Step 8: Synthesis of 4-(5-(3-phenyl-1H-pyrazol-1-yl)-2-(tetrahydrofuran-2-yl)pyrazolo[1,5-a]pyrimidin-7-yl)morpholin

A mixture of 4-(5-chloro-2-(tetrahydrofuran-2-yl)pyrazolo[1,5-a]pyrimidin-7-yl)morpholine (0.1 g, 0.32 mmol), 3-phenyl-1H-pyrazole (47 mg, 0.32 mmol) and cesium carbonate (0.32 g, 0.97 mmol) in dry N,N-dimethylformamide (10 mL) was stirred at 80° C. for 5 h under nitrogen atmosphere. The reaction was cooled down and then diluted with ethyl acetate/water (20 mL/20 mL). The organic layer was collected by separation and aqueous layer was extracted with ethyl acetate (20 mL) twice. The combined organic phase was washed with brine (40 mL), dried over sodium sulfate, filtered and concentrated. The residue was subjected to prep-HPLC (base) to obtain 4-(5-(3-phenyl-1H-pyrazol-1-yl)-2-(tetrahydrofuran-2-yl)pyrazolo[1,5-a]pyrimidin-7-yl)morpholine (75 mg, 55.6%) as white solid. ¹H NMR (500 MHz, DMSO-d₆) δ 8.70 (d, J=3 Hz, 1H), 8.01 (d, J=7.5 Hz, 2H), 7.50 (t, J=7.5 Hz, 2H), 7.41 (t, J=7.5 Hz, 1H), 7.14 (d, J=3 Hz, 1H), 6.97 (s, 1H), 6.45 (s, 1H), 5.02 (t, J=6.5 Hz, 1H), 3.99-3.92 (m, 1H), 3.91-3.78 (m, 9H), 2.34-2.23 (m, 1H), 2.12-1.91 (m, 3H); LCMS (ESI) m/z: 417.4 [M+H]⁺.

The following compounds were synthesized according to the protocol described above:

| Name | Structure | NMR, MS | Compd # |
|---|---|---|---|
| 4-(5-(3-(2- fluorophenyl)-1H- pyrazol-1-yl)-2- (tetrahydrofuran- 2-yl)pyrazolo[1,5- a]pyrimidin-7- yl)morpholine | 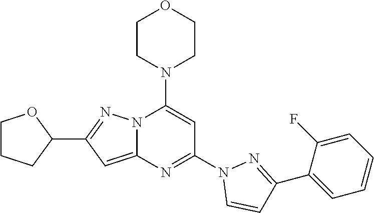 [SMILES: Fc1ccccc1-c1ccn(-c2cc(N3CCOCC3)n3nc(C4CCCO4)cc3n2)n1] | ¹H NMR (400 MHz, DMSO-d₆) δ 8.74 (d, J = 2.8 Hz, 1H), 8.20-8.10 (m, 1H), 7.53-7.44 (m, 1H), 7.41- 7.31 (m, 2H), 6.98 (t, J = 3.2 Hz, 1H), 6.96 (s, 1H), 6.50 (s, 1H), 5.02 (t, J = 6.8 Hz, 1H), 4.01-3.92 (m, 1H), 3.92-3.76 (m, 9H), 2.37- 2.22 (m, 1H), 2.14-1.90 (m, 3H); LCMS (ESI) m/z: 435.2 [M + H]⁺. | 91 |
| 4-(5-(3-(pyridin-3- yl)-1H-pyrazol-1- yl)-2- (tetrahydrofuran- 2-yl)pyrazolo[1,5- a]pyrimidin-7- yl)morpholine | 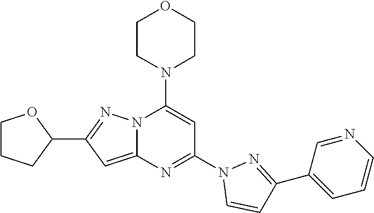 [SMILES: c1cncc(-c2ccn(-c3cc(N4CCOCC4)n4nc(C5CCCO5)cc4n3)n2)c1] | ¹H NMR (500 MHz, DMSO-d₆) δ 9.23 (d, J = 1.5 Hz, 1H), 8.74 (d, J = 3 Hz, 1H), 8.62 (dd, J = 4.5 Hz, 1 Hz, 1H), 8.37 (dt, J = 7.5 Hz, 2 Hz, 1H), 7.53 (dd, J = 7.5 Hz. 5 Hz, 1H), 7.25 (d, J = 2 Hz, 1H), 6.98 (s, 1H), 6.45 (s, 1H), 5.02 (t, J = 6.5 Hz, 1H), 4.00-3.93 (m, 1H), 3.90-3.76 (m, 9H), 2.34- 2.24 (m, 1H), 2.12-1.92 (m, 3H); LCMS (ESI) m/z: 418.2 [M + H]⁺. | 92 |
| 4-(5-(3-(3- methoxyphenyl)- 1H-pyrazol-1-yl)- 2- (tetrahydrofuran- 2-yl)pyrazolo[1,5- a]pyrimidin-7- yl)morpholine | 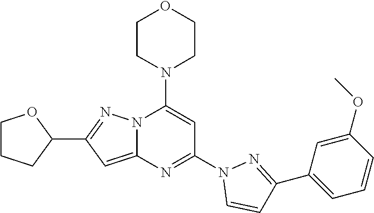 [SMILES: COc1cccc(-c2ccn(-c3cc(N4CCOCC4)n4nc(C5CCCO5)cc4n3)n2)c1] | ¹H NMR (500 MHz, DMSO-d₆) δ 8.70 (d, J = 3 Hz, 1H), 7.59 (d, J = 7.5 Hz, 1H), 7.54 (s, 1H), 7.41 (t, J = 8 Hz, 1H), 7.16 (d, J = 3 Hz, 1H), 7.00 (dd, J = 8.5 Hz, 2.5 Hz, 1H), 6.96 (s, 1H), 6.45 (s, 1H), 5.02 (t, J = 6.5 Hz, 1H), 4.00-3.93 (m, 1H), 3.90-3.78 (m, 12H), 2.35-2.23 (m, 1H), 2.12-1.91 (m, 3H); LCMS (ESI) m/z: 447.3 [M + H]⁺. | 93 |
| 4-(5-(3-(3- fluorophenyl)-1H- pyrazol-1-yl)-2- (tetrahydrofuran- 2-yl)pyrazolo[1,5- a]pyrimidin-7- yl)morpholine | 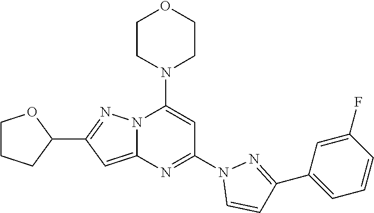 [SMILES: Fc1cccc(-c2ccn(-c3cc(N4CCOCC4)n4nc(C5CCCO5)cc4n3)n2)c1] | ¹H NMR (400 MHz, DMSO-d₆) δ 8.72 (d, J = 2.8 Hz, 1H), 7.89-7.82 (m, 2H), 7.59-7.50 (m, 1H), 7.29- 7.18 (m, 2H), 6.98 (s, 1H), 6.46 (s, 1H), 5.02 (t, J = 6.4 Hz, 1H), 4.00-3.93 (m, 1H), 3.90-3.76 (m, 9H), 2.35-2.23 (m, 1H), 2.12- 1.90 (m, 3H); LCMS (ESI) m/z: 435.2 [M + H]⁺. | 94 |
| 4-(5-(3- (tetrahydro-2H- pyran-4-yl)-1H- pyrazol-1-yl)-2- (tetrahydrofuran- 2-yl)pyrazolo[1,5- a]pyrimidin-7- yl)morpholine | 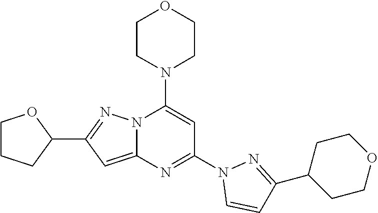 [SMILES: c1cn(-c2cc(N3CCOCC3)n3nc(C4CCCO4)cc3n2)nc1C1CCOCC1] | ¹H NMR (400 MHz, DMSO-d₆) δ 8.53 (d, J = 2 Hz, 1H), 6.80 (s, 1H), 6.54 (d, J = 2 Hz, 1H), 6.40 (s, 1H), 5.00 (t, J = 5.2 Hz, 1H), 3.99-3.90 (m, 3H), 3.88-3.74 (m, 9H), 3.46 (dt, J = 9.2 Hz, 1.2 Hz, 2H), 3.02- 2.92 (m, 1H), 2.33-2.22 (m, 1H), 2.09-1.66 (m, 7H); LCMS (ESI) m/z: 425.3 [M + H]⁺. | 95 |

### Synthesis of 4-(5-(1-phenyl-1H-pyrazol-3-yl)-2-(tetrahydrofuran-2-yl)pyrazolo[1,5-a]pyrimidin-7-yl)morpholine (Compound 96)

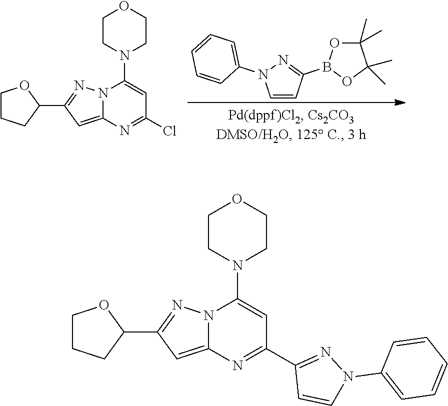 [SMILES: Clc1cc(N2CCOCC2)n2nc(C3CCCO3)cc2n1 ; CC1(C)OB(c2ccn(-c3ccccc3)n2)OC1(C)C ; c1ccc(-n2ccc(-c3cc(N4CCOCC4)n4nc(C5CCCO5)cc4n3)n2)cc1]

A mixture of 4-(5-chloro-2-(tetrahydrofuran-2-yl)pyrazolo[1,5-a]pyrimidin-7-yl)morpholine (0.12 g, 0.39 mmol), 1-phenyl-3-(4,4,5,5-tetramethyl-1,3,2-dioxaborolan-2-yl)-1H-pyrazole (0.13 g, 0.47 mmol), 1,1′-Bis(diphenylphosphino)ferrocene-palladium(II) dichloride dichloromethane complex (32 mg, 0.04 mmol) and cesium carbonate (0.38 g, 1.17 mmol) in DMSO/water (5 mL/1 mL) was stirred at 125° C. for 3 h under nitrogen atmosphere. The reaction mixture was cooled, and the mixture was diluted with ethyl acetate/water (20 mL/20 mL). The organic layer was separated, and the aqueous phase was extracted with ethyl acetate (20 mL) twice. The combined organic phase was washed with brine (40 mL), dried over sodium sulfate and concentrated. The residue was subjected to prep-HPLC (base) to afford 4-(5-(1-phenyl-1H-pyrazol-3-yl)-2-(tetrahydrofuran-2-yl)pyrazolo[1,5-a]pyrimidin-7-yl)morpholine (74.5 mg, 46%) as white solid. ¹H NMR (500 MHz, DMSO-d₆) δ 8.65 (d, J=2.5 Hz, 1H), 7.99 (d, J=8.5 Hz, 2H), 7.57 (t, J=7.5 Hz, 2H), 7.38 (t, J=7.5 Hz, 1H), 7.17 (d, J=2.5 Hz, 1H), 7.00 (s, 1H), 6.54 (s, 1H), 5.03 (t, J=6.5 Hz, 1H), 4.00-3.93 (m, 1H), 3.90-3.74 (m, 9H), 2.34-2.24 (m, 1H), 2.12-1.92 (m, 3H); LCMS (ESI) m/z: 417.3 [M+H]⁺.

### Synthesis of 4-(5-(2-phenylpyrimidin-4-yl)-2-(tetrahydrofuran-2-yl)pyrazolo[1,5-a]pyrimidin-7-yl)morpholine (Compound 97)

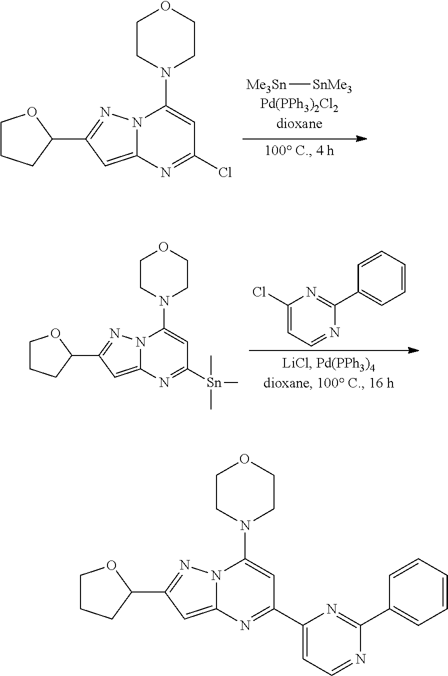

### Step 1: Synthesis of 4-(2-(tetrahydrofuran-2-yl)-5-(trimethylstannyl)pyrazolo[1,5-a]pyrimidin-7-yl)morphoine

A mixture of 4-(5-chloro-2-(tetrahydrofuran-2-yl)pyrazolo[1,5-a]pyrimidin-7-yl)morpholine (0.3 g, 1 mmol), hexamethyldistannane (0.5 mL, 24 mmol) and bis(triphenylphosphine)palladium(II) chloride (0.17 g, 0.24 mmol) in dry dioxane (20 mL) was stirred at 100° C. for 4 h under nitrogen atmosphere. It was cooled and the mixture was filtered through a pad of celite, and filtrate was concentrated. The residue was re-dissolved in dichloromethane (60 mL), washed successively with a solution of saturated aqueous potassium fluoride (50 mL), brine (50 mL), dried over sodium sulfate and concentrated. The product 4-(2-(tetrahydrofuran-2-yl)-5-(trimethylstannyl)pyrazolo [1,5-a]pyrimidin-7-yl)morpholine (0.36 g, 84.6%) was isolated as dark viscous liquid, which was used directly in next step without further purification. LCMS (ESI) m/z: 439.1 [M+H]⁺.

### Step 2: Synthesis of 4-(5-(2-phenylpyrimidin-4-yl)-2-(tetrahydrofuran-2-yl)pyrazolo[1,5-a]pyrimidin-7-yl)morphoine

A mixture of 4-(2-(tetrahydrofuran-2-yl)-5-(trimethylstannyl)pyrazolo[1,5-a]pyrimidin-7-yl)morpholine (0.26 g, 0.59 mmol), 4-chloro-2-phenylpyrimidine (140 mg, 0.71 mmol), lithium chloride (30 mg, 0.71 mmol) and tetrakis(triphenylphosphine)palladium (70 mg, 0.71 mmol) in dry dioxane (20 mL) was stirred at 100° C. for 15 h under nitrogen atmosphere. The mixture was cooled, filtered through a pad of celite and the filtrate was concentrated. The residue was diluted with ethyl acetate/water (20 mL/20 mL), the organic layer separated, and the aqueous phase was extracted with ethyl acetate (20 mL) twice. The combined organic phase was washed with brine (40 mL), dried over sodium sulfate and concentrated. The residue was subjected to prep-HPLC (base) to obtain 4-(5-(2-phenylpyrimidin-4-yl)-2-(tetrahydrofuran-2-yl)pyrazolo[1,5-a]pyrimidin-7-yl)morpholine (85 mg, 33.5%) as yellow solid. ¹H NMR (400 MHz, DMSO-d₆) δ 9.08 (d, J=4 Hz, 1H), 8.61-8.54 (m, 2H), 8.29 (d, J=4 Hz, 1H), 7.63-7.57 (m, 3H), 7.49 (s, 1H), 6.69 (s, 1H), 5.07 (t, J=5.2 Hz, 1H), 4.03-3.95 (m, 1H), 3.90 (s, 8H), 3.87-3.80 (m, 1H), 2.37-2.25 (m, 1H), 2.15-1.92 (m, 3H); LCMS (ESI) m/z: 429.1 [M+H]⁺.

### Synthesis of 4-(5-(3-phenyl-1H-pyrazol-1-yl)-2-(tetrahydro-2H-pyran-4-yl)pyrazolo[1,5-a]pyrimidin-7-yl)morpholine (Compound 98)

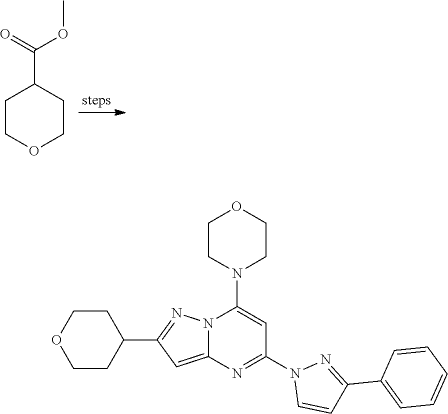

Compound 98 was synthesized according to the protocol described for compound 90. Compound 98 was isolated as white solid. ¹H NMR (400 MHz, Chloroform-d) δ 8.62 (d, J=2.7 Hz, 1H), 7.93 (d, J=3.2, 1.6 Hz, 2H), 7.51-7.33 (m, 3H), 7.01 (s, 1H), 6.81 (d, J=2.7 Hz, 1H), 6.30 (s, 1H), 4.10-4.07 (m, 2H), 4.08-3.98 (m, 4H), 3.92-3.81 (m, 4H), 3.63-3.55 (m, 2H), 3.13-3.03 (m, 1H), 2.06-1.86 (m, 4H); LCMS (ESI) m/z: 431.3 [M+H]+.

### Synthesis of 2-(7-morpholino-2-(tetrahydrofuran-2-yl)pyrazolo[1,5-a]pyrimidin-5-yl)-2,4,6,7-tetrahydropyrano[4,3-c]pyrazole (Compound 99) and 1-(7-morpholino-2-(tetrahydrofuran-2-yl)pyrazolo[1,5-a]pyrimidin-5-yl)-1,4,6,7-tetrahydropyrano[4,3-c]pyrazole (Compound 100)

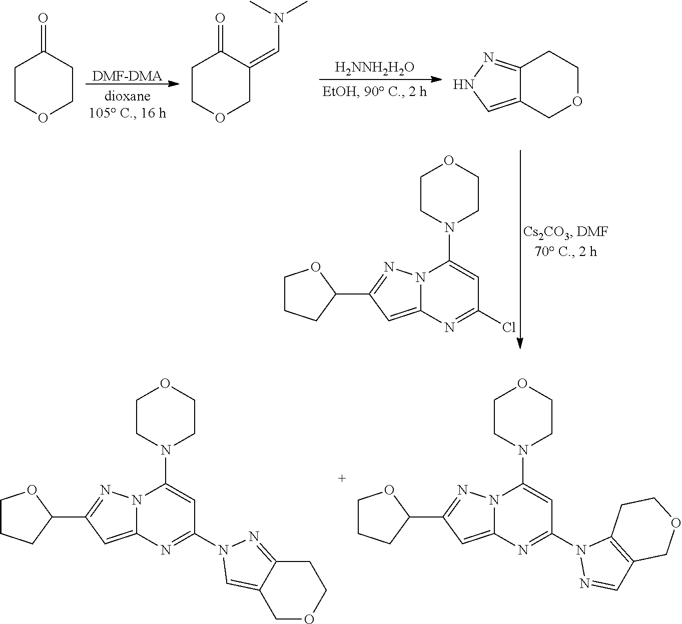

### Step 1: Synthesis of (Z)-3-((dimethylamino)methylene)dihydro-2H-pyran-4(3H)one

To a solution of dihydro-2H-pyran-4(3H)-one (2.0 g, 20.0 mmol) in dioxane (15 mL) was added N,N-dimethylformamide dimethyl acetal (9.53 g, 80.0 mmol). Then the mixture was heated to 90° C. and stirred for 16 h. It was then concentrated, and the residue was subjected to silica gel column chromatography (dichloromethane:/methanol=20:1) to obtain (Z)-3-((dimethylamino)methylene)dihydro-2H-pyran-4(3H)-one (0.545 g, crude) as yellow oil. LCMS (ESI) m/z: 156.1 [M+H]+.

### Step 2: Synthesis of 2,4,6,7-tetrahydropyrano[4,3-c]pyraole

A solution of (Z)-3-((dimethylamino)methylene)dihydro-2H-pyran-4(3H)-one (0.545 g, 3.514 mmol), hydrazine hydrate (0.352 g, 7.028 mmol) and acetic acid (211 mg, 3.514 mmol) in ethanol (10 mL) was heated to 90° C. and stirred for 2 h. The mixture was concentrated, and the residue was subjected to silica gel column chromatography (dichloromethane:/methanol=20:1) to obtain 2,4,6,7-tetrahydropyrano[4,3-c]pyrazole (0.287 g) as pale-yellow oil. LCMS (ESI) m/z: 125.2 [M+H]+.

### Step 3: Syntheses of compound 99 and compound 10

To a solution of 2,4,6,7-tetrahydropyrano[4,3-c]pyrazole (121 mg, 0.974 mmol), 4-(5-chloro-2-(tetrahydrofuran-2-yl)pyrazolo[1,5-a]pyrimidin-7-yl)morpholine (150 mg, 0.487 mmol) in dimethyl formamide (5 mL) was added cesium carbonate (476 mg, 1.461 mmol). Then contents were heated to 70° C. and stirred for 2 h. The mixture was concentrated, and the residue was subjected to prep-HPLC (SunFire C18, 4.6*50 mm, 3.5 um column Xbridge C18 3.5 μm 4.6×50 mm column. The elution system used was a gradient of 5%-95% over 1.5 min at 2 ml/min and the solvent was acetonitrile/0.01% aqueous NH₄HCO₃) to obtain 2-(7-morpholino-2-(tetrahydrofuran-2-yl)pyrazolo[1,5-a]pyrimidin-5-yl)-2,4,6,7-tetrahydropyrano[4,3-c]pyrazole (1.5 mg, 0.8%) and 1-(7-morpholino-2-(tetrahydrofuran-2-yl)pyrazolo[1,5-a]pyrimidin-5-yl)-1,4,6,7-tetrahydropyrano[4,3-c]pyrazole (24.5 mg, 13%) as white solids.

Compound 99: ¹H NMR (500 MHz, DMSO) δ 7.64 (s, 1H), 6.83 (s, 1H), 6.40 (s, 1H), 5.10-4.88 (m, 1H), 4.63 (s, 2H), 3.94 (dd, J=13.9, 7.5 Hz, 1H), 3.90-3.63 (m, 11H), 3.26 (d, J=6.9 Hz, 2H), 2.32-2.22 (m, 1H), 2.10-1.90 (m, 3H). LCMS (ESI) m/z: 397.1 [M+H]+.

Compound 100: ¹H NMR (400 MHz, DMSO) δ 8.37 (s, 1H), 6.78 (s, 1H), 6.39 (s, 1H), 5.06-4.93 (m, 1H), 4.68 (s, 2H), 4.08-3.68 (m, 11H), 2.79 (t, J=5.6 Hz, 2H), 2.35-2.22 (m, 1H), 2.11-1.86 (m, 3H); LCMS (ESI) m/z: 397.2 [M+H]+.

### Synthesis of 4-(2-(pyridin-4-yl)-5-vinylpyrazolo[1,5-a]pyrimidin-7-yl)morpholine (Compound 101)

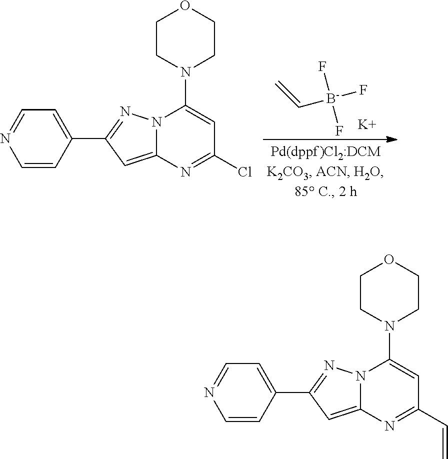

To a solution of 4-(5-chloro-2-(pyridin-4-yl)pyrazolo[1,5-a]pyrimidin-7-yl)morpholine (2.5 g, 7.9 mmol) in acetonitrile (66 mL) and water (33 mL) were added potassium trifluoro(vinyl)borate (2.1 g, 15.8 mmol), potassium carbonate (3.3 g, 23.8 mmol) and 1,1′-bis(diphenylphosphino)ferrocene-palladium(II)dichloride dichloromethane complex (700 mg, 0.8 mmol) at 25° C. under argon atmosphere. The mixture was stirred at 85° C. for 2 h, cooled and filtered to remove the solids. The filtrate was concentrated, and the residue was slurred with ethyl acetate (50 mL) to obtain 4-(2-(pyridin-4-yl)-5-vinylpyrazolo[1,5-a]pyrimidin-7-yl)morpholine as white solid (1.7 g, 70%). ¹H NMR (400 MHz, DMSO-d₆) δ 8.69 (d, J=6.0 Hz, 2H), 7.98 (d, J=6.0 Hz, 2H), 7.23 (s, 1H), 6.78 (dd, J=17.5, 10.8 Hz, 1H), 6.67 (s, 1H), 6.42 (d, J=17.5 Hz, 1H), 5.68 (d, J=11.6 Hz, 1H), 3.87 (d, J=5.3 Hz, 4H), 3.83 (d, J=5.3 Hz, 4H).; LCMS (ESI) m/z: 308.1 [M+H]+.

### Syntheses of 7-morpholino-2-(pyridin-4-yl)pyrazolo[1,5-a]pyrimidine-5-carboxamide (Compound 102), 7-morpholino-2-(pyridin-4-yl)pyrazolo[1,5-a]pyrimidine-5-carbonitrile (Compound 103), (7-morpholino-2-(pyridin-4-yl)pyrazolo[1,5-a]pyrimidin-5-yl)methanamine (Compound 104) and tert-butyl ((7-morpholino-2-(pyridin-4-yl)pyrazolo[1,5-a]pyrimidin-5-yl)methyl)carbamate (Compound 105)

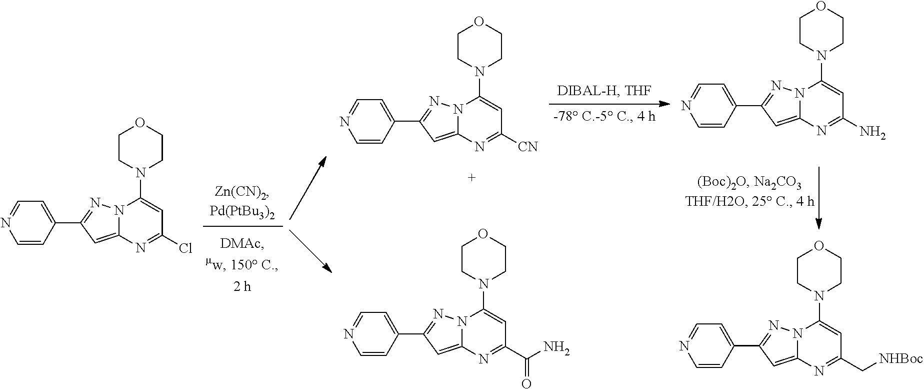 [SMILES: Clc1cc(N2CCOCC2)n2nc(-c3ccncc3)cc2n1 ; N#Cc1cc(N2CCOCC2)n2nc(-c3ccncc3)cc2n1 ; NC(=O)c1cc(N2CCOCC2)n2nc(-c3ccncc3)cc2n1 ; CC(C)(C)OC(=O)NCc1cc(N2CCOCC2)n2nc(-c3ccncc3)cc2n1 ; Nc1cc(N2CCOCC2)n2nc(-c3ccncc3)cc2n1]

### Step 1: Syntheses of 7-morpholino-2-(pyridin-4-yl)pyrazolo[1,5-a]pyrimidine-5-carboxamide and 7-morpholino-2-(pyridin-4-yl)pyrazolo[1,5-a]pyrimidine-5-carbonitile

To a solution of 4-(5-chloro-2-(pyridin-4-yl)pyrazolo[1,5-a]pyrimidin-7-yl)morpholine (0.5 g, 1.59 mmol) in DMAc (10 mL) were added zinc cyanide (0.15 g, 1.27 mmol) and bis(tri-tert-butylphosphine)palladium(0) (0.08 g, 0.16 mmol) and the mixture was stirred at 150° C. for 2 h in a microwave reactor. The mixture was filtered to remove the solids and the filtrate was subjected to pre-HPLC conditions (BOSTON pHlex ODS 10 μm 21.2×250 mm 120 A. The mobile phase was acetonitrile/0.1% Ammonium bicarbonate) to obtain 7-morpholino-2-(pyridin-4-yl)pyrazolo[1,5-a]pyrimidine-5-carbonitrile (22.5 mg) as pink colored solid and 7-morpholino-2-(pyridin-4-yl)pyrazolo[1,5-a]pyrimidine-5-carboxamide (42.8 mg) as white solid.

Compound 102: ¹H NMR (400 MHz, DMSO-d₆) δ 8.72 (dd, J=4.5, 1.5 Hz, 2H), 8.19 (s, 1H), 8.04 (dd, J=4.5, 1.6 Hz, 2H), 7.80 (s, 1H), 7.36 (s, 1H), 6.97 (s, 1H), 3.89 (s, 8H); LCMS (ESI) m/z: 325.2 [M+H]+.

Compound 103: ¹H NMR (400 MHz, DMSO-d₆) δ 8.73 (dd, J=4.5, 1.6 Hz, 2H), 8.03 (dd, J=4.5, 1.6 Hz, 2H), 7.49 (s, 1H), 7.08 (s, 1H), 4.08-3.97 (m, 4H), 3.91-3.77 (m, 4H); LCMS (ESI) m/z: 307.1 [M+H]+.

### Step 2: Synthesis of (7-morpholino-2-(pyridin-4-yl)pyrazolo[1,5-a]pyrimidin-5-yl)methanaine

Diisobutylaluminium hydride (26.1 mL, 26.1 mmol) was added a solution of 7-morpholino-2-(pyridin-4-yl)pyrazolo[1,5-a]pyrimidine-5-carbonitrile (2 g, 6.54 mmol) in 1,2-dimethoxyethane (87 mL) at −78° C. and the resultant mixture was stirred between −78° C.-5° C. under nitrogen for 4 h. The mixture was then poured into ice-water, followed by the addition of 1N hydrochloric acid (27 mL) and stirred for 10 min.

A saturated solution of aqueous sodium bicarbonate was added to the mixture until pH˜7. It was then extracted with ethyl acetate (20 mL×3), the combined organic phase was dried over sodium sulfate and concentrated to obtain the title compound. ¹H NMR (400 MHz, DMSO-d₆) δ 8.93 (d, J=6.4 Hz, 2H), 8.54 (s, 3H), 8.48 (d, J=6.0 Hz, 2H), 7.53 (s, 1H), 6.74 (s, 1H), 4.23 (d, J=5.6 Hz, 2H), 3.88 (s, 8H); LCMS (ESI) m/z: 311.1 [M+H]+.

### Step 3: Synthesis of tert-butyl ((7-morpholino-2-(pyridin-4-yl)pyrazolo[1,5-a]pyrimidin-5-yl)methyl)carbaate

To a solution of (7-morpholino-2-(pyridin-4-yl)pyrazolo[1,5-a]pyrimidin-5-yl)methanamine from step-2 and sodium bicarbonate (1.62 g, 13.08 mmol) in tetrahydrofuran (60 mL) and water (30 mL) was added di-tert-butyl dicarbonate (2.14 g, 9.81 mmol). The resultant mixture was stirred at 25° C. under nitrogen for 4 h. The mixture was filtered, the filtrate was extracted with ethyl acetate (100 mL*2), washed with brine (100 mL), dried, concentrated. The residue was subjected to flash column chromatography (petroleum ether: ethyl acetate=3:2) to obtain tert-butyl ((7-morpholino-2-(pyridin-4-yl)pyrazolo[1,5-a]pyrimidin-5-yl)methyl)carbamate (340 mg, 16.7%) as white solid. ¹H NMR (400 MHz, DMSO-d₆) δ 8.69 (d, J=5.4 Hz, 2H), 7.98 (d, J=5.4 Hz, 2H), 7.46 (t, J=6.0 Hz, 1H), 7.19 (s, 1H), 6.37 (s, 1H), 4.21 (d, J=6.0 Hz, 2H), 3.87 (d, J=4.6 Hz, 4H), 3.77 (d, J=4.4 Hz, 4H), 1.42 (s, 8H), 1.30 (s, 1H); LCMS (ESI) m/z: 411.2 [M+H]+.

### Synthesis of N′-benzoyl-7-morpholino-2-(pyridin-4-yl)pyrazolo[1,5-a]pyrimidine-5-carbohydrazide (Compound 106)

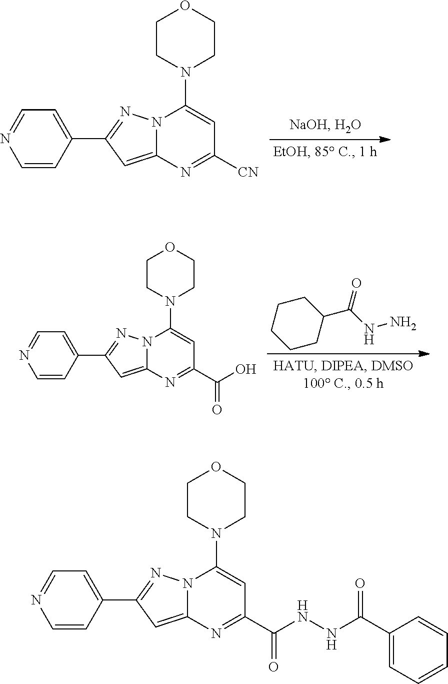 [SMILES: N#Cc1cc(N2CCOCC2)n2nc(-c3ccncc3)cc2n1 ; O=C(O)c1cc(N2CCOCC2)n2nc(-c3ccncc3)cc2n1 ; NNC(=O)C1CCCCC1 ; O=C(NNC(=O)c1cc(N2CCOCC2)n2nc(-c3ccncc3)cc2n1)c1ccccc1]

### Step 1: Synthesis of 7-morpholino-2-(pyridin-4-yl)pyrazolo[1,5-a]pyrimidine-5-carboxylic acid

To a suspension of 7-morpholino-2-(pyridin-4-yl)pyrazolo[1,5-a]pyrimidine-5-carbonitrile (2 g, 6.54 mmol) in ethanol (60 mL) was added 10% of sodium hydroxide in water (60 mL) and the mixture was stirred at 85° C. for 1.5 h. The reaction mixture was concentrated, diluted with water (20 mL) and the pH of the mixture was brought to ˜2 using 5N hydrochloric acid. The resultant precipitate was collected by filtration and vacuum dried to obtain 7-morpholino-2-(pyridin-4-yl)pyrazolo[1,5-a]pyrimidine-5-carboxylic acid (1.5 g, crude) as yellow solid. LCMS (ESI) m/z: 326.1 [M+H]+.

### Step 2: Synthesis of N′-benzoyl-7-morpholino-2-(pyridin-4-yl)pyrazolo[1,5-a]pyrimidine-5-carbohydrazide

A solution of 7-morpholino-2-(pyridin-4-yl)pyrazolo[1,5-a]pyrimidine-5-carboxylic acid (200 mg, 0.62 mmol), benzohydrazide (335 mg, 2.5 mmol), 1-[Bis(dimethylamino)methylene]-1H-1,2,3-triazolo[4,5-b]pyridinium 3-oxid hexafluorophosphate (353 mg, 0.93 mmol) and N,N-diisopropylethylamine (240 mg, 1.86 mmol) in dimethyl sulfoxide (40 mL) was stirred at 100° C. for 0.5 h under nitrogen atmosphere. The reaction mixture was then extracted with ethyl acetate (40 mL*2), the combined organic phase was washed with brine (40 mL), dried over sodium sulfate, filtered and concentrated. The residue was subjected to prep-HPLC (BOSTON pHlex ODS 10 μm 21.2×250 mm 120 A. The mobile phase was acetonitrile/0.1% aqueous formic acid) to obtain N′-benzoyl-7-morpholino-2-(pyridin-4-yl)pyrazolo[1,5-a]pyrimidine-5-carbohydrazide (8.8 mg, 3.2%) as yellow solid. ¹H NMR (400 MHz, DMSO-d₆) δ 8.73 (d, J=5.8 Hz, 2H), 8.40 (s, 1H), 8.07 (s, 2H), 7.94 (d, J=7.2 Hz, 2H), 7.60 (t, J=7.2 Hz, 1H), 7.53 (t, J=7.4 Hz, 2H), 7.44 (s, 1H), 6.97 (s, 1H), 3.93 (s, 4H), 3.91 (s, 4H); LCMS (ESI) m/z: 444.1 [M+H]+.

### Synthesis of 4-(5-(nitromethyl)-2-(pyridin-4-yl)pyrazolo[1,5-a]pyrimidin-7-yl)morpholine (Compound 107)

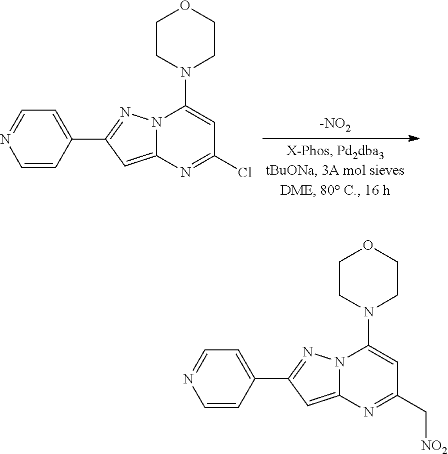 [SMILES: Clc1cc(N2CCOCC2)n2nc(-c3ccncc3)cc2n1 ; O=[N+]([O-])Cc1cc(N2CCOCC2)n2nc(-c3ccncc3)cc2n1]

A mixture of 4-(5-chloro-2-(pyridin-4-yl)pyrazolo[1,5-a]pyrimidin-7-yl)morpholine (0.5 g, 1.59 mmol), nitromethane (1 mL, 19 mmol), 2-(dicyclohexylphosphino)-2′,4′,6′-tri-i-propyl-1,1′-biphenyl (90 mg, 0.19 mmol), tris(dibenzylideneacetone)dipalladium (73 mg, 0.08 mmol), sodium t-butoxide (0.18 g, 1.9 mmol) and 3 Å molecular sieves (˜0.3 g) in dry 1,2-dimethoxyethane (40 mL) was stirred at 80° C. under nitrogen atmosphere for 16 h. The mixture was then filtered hot and concentrated. The residue was slurred in a mixture of petroleum ether/ethyl acetate (25 mL:1 mL), the resultant precipitate was collected by filtration and vacuum dried to obtain the crude product. It was further purified by prep-HPLC (SunFire C18, 4.6*50 mm, 3.5 um column Xbridge C18 3.5 μm 4.6×50 mm column. The elution system used was a gradient of 5%-95% over 1.5 min at 2 ml/min and the solvent was acetonitrile/0.01% aqueous NH₄HCO₃.) to obtain 4-(5-(nitromethyl)-2-(pyridin-4-yl)pyrazolo[1,5-a]pyrimidin-7-yl)morpholine (60 mg, 11%) as yellow solid. ¹H NMR (400 MHz, DMSO-d₆) δ 8.71 (dd, J=4.4, 1.6 Hz, 2H), 8.01 (dd, J=4.4, 1.6 Hz, 2H), 7.33 (s, 1H), 6.71 (s, 1H), 5.87 (s, 2H), 3.88 (s, 8H); LCMS (ESI) m/z: 341.1 [M+H]⁺.

### Synthesis of 1-(7-morpholino-2-(pyridin-4-yl)pyrazolo[1,5-a]pyrimidin-5-yl)ethenone (Compound 108)

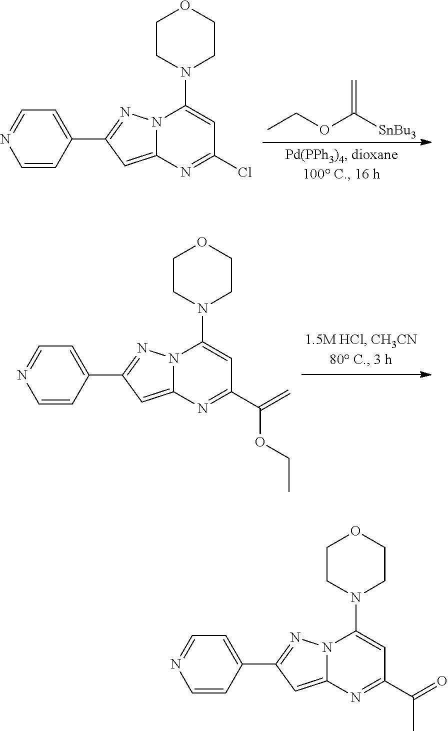

### Step 1: Synthesis of 4-(5-(1-ethoxyvinyl)-2-(pyridin-4-yl)pyrazolo[1,5-a]pyrimidin-7-yl)morpholine

A mixture of 4-(5-chloro-2-(pyridin-4-yl)pyrazolo[1,5-a]pyrimidin-7-yl)morpholine (3 g, 9.52 mmol), tributyl(1-ethoxyvinyl)stannane (4.8 mL, 14.28 mmol) and tetrakis(triphenylphosphine)palladium (1.1 g, 0.95 mmol) in dry 1,4-dioxane (200 mL) was stirred at 100° C. for 16 h under nitrogen atmosphere. The resultant reaction mixture was filtered through a pad of celite, and the filtrate was concentrated. The residue was slurred in a mixture solution of petroleum ether/ethyl acetate (50 mL/5 mL) and precipitate formed was collected by filtration and dried in vacuo to afford 4-(5-(1-ethoxyvinyl)-2-(pyridin-4-yl)pyrazolo[1,5-a]pyrimidin-7-yl)morpholine (3.3 g, 9.4 mmol) as yellow solid, which was used directly in next step.

### Step 2: Synthesis of 1-(7-morpholino-2-(pyridin-4-yl)pyrazolo[1,5-a]pyrimidin-5-yl)ethenone

To a solution of 4-(5-(1-ethoxyvinyl)-2-(pyridin-4-yl)pyrazolo[1,5-a]pyrimidin-7-yl)morpholine (3.2 g, 9.12 mmol) in acetonitrile (300 mL) was added aqueous HCl solution (1.5M, 40 mL) and then the mixture was stirred at 80° C. for 3 h. The precipitate was collected by filtration and the solids were slurred in a mixture of ethanol/sodium bicarbonate (aq. solution) (100 mL/100 mL). The solids from the slurry were collected by filtration, washed successively with water and ethyl acetate and dried under vacuum to obtain 1-(7-morpholino-2-(pyridin-4-yl)pyrazolo[1,5-a]pyrimidin-5-yl)ethanone (2.3 g, 74.9% for two steps) as yellow solid. A portion of this product (80 mg) was subjected to prep-HPLC (SunFire C18, 4.6*50 mm, 3.5 um column Xbridge C18 3.5 μm 4.6×50 mm column. The elution system used was a gradient of 5%-95% over 1.5 min at 2 ml/min and the solvent was acetonitrile/0.01% aqueous ammonium bicarbonate) to obtain 35 mg of the pure product. ¹H NMR (400 MHz, DMSO-d₆) δ 8.73 (d, J=5.6 Hz, 2H), 8.03 (d, J=6 Hz, 2H), 7.58 (s, 1H), 6.84 (s, 1H), 3.89 (s, 8H), 2.67 (s, 3H); LCMS (ESI) m/z: 324.1 [M+H]⁺.

### Synthesis of (E)-3-(7-morpholino-2-(pyridin-4-yl)pyrazolo[1,5-a]pyrimidin-5-yl)-1-phenylprop-2-en-1-one (Compound 109) and 2-((7-morpholino-2-(pyridin-4-yl)pyrazolo[1,5-a]pyrimidin-5-yl)methyl)-1,4-diphenylbutane-1,4-dione (Compound 110)

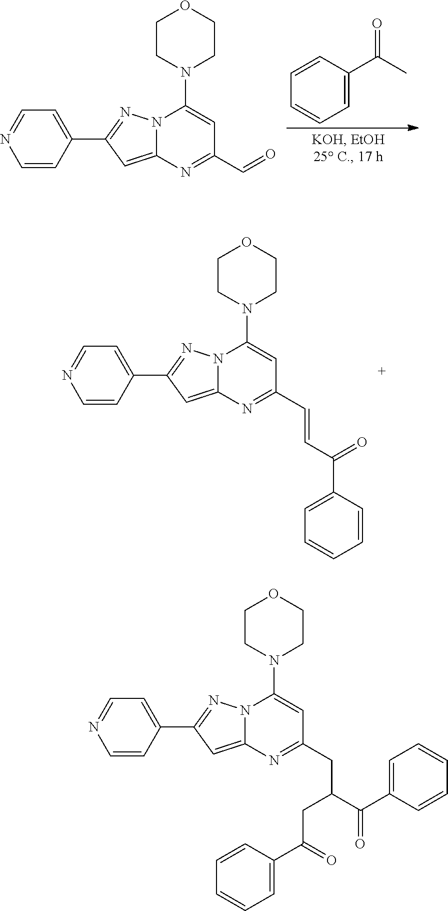

To a solution of 7-morpholino-2-(pyridin-4-yl)pyrazolo[1,5-a]pyrimidine-5-carbaldehyde (200 mg, 0.65 mmol) and acetophenone (78 mg, 0.65 mmol) in ethanol (6 mL) was added a solution of potassium hydroxide (37 mg, 0.65 mmol) in water (2 mL) at 0° C. The mixture was stirred at 25° C. for 17 h under argon atmosphere. It was then treated with 1.0 M HCl until it was slightly acidic (pH=5), the precipitate formed was collected by filtration and dried. It was further purified by subjecting it to prep-HPLC (Boston C18 21*250 mm 10 μm column. The mobile phase was acetonitrile/10 mM formic acid aqueous solution.) to obtain (E)-3-(7-morpholino-2-(pyridin-4-yl)pyrazolo[1,5-a]pyrimidin-5-yl)-1-phenylprop-2-en-1-one as yellow solid (15.3 mg, 6%). ¹H NMR (400 MHz, DMSO-d₆) δ 8.71 (d, J=5.9 Hz, 2H), 8.24 (d, J=15.6 Hz, 1H), 8.16 (d, J=7.2 Hz, 2H), 8.05-7.99 (m, 2H), 7.73 (t, J=7.3 Hz, 1H), 7.65 (s, 1H), 7.62 (d, J=8.1 Hz, 2H), 7.37 (s, 1H), 7.08 (s, 1H), 3.91 (s, 8H).; LCMS (ESI) m/z: 412.2 [M+H]+. And 2-((7-morpholino-2-(pyridin-4-yl)pyrazolo[1,5-a]pyrimidin-5-yl)methyl)-1,4-diphenylbutane-1,4-dione as yellow solid (9.4 mg, 3%). ¹H NMR (400 MHz, DMSO-d₆) δ 8.66 (d, J=5.9 Hz, 2H), 7.99 (d, J=7.2 Hz, 4H), 7.92 (d, J=6.0 Hz, 2H), 7.64 (t, J=7.4 Hz, 2H), 7.52 (t, J=7.6 Hz, 4H), 7.05 (s, 1H), 6.54 (s, 1H), 4.11-4.04 (m, 1H), 3.85 (d, J=4.9 Hz, 4H), 3.78 (d, J=5.0 Hz, 5H), 3.72 (s, 1H), 3.46 (d, J=6.2 Hz, 1H), 3.41 (d, J=6.3 Hz, 1H).; LCMS (ESI) m/z: 532.2 [M+H]+.

### Synthesis of 4-(5-(2-phenyl-3,4-dihydro-2H-pyrrol-4-yl)-2-(pyridin-4-yl)pyrazolo[1,5-a]pyrimidin-7-yl)morpholine (Compound 111) and 4-(5-(5-phenylpyrrolidin-3-yl)-2-(pyridin-4-yl)pyrazolo[1,5-a]pyrimidin-7-yl)morpholine (Compound 112)

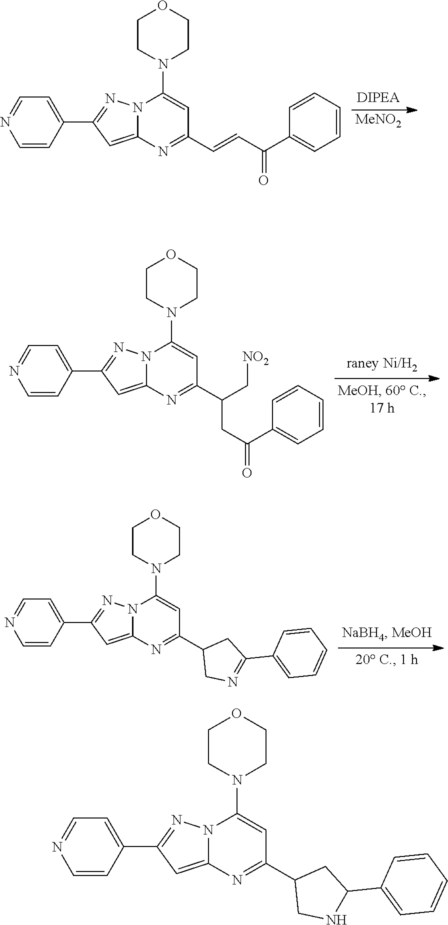

### Step 1: Synthesis of 3-(7-morpholino-2-(pyridin-4-yl)pyrazolo[1,5-a]pyrimidin-5-yl)-4-nitro-1-phenylbutan-1-one

To a solution of 3-(7-morpholino-2-(pyridin-4-yl)pyrazolo[1,5-a]pyrimidin-5-yl)-1-phenylprop-2-en-1-one (0.4 g, 0.97 mmol) and nitromethane (1.2 g, 19.4 mmol) in dimethyl sulfoxide (12 mL) was added diisopropylethylamine (0.1 g, 0.97 mmol) at 0° C. The mixture was stirred at 25° C. for 3 h, then poured into water(100 mL) and extracted with ethyl acetate (20 mL*3). The combined organic phase was concentrated to obtain 3-(7-morpholino-2-(pyridin-4-yl)pyrazolo[1,5-a]pyrimidin-5-yl)-4-nitro-1-phenylbutan-1-one as yellow solid (260 mg, 57%). LCMS (ESI) m/z: 473.4 [M+H]+.

### Step 2: Synthesis of 4-(5-(5-phenyl-3,4-dihydro-2H-pyrrol-3-yl)-2-(pyridin-4-yl)pyrazolo[1,5-a]pyrimidin-7-yl)morpholine

To a solution of 3-(7-morpholino-2-(pyridin-4-yl)pyrazolo[1,5-a]pyrimidin-5-yl)-4-nitro-1-phenylbutan-1-one (0.02 g, 0.04 mmol) in methanol (2 mL) was added Raney Ni(0.1 g) at 25° C. and the reaction flask was thoroughly flushed and filled with hydrogen. The reaction mixture was then stirred at 60° C. for 17 h under hydrogen atmosphere. The mixture was then filtered to remove the solids and the filtrate was concentrated. The residue was subjected to prep-HPLC (Boston C18 21*250 mm 10 μm column. The mobile phase was acetonitrile/10 mM Formic acid aqueous solution.) to obtain 4-(5-(5-phenyl-3,4-dihydro-2H-pyrrol-3-yl)-2-(pyridin-4-yl)pyrazolo[1,5-a]pyrimidin-7-yl)morpholine as a white solid.(2.7 mg, 15%)¹H NMR (400 MHz, DMSO-d₆) δ 8.68 (d, J=5.6 Hz, 2H), 7.95 (d, J=5.8 Hz, 2H), 7.90 (d, J=5.9 Hz, 2H), 7.49 (s, 2H), 7.48 (s, 1H), 7.16 (s, 1H), 6.48 (s, 1H), 4.47 (dd, J=16.2, 8.8 Hz, 1H), 4.12 (d, J=16.7 Hz, 1H), 3.90-3.75 (m, 9H), 3.44 (d, J=8.1 Hz, 2H).; LCMS (ESI) m/z: 425.3 [M+H]+.

### Step 1: Synthesis of 4-(5-(5-phenylpyrrolidin-3-yl)-2-(pyridin-4-yl)pyrazolo[1,5-a]pyrimidin-7-yl)morpholine

To a solution of 4-(5-(5-phenyl-3,4-dihydro-2H-pyrrol-3-yl)-2-(pyridin-4-yl)pyrazolo[1,5-a]pyrimidin-7-yl)morpholine (0.04 g, 0.08 mmol) in methanol (2 mL) was added sodium borohydride (0.01 g, 0.24 mmol) at 20° C. and the resultant mixture was stirred for 1 h under argon atmosphere. It was filtered to remove the solids; the filtrate was concentrated, and the residue was subjected to prep-HPLC (Boston C18 21*250 mm 10 μm column. The mobile phase was acetonitrile/10 mM Formic acid aqueous solution.) to obtain 4-(5-(5-phenylpyrrolidin-3-yl)-2-(pyridin-4-yl)pyrazolo[1,5-a]pyrimidin-7-yl)morpholine as a white solid.(5.0 mg, 12%)¹H NMR (400 MHz, DMSO-d₆) δ 8.68 (s, 2H), 7.96 (d, J=5.6 Hz, 2H), 7.45 (t, J=9.1 Hz, 2H), 7.33 (t, J=7.4 Hz, 2H), 7.25-7.19 (m, 1H), 7.16 (s, 1H), 6.43 (s, 1H), 4.25 (d, J=16 Hz, 1H), 3.86 (bs, 4H), 3.76 (bs, 4H), 3.51 (d, J=8.0 Hz, 1H), 3.23 (d, J=6.4 Hz, 1H), 2.61 (s, 1H), 2.02-1.86 (m, 1H).; LCMS (ESI) m/z: 427.2 [M+H]+.

### Synthesis of 4-(7-morpholino-2-(pyridin-4-yl)pyrazolo[1,5-a]pyrimidin-5-yl)-1-phenylbutan-1-one (Compound 113)

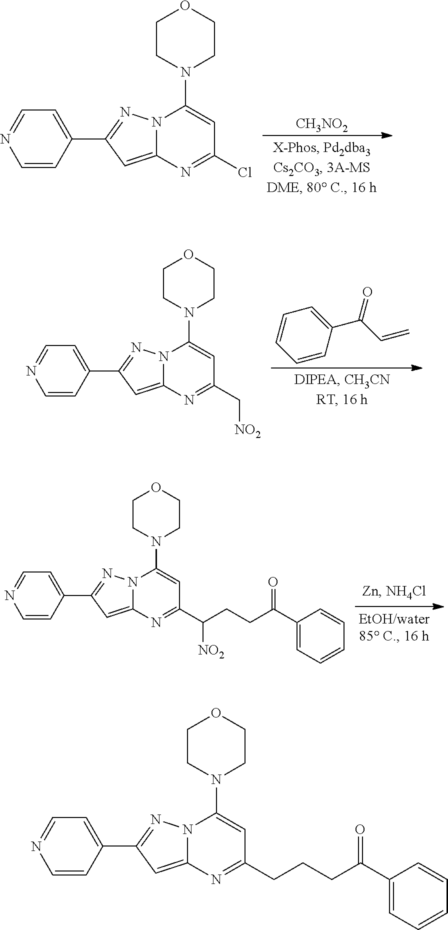

### Step 1: Synthesis of 4-(5-(nitromethyl)-2-(pyridin-4-yl)pyrazolo[1,5-a]pyrimidin-7-yl)morpholine

A mixture of 4-(5-chloro-2-(pyridin-4-yl)pyrazolo[1,5-a]pyrimidin-7-yl)morpholine (3 g, 9.52 mmol), nitromethane (6 mL, 114.3 mmol), 2-(dicyclohexylphosphino)-2′,4′,6′-tri-i-propyl-1,1′-biphenyl (540 mg, 1.14 mmol), tris(dibenzylideneacetone)dipalladium (440 mg, 0.48 mmol), sodium t-butoxide (1.08 g, 11.4 mmol) and 3 Å molecular (˜1 g) in dry 1,2-dimethoxyethane (200 mL) was stirred at 80° C. under nitrogen atmosphere for 16 h. It was concentrated, the residue was slurred in a mixture of petroleum ether/ethyl acetate (25 mL:1 mL) and the precipitate formed was collected by filtration and vacuum dried to obtain 4-(5-(nitromethyl)-2-(pyridin-4-yl)pyrazolo[1,5-a]pyrimidin-7-yl)morpholine (1.5 g, 46.4%) as yellow solid, which was used directly in next step. LCMS (ESI) m/z: 341.1 [M+H]+.

### Step 2: Synthesis of 4-(7-morpholino-2-(pyridin-4-yl)pyrazolo[1,5-a]pyrimidin-5-yl)-4-nitro-1-phenylbutan-1-one

To a solution of 4-(5-(nitromethyl)-2-(pyridin-4-yl)pyrazolo[1,5-a]pyrimidin-7-yl)morpholine (1.5 g, 4.41 mmol) in dry dimethyl sulfoxide (30 mL) was added 1-phenylprop-2-en-1-one (0.48 g, 3.67 mmol), followed by diisopropylethylamine (37 mg, 0.36 mmol) at 20° C. It was stirred at 20° C. for 32 h, then diluted with water (20 mL)/ethyl acetate (20 mL). The organic layer was removed and the aqueous phase which contained the title compound was used directly (as a DMSO/water solution) in next step without further purification.

### Step 3: Synthesis of 4-(7-morpholino-2-(pyridin-4-yl)pyrazolo[1,5-a]pyrimidin-5-yl)-1-phenylbutan-1-one

A mixture of 4-(7-morpholino-2-(pyridin-4-yl)pyrazolo[1,5-a]pyrimidin-5-yl)-4-nitro-1-phenylbutan-1-one (˜0.4 g, 0.85 mmol, from the previous step), zinc powder (0.55 g, 8.5 mmol) and ammonium chloride (0.45 g, 8.5 mmol) in ethanol (50 mL)/water (5 mL) was stirred at 80° C. for 16 h. The reaction mixture was filtered through a pad of celite, and the filtrate was concentrated. The residue was then subjected to prep-HPLC (SunFire C18, 4.6*50 mm, 3.5 um column Xbridge C18 3.5 μm 4.6×50 mm column. The elution system used was a gradient of 5%-95% over 1.5 min at 2 ml/min and the solvent was acetonitrile/0.01% aqueous NH4HCO3.) to obtain 4-(7-morpholino-2-(pyridin-4-yl)pyrazolo[1,5-a]pyrimidin-5-yl)-1-phenylbutan-1-one (30 mg, 9%) as white solid. ¹H NMR (400 MHz, DMSO-d₆) δ 8.69 (d, J=5.6 Hz, 2H), 7.97 (d, J=6.4 Hz, 4H), 7.64 (t, J=7.2 Hz, 1H), 7.53 (t, J=8 Hz, 2H), 7.15 (s, 1H), 6.39 (s, 1H), 3.86 (d, J=4.8 Hz, 4H), 3.77 (d, J=4.4 Hz, 4H), 3.13 (t, J=7.2 Hz, 2H), 2.82 (t, J=7.2 Hz, 2H), 2.16-2.03 (m, 2H); LCMS (ESI) m/z: 428.3 [M+H]⁺.

### Synthesis of 1-(7-morpholino-2-(pyridin-4-yl)pyrazolo[1,5-a]pyrimidin-5-yl)-3-phenylprop-2-en-1-one (Compound 114)

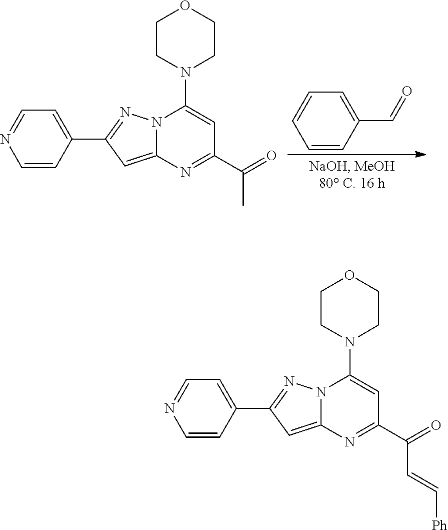

To a solution of benzaldehyde (250 mg, 1.55 mmol) in methanol (5 mL), cooled to 0° C., was added an aqueous solution of sodium hydroxide (10%, 1 mL, 2.32 mmol), followed by 1-(7-morpholino-2-(pyridin-4-yl)pyrazolo[1,5-a]pyrimidin-5-yl)ethanone (500 mg, 1.55 mmol) in portions. After the addition, the reaction was stirred at 80° C. for 16 h and concentrated. The residue was subjected to prep-HPLC (SunFire C18, 4.6*50 mm, 3.5 um column Xbridge C18 3.5 μm 4.6×50 mm column. The elution system used was a gradient of 5%-95% over 1.5 min at 2 ml/min and the solvent was acetonitrile/0.01% aqueous ammonium bicarbonate) to obtain 1-(7-morpholino-2-(pyridin-4-yl)pyrazolo[1,5-a]pyrimidin-5-yl)-3-phenylprop-2-en-1-one (15 mg, 5%) as yellow solid. ¹H NMR (400 MHz, DMSO-d₆) δ 8.74 (d, J=5.2 Hz, 2H), 8.26 (d, J=16.4 Hz, 1H), 8.06 (d, J=6 Hz, 2H), 7.92 (d, J=16 Hz, 1H), 7.88-7.83 (m, 2H), 7.63 (s, 1H), 7.55-7.48 (m, 3H), 7.00 (s, 1H), 3.92 (d, J=4.8 Hz, 8H); LCMS (ESI) m/z: 412.2 [M+H]⁺.

### Synthesis of tert-butyl imino(7-morpholino-2-(pyridin-4-yl)pyrazolo[1,5-a]pyrimidin-5-yl)methylcarbamate (Compound 115)

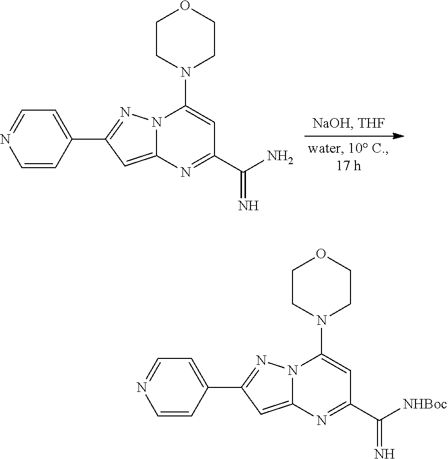 [SMILES: N=C(N)c1cc(N2CCOCC2)n2nc(-c3ccncc3)cc2n1 ; CC(C)(C)OC(=O)NC(=N)c1cc(N2CCOCC2)n2nc(-c3ccncc3)cc2n1]

To a solution of 7-morpholino-2-(pyridin-4-yl)pyrazolo[1,5-a]pyrimidine-5-carboximidamide (1.6 g, 4.95 mmol) in tetrahydrofuran (15 mL) were added di-tert-butyl dicarbonate (4.3 g, 19.8 mmol) and sodium hydroxide (792 mg, 19.8 mmol) in water (15 mL). The mixture was stirred at 10° C. for 17 h, then diluted with water (20 mL) and extracted with dichloromethane (20 mL*2). The combined organic phase was washed with brine (20 mL), dried over sodium sulfate, filtered and concentrated. The residue was subjected to prep-HPLC (Boston C18 21*250 mm 10 μm column. The mobile phase was acetonitrile/0.01% aqueous FA.) to obtain tert-butyl imino(7-morpholino-2-(pyridin-4-yl)pyrazolo[1,5-a]pyrimidin-5-yl)methylcarbamate (1.3 g, 62.2%) as yellow solid. ¹H NMR (400 MHz, DMSO) δ 8.95 (bs, 2H), 8.72 (dd, J=4.6, 1.5 Hz, 2H), 8.03 (t, J=8.9 Hz, 2H), 7.41 (s, 1H), 7.11 (s, 1H), 3.89 (s, 8H), 1.48 (s, 9H); LCMS (ESI) m/z: 424.2 [M+H]+.

### Syntheses of tert-butyl 3-benzyl-4-(7-morpholino-2-(pyridin-4-yl)pyrazolo[1,5-a]pyrimidin-5-yl)piperazine-1-carboxylate (Compound 116), 4-(5-(2-benzylpiperazin-1-yl)-2-(pyridin-4-yl)pyrazolo[1,5-a]pyrimidin-7-yl)morpholine (Compound 117) and 4-(5-(2-benzyl-4-methylpiperazin-1-yl)-2-(pyridin-4-yl)pyrazolo[1,5-a]pyrimidin-7-yl)morpholine (Compound 118)

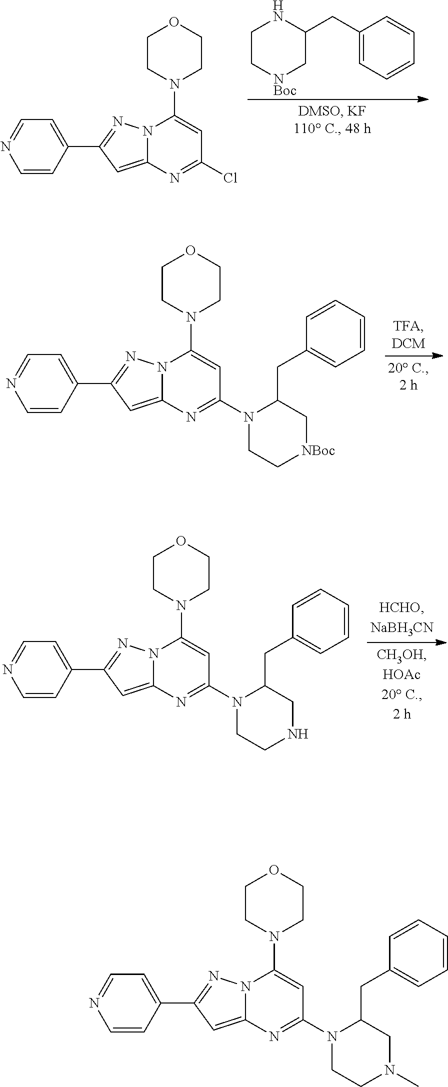 [SMILES: Clc1cc(N2CCOCC2)n2nc(-c3ccncc3)cc2n1 ; CC(C)(C)OC(=O)N1CCNC(Cc2ccccc2)C1 ; CC(C)(C)OC(=O)N1CCN(c2cc(N3CCOCC3)n3nc(-c4ccncc4)cc3n2)C(Cc2ccccc2)C1 ; c1ccc(CC2CNCCN2c2cc(N3CCOCC3)n3nc(-c4ccncc4)cc3n2)cc1 ; CN1CCN(c2cc(N3CCOCC3)n3nc(-c4ccncc4)cc3n2)C(Cc2ccccc2)C1]

### Step 1: Synthesis of tert-butyl 3-benzyl-4-(7-morpholino-2-(pyridin-4-yl)pyrazolo[1,5-a]pyrimidin-5-yl)piperazine-1-carboxylate

To a solution of 4-(5-chloro-2-(pyridin-4-yl)pyrazolo[1,5-a]pyrimidin-7-yl)morpholine (630 mg, 1.999 mmol) and tert-butyl 2-benzylpiperazine-1-carboxylate (580 mg, 1.999 mmol) in DMSO (12 mL) was added potassium fluoride (406 mg, 6.997 mmol). The mixture was heated to 110° C. and stirred for 48 h. It was then diluted with EtOAc (100 mL), washed with brine, dried over sodium sulfate and concentrated. The residue was subjected to silica gel column chromatography(dichloromethane:/methanol=20:1) to obtain tert-butyl 3-benzyl-4-(7-morpholino-2-(pyridin-4-yl)pyrazolo[1,5-a]pyrimidin-5-yl)piperazine-1-carboxylate (600 mg) as yellow solid. ¹H NMR (400 MHz, DMSO-d₆) δ 8.64 (dd, J=4.6, 1.4 Hz, 2H), 7.89 (d, J=6.0 Hz, 2H), 7.31 (s, 5H), 6.73 (s, 1H), 5.87 (bs, 1H), 4.68 (bs, 1H), 4.31 (s, 1H), 3.86-3.83 (m, 6H), 3.61 (s, 4H), 3.25 (s, 1H), 3.10-2.75 (m, 4H), 1.45 (s, 9H); LCMS (ESI) m/z: 556.3 [M+H]+.

### Step 2: Synthesis of 4-(5-(2-benzylpiperazin-1-yl)-2-(pyridin-4-yl)pyrazolo[1,5-a]pyrimidin-7-yl)morpholine

To a solution of tert-butyl 3-benzyl-4-(7-morpholino-2-(pyridin-4-yl)pyrazolo[1,5-a]pyrimidin-5-yl)piperazine-1-carboxylate (450 mg, 0.810 mmol) in dichloromethane (2 mL) was added trifluoroacetic acid (2 mL). The resultant mixture was stirred at 20° C. for 2 h. The reaction mixture was then concentrated, and the residue was subjected to silica gel column chromatography (dichloromethane:/methanol=20:1) to obtain 4-(5-(2-benzylpiperazin-1-yl)-2-(pyridin-4-yl)pyrazolo[1,5-a]pyrimidin-7-yl)morpholine (280 mg, 76%) as light-yellow solid. ¹H NMR (400 MHz, DMSO) δ 8.64 (dd, J=4.5, 1.5 Hz, 2H), 7.87 (dd, J=4.6, 1.4 Hz, 2H), 7.30-7.13 (m, 5H), 6.65 (s, 1H), 5.70 (s, 1H), 4.50 (s, 1H), 4.24 (s, 1H), 3.84 (t, J=4.5 Hz, 4H), 3.59-3.50 (m, 4H), 3.21-2.99 (m, 3H), 2.92 (m, 1H), 2.81 (d, J=12.0 Hz, 1H), 2.70-2.56 (m, 2H); LCMS (ESI) m/z: 456.3 [M+H]+.

### Step 3: Synthesis of 4-(5-(2-benzyl-4-methylpiperazin-1-yl)-2-(pyridin-4-yl)pyrazolo[1,5-a]pyrimidin-7-yl)morpholine

To a solution of 4-(5-(2-benzylpiperazin-1-yl)-2-(pyridin-4-yl)pyrazolo[1,5-a]pyrimidin-7-yl)morpholine (120 mg, 0.264 mmol) and formalin (198 mg, 2.64 mmol) in methanol (5 mL) was added a drop of acetic acid. The mixture was stirred at 20° C. for 1 h and to the mixture was added sodium cyanoborohydride (58 mg, 0.924 mmol). It was stirred further for another 1 h and concentrated. The residue was subjected to prep-HPLC (SunFire C18, 4.6*50 mm, 3.5 um column Xbridge C18 3.5 μm 4.6×50 mm column. The elution system used was a gradient of 5%-95% over 1.5 min at 2 ml/min and the solvent was acetonitrile/0.01% aqueous NH₄HCO₃) to obtain 4-(5-(2-benzyl-4-methylpiperazin-1-yl)-2-(pyridin-4-yl)pyrazolo[1,5-a]pyrimidin-7-yl)morpholine (13.7 mg, 11%) as white solid. ¹H NMR (400 MHz, DMSO-d₆) δ 8.63 (dd, J=4.6, 1.5 Hz, 2H), 7.87 (d, J=6.0 Hz, 2H), 7.47-6.97 (m, 5H), 6.65 (s, 1H), 5.71 (s, 1H), 4.62 (s, 1H), 4.34 (s, 1H), 3.83 (t, J=4.6 Hz, 4H), 3.62-3.50 (m, 4H), 3.31-3.22 (m, 1H), 3.06 (m, 1H), 2.91 (m, 2H), 2.73 (m, 1H), 2.22 (s, 3H), 1.96 (m, 2H); LCMS (ESI) m/z: 470.0 [M+H]+.

### Syntheses of tert-butyl 3-(7-morpholino-2-(pyridin-4-yl)pyrazolo[1,5-a]pyrimidin-5-yl)azetidine-1-carboxylate (Compound 119) and 4-(5-(1-phenylazetidin-3-yl)-2-(pyridin-4-yl)pyrazolo[1,5-a]pyrimidin-7-yl)morpholine (Compound 120)

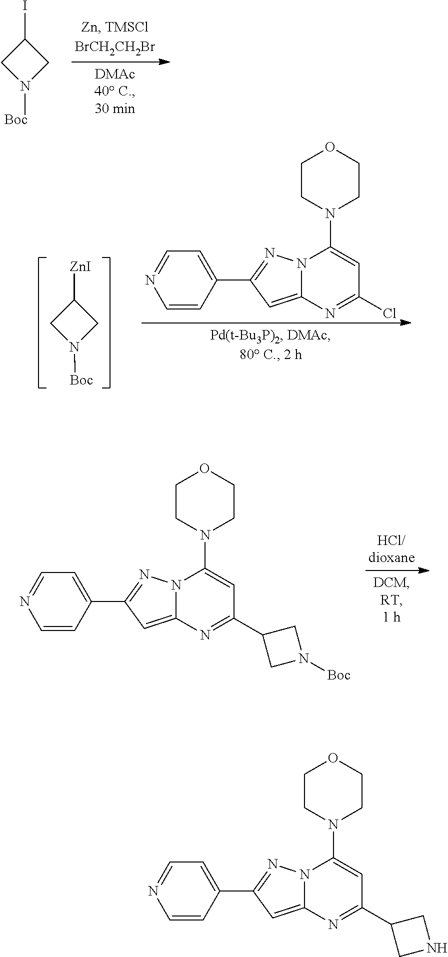

### Step 1: Synthesis of tert-butyl 3-(7-morpholino-2-(pyridin-4-yl)pyrazolo[1,5-a]pyrimidin-5-yl)azetidine-1-carboxylate

An oven-dried, nitrogen-filled flask was charged with zinc dust (2.62 g, 40 mmol) and DMAc (40 mL). This grey suspension was heated to 40° C. and to it were added solutions of 1,2-dibromoethane (0.56 mL, 6.32 mmol) and TMS-CI (0.95 mmol, 2.65 mmol) dropwise and the stirring was continued for 30 min. Then a solution of tert-butyl 3-iodoazetidine-1-carboxylate (5.66 g, 20 mmol) in DMAc was added and stirred for an additional 30 min. The resultant organozinc reagent was used in the next step. To a solution of 4-(5-chloro-2-(pyridin-4-yl)pyrazolo[1,5-a]pyrimidin-7-yl)morpholine (630 mg, 2 mmol) and Pd(t-Bu3P)₂ (102 mg, 0.2 mmol) in dry DMAc (20 mL) was added a solution of (1-(tert-butoxycarbonyl)azetidin-3-yl)zinc(II) iodide (40 mL, 20 mmol) over 5 min at 20° C. under argon atmosphere. The mixture was then heated and stirred at 80° C. for 2 h. It was cooled, then quenched with saturated aqueous NH₄Cl solution and extracted with EtOAc (200 mL×2. The combined organic phase was washed with brine (200 mL), dried over Na₂SO₄ and concentrated. The residue was subjected to SGC (PE:EA=10:1) and then to prep-HPLC (NH₄HCO₃/MeCN) to obtain tert-butyl 3-(7-morpholino-2-(pyridin-4-yl)pyrazolo[1,5-a]pyrimidin-5-yl)azetidine-1-carboxylate (300 mg, 34.40%) as white solid. ¹H NMR (400 MHz, DMSO-d₆) δ 8.69 (dd, J=4.5, 1.6 Hz, 2H), 7.98 (dd, J=4.5, 1.6 Hz, 2H), 7.25 (s, 1H), 6.42 (s, 1H), 4.19-4.10 (m, 4H), 4.00-3.91 (m, 1H), 3.86-3.81 (m, 8H), 1.38 (s, 9H); LCMS (ESI) m/z: 437.3 [M+H]⁺.

### Step 2: Synthesis of 4-(5-(azetidin-3-yl)-2-(pyridin-4-yl)pyrazolo[1,5-a]pyrimidin-7-yl)morpholine

To a solution of tert-butyl 3-(7-morpholino-2-(pyridin-4-yl)pyrazolo[1,5-a]pyrimidin-5-yl)azetidine-1-carboxylate (150 mg, 0.344 mmol) in DCM (2 mL) under argon atmosphere was added HCl in dioxane (2 mL). The mixture was stirred at room temperature for 1 h and concentrated. The residue was subjected to prep-HPLC (formic acid/MeCN) to obtain 4-(5-(azetidin-3-yl)-2-(pyridin-4-yl)pyrazolo[1,5-a]pyrimidin-7-yl)morpholine (90 mg, 68.46%) as white solid. ¹H NMR (400 MHz, DMSO-d₆) δ 8.70 (d, J=6.0 Hz, 2H), 7.99 (d, J=6.0 Hz, 2H), 7.24 (s, 1H), 6.49 (s, 1H), 4.18-4.11 (m, 5H), 3.87-3.83 (m, 8H); LCMS (ESI) m/z: 337.2 [M+H]⁺.

### Synthesis of 4-(5-(1-phenylazetidin-3-yl)-2-(pyridin-4-yl)pyrazolo[1,5-a]pyrimidin-7-yl)morpholine (Compound 121)

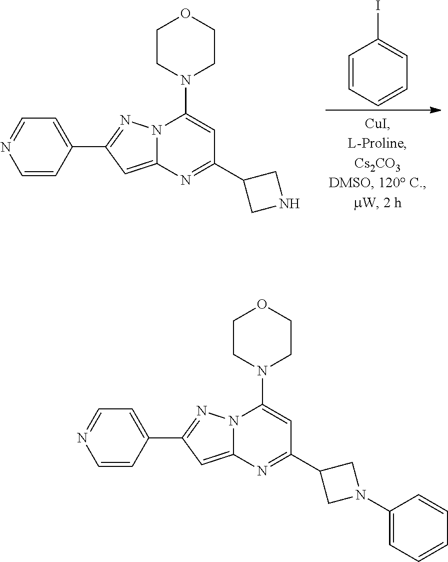 [SMILES: c1cc(-c2cc3nc(C4CNC4)cc(N4CCOCC4)n3n2)ccn1 ; Ic1ccccc1 ; c1ccc(N2CC(c3cc(N4CCOCC4)n4nc(-c5ccncc5)cc4n3)C2)cc1]

To a solution of 4-(5-(azetidin-3-yl)-2-(pyridin-4-yl)pyrazolo[1,5-a]pyrimidin-7-yl)morpholine (70 mg, 0.21 mmol), iodobenzene (84 mg, 0.42 mmol) and cesium carbonate (140 mg, 0.42 mmol) in dimethyl sulfoxide (8 mL) were added L-proline (21 mg, 0.21 mmol) and cuprous iodide (42 mg, 0.21 mmol). The resultant mixture was stirred at 120° C. for 2 h in a microwave reactor. The mixture was cooled, filtered to remove the solids and the filtrate was concentrated. The residue was subjected to prep-HPLC (SunFire C18, 4.6*50 mm, 3.5 um column Xbridge C18 3.5 μm 4.6×50 mm column. The elution system used was a gradient of 5%-95% over 1.5 min at 2 ml/min and the solvent was acetonitrile/0.01% aqueous NH4HCO3) twice to obtain 4-(5-(1-phenylazetidin-3-yl)-2-(pyridin-4-yl)pyrazolo[1,5-a]pyrimidin-7-yl)morpholine (6.5 mg, 7.5%) as white solid. ¹H NMR (400 MHz, DMSO-d₆) δ 8.69 (d, J=6.0 Hz, 2H), 7.96 (d, J=5.6 Hz, 2H), 7.26-7.16 (m, 3H), 6.71 (t, J=7.2 Hz, 1H), 6.54-6.46 (m, 3H), 4.25-4.18 (m, 2H), 4.18-4.09 (m, 1H), 4.08-4.00 (m, 2H), 3.86 (d, J=4.4 Hz, 4H), 3.81 (d, J=5.6 Hz, 8H); LCMS (ESI) m/z: 413.3 [M+H]⁺.

### Synthesis of 4-(5-((2-phenylpyrrolidin-1-yl)methyl)-2-(pyridin-4-yl)pyrazolo[1,5-a]pyrimidin-7-yl)morpholine (Compound 122)

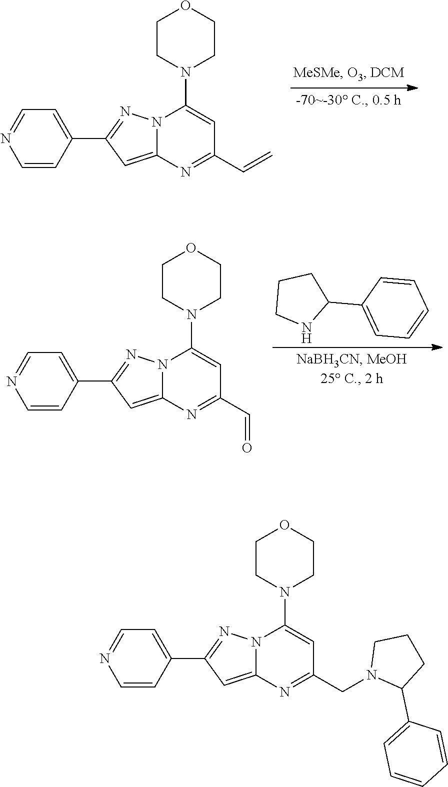 [SMILES: C=Cc1cc(N2CCOCC2)n2nc(-c3ccncc3)cc2n1 ; O=Cc1cc(N2CCOCC2)n2nc(-c3ccncc3)cc2n1 ; c1ccc(C2CCCN2)cc1 ; c1ccc(C2CCCN2Cc2cc(N3CCOCC3)n3nc(-c4ccncc4)cc3n2)cc1]

### Step 1: Synthesis of 7-morpholino-2-(pyridin-4-yl)pyrazolo[1,5-a]pyrimidine-5-carbaldehyde

A solution of 4-(2-(pyridin-4-yl)-5-vinylpyrazolo[1,5-a]pyrimidin-7-yl)morpholine (300 mg, 1 mmol) in dichloromethane (20 mL) was cooled to −78° C. Ozone was then bubbled through the reaction mixture for 0.5 h at temperature −78° C.˜−30° C. The reaction mixture was then purged with oxygen and then dimethyl sulfide (0.18 g, 2.9 mmol) was added. It was stirred for 1 h and then concentrated. The residue was then subjected to silica gel chromatography (DCM: MeOH=20:1) to obtain 7-morpholino-2-(pyridin-4-yl)pyrazolo[1,5-a]pyrimidine-5-carbaldehyde as white solid (120 mg, 40%). LCMS (ESI) m/z: 310.2 [M+H]+.

### Step 2: Synthesis of 4-(5-((2-phenylpyrrolidin-1-yl)methyl)-2-(pyridin-4-yl)pyrazolo[1,5-a]pyrimidin-7-yl)morpholine

To a solution of 7-morpholino-2-(pyridin-4-yl)pyrazolo[1,5-a]pyrimidine-5-carbaldehyde (100 mg, 0.3 mmol) and 2-phenylpyrrolidine (71 mg, 0.48 mmol) in methanol (3 mL) were added acetic acid (10 mg, 0.16 mmol) and sodium cyanoborohydride (61 mg, 1 mmol) at 25° C. The mixture was stirred at 25° C. for 2 h, then filtered to remove the solids and the filtrate was concentrated. The residue was then subjected to prep-HPLC (Boston C18 21*250 mm 10 μm column. The mobile phase was acetonitrile/10 mM ammonium bicarbonate aqueous solution.) to obtain 4-(5-((2-phenylpyrrolidin-1-yl)methyl)-2-(pyridin-4-yl)pyrazolo[1,5-a]pyrimidin-7-yl)morpholine as a yellow solid (20 mg, 15%). ¹H NMR (400 MHz, DMSO-d₆) δ 8.68 (d, J=6.0 Hz, 2H), 7.96 (d, J=6.1 Hz, 2H), 7.43 (d, J=7.0 Hz, 2H), 7.31 (t, J=7.4 Hz, 2H), 7.22 (t, J=7.3 Hz, 1H), 7.15 (s, 1H), 6.34 (s, 1H), 3.87 (t, J=4.6 Hz, 4H), 3.78-3.68 (m, 5H), 3.51 (t, J=8.1 Hz, 1H), 3.36 (s, 1H), 3.21 (s, 1H), 2.38 (d, J=8.7 Hz, 1H), 2.19 (s, 1H), 1.81 (s, 2H), 1.65 (s, 1H).; LCMS (ESI) m/z: 441.3 [M+H]+.

### Synthesis of 4-(5-((2-phenylazetidin-1-yl)methyl)-2-(pyridin-4-yl)pyrazolo[1,5-a]pyrimidin-7-yl)morpholine (Compound 123)

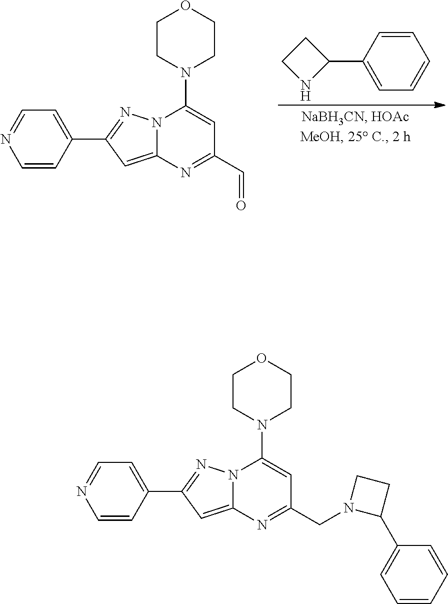 [SMILES: O=Cc1cc(N2CCOCC2)n2nc(-c3ccncc3)cc2n1 ; c1ccc(C2CCN2)cc1 ; c1ccc(C2CCN2Cc2cc(N3CCOCC3)n3nc(-c4ccncc4)cc3n2)cc1]

To a solution of 7-morpholino-2-(pyridin-4-yl)pyrazolo[1,5-a]pyrimidine-5-carbaldehyde (120 mg, 0.4 mmol) and 2-phenylazetidine (62 mg, 0.46 mmol) in methanol (4 mL) were added acetic acid (10 mg, 0.16 mmol) and sodium cyanoborohydride (73 mg, 1.2 mmol) at 25° C. The mixture was stirred at 25° C. for 2 h and concentrated. The residue was then subjected to prep-HPLC (Boston C18 21*250 mm 10 μm column. The mobile phase was acetonitrile/10 mM ammonium bicarbonate aqueous solution) to obtain 4-(5-((2-phenylazetidin-1-yl)methyl)-2-(pyridin-4-yl)pyrazolo[1,5-a]pyrimidin-7-yl)morpholine as yellow solid (50.5 mg, 31%). ¹H NMR (400 MHz, DMSO-d₆) δ 8.67 (dd, J=4.5, 1.5 Hz, 2H), 7.95 (dd, J=4.5, 1.6 Hz, 2H), 7.40 (d, J=6.9 Hz, 2H), 7.27 (t, J=7.2 Hz, 2H), 7.21 (t, J=7.2 Hz, 1H), 7.14 (s, 1H), 6.30 (s, 1H), 4.21 (t, J=8.0 Hz, 1H), 3.82 (t, J=4.6 Hz, 4H), 3.72 (s, 2H), 3.61 (d, J=4.3 Hz, 4H), 3.47 (t, J=6.5 Hz, 1H), 3.14-3.00 (m, 1H), 2.35 (d, J=7.9 Hz, 1H), 2.17-2.03 (m, 1H).; LCMS (ESI) m/z: 427.2 [M+H]+.

### Synthesis of 4-(5-(1-benzylpyrrolidin-2-yl)-2-(pyridin-4-yl)pyrazolo[1,5-a]pyrimidin-7-yl)morpholine (Compound 124)

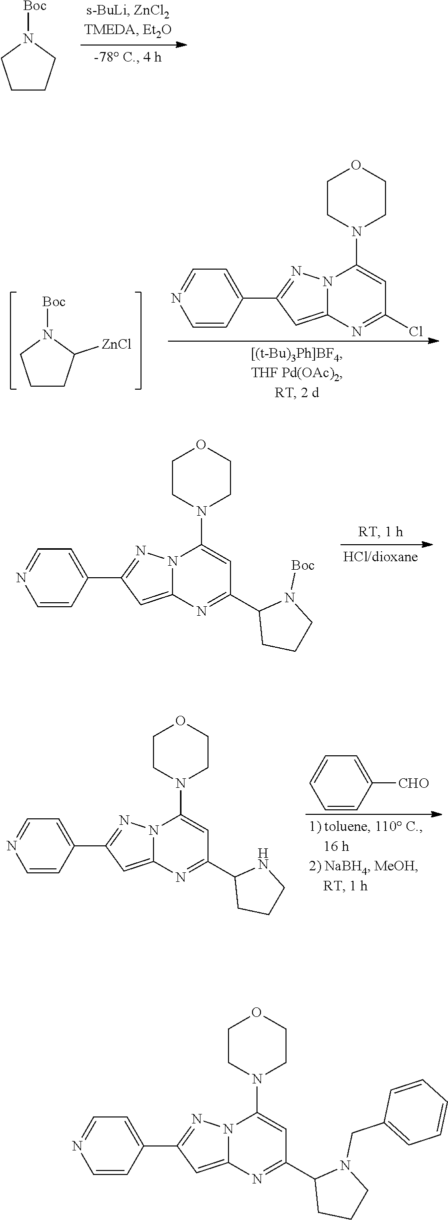

### Step 1: Synthesis of tert-butyl 2-(7-morpholino-2-(pyridin-4-yl)pyrazolo[1,5-a]pyrimidin-5-yl)pyrrolidine-1-carboxylate

A solution of tert-butyl pyrrolidine-1-carboxylate (855 mg, 5 mmol) and tetramethylethylenediamine (1.16 g, 10 mmol) in ether (20 mL) was cooled to −78° C. And then sec-butyllithium (5 mL, 6.5 mmol) was added dropwise and the reaction mixture was stirred for 3.5 h at ˜78° C. Zinc chloride (6.5 mL, 6.5 mmol, 1 M in tetrahydrofuran) was added to the reaction mixture and it was stirred for 30 min at room temperature. To the resultant mixture were added 4-(5-chloro-2-(pyridin-4-yl)pyrazolo[1,5-a]pyrimidin-7-yl)morpholine (945 mg, 3 mmol), palladium (II) acetate (112 mg, 0.5 mmol), and tri-tert-butylphosphine tetrafluoroborate (289 mg, 1 mmol) in tetrahydrofuran (10 mL). The mixture was further stirred for 2 d at room temperature, then diluted with water (20 mL) and extracted with dichloromethane (250 mL×3). The combined organic layer was dried and concentrated. The residue was subjected to silica gel chromatography (petroleum ether/ethyl acetate/methanol=5/5/1) to obtain tert-butyl 2-(7-morpholino-2-(pyridin-4-yl)pyrazolo[1,5-a]pyrimidin-5-yl)pyrrolidine-1-carboxylate as yellow solid (1.35 g, crude). This product was used in the next step without further purification. LCMS (ESI) m/z: 451.2 [M+H]+.

### Step 2: Synthesis of 4-(2-(pyridin-4-yl)-5-(pyrrolidin-2-yl)pyrazolo[1,5-a]pyrimidin-7-yl)morpholine

To a solution of tert-butyl 2-(7-morpholino-2-(pyridin-4-yl)pyrazolo[1,5-a]pyrimidin-5-yl)pyrrolidine-1-carboxylate (2.25 g, 5 mmol) in dichloromethane (3 mL) was added HCl in dioxane (3 mL, 4N) and the mixture was stirred at 25° C. for 1 h. Then a solution of aqueous sodium bicarbonate (20 mL) was added to the reaction mixture, and it was extracted with dichloromethane (20 mL×3). The combined organic layer was dried over sodium sulfate and concentrated. The residue was subjected to silica gel chromatography (dichloromethane/methanol=10/1) to obtain 4-(2-(pyridin-4-yl)-5-(pyrrolidin-2-yl)pyrazolo[1,5-a]pyrimidin-7-yl)morpholine (35 mg, 2%). LCMS (ESI) m/z: 351.1 [M+H]+.

### Step 3: Synthesis of 4-(5-(1-benzylpyrrolidin-2-yl)-2-(pyridin-4-yl)pyrazolo[1,5-a]pyrimidin-7-yl)morpholine

To a solution of 4-(2-(pyridin-4-yl)-5-(pyrrolidin-2-yl)pyrazolo[1,5-a]pyrimidin-7-yl)morpholine (50 mg, 0.14 mmol) and benzaldehyde (26 mg, 0.28 mmol) in toluene (5 ml) was added 4 Å molecular sieves (1 g) and the mixture was stirred at 110° C. overnight. It was then concentrated, and the residue was dissolved in methanol (5 ml). To this, sodium borohydride (21 mg, 0.56 mmol) was added and the mixture was stirred at room temperature for 1 h. The mixture was concentrated, and the residue was subjected to flash column chromatography (dichloromethane: methanol=10:1) to obtain 4-(5-(1-benzylpyrrolidin-2-yl)-2-(pyridin-4-yl)pyrazolo[1,5-a]pyrimidin-7-yl)morpholine as yellow oil (5 mg, 8%). ¹H NMR (400 MHz, DMSO-d₆) δ 8.68 (dd, J=4.8 Hz, 2H), 7.97 (dd, J=4.8 Hz, 2H), 7.30-7.17 (m, 6H), 6.58 (s, 1H), 3.87-3.89 (m, 4H), 3.71-3.81 (m, 5H), 3.52-3.58 (m, 1H), 3.33-3.39 (m, 1H), 3.01-3.05 (m, 1H), 2.33-2.49 (m, 2H), 1.78-1.82 (m, 3H); LCMS (ESI) m/z: 441.3 [M+H]+.

### Syntheses of 2-((7-morpholino-2-(pyridin-4-yl)pyrazolo[1,5-a]pyrimidin-5-yl)oxy)-1-(m-tolyl)ethan-1-one (Compound 125) and N-methyl-2-((7-morpholino-2-(pyridin-4-yl)pyrazolo[1,5-a]pyrimidin-5-yl)oxy)-1-(m-tolyl)ethan-1-amine (Compound 126)

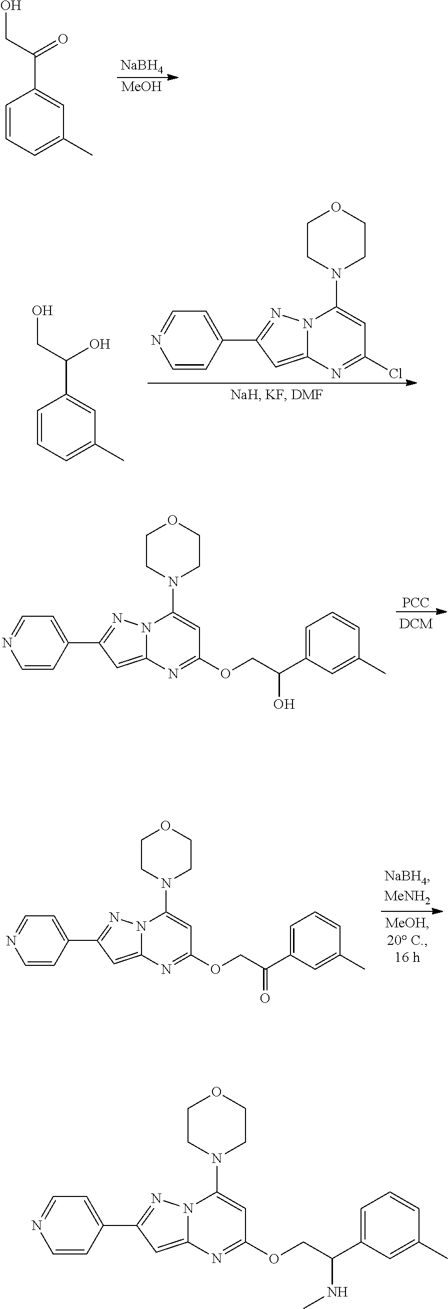

### Step 1: Synthesis of 1-(m-tolyl)ethane-1,2-diol

To a solution of 2-hydroxy-1-(m-tolyl)ethan-1-one (0.9 g, 6 mmol) in methanol (10 mL) were added sodium borohydride (0.09 g, 2.4 mmol) and a few drops of 1 M aqueous HCl at 0° C. The resultant reaction mixture was stirred at 0° C. for 1 h, then diluted with water (30 mL) and extracted with dichloromethane (30 mL*3). The combined organic phase was dried over anhydrous sodium sulphate and concentrated to obtain 1-(m-tolyl)ethane-1,2-diol as colorless oil (0.65 g, 71%) LCMS (ESI) m/z: 135.32 [M+H-18]⁺.

### Step 2: Synthesis of 2-((7-morpholino-2-(pyridin-4-yl)pyrazolo[1,5-a]pyrimidin-5-yl)oxy)-1-(m-tolyl)ethan-1-ol

To a solution of 1-(m-tolyl)ethane-1,2-diol (0.53 g, 3.5 mmol) in N,N-dimethylformamide (40 mL) was added sodium hydride (0.27 g, 6.7 mmol) at 0° C. The mixture was stirred at 25° C. for 0.5 h. To the resultant mixture were added 4-(5-chloro-2-(pyridin-4-yl)pyrazolo[1,5-a]pyrimidin-7-yl)morpholine (1 g, 0.32 mmol) and potassium fluoride (0.55 g,9.5 mmol) at 25° C. Then the mixture was heated up to 60° C. and stirred further for 4 h. The mixture was filtered, the filtrate was concentrated, and the residue was subjected to silica gel column chromatography to obtain 2-((7-morpholino-2-(pyridin-4-yl)pyrazolo[1,5-a]pyrimidin-5-yl)oxy)-1-(m-tolyl)ethan-1-ol as off-white solid (220 mg, 16%). LCMS (ESI) m/z: 432.1 [M+H]+.

### Step 3: Synthesis of 2-((7-morpholino-2-(pyridin-4-yl)pyrazolo[1,5-a]pyrimidin-5-yl)oxy)-1-(m-tolyl)ethan-1-one

To a solution of 2-((7-morpholino-2-(pyridin-4-yl)pyrazolo[1,5-a]pyrimidin-5-yl)oxy)-1-(m-tolyl)ethan-1-ol (0.2 g, 0.5 mmol) in dichloromethane (30 mL) were added magnesium sulphate (1.5 g) and pyridinium chlorochromate (0.16 g, 0.75 mmol) at 20° C. The mixture was stirred at 20° C. for 17 h and filtered to remove the solids. The filtrate was concentrated to obtain the crude product as brown solid (0.2 g,100%), 40 mg of this crude product was subjected to prep-HPLC (Boston C18 21*250 mm 10 μm column. The mobile phase was acetonitrile/10 mM ammonium bicarbonate aqueous solution) to obtain 2-((7-morpholino-2-(pyridin-4-yl)pyrazolo[1,5-a]pyrimidin-5-yl)oxy)-1-(m-tolyl)ethan-1-one (8 mg,20%) as white solid. ¹H NMR (400 MHz, DMSO-d₆) δ 8.65 (s, 2H), 7.89-7.83 (m, 4H), 7.53-7.48 (m, 2H), 6.89 (s, 1H), 6.08 (s, 1H), 5.81 (s, 2H), 3.87 (s, 4H), 3.77 (s, 4H), 2.42 (s, 3H).; LCMS (ESI) m/z: 430.2 [M+H]+.

### Step 4: Synthesis of N-methyl-2-((7-morpholino-2-(pyridin-4-yl)pyrazolo[1,5-a]pyrimidin-5-yl)oxy)-1-(m-tolyl)ethan-1-amine

To a solution of 2-((7-morpholino-2-(pyridin-4-yl)pyrazolo[1,5-a]pyrimidin-5-yl)oxy)-1-(m-tolyl)ethan-1-one (0.12 g, 0.03 mmol) in methanol (6 mL) and dichloromethane (6 mL) was added solution of methanamine (0.3 mL, w/w=35%) and acetic acid (0.05 mL) at 20° C. The reaction mixture was stirred at that temperature for 14 h. To the resultant mixture was added sodium borohydride (9 mg, 0.3 mmol) and the reaction mixture was stirred at 20° C. for another 2 h. It was filtered to remove the solids; the filtrate was concentrated, and the residue was subjected to prep-HPLC (Boston C18 21*250 mm 10 μm column.

The mobile phase was acetonitrile/10 mM formic acid aqueous solution.) to obtain N-methyl-2-((7-morpholino-2-(pyridin-4-yl)pyrazolo[1,5-a]pyrimidin-5-yl)oxy)-1-(m-tolyl)ethan-1-amine as brown solid (13 mg, 10%). ¹H NMR (400 MHz, DMSO-d₆) δ 8.67 (d, J=5.6 Hz, 2H), 8.35 (s, 1H), 7.92 (d, J=5.8 Hz, 2H), 7.24 (d, J=7.9 Hz, 3H), 7.10 (d, J=7.3 Hz, 1H), 6.97 (s, 1H), 5.88 (s, 1H), 4.34 (d, J=6.4 Hz, 2H), 3.88 (d, J=6.0 Hz, 1H), 3.84 (s, 4H), 3.70 (s, 4H), 2.32 (s, 3H), 2.18 (s, 3H).; LCMS (ESI) m/z: 445.2 [M+H]+.

### Synthesis of 4-(5-(3-phenyl-1,2,4-thiadiazol-5-yl)-2-(pyridin-4-yl)pyrazolo[1,5-a]pyrimidin-7-yl)morpholine (Compound 127)

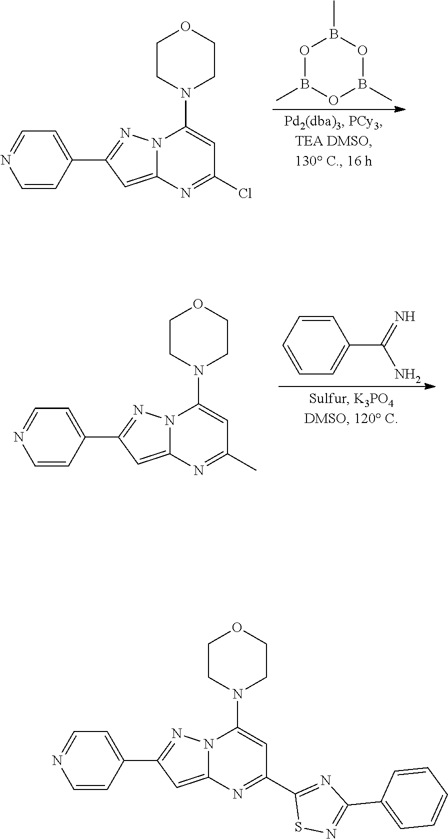

### Step 1: Synthesis of 4-(5-methyl-2-(pyridin-4-yl)pyrazolo[1,5-a]pyrimidin-7-yl)morpholine

A mixture of 4-(5-chloro-2-(pyridin-4-yl)pyrazolo[1,5-a]pyrimidin-7-yl)morpholine (0.5 g, 1.6 mmol), 2,4,6-trimethyl-1,3,5,2,4,6-trioxatriborinane (0.8 g, 3.2 mmol), tris(dibenzylideneacetone)dipalladium (0.15 g, 0.16 mmol), tricyclohexyl phosphine (90 mg, 0.32 mmol) and cesium carbonate (1 g, 3.2 mmol) in dry dimethyl sulfoxide (25 mL) was stirred at 130° C. under nitrogen atmosphere for 16 h. The reaction was cooled, the mixture filtered through a pad of celite, and the filtrate was diluted with ethyl acetate/water (30 mL/30 mL). The organic layer was separated, and the aqueous layer was extracted with ethyl acetate (40 mL) twice. The combined organic phase was washed with brine (40 mL), dried over sodium sulfate, filtered and concentrated. The residue was slurred in a mixture of ethyl acetate/petroleum ether (5 mL/40 mL) and the resultant precipitate was collected by filtration. It was then vacuum dried to obtain 4-(5-methyl-2-(pyridin-4-yl)pyrazolo[1,5-a]pyrimidin-7-yl)morpholine (0.4 g, 85%) as yellow solid. LCMS (ESI) m/z: 296.3 [M+H]⁺.

### Step 2: Synthesis of 4-(5-(3-phenyl-1,2,4-thiadiazol-5-yl)-2-(pyridin-4-yl)pyrazolo[1,5-a]pyrimidin-7-yl)morpholine

A mixture of 4-(5-methyl-2-(pyridin-4-yl)pyrazolo[1,5-a]pyrimidin-7-yl)morpholine (0.3 g, 1 mmol), benzimidamide hydrochloride (0.32 g, 2 mmol), sulfur (160 mg, 5 mmol) and potassium phosphate (0.64 g, 3 mmol) in dry dimethyl sulfoxide (10 mL) was stirred at 120° C. for 16 h. The reaction was cooled down and the mixture was filtered through a pad of Celite. The filtrate was diluted with ethyl acetate/water (10 mL/10 mL), the organic layer separated, and the aqueous layer was extracted with ethyl acetate (20 mL) twice. The combined organic phase was washed with brine (20 mL), dried over sodium sulfate, filtered and concentrated. The residue was subjected to prep-HPLC (base) to obtain 4-(5-(3-phenyl-1,2,4-thiadiazol-5-yl)-2-(pyridin-4-yl)pyrazolo[1,5-a]pyrimidin-7-yl)morpholine (20 mg, 5%) as yellow solid. ¹H NMR (400 MHz, CDCl₃) δ 8.76 (dd, J=4.4 Hz, 1.6 Hz, 2H), 8.47-8.36 (m, 2H), 7.88 (dd, J=4.4 Hz, 1.6 Hz, 2H), 7.59-7.50 (m, 3H), 7.20 (s, 1H), 7.11 (s, 1H), 4.17-3.93 (m, 8H); LCMS (ESI) m/z: 442.2 [M+H]⁺.

### Synthesis of 4-(5-(5-phenyl-1,2,4-thiadiazol-3-yl)-2-(pyridin-4-yl)pyrazolo[1,5-a]pyrimidin-7-yl)morpholine (Compound 128)

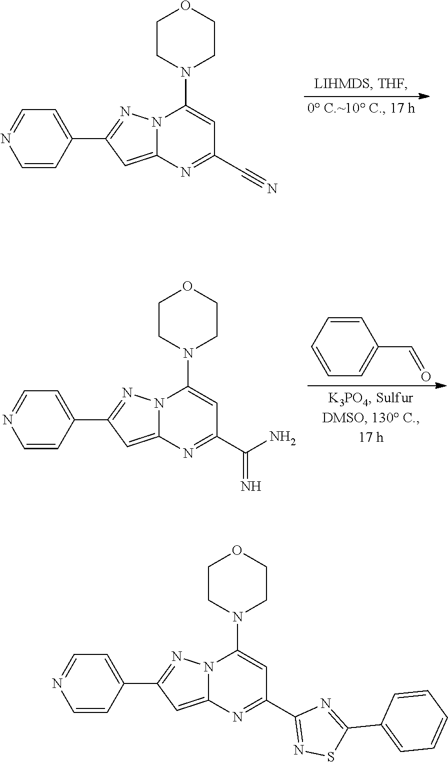 [SMILES: N#Cc1cc(N2CCOCC2)n2nc(-c3ccncc3)cc2n1 ; N=C(N)c1cc(N2CCOCC2)n2nc(-c3ccncc3)cc2n1 ; O=Cc1ccccc1 ; c1ccc(-c2nc(-c3cc(N4CCOCC4)n4nc(-c5ccncc5)cc4n3)ns2)cc1]

### Step 1: Synthesis of 7-morpholino-2-(pyridin-4-yl)pyrazolo[1,5-a]pyrimidine-5-carboximidamide

To a solution of 7-morpholino-2-(pyridin-4-yl)pyrazolo[1,5-a]pyrimidine-5-carbonitrile (1.9 g, 6.21 mmol) in tetrahydrofuran (30 mL) under argon atmosphere at 0° C. was added lithium bis(trimethylsilyl)amide (31 mL, 31.0 mml) and the mixture was stirred at 10° C. for 17 h. The reaction was quenched by the addition of cold aqueous ammonium chloride solution and the resultant precipitate was filtered off. The filtrate was concentrated, and the residue was triturated with ethanol to obtain 7-morpholino-2-(pyridin-4-yl)pyrazolo[1,5-a]pyrimidine-5-carboximidamide as yellow solid (1.3 g, 65%).

### Step 2: Synthesis of 4-(5-(5-phenyl-1,2,4-thiadiazol-3-yl)-2-(pyridin-4-yl)pyrazolo[1,5-a]pyrimidin-7-yl)morpholine

To a solution of 7-morpholino-2-(pyridin-4-yl)pyrazolo[1,5-a]pyrimidine-5-carboximidamide (300 mg, 0.93 mmol) in DMSO (4 mL) were added benzaldehyde (197.2 mg, 1.86 mmol) potassium phosphate tribasic (591.5 mg, 2.79 mmol) and sulfur (178.6 mg, 5.58 mmol). The resultant mixture was stirred at 130° C. for 17 h and cooled. The mixture was filtered to remove the insoluble materials and the filtrate was concentrated. The residue was subjected to prep-HPLC (Boston C18 21*250 mm 10 μm column. The mobile phase was acetonitrile/0.01% aqueous FA.) to obtain 4-(5-(5-phenyl-1,2,4-thiadiazol-3-yl)-2-(pyridin-4-yl)pyrazolo[1,5-a]pyrimidin-7-yl)morpholine as yellow solid (26.9 mg, 6.6%). ¹H NMR (400 MHz, DMSO) δ 8.73 (d, J=5.9 Hz, 2H), 8.18 (t, J=8.9 Hz, 2H), 8.05 (d, J=5.9 Hz, 2H), 7.69-7.65 (m, 3H), 7.50 (s, 1H), 7.31 (s, 1H), 3.94 (s, 8H); LCMS (ESI) m/z: 442.1 [M+H]+.

### Synthesis of 4-(5-(3-phenyl-1,2,4-oxadiazol-5-yl)-2-(pyridin-4-yl)pyrazolo[1,5-a]pyrimidin-7-yl)morpholine (Compound 129)

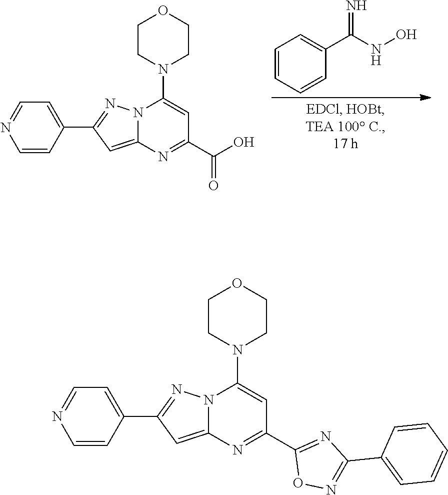 [SMILES: O=C(O)c1cc(N2CCOCC2)n2nc(-c3ccncc3)cc2n1 ; N=C(NO)c1ccccc1 ; c1ccc(-c2noc(-c3cc(N4CCOCC4)n4nc(-c5ccncc5)cc4n3)n2)cc1]

A solution of 7-morpholino-2-(pyridin-4-yl)pyrazolo[1,5-a]pyrimidine-5-carboxylic acid (65 mg, 0.2 mmol), N-hydroxybenzimidamide (33 mg, 0.24 mmol), HOBt (32 mg, 0.24 mmol), EDCl·HCl (46 mg, 0.24 mmol) and triethylamine (31 mg, 0.24 mmol) in N,N-dimethylformamide (9 mL) was stirred at 100° C. for 17 h under nitrogen atmosphere. The reaction mixture was then diluted with ethyl acetate (60 mL), washed with brine (30 mL), dried over sodium sulfate and concentrated. The residue was subjected to prep-HPLC (BOSTON pHlex ODS 10 μm 21.2×250 mm 120 A. The mobile phase was acetonitrile/0.1% aqueous formic acid) to obtain 4-(5-(3-phenyl-1,2,4-oxadiazol-5-yl)-2-(pyridin-4-yl)pyrazolo[1,5-a]pyrimidin-7-yl)morpholine (9.7 mg, 11.4%) as yellow solid. ¹H NMR (400 MHz, DMSO-d₆) δ 8.74 (d, J=6.0 Hz, 2H), 8.16 (dd, J=7.7, 1.8 Hz, 2H), 8.09-8.02 (m, 2H), 7.66-7.61 (m, 3H), 7.60 (s, 1H), 7.18 (s, 1H), 4.02 (d, J=4.9 Hz, 4H), 3.93 (d, J=5.0 Hz, 4H); LCMS (ESI) m/z: 426.1 [M+H]+.

### Synthesis of 4-(5-(5-phenyl-1,3,4-oxadiazol-2-yl)-2-(pyridin-4-yl)pyrazolo[1,5-a]pyrimidin-7-yl)morpholine (Compound 130)

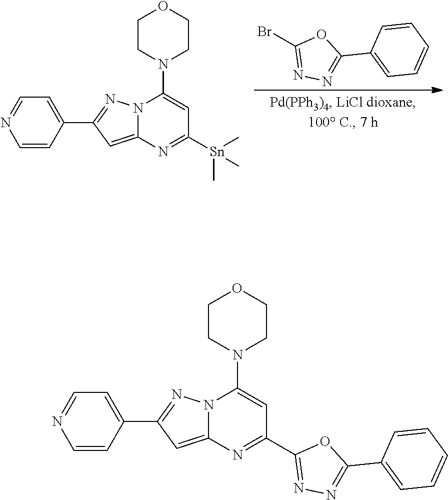 [SMILES: [CH3][Sn]([CH3])([CH3])[c]1cc(N2CCOCC2)n2nc(-c3ccncc3)cc2n1 ; Brc1nnc(-c2ccccc2)o1 ; c1ccc(-c2nnc(-c3cc(N4CCOCC4)n4nc(-c5ccncc5)cc4n3)o2)cc1]

To a solution of 4-(2-(pyridin-4-yl)-5-(trimethylstannyl)pyrazolo[1,5-a]pyrimidin-7-yl)morpholine (130 mg, 0.29 mmol), 2-bromo-5-phenyl-1,3,4-oxadiazole (78 mg, 0.35 mmol) and lithium chloride (30 mg, 0.73 mmol) in dioxane (18 mL) was added tetrakis(triphenylphosphine)palladium (34 mg, 0.029 mmol) and the mixture was stirred at 100° C. for 7 h under argon atmosphere. It was then diluted with ethyl acetate (100 mL), washed with brine (50 mL), dried and concentrated. The residue was first triturated with DMF, then with ethanol (15 mL) while heating. The mixture was then cooled, and the resultant precipitate was collected by filtration and vacuum dried to obtain 4-(5-(5-phenyl-1,3,4-oxadiazol-2-yl)-2-(pyridin-4-yl)pyrazolo[1,5-a]pyrimidin-7-yl)morpholine as white solid (19.7 mg, 16%). ¹H NMR (400 MHz, TFA) δ 9.01 (d, J=6.7 Hz, 2H), 8.73 (d, J=6.7 Hz, 2H), 8.19 (d, J=7.5 Hz, 2H), 7.81 (t, J=7.5 Hz, 1H), 7.69 (t, J=7.8 Hz, 2H), 7.62 (s, 1H), 7.56 (s, 1H), 4.84 (s, 4H), 4.40 (s, 4H); LCMS (ESI) m/z: 426.1 [M+H]+.

The following compound was synthesized according to the protocol described above:

| Name | Structure | NMR, LC-MS | Comp # |
|---|---|---|---|
| 4-(5-(5-phenyl- 1,3,4-thiadiazol- 2-yl)-2-(pyridin-4- yl)pyrazolo[1,5- a]pyrimidin-7- yl)morpholine | 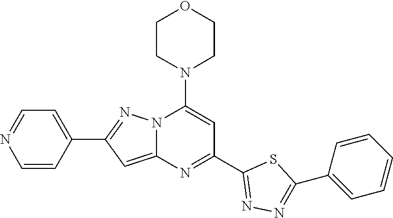 [SMILES: c1ccc(-c2nnc(-c3cc(N4CCOCC4)n4nc(-c5ccncc5)cc4n3)s2)cc1] | ¹H NMR (400 MHz, DMSO-d₆) δ 8.73 (d, J = 6.0 Hz, 2H), 8.43 (s, 1H), 8.14 − 8.06 (m, 2H), 8.03 (dd, J = 4.5, 1.6 Hz, 2H), 7.67 − 7.56 (m, 3H), 7.48 (s, 1H), 7.21 (s, 1H), 3.98 (d, J = 5.3 Hz, 4H), 3.93 (d, J = 5.0 Hz, 4H); LCMS (ESI) m/z: 442.1 [M + H]⁺. | 131 |

### Synthesis of 4-(5-(1-phenylazetidin-3-yl)-2-(pyridin-4-yl)pyrazolo[1,5-a]pyrimidin-7-yl)morpholine (Compound 132)

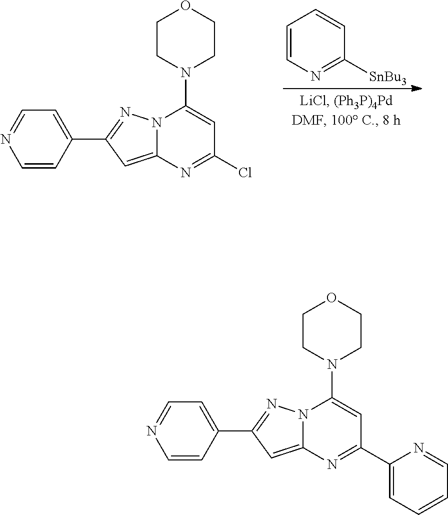

To a suspension of 4-(5-chloro-2-(pyridin-4-yl)pyrazolo[1,5-a]pyrimidin-7-yl)morpholine (95 mg, 03 mmol) in dry DMF (5 mL) was added 2-(tributylstannyl)pyridine (166 mg, 0.45 mmol), LiCl (38 mg, 0.9 mmol) and (Ph₃P)₄Pd (35 mg, 0.03 mmol). The mixture was stirred at 100° C. for 8 h under argon atmosphere. It was cooled and slowly added to ice-water (50 mL) and solid precipitated was collected by filtration. The solids were then triturated with EtOH (5 mL) to obtain 4-(5-(pyridin-2-yl)-2-(pyridin-4-yl)pyrazolo[1,5-a]pyrimidin-7-yl)morpholine (45 mg, 41.88%) as grey solid. ¹H NMR (400 MHz, DMSO-d₆) δ 8.76 (d, J=4.8 Hz, 1H), 8.72 (d, J=6.0 Hz, 2H), 8.47 (d, J=8.0 Hz, 1H), 8.03 (d, J=5.9 Hz, 3H), 7.60-7.51 (m, 1H), 7.45 (s, 1H), 7.40 (s, 1H), 3.91 (d, J=4.1 Hz, 8H); LCMS (ESI) m/z: 359.1 [M+H]+.

### Synthesis of ethyl 2-(7-morpholino-2-(pyridin-4-yl)pyrazolo[1,5-a]pyrimidin-5-yl)acetate (Compound 133) and 2-(7-morpholino-2-(pyridin-4-yl)pyrazolo[1,5-a]pyrimidin-5-yl)ethan-1-ol (Compound 134)

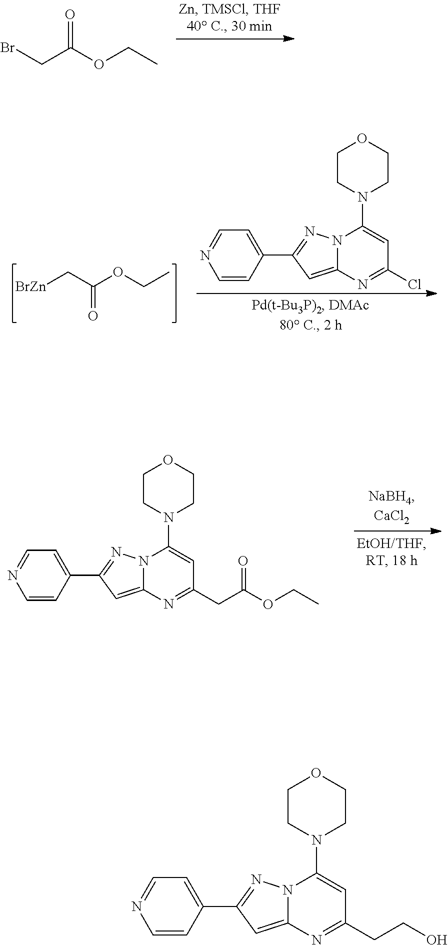

### Step 1: Synthesis of ethyl 2-(7-morpholino-2-(pyridin-4-yl)pyrazolo[1,5-a]pyrimidin-5-yl)acetate

To a stirred suspension of zinc powder (2.62 g, 40 mmol) in dry tetrahydrofuran (20 mL) under an inner atmosphere, was added trimethylsilyl chloride (0.37 mL, 0.9 mmol) at room temperature and the mixture was stirred for 30 min. It was heated to 40° C. and ethyl bromoacetate (2.2 mL, 20 mmol) was added dropwise over 15 min to the mixture and stirring was continued for another 30 min. From the resultant heterogeneous mixture, the supernatant was collected by decanting the flask and used in the next step. To a stirred solution of 4-(5-chloro-2-(pyridin-4-yl)pyrazolo[1,5-a]pyrimidin-7-yl)morpholine (630 mg, 2 mmol) and Pd(t-Bu₃P)₂(102 mg, 0.2 mmol) in dry DMAc (20 mL) was dropwise added a solution of (2-ethoxy-2-oxoethyl)zinc(II) bromide (20 mL, 20 mmol, prepared as above) over 5 min at 20° C. under argon atmosphere. The reaction mixture was heated to 80° C. and stirred for 2 h and then cooled. The reaction was then quenched with saturated aqueous NH₄Cl solution, and the mixture was extracted with EtOAc (200 mL×2). The combined organic phase was washed with brine (200 mL), dried over Na₂SO₄ and concentrated. The residue was triturated with Et₂O (20 mL) and EtOH (10 mL) to obtain ethyl 2-(7-morpholino-2-(pyridin-4-yl)pyrazolo[1,5-a]pyrimidin-5-yl)acetate (538 mg, 73.26%) as pale-yellow solid. ¹H NMR (400 MHz, DMSO-d₆) δ 8.69 (d, J=5.9 Hz, 2H), 7.98 (d, J=6.0 Hz, 2H), 7.20 (s, 1H), 6.50 (s, 1H), 4.14 (q, J=7.1 Hz, 2H), 3.97-3.75 (m, 10H), 1.21 (t, J=7.1 Hz, 3H); LCMS (ESI) m/z: 368.2 [M+H]⁺.

### Step 2: Synthesis of 2-(7-morpholino-2-(pyridin-4-yl)pyrazolo[1,5-a]pyrimidin-5-yl)ethan-1-ol

To a solution of ethyl 2-(7-morpholino-2-(pyridin-4-yl)pyrazolo[1,5-a]pyrimidin-5-yl)acetate (320 mg, 0.87 mmol) and CaCl2 (484 mg, 4.36 mmol) in EtOH (20 mL) and THE (20 mL) was added NaBH4 (323 mg, 8.72 mmol) in portions. The reaction mixture was stirred at room temperature for 18 h, then quenched with concentrated HCl (1 mL) and stirred for 1 h. The pH of the resultant mixture was adjusted to ˜8 with aqueous K₂CO₃ solution and it was extracted with EtOAc (100 mL×3). The combined organic phase was washed with brine (100 mL), dried Na₂SO₄ and concentrated. The residue was subjected to SGC (DCM: MeOH=20:1) to obtain 2-(7-morpholino-2-(pyridin-4-yl)pyrazolo[1,5-a]pyrimidin-5-yl)ethan-1-ol (110 mg, 38.89%) as white solid. ¹H NMR (400 MHz, DMSO-d₆) δ 8.68 (d, J=5.8 Hz, 2H), 8.08-7.89 (m, 2H), 7.14 (s, 1H), 6.41 (s, 1H), 4.70 (s, 1H), 3.88-3.76 (m, 10H), 2.88 (t, J=6.7 Hz, 2H); LCMS (ESI) m/z: 326.3 [M+H]⁺.

### Synthesis of 4-(7-morpholino-2-(pyridin-4-yl)pyrazolo[1,5-a]pyrimidin-5-yl)-6-phenylmorpholin-3-one (Compound 135)

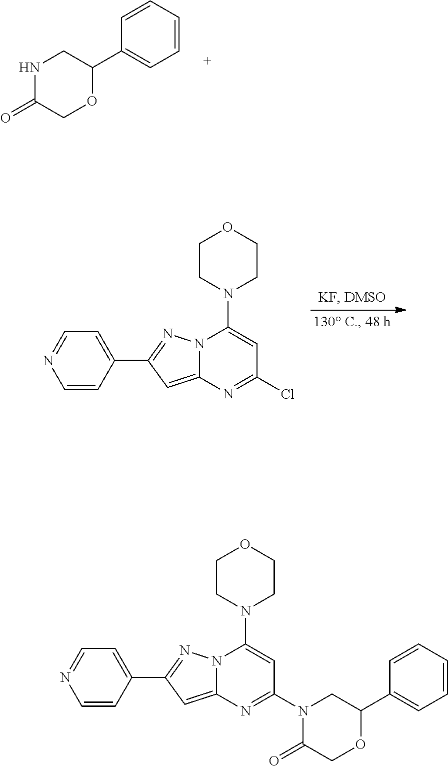 [SMILES: O=C1COC(c2ccccc2)CN1 ; Clc1cc(N2CCOCC2)n2nc(-c3ccncc3)cc2n1 ; O=C1COC(c2ccccc2)CN1c1cc(N2CCOCC2)n2nc(-c3ccncc3)cc2n1]

To a solution of 6-phenylmorpholin-3-one (315 mg, 1 mmol) and 4-(5-chloro-2-(pyridin-4-yl)pyrazolo[1,5-a]pyrimidin-7-yl)morpholine (177 mg, 1 mmol) in dimethyl sulfoxide (10 mL) was added potassium fluoride (174 mg, 3 mmol) and the resultant mixture was stirred at 130° C. for 48 h. The reaction mixture was then diluted with ethyl acetate (160 ml), washed successively with water (60 ml) and brine (60 ml). The organic phase was then dried over sodium sulfate and concentrated. The residue was subjected to prep-HPLC (BOSTON pHlex ODS 10 μm 21.2×250 mm 120 A. The mobile phase was acetonitrile/0.1% Ammonium bicarbonate) to obtain 4-(7-morpholino-2-(pyridin-4-yl)pyrazolo[1,5-a]pyrimidin-5-yl)-6-phenylmorpholin-3-one as white solid (20 mg, 4.4%). ¹H NMR (400 MHz, DMSO-d₆) δ 8.68 (dd, J=4.6, 1.6 Hz, 2H), 7.95 (dd, J=4.4, 1.6 Hz, 2H), 7.50 (d, J=7.0 Hz, 2H), 7.46-7.38 (m, 3H), 7.36 (s, 1H), 7.15 (s, 1H), 5.11 (dd, J=10.8, 2.8 Hz, 1H), 4.56 (d, J=2.0 Hz, 2H), 4.43 (dd, J=12.8, 2.8 Hz, 1H), 3.89 (t, J=4.6 Hz, 4H), 3.84-3.69 (m, 5H); LCMS (ESI) m/z: 457.2 [M+H]+.

### Synthesis of 4-methyl-1-(7-morpholino-2-(pyridin-4-yl)pyrazolo[1,5-a]pyrimidin-5-yl)-5-phenylpiperazin-2-one (Compound 136)

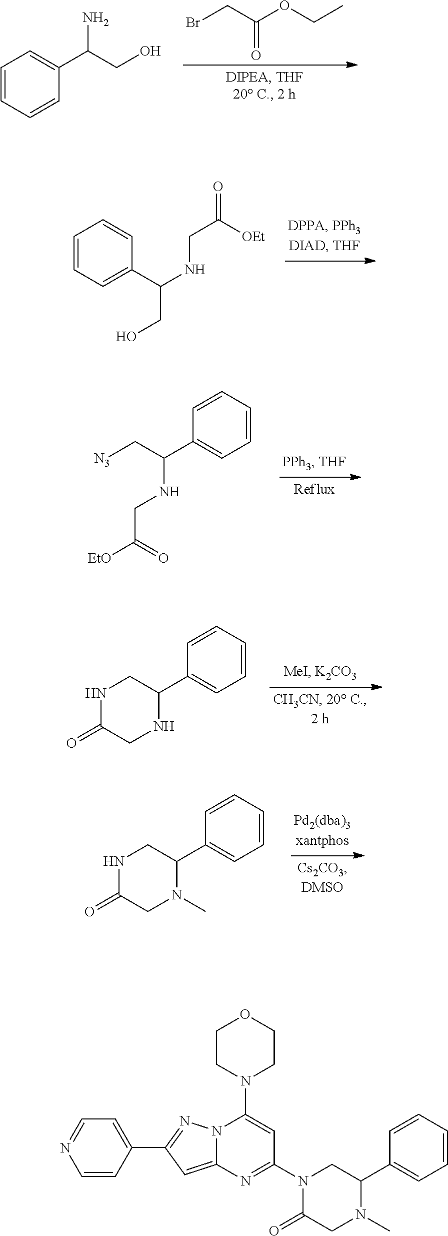

### Step 1: Synthesis of ethyl 2-(2-hydroxy-1-phenylethylamino)acetate

To a solution of 2-amino-2-phenylethanol (5.0 g, 36.45 mmol) and diisopropylethylamine (14.13 g, 109.35 mmol) in tetrahydrofuran (100 mL) was added a solution of ethyl 2-bromoacetate (6.09 g, 36.45 mmol) in tetrahydrofuran (100 mL). Then mixture was stirred at 20° C. for 2 h and concentrated. The residue was then subjected to silica gel column chromatography (petroleum ether/acetic ester=10:1 to 1:2) to obtain ethyl 2-(2-hydroxy-1-phenylethylamino)acetate (5.28 g) as light-yellow oil. LCMS (ESI) m/z: 224.1 [M+H]+.

### Step 2: Synthesis of ethyl 2-(2-azido-1-phenylethylamino)acetate

To a solution of ethyl 2-(2-hydroxy-1-phenylethylamino)acetate (1.0 g, 4.482 mmol), diphenyl phosphoryl azide (2.47 g, 8.964 mmol) and triphenylphosphine (2.34 g, 8.964 mmol) in tetrahydrofuran (50 mL) was added diisopropyl azodicarboxylate (1.81 g, 8.964 mmol) at 0° C. The mixture was stirred at 20° C. for 4 h and then diluted with ethyl acetate (100 mL). The organic layer was washed with brine (100 mL), dried and concentrated to obtain the desired product. It was used in the next step without further purification. LCMS (ESI) m/z: 249.1 [M+H]+.

### Step 3: Synthesis of 5-phenylpiperazin-2-one

To a solution of ethyl 2-(2-azido-1-phenylethylamino)acetate (1.1 g, 4.433 mmol) in tetrahydrofuran (50 mL) was added triphenylphosphine (1.39 g, 5.319 mmol) at 20° C. The mixture was heated to 90° C. and stirred for another 4 h. It was then diluted with ethyl acetate (100 mL), the organic layer was washed with brine (100 mL), dried and concentrated. The residue was subjected to prep-HPLC (SunFire C18, 4.6*50 mm, 3.5 um column Xbridge C18 3.5 μm 4.6×50 mm column. The elution system used was a gradient of 5%-95% over 1.5 min at 2 ml/min and the solvent was acetonitrile/0.01% aqueous NH₄HCO₃) to obtain 5-phenylpiperazin-2-one (130 mg, 17%) as white solid. ¹H NMR (500 MHz, DMSO) δ 7.75 (s, 1H), 7.41 (d, J=7.5 Hz, 2H), 7.34 (t, J=7.5 Hz, 2H), 7.27 (t, J=7.3 Hz, 1H), 3.91 (dd, J=10.4, 3.9 Hz, 1H), 3.37 (m, 1H), 3.29-3.24 (m, 2H), 3.11 (t, J=11.0 Hz, 1H). LCMS (ESI) m/z: 177.1 [M+H]+.

### Step 4: Synthesis of 4-methyl-5-phenylpiperazin-2-one

To a solution of 5-phenylpiperazin-2-one (130 mg, 0.738 mmol) and potassium carbonate (205 mg, 1.476 mmol) in acetonitrile (10 mL) was added methyl iodide (115 mg, 0.812 mmol) at 20° C. The mixture was stirred for 4 h, then filtered to remove the solids and the filtrate was concentrated. The residue was subjected to prep-HPLC (SunFire C18, 4.6*50 mm, 3.5 um column Xbridge C18 3.5 μm 4.6×50 mm column. The elution system used was a gradient of 5%-95% over 1.5 min at 2 ml/min and the solvent was acetonitrile/0.01% aqueous NH₄HCO₃) to obtain 4-methyl-5-phenylpiperazin-2-one (48 mg, 34%) as white solid. ¹H NMR (400 MHz, DMSO) δ 7.89 (s, 1H), 7.52-7.18 (m, 5H), 3.38 (s, 1H), 3.26-3.19 (m, 1H), 3.14-3.11 (m, 1H), 2.83 (d, J=16.7 Hz, 1H), 1.90 (s, 3H); LCMS (ESI) m/z: 191.1 [M+H]+.

### Step 5: Synthesis of 4-methyl-1-(7-morpholino-2-(pyridin-4-yl)pyrazolo[1,5-a]pyrimidin-5-yl)-5-phenylpiperazin-2-one

To a solution of 4-methyl-5-phenylpiperazin-2-one (28 mg, 0.147 mmol), 4-(5-chloro-2-(pyridin-4-yl)pyrazolo[1,5-a]pyrimidin-7-yl)morpholine (46.4 mg, 0.147 mmol), 4,5-bis(diphenylphosphino)-9,9-dimethylxanthene (17.4 mg, 0.030 mmol) in dimethyl sulfoxide (5 mL) was added tris(dibenzylideneacetone)dipalladium(0)(13.5 mg, 0.015 mmol). Then the reaction mixture was heated to 130° C. and stirred for 6 h. The mixture was cooled, then diluted with dichloromethane (100 mL), washed with brine, dried and concentrated. The residue was subjected to prep-HPLC (SunFire C18, 4.6*50 mm, 3.5 um column Xbridge C18 3.5 μm 4.6×50 mm column. The elution system used was a gradient of 5%-95% over 1.5 min at 2 ml/min and the solvent was acetonitrile/0.01% aqueous NH₄HCO₃) to obtain 4-methyl-1-(7-morpholino-2-(pyridin-4-yl)pyrazolo[1,5-a]pyrimidin-5-yl)-5-phenylpiperazin-2-one (0.6 mg, 0.6%) as white solid. ¹H NMR (400 MHz, DMSO) δ 8.67 (d, J=5.7 Hz, 2H), 7.91 (d, J=5.9 Hz, 2H), 7.58-7.34 (m, 5H), 7.26 (s, 1H), 7.10 (s, 1H), 4.18-4.13 (m, 1H), 3.90-3.70 (m, 11H), 3.62 (d, J=6.8 Hz, 1H), 2.03 (s, 3H); LCMS (ESI) m/z: 470.3 [M+H]+.

### Synthesis of 4-(5-(2-(3-fluorophenoxy)pyridin-3-yl)-2-(pyridin-4-yl)pyrazolo[1,5-a]pyrimidin-7-yl)morpholine (Compound 137)

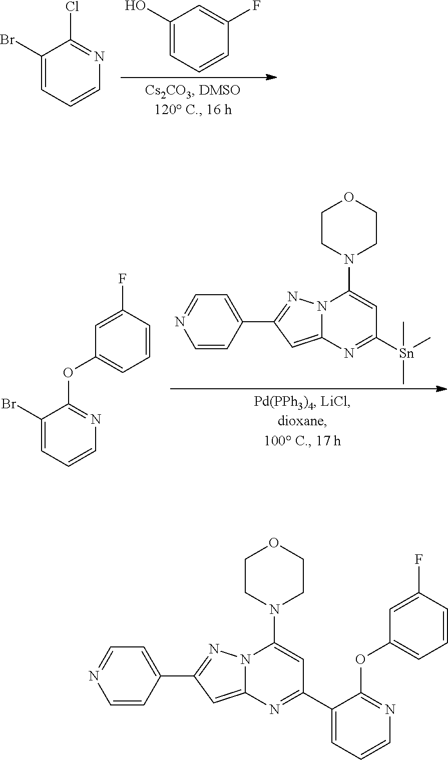

### Step 1: Synthesis of 3-bromo-2-(3-fluorophenoxy)pyridine

A mixture of 3-bromo-2-chloropyridine (573 mg, 3 mmol), 3-fluorophenol (336 mg, 3 mmol) and cesium carbonate (1.95 g, 6 mmol) in dimethyl sulfoxide (10 mL) was stirred at 120° C. for 16 h. It was then diluted with water (30 mL) and extracted with ethyl acetate (30 mL*3). The combined organic phase was concentrated, and the residue was subjected to flash chromatography eluting with 0-50% ethyl acetate in petroleum ether to obtain 3-bromo-2-(3-fluorophenoxy)pyridine as yellow solid. (600 mg, 75%). LCMS (ESI) m/z: 268.0 [M+H]+.

### Step 2: Synthesis of 4-(5-(2-(3-fluorophenoxy)pyridin-3-yl)-2-(pyridin-4-yl)pyrazolo[1,5-a]pyrimidin-7-yl)morpholine

To a solution of 4-(2-(pyridin-4-yl)-5-(trimethylstannyl)pyrazolo[1,5-a]pyrimidin-7-yl)morpholine (0.22 g, 0.5 mmol) in dioxane (10 mL) were added 3-bromo-2-(3-fluorophenoxy)pyridine (0.27 g, 1 mmol), lithium chloride (53 mg, 1.25 mmol) and tetrakis(triphenylphosphine)palladium (58 mg, 0.05 mmol) at 25° C. The resultant mixture was stirred at 100° C. for 17 h under argon atmosphere. It was filtered to remove the solids; the filtrate was concentrated, and the residue was subjected to prep-HPLC (BOSTON pHlex ODS 10 um 21.2×250 mm120 A. The mobile phase was acetonitrile/0.1% Ammonium bicarbonate) to obtain 4-(5-(2-(3-fluorophenoxy)pyridin-3-yl)-2-(pyridin-4-yl)pyrazolo[1,5-a]pyrimidin-7-yl)morpholine (100 mg, 43%) as yellow solid. ¹H NMR (400 MHz, DMSO-d₆) δ 8.71 (d, J=6.0 Hz, 2H), 8.39 (dd, J=7.6, 2.0 Hz, 1H), 8.28 (dd, J=4.8, 2.0 Hz, 1H), 8.02 (d, J=6.0 Hz, 2H), 7.48-7.46 (m, 1H), 7.39-7.36 (m, 2H), 7.22-7.20 (m, 1H), 7.11-7.02 (m, 3H), 3.86-3.85 (m, 8H). LCMS (ESI) m/z: 469.2 [M+H]+.

The following compound was synthesized according to the protocol described above:

| Name | Structure | NMR, MS | Compd # |
|---|---|---|---|
| 4-(5-(2-(4- fluorophenoxy) pyridin-3-yl)-2- (pyridin-4- yl)pyrazolo[1,5- a]pyrimidin-7- yl)morpholine | 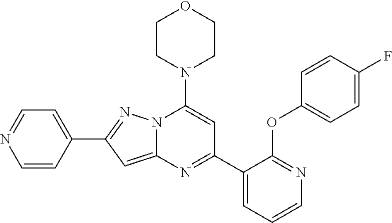 [SMILES: Fc1ccc(Oc2ncccc2-c2cc(N3CCOCC3)n3nc(-c4ccncc4)cc3n2)cc1] | ¹H NMR (400 MHz, DMSO-d₆) δ 8.71 (d, J = 5.6 Hz, 2H), 8.36 (dd, J = 7.6, 2.0 Hz, 1H), 8.24 (dd, J = 4.8, 2.0 Hz, 1H), 8.02 (d, J = 5.2 Hz, 2H), 7.36- 7.27 (m, 6H), 7.11 (s, 1H), 3.86-3.84 (m, 8H). LCMS (ESI) m/z: 469.2 [M + H]⁺. | 138 |

### Synthesis of 4-(5-(2-(3-fluorophenyl)pyrimidin-4-yl)-2-(pyridin-4-yl)pyrazolo[1,5-a]pyrimidin-7-yl)morpholine (Compound 139)

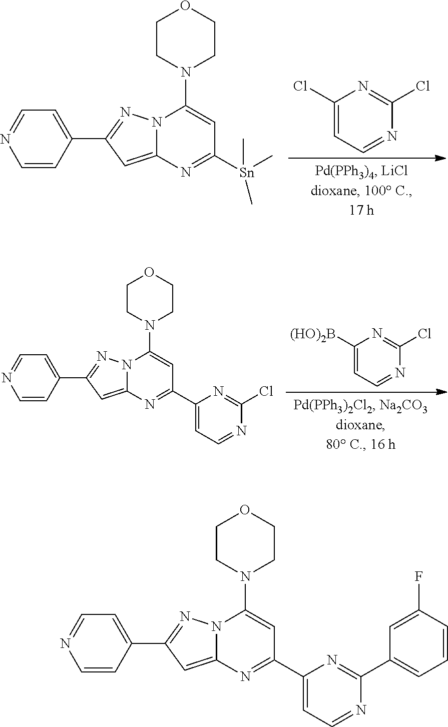 [SMILES: [CH3][Sn]([CH3])([CH3])[c]1cc(N2CCOCC2)n2nc(-c3ccncc3)cc2n1 ; Clc1ccnc(Cl)n1 ; Clc1nccc(-c2cc(N3CCOCC3)n3nc(-c4ccncc4)cc3n2)n1 ; OB(O)c1ccnc(Cl)n1 ; Fc1cccc(-c2nccc(-c3cc(N4CCOCC4)n4nc(-c5ccncc5)cc4n3)n2)c1]

### Step 1: Synthesis of 4-(5-(2-chloropyrimidin-4-yl)-2-(pyridin-4-yl)pyrazolo[1,5-a]pyrimidin-7-yl)morpholine

To a solution of 4-(2-(pyridin-4-yl)-5-(trimethylstannyl)pyrazolo[1,5-a]pyrimidin-7-yl)morpholine (1.335 g, 3 mmol) in dioxane (30 mL) were added 2,4-dichloropyrimidine (0.666 g, 4.5 mmol), lithium chloride (378 mg, 9 mmol) and tetrakis(triphenylphosphine)palladium(O) (345 mg, 0.3 mmol) at 25° C. The resultant mixture was stirred at 100° C. for 17 h under argon atmosphere. The mixture was then filtered to remove the solids, the filtrate was concentrated, and the residue was subjected to prep-TLC (Silica, UV254, DCM/MeOH=25/1) to obtain 4-(5-(2-chloropyrimidin-4-yl)-2-(pyridin-4-yl)pyrazolo[1,5-a]pyrimidin-7-yl)morpholine as yellow solid.(400 mg, 33%). LCMS (ESI) m/z: 394.2 [M+H]+.

### Step 2: Synthesis of 4-(5-(2-(3-fluorophenyl)pyrimidin-4-yl)-2-(pyridin-4-yl)pyrazolo[1,5-a]pyrimidin-7-yl)morpholine

A mixture of 4-(5-(2-chloropyrimidin-4-yl)-2-(pyridin-4-yl)pyrazolo[1,5-a]pyrimidin-7-yl)morpholine (39 mg, 0.1 mmol), 3-fluorophenylboronic acid (28 mg, 0.2 mmol), bis(triphenylphosphine)palladium(l1) chloride (7 mg, 0.01 mmol) and sodium carbonate (32 mg, 0.3 mmol) in dioxane (10 mL) and water (5 mL) was stirred at 80° C. for 16 h under argon atmosphere. The mixture was concentrated, and the residue was triturated with methanol (5 mL). The resultant precipitate was collected by filtration, and it was subjected to prep-HPLC (BOSTON pHlex ODS 10 μm 21.2×250 mm120 A. The mobile phase was acetonitrile/0.1% Ammonium bicarbonate) to obtain 4-(5-(2-(3-fluorophenyl)pyrimidin-4-yl)-2-(pyridin-4-yl)pyrazolo[1,5-a]pyrimidin-7-yl)morpholine as yellow solid. (5 mg, 11%). ¹H NMR (400 MHz, DMSO-d₆) δ 8.99 (d, J=5.2 Hz, 1H), 8.74 (d, J=5.6 Hz, 2H), 8.38-8.36 (m, 2H), 8.28-8.25 (m, 1H), 7.89 (d, J=5.6 Hz, 2H), 7.59 (s, 1H), 7.54-7.52 (m, 1H), 7.26-7.24 (m, 1H), 7.12 (s, 1H), 4.11-4.09 (m, 4H), 4.00-3.98 (m, 4H); LCMS (ESI) m/z: 454.2 [M+H]+.

### Synthesis of 4-(5-(2-benzylpyrimidin-4-yl)-2-(pyridin-4-yl)pyrazolo[1,5-a]pyrimidin-7-yl)morpholine (Compound 140)

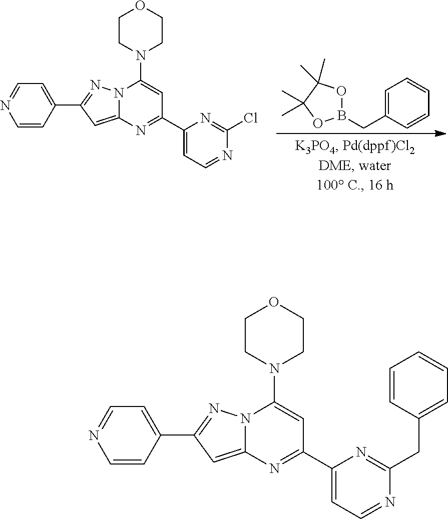

To a solution of 4-(5-(2-chloropyrimidin-4-yl)-2-(pyridin-4-yl)pyrazolo[1,5-a]pyrimidin-7-yl)morpholine (39 mg, 0.1 mmol) in 1,2-dimethyloxyethane (10 mL) and water (5 mL) were added 2-benzyl-4,4,5,5-tetramethyl-1,3,2-dioxaborolane (44 mg, 0.2 mmol), [1,1′-Bis(diphenylphosphino)ferrocene]dichloropalladium (II) (8 mg, 0.01 mmol) and tribasic potassium phosphate (63 mg, 0.3 mmol) at 25° C. The resulting mixture was stirred at 100° C. for 16 h under argon atmosphere and then concentrated. The residue was diluted with methanol (5 mL), the insoluble were filtered off and the filtrate was concentrated. The resultant residue was subjected to prep-HPLC (BOSTON pHlex ODS 10 μm 21.2×250 mm120 A. The mobile phase was acetonitrile/0.01% Formic acid) to obtain 4-(5-(2-benzylpyrimidin-4-yl)-2-(pyridin-4-yl)pyrazolo[1,5-a]pyrimidin-7-yl)morpholine as yellow solid. (8 mg, 18%). ¹H NMR (400 MHz, DMSO-de) δ 8.95 (d, J=5.2 Hz, 1H), 8.72 (dd, J=4.4, 2.4 Hz, 2H), 8.48 (s, 1H), 8.22 (d, J=4.8 Hz, 1H), 8.03 (dd, J=4.8 Hz, 2H), 7.46-7.34 (m, 7H), 4.36 (s, 2H), 3.92-3.89 (m, 8H); LCMS (ESI) m/z: 450.3 [M+H]+.

### Synthesis of −(5-(2-phenoxypyrimidin-4-yl)-2-(pyridin-4-yl)pyrazolo[1,5-a]pyrimidin-7-yl)morpholine (Compound 141)

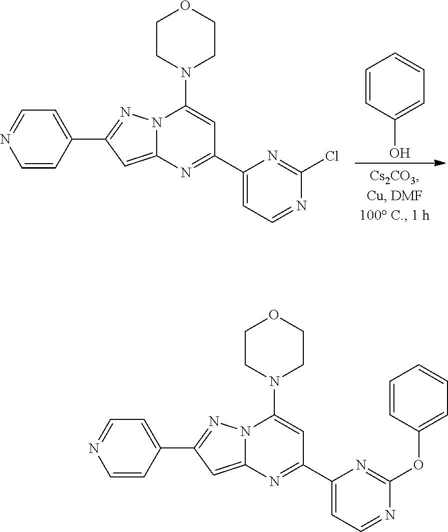 [SMILES: Clc1nccc(-c2cc(N3CCOCC3)n3nc(-c4ccncc4)cc3n2)n1 ; Oc1ccccc1 ; c1ccc(Oc2nccc(-c3cc(N4CCOCC4)n4nc(-c5ccncc5)cc4n3)n2)cc1]

To a solution of 4-(5-(2-chloropyrimidin-4-yl)-2-(pyridin-4-yl)pyrazolo[1,5-a]pyrimidin-7-yl)morpholine (39 mg, 0.1 mmol) in N,N-dimethylformamide (5 mL) were added phenol (19 mg, 0.2 mmol), Copper (3 mg, 0.05 mmol) and cesium carbonate (98 mg, 0.3 mmol) at 25° C. The resultant mixture was stirred at 100° C. for 1 h in a tube and then concentrated. The residue was dissolved in methanol (5 mL), the insoluble were filtered off and the filtrate was concentrated. The resultant residue was subjected to prep-HPLC (BOSTON pHlex ODS 10 μm 21.2×250 mm120 A. The mobile phase was acetonitrile/0.1% Ammonium bicarbonate) to obtain 4-(5-(2-phenoxypyrimidin-4-yl)-2-(pyridin-4-yl)pyrazolo[1,5-a]pyrimidin-7-yl)morpholine as yellow solid. (18 mg, 40%). ¹H NMR (400 MHz, DMSO-d₆) δ 8.82 (d, J=4.8 Hz, 1H), 8.73 (d, J=4.4 Hz, 2H), 8.12 (d, J=4.8 Hz, 1H), 8.03 (d, J=5.6 Hz, 2H), 7.53-7.48 (m, 3H), 7.35-7.31 (m, 3H), 7.18 (s, 1H), 3.87-3.86 (m, 8H); LCMS (ESI) m/z: 452.3 [M+H]+.

### Synthesis of 4-(5-(3-(1-methyl-1H-pyrazol-3-yl)phenyl)-2-(pyridin-4-yl)pyrazolo[1,5-a]pyrimidin-7-yl)morpholine (Compound 142)

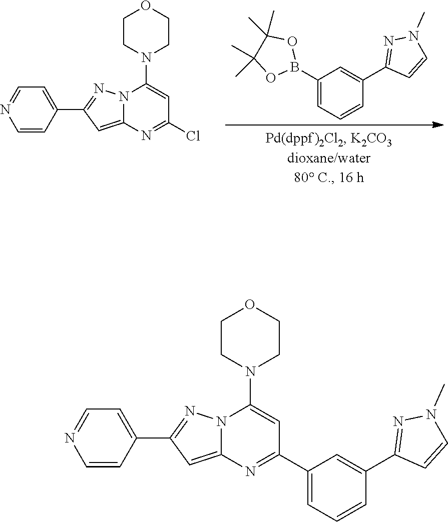 [SMILES: Clc1cc(N2CCOCC2)n2nc(-c3ccncc3)cc2n1 ; Cn1ccc(-c2cccc(B3OC(C)(C)C(C)(C)O3)c2)n1 ; Cn1ccc(-c2cccc(-c3cc(N4CCOCC4)n4nc(-c5ccncc5)cc4n3)c2)n1]

A mixture of 4-(5-chloro-2-(pyridin-4-yl)pyrazolo[1,5-a]pyrimidin-7-yl)morpholine (100 mg, 0.32 mmol), 1-methyl-3-(3-(4,4,5,5-tetramethyl-1,3,2-dioxaborolan-2-yl)phenyl)-1H-pyrazole (108 mg, 0.38 mol), [1,1′-bis(diphenylphosphino)ferrocene]dichloropalladium(II) (23 mg, 0.032 mol) and potassium carbonate (88 mg, 0.64 mmol) in 1,4-dioxane (1 mL) and water (0.0 mL) was stirred at 80° C. for 16 h under argon atmosphere. The contents were then partitioned between ethyl acetate (50 mL) and water (50 mL). The organic layer was washed with brine and evaporated to dryness. The crude product was chromatographed on silica gel (petroleum ether/ethyl acetate 10:1->1:1) to obtain 4-(5-(3-(1-methyl-1H-pyrazol-3-yl)phenyl)-2-(pyridin-4-yl)pyrazolo[1,5-a]pyrimidin-7-yl)morpholine (112 mg, 0.256 mmol, 80% yield) as white solid. ¹H NMR (400 MHz, DMSO-d₆) δ 8.72 (d, J=5.9 Hz, 2H), 8.60 (s, 1H), 8.12 (d, J=7.9 Hz, 1H), 8.02 (d, J=6.0 Hz, 2H), 7.92 (d, J=7.7 Hz, 1H), 7.79 (d, J=2.1 Hz, 1H), 7.56 (t, J=7.7 Hz, 1H), 7.35 (s, 1H), 6.97 (s, 1H), 6.83 (d, J=2.2 Hz, 1H), 3.93 (d, J=4.3 Hz, 11H). LCMS (ESI) m/z: 438.1 [M+H]⁺.

### Synthesis of 4-(5-(3-phenyl-1H-pyrazol-1-yl)-2-vinylpyrazolo[1,5-a]pyrimidin-7-yl)morpholine (Compound 143)

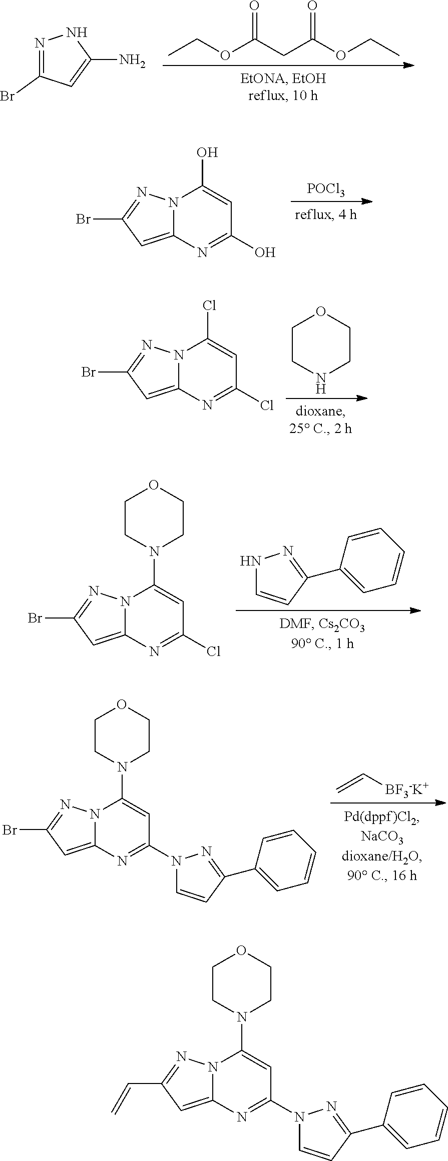

### Step 1: Synthesis of 2-bromopyrazolo[1,5-a]pyrimidine-5,7-diol

To a solution of 3-bromo-1H-pyrazol-5-amine (2.0 g, 12.0 mmol) in ethanol (30 mL) was added sodium ethoxide (20% in ethanol, 15 mL, 36.0 mmol) and diethyl malonate (5.76 g, 36 mmol). The resultant mixture was stirred for 10 h at reflux temperature. The reaction was cooled down and the mixture was filtered to collect the solid. It was washed with diethyl ether and dried to obtain 2-bromopyrazolo[1,5-a]pyrimidine-5,7-diol (2.7 g, 11.8 mmol) as yellow solid, which was used directly in next step without further purification. LCMS (ESI) m/z: 231.9 [M+H]+.

### Step 2: Synthesis of 2-bromo-5,7-dichloropyrazolo[1,5-a]pyrimidine

A mixture of 2-bromopyrazolo[1,5-a]pyrimidine-5,7-diol (2.7 g, 11.7 mmol) in phosphoryl trichloride (30 mL) was refluxed for 4 h and concentrated. The residue was diluted with ethanol with ice-bath cooling and stirred for 15 min. The mixture was concentrated again, and the residue was subjected to flash chromatography (Biotage, 40 g silica gel, eluted with methanol/methylene chloride=1:50 to 1:20) to obtain 2-bromo-5,7-dichloropyrazolo[1,5-a]pyrimidine (1.6 g, 50% over two steps) as yellow solid. LCMS (ESI) m/z: 267.9 [M+H]+.

### Step 3: Synthesis of 4-(2-bromo-5-chloropyrazolo[1,5-a]pyrimidin-7-yl)morpholine

To a solution of 2-bromo-5,7-dichloropyrazolo[1,5-a]pyrimidine (2.7 g, 10.0 mmol) in 1,4-dioxane (40 mL) was added morpholine (1.3 g, 15.0 mmol) and the mixture was stirred at 25° C. for 2 h. The resultant suspension was concentrated, the concentrate was diluted with water (40 mL) and then the mixture was extracted with ethyl acetate (60 mL) twice. The resulting extracts were combined, dried over anhydrous sodium sulfate, filtered and concentrated. The resulting solid was washed with diethyl ether to obtain 4-(2-bromo-5-chloropyrazolo[1,5-a]pyrimidin-7-yl)morpholine (1.6 g, 50%) as yellow solid. LCMS (ESI) m/z: 319.0 [M+H]+.

### Step 4: Synthesis of 4-(2-bromo-5-(3-phenyl-1H-pyrazol-1-yl)pyrazolo[1,5-a]pyrimidin-7-yl)morpholine

To a solution of 4-(2-bromo-5-chloropyrazolo[1,5-a]pyrimidin-7-yl)morpholine (200 mg, 0.63 mmol) in DMF (8 mL) were added 3-phenyl-1H-pyrazole (110 mg, 0.76 mmol) and cesium carbonate (410 mg, 0.76 mmol) and the mixture was stirred at 90° C. under nitrogen atmosphere for 1 h. It was then diluted with ethyl acetate (80 mL), washed with brine (50 mL*3), dried over sodium sulfate and concentrated. The residue was subjected to flash chromatography on silica gel (petroleum ether: ethyl acetate=4:1) to obtain 4-(2-bromo-5-(3-phenyl-1H-pyrazol-1-yl)pyrazolo[1,5-a]pyrimidin-7-yl)morpholine (170 mg, 63%) as white solid. LCMS (ESI) m/z: 426.9 [M+H]⁺.

### Step 5: Synthesis of 4-(5-(3-phenyl-1H-pyrazol-1-yl)-2-vinylpyrazolo[1,5-a]pyrimidin-7-yl)morpholine

A mixture of 4-(2-bromo-5-(3-phenyl-1H-pyrazol-1-yl)pyrazolo[1,5-a]pyrimidin-7-yl)morpholine (170 mg, 0.4 mmol), trifluoro(vinyl)-14-borane, potassium salt (107 mg, 0.8 mmol), 1,1′-bis(diphenylphosphino)ferrocene-palladium(II)dichloride dichloromethane complex (35 mg, 0.04 mmol) and potassium carbonate (130 mg, 1.2 mmol) in dioxane (5 mL) and water (1 mL) was stirred at 90° C. for 16 h. The mixture was cooled to 25° C. and diluted with ethyl acetate (40 mL). The organic mixture was washed with water (20 mL×2), brine (20 mL), dried over sodium sulfate and concentrated. The residue was subjected to prep-HPLC (SunFire C18, 4.6*50 mm, 3.5 um column Xbridge C18 3.5 μm 4.6×50 mm column. The elution system used was a gradient of 5%-95% over 1.5 min at 2 ml/min and the solvent was acetonitrile/0.01% aqueous NH₄HCO₃.) to obtain 4-(5-(3-phenyl-1H-pyrazol-1-yl)-2-vinylpyrazolo[1,5-a]pyrimidin-7-yl)morpholine (60 mg, 40%) as white solid. ¹H NMR (400 MHz, DMSO-d₆) δ 8.71 (d, J=2.7 Hz, 1H), 8.07-7.97 (m, 2H), 7.52-7.41 (m, 3H), 7.15 (d, J=2.7 Hz, 1H), 6.97 (s, 1H), 6.88-6.73 (m, 2H), 6.10 (d, J=17.6 Hz, 1H), 5.56 (d, J=12.3 Hz, 1H), 3.87 (s, 8H); LCMS (ESI) m/z: 373.0 [M+H]⁺.

### Synthesis of 1-(7-morpholino-5-(3-phenyl-1H-pyrazol-1-yl)pyrazolo[1,5-a]pyrimidin-2-yl)ethane-1,2-diol (Compound 144)

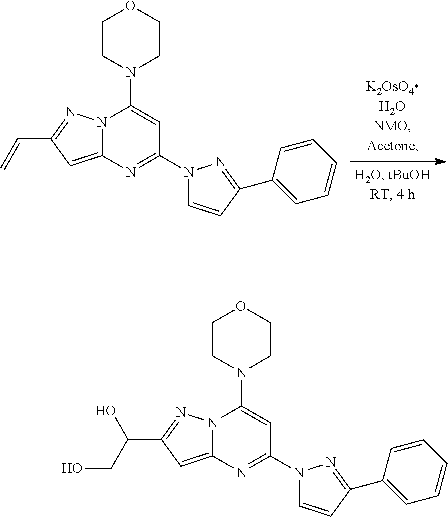 [SMILES: C=Cc1cc2nc(-n3ccc(-c4ccccc4)n3)cc(N3CCOCC3)n2n1 ; OCC(O)c1cc2nc(-n3ccc(-c4ccccc4)n3)cc(N3CCOCC3)n2n1]

A solution of 4-(5-(3-phenyl-1H-pyrazol-1-yl)-2-vinylpyrazolo[1,5-a]pyrimidin-7-yl)morpholine (130 mg, 0.35 mmol) in acetone (9 mL), water (2.2 mL) and 2-methylpropn-2-ol (2.2 mL) were added potassium osmate(VI) dehydrate (26 mg, 0.07 mmol) and 4-methylmorpholine N-oxide (120 mg, 1.06 mmol) at 25° C. The resultant mixture was stirred for 4 h, then the insoluble materials were removed by filtration and the filtrate was concentrated. The residue was subjected to by prep-HPLC (BOSTON pHlex ODS 10 μm 21.2×250 mm 120 A. The mobile phase was acetonitrile/0.1% Ammonium bicarbonate) to obtain 5-chloro-N-(5-(1,2-dihydroxyethyl)pyridin-2-yl)-1H-indole-2-carboxamide as white solid (54 mg, 38%). ¹H NMR (400 MHz, DMSO-d₆) δ 8.71 (d, J=2.7 Hz, 1H), 8.01 (d, J=7.2 Hz, 2H), 7.52-7.39 (m, 3H), 7.14 (d, J=2.7 Hz, 1H), 6.95 (s, 1H), 6.44 (s, 1H), 5.41 (d, J=5.2 Hz, 1H), 4.77-4.68 (m, 2H), 3.87 (s, 8H) 3.73-3.59 (m, 2H); LCMS (ESI) m/z: 407.1 [M+H]+.

### Synthesis of 1-(5-(3-methoxy-4-phenyl-1H-pyrazol-1-yl)-7-morpholinopyrazolo[1,5-a]pyrimidin-2-yl)ethane-1,2-diol (Compound 145)

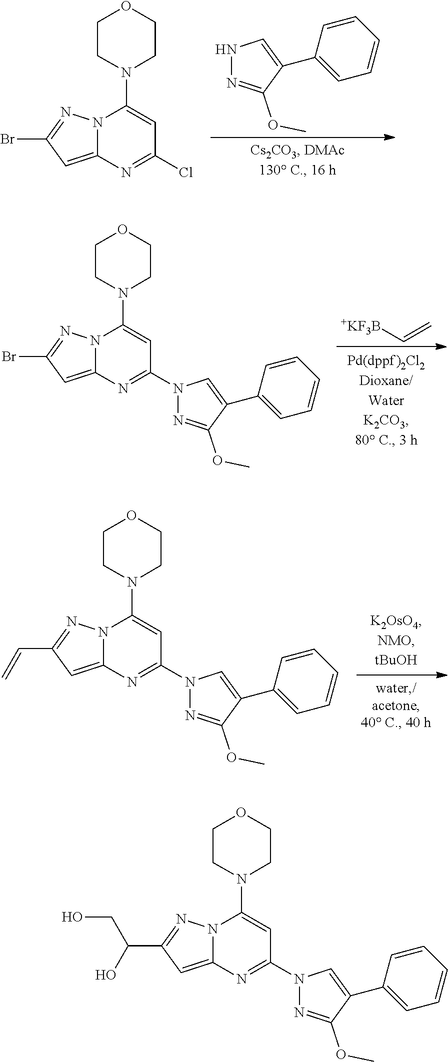

### Step 1: Synthesis of 4-(2-bromo-5-(3-methoxy-4-phenyl-1H-pyrazol-1-yl)pyrazolo[1,5-a]pyrimidin-7-yl)morpholine

A mixture of 4-(2-bromo-5-chloropyrazolo[1,5-a]pyrimidin-7-yl)morpholine (317 mg, 1 mmol), 3-methoxy-4-phenyl-1H-pyrazole (156 mg, 0.9 mmol) and cesium carbonate (975 mg, 3 mmol) in N,N-dimethylacetamide (5 mL) was stirred at 130° C. under argon atmosphere for 16 h. The mixture was filtered to remove the insoluble materials and the filtrate was concentrated. The residue was subjected to flash chromatography on silica gel (eluted with dichloromethane/methanol=10/1) to obtain 4-(2-bromo-5-(3-methoxy-4-phenyl-1H-pyrazol-1-yl)pyrazolo[1,5-a]pyrimidin-7-yl)morpholine (300 mg, 66%) as white solid.

LCMS (ESI) m/z: 456.9 [M+H]⁺.

### Step 2: Synthesis of 4-(5-(3-methoxy-4-phenyl-1H-pyrazol-1-yl)-2-vinylpyrazolo[1,5-a]pyrimidin-7-yl)morpholine

A mixture of 4-(2-bromo-5-(3-methoxy-4-phenyl-1H-pyrazol-1-yl)pyrazolo[1,5-a]pyrimidin-7-yl)morpholine (102 mg, 0.22 mmol), potassium ethenyltrifluoroborate (43 mg, 0.33 mmol), [1,1′-bis(diphenylphosphino)ferrocene]dichloropalladium(II) (16 mg, 0.022 mmol), and potassium carbonate (91 mg, 0.66 mmol) in 1,4-dioxane (5 mL) and water (0.5 mL) was stirred at 80° C. under argon atmosphere for 3 h. The mixture was filtered to remove the solids and the filtrate was concentrated. The crude product was purified by flash chromatography on silica gel (eluted with dichloromethane/methanol=10/1) to obtain 4-(5-(3-methoxy-4-phenyl-1H-pyrazol-1-yl)-2-vinylpyrazolo[1,5-a]pyrimidin-7-yl)morpholine (250 mg, 64%) as white solid. LCMS (ESI) m/z: 403.0 [M+H]⁺.

### Step 3: Synthesis of 1-(5-(3-methoxy-4-phenyl-1H-pyrazol-1-yl)-7-morpholinopyrazolo[1,5-a]pyrimidin-2-yl)ethane-1,2-diol

To a solution of 4-(5-(3-methoxy-4-phenyl-1H-pyrazol-1-yl)-2-vinylpyrazolo[1,5-a]pyrimidin-7-yl)morpholine (10 mg, 0.025 mmol) in acetone (3 mL) were added potassium osmate(VI) dehydrate (1.65 mg, 0.005 mmol), 4-methylmorpholine N-oxide (4 mg, 0.037 mmol), 2-methylpropan-2-ol (1 mL) and water (1 mL). The reaction mixture was stirred at 25° C. for 4 h under nitrogen atmosphere. The reaction mixture was then filtered to remove the solids and the filtrate was concentrated. The residue was subjected to prep-HPLC (BOSTON pHlex ODS 10 μm 21.2×250 mm 120 A. The mobile phase was acetonitrile/0.1% ammonium bicarbonate) to obtain 1-(5-(3-methoxy-4-phenyl-1H-pyrazol-1-yl)-7-morpholinopyrazolo[1,5-a]pyrimidin-2-yl)ethane-1,2-diol (3.1 mg, 28%) as white solid. ¹H NMR (400 MHz, DMSO-d₆) δ 8.95 (s, 1H), 7.83 (d, J=7.8 Hz, 2H), 7.40 (t, J=7.7 Hz, 2H), 7.25 (dd, J=17.6, 10.6 Hz, 1H), 6.73 (s, 1H), 6.38 (s, 1H), 4.68 (d, J=5.0 Hz, 1H), 4.10 (s, 3H), 3.84 (d, J=9.9 Hz, 8H), 3.68 (d, J=4.8 Hz, 1H), 3.62 (d, J=6.7 Hz, 1H). LCMS (ESI) m/z: 437.1 [M+H]⁺.

### Synthesis of 4-(5-(1-phenyl-1H-pyrazol-5-yl)-2-(pyridin-4-yl)pyrazolo[1,5-a]pyrimidin-7-yl)morpholine (Compound 146)

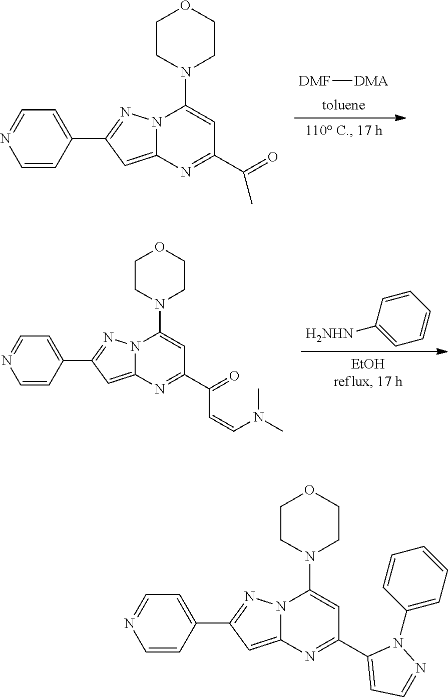

### Step 1: Synthesis of (Z)-3-(dimethylamino)-1-(7-morpholino-2-(pyridin-4-yl)pyrazolo[1,5-a]pyrimidin-5-yl)prop-2-en-1-one

To a solution of 1-(7-morpholino-2-(pyridin-4-yl)pyrazolo[1,5-a]pyrimidin-5-yl)ethanone (323 mg, 1 mmol) in toluene (10 mL) was added N,N-dimethylformamide dimethyl acetal (595 mg, 5 mmol) and the mixture was stirred at 110° C. for 17 h. It was then concentrated to obtain (Z)-3-(dimethylamino)-1-(7-morpholino-2-(pyridin-4-yl)pyrazolo[1,5-a]pyrimidin-5-yl)prop-2-en-1-one as yellow solid (378 mg, 99%).

LCMS (ESI) m/z: 379.2 [M+H]+.

### Step 2: Synthesis of 4-(5-(1-phenyl-1H-pyrazol-5-yl)-2-(pyridin-4-yl)pyrazolo[1,5-a]pyrimidin-7-yl)morpholine

A mixture of (Z)-3-(dimethylamino)-1-(7-morpholino-2-(pyridin-4-yl)pyrazolo[1,5-a]pyrimidin-5-yl)prop-2-en-1-one (189 mg, 0.5 mmol) and phenylhydrazine (108 mg, 1 mmol) in ethanol (10 mL) was stirred at 80° C. for 16 h. It was then concentrated, and the residue was subjected to prep-HPLC (BOSTON pHlex ODS 10 um 21.2×250 mm120 A. The mobile phase was acetonitrile/0.1% Formic acid) to obtain 4-(5-(1-phenyl-1H-pyrazol-5-yl)-2-(pyridin-4-yl)pyrazolo[1,5-a]pyrimidin-7-yl)morpholine as white solid (5 mg, 2%).1H NMR (400 MHz, CDCl₃) δ 8.70 (dd, J=4.4 Hz, 2H), 7.82 (dd, J=4.4 Hz, 2H), 7.79 (d, J=2.0 Hz, 1H), 7.42 (s, 5H), 6.96-6.95 (m, 2H), 5.84 (s, 1H), 3.87 (m, 4H), 3.56-3.54 (m, 4H); LCMS (ESI) m/z: 424.2 [M+H]+.

### Synthesis of 4-(5-(5-(3-fluorophenyl)-1H-pyrazol-1-yl)-2-(pyridin-4-yl)pyrazolo[1,5-a]pyrimidin-7-yl)morpholine (Compound 147)

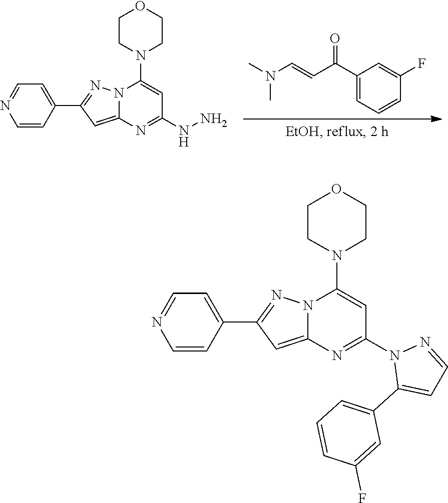

A solution of 4-(5-hydrazinyl-2-(pyridin-4-yl)pyrazolo[1,5-a]pyrimidin-7-yl)morpholine (155 mg, 0.5 mmol), (E)˜3-(dimethylamino)-1˜(3-fluorophenyl)prop-2-en-1-one (193 mg, 1 mmol) in ethanol (10 mL) was stirred at 90° C. for 2 h. The mixture was then concentrated, and the residue was subjected to prep-HPLC (BOSTON pHlex ODS 10 um 21.2×250 mm120 A. The mobile phase was acetonitrile/0.1% Formic acid) to obtain 4-(5-(5-(3-fluorophenyl)-1H-pyrazol-1-yl)-2-(pyridin-4-yl)pyrazolo[1,5-a]pyrimidin-7-yl)morpholine as white solid (80 mg, 36%). ¹H NMR (400 MHz, DMSO-d₆) δ 8.67 (dd, J=4.4, 2.0 Hz, 2H), 7.97 (dd, J=4.8, 1.6 Hz, 2H), 7.90 (d, J=2.0 Hz, 1H), 7.42-7.38 (m, 1H), 7.30-7.19 (m, 3H), 7.01 (s, 1H), 6.80 (s, 1H), 3.89 (s, 8H); LCMS (ESI) m/z: 442.2 [M+H]+.

### Synthesis of N,N-dimethyl-3-(7-morpholino-5-(3-(m-tolyl)-1H-pyrazol-1-yl)pyrazolo[1,5-a]pyrimidin-2-yl)propenamide (Compound 149)

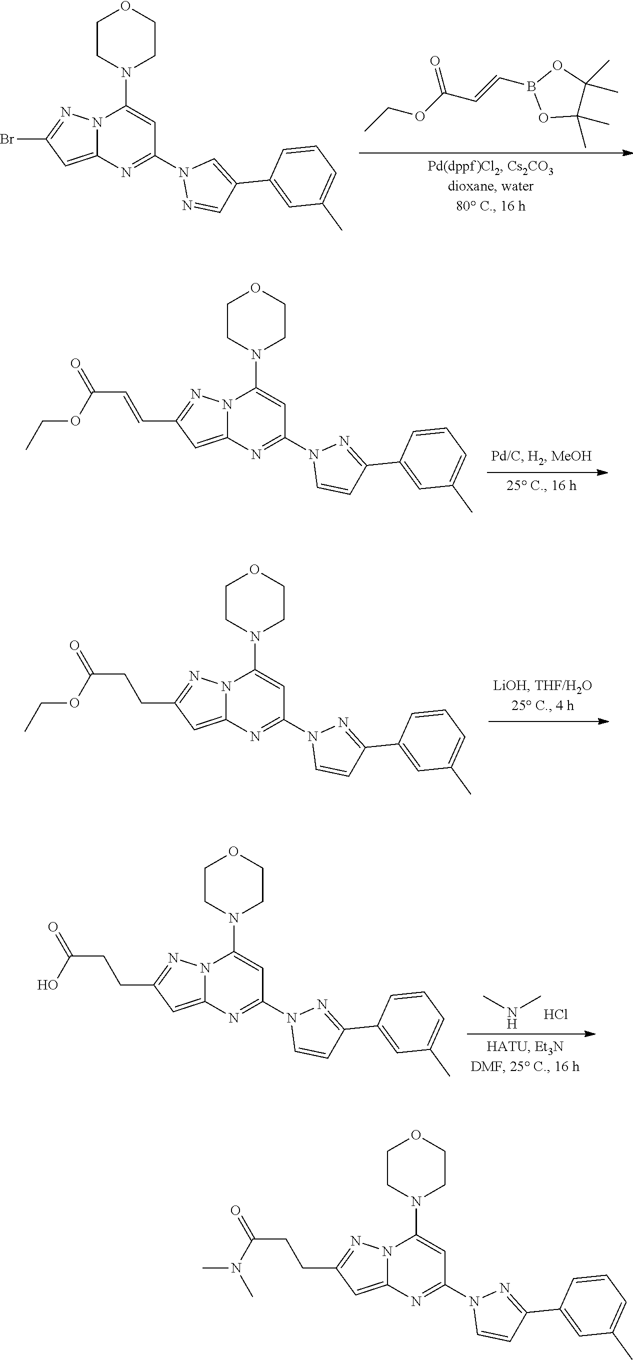

### Step 1: Synthesis of ethyl (E)-3-(7-morpholino-5-(3-(m-tolyl)-1H-pyrazol-1-yl)pyrazolo[1,5-a]pyrimidin-2-yl)acrylate

To a solution of 4-(2-bromo-5-(4-(m-tolyl)-1H-pyrazol-1-yl)pyrazolo[1,5-a]pyrimidin-7-yl)morpholine (0.6 g, 1.37 mmol) in 1,4-dioxane/water (20 mL/5 mL) was added ethyl (E)-3-(4,4,5,5-tetramethyl-1,3,2-dioxaborolan-2-yl)acrylate (0.62 g, 2.73 mmol), cesium carbonate (1.33 g, 4.1 mmol) and [1,1′-bis(diphenylphosphino)ferrocene]dichloro palladium(II) (0.1 g, 0.14 mmol) at 25° C. and the resultant mixture was stirred at 70° C. for 16 h under argon atmosphere. The reaction mixture was concentrated and the residue was subjected to flash column chromatography (methanol/dichloromethane=0%-8%) to obtain ethyl (E)-3-(7-morpholino-5-(3-(m-tolyl)-1H-pyrazol-1-yl)pyrazolo[1,5-a]pyrimidin-2-yl)acrylate (0.63 g, 46%) as yellow solid. LCMS (ESI) m/z: 459.1 [M+H]⁺.

### Step 2: Synthesis of ethyl 3-(7-morpholino-5-(3-(m-tolyl)-1H-pyrazol-1-yl)pyrazolo[1,5-a]pyrimidin-2-yl)propanoate

A mixture of ethyl (E)-3-(7-morpholino-5-(3-(m-tolyl)-1H-pyrazol-1-yl)pyrazolo[1,5-a]pyrimidin-2-yl)acrylate (160 mg, 0.35 mmol) and palladium on activated carbon (10% Pd,160 mg) in methanol (20 mL) was stirred at 25° C. for 16 h under hydrogen atmosphere. The reaction mixture was filtered and the filtrate was concentrated to obtain ethyl 3-(7-morpholino-5-(3-(m-tolyl)-1H-pyrazol-1-yl)pyrazolo[1,5-a]pyrimidin-2-yl)propanoate (150 mg, 93%) as white solid. LCMS (ESI) m/z: 461.1 [M+H]⁺.

### Step 3: Synthesis of 3-(7-morpholino-5-(3-(m-tolyl)-1H-pyrazol-1-yl)pyrazolo[1,5-a]pyrimidin-2-yl)propanoic acid

A mixture of ethyl 3-(7-morpholino-5-(3-(m-tolyl)-1H-pyrazol-1-yl)pyrazolo[1,5-a]pyrimidin-2-yl)propanoate (0.15 g, 0.33 mmol) and lithium hydroxide hydrate (0.028 g, 0.65 mmol) in water (3 mL) and tetrahydrofuran (12 mL) was stirred at 25° C. for 5 h. The resultant reaction mixture was concentrated to obtain 3-(7-morpholino-5-(3-(m-tolyl)-1H-pyrazol-1-yl)pyrazolo[1,5-a]pyrimidin-2-yl)propanoic acid (0.14 g, 99%) as yellow solid. LCMS (ESI) m/z: 433.1 [M+H]⁺.

### Step 4: Synthesis of N,N-dimethyl-3-(7-morpholino-5-(3-(m-tolyl)-1H-pyrazol-1-yl)pyrazolo[1,5-a]pyrimidin-2-yl)propenamide

A solution of 3-(7-morpholino-5-(3-(m-tolyl)-1H-pyrazol-1-yl)pyrazolo[1,5-a]pyrimidin-2-yl)propanoic acid (230 mg, 0.53 mmol), dimethylamine hydrochloride (220 mg, 2.7 mmol), triethylamine (320 mg, 3.2 mmol) and 1-[bis(dimethylamino)methylene]-1H-1,2,3-triazolo[4,5-b]pyridinium 3-oxid hexafluorophosphate (320 mg, 0.8 mmol) in N,N-dimethylformamide (15 mL) was stirred at 25° C. for 16 h.

The reaction mixture was filtered, and the filtrate was purified by prep-HPLC (BOSTON pHlex ODS 1 Oum 21.2×250 mm 120 A. The mobile phase was acetonitrile/0.1% ammonium bicarbonate) to obtain N,N-dimethyl-3-(7-morpholino-5-(3-(m-tolyl)-1H-pyrazol-1-yl)pyrazolo[1,5-a]pyrimidin-2-yl)propanamide (85.6 mg, 35%) as white solid. ¹H NMR (500 MHz, DMSO-d₆) δ 8.68 (d, J=2.7 Hz, 1H), 7.81 (s, 1H), 7.79 (d, J=8.0 Hz, 1H), 7.37 (t, J=7.6 Hz, 1H), 7.22 (d, J=7.4 Hz, 1H), 7.11 (d, J=2.7 Hz, 1H), 6.90 (s, 1H), 6.37 (s, 1H), 3.86 (s, 8H), 3.00 (s, 3H), 2.98 (d, J=7.5 Hz, 2H), 2.84 (s, 3H), 2.76 (t, J=7.4 Hz, 2H), 2.40 (s, 3H). LCMS (ESI) m/z: 460.0 [M+H]⁺.

### Synthesis of 1-(7-morpholino-5-(3-(m-tolyl)-1H-pyrazol-1-yl)pyrazolo[1,5-a]pyrimidin-2-yl)ethane-1,2-diol (Compound 150)

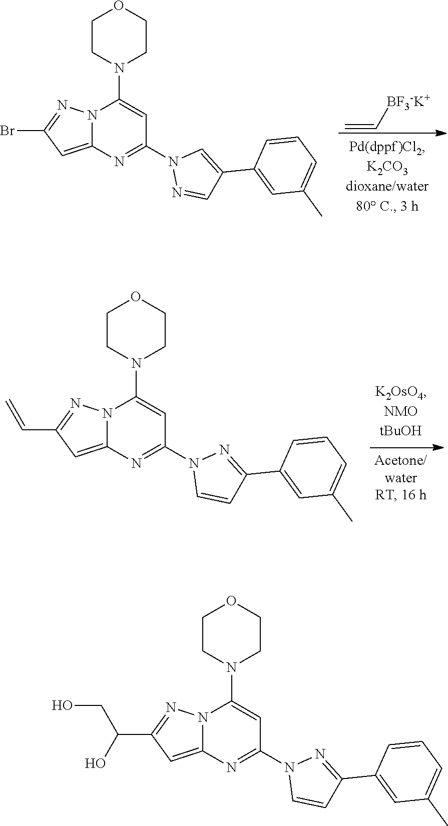

### Step 1: Synthesis of 4-(5-(3-(m-tolyl)-1H-pyrazol-1-yl)-2-vinylpyrazolo[1,5-a]pyrimidin-7-yl)morpholine

A mixture of 4-(2-bromo-5-(4-(m-tolyl)-1H-pyrazol-1-yl)pyrazolo[1,5-a]pyrimidin-7-yl)morpholine (439 mg, 1 mmol), 4,4,4′,4′,5,5,5′,5′-octamethyl-2,2′-bi(1,3,2-dioxaborolane) (200 mg, 1.5 mmol), [1,1′-bis(diphenylphosphino)ferrocene]dichloropalladium(II) (73 mg, 0.1 mmol), and potassium carbonate (414 mg, 3 mmol) in 1,4-dioxane (5 mL) was stirred at 80° C. under argon atmosphere for 3 h. The mixture was filtered, and the filtrate was concentrated. The residue was subjected to silica gel column chromatography (dichloromethane/methanol=10/1) to obtain 4-(5-(3-(m-tolyl)-1H-pyrazol-1-yl)-2-vinylpyrazolo[1,5-a]pyrimidin-7-yl)morpholine (250 mg, 64%) as white solid. LCMS (ESI) m/z: 387.1 [M+H]⁺.

### Step 2: Synthesis of 1-(7-morpholino-5-(3-(m-tolyl)-1H-pyrazol-1-yl)pyrazolo[1,5-a]pyrimidin-2-yl)ethane-1,2-diol

To a solution of 4-(5-(3-(m-tolyl)-1H-pyrazol-1-yl)-2-vinylpyrazolo[1,5-a]pyrimidin-7-yl)morpholine (200 mg, 0.52 mmol) in acetone (6 mL) was added potassium osmate(VI) dehydrate (34 mg, 0.1 mmol), 4-methylmorpholine n-oxide (91 mg, 0.78 mmol), 2-methylpropan-2-ol (2 mL) and water (2 mL) and the reaction mixture was stirred at 25° C. for 4 h under nitrogen atmosphere. The reaction mixture was then filtered and the filtrate was concentrated. The residue was subjected to prep-HPLC (BOSTON pHlex ODS 10 um 21.2×250 mm 120 A, with mobile phase acetonitrile/0.1% ammonium bicarbonate) to obtain 1-(7-morpholino-5-(3-(m-tolyl)-1H-pyrazol-1-yl)pyrazolo[1,5-a]pyrimidin-2-yl)ethane-1,2-diol (49.7 mg, 23%) as white solid. ¹H NMR (400 MHz, DMSO-d₆) δ 8.70 (d, J=2.6 Hz, 1H), 7.80 (d, J=11.0 Hz, 2H), 7.37 (t, J=7.6 Hz, 1H), 7.23 (d, J=7.4 Hz, 1H), 7.12 (d, J=2.7 Hz, 1H), 6.94 (s, 1H), 6.44 (s, 1H), 4.70 (dd, J=7.0, 4.8 Hz, 1H), 3.87 (s, 8H), 3.75-3.56 (m, 3H), 2.40 (s, 3H). LCMS (ESI) m/z: 421.0 [M+H]⁺.

### Synthesis of 1-methyl-4-(7-morpholino-5-(3-(m-tolyl)-1H-pyrazol-1-yl)pyrazolo[1,5-a]pyrimidin-2-yl)piperidin-2-one (Compound 151)

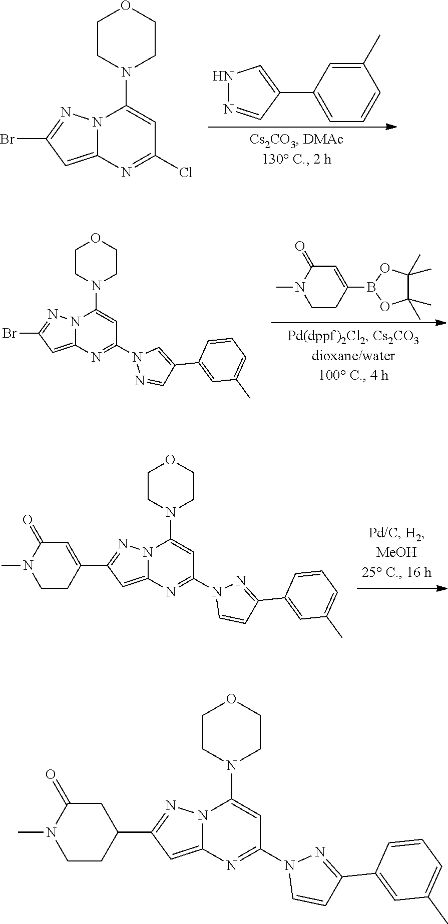

### Step 1: Synthesis of 4-(2-bromo-5-(4-(m-tolyl)-1H-pyrazol-1-yl)pyrazolo[1,5-a]pyrimidin-7-yl)morpholine

A mixture of 4-(2-bromo-5-chloropyrazolo[1,5-a]pyrimidin-7-yl)morpholine (1.2 g, 3.8 mmol), 4-(m-tolyl)-1H-pyrazole (600 mg, 3.8 mmol) and cesium carbonate (2.5 g, 7.6 mmol) in N,N-dimethylacetamide (30 mL) was stirred at 130° C. for 2 h. The reaction was cooled down and the mixture was diluted with ethyl acetate/water (50 mL/100 mL), the organic layer was separated and the aqueous layer was extracted with ethyl acetate (100 mL) twice. The combined organic phase was washed with brine (30 mL), dried over sodium sulfate, filtered and concentrated. The residue was subjected to flash column chromatography (methanol/dichloromethane=0%-5%) to obtain 4-(2-bromo-5-(4-(m-tolyl)-1H-pyrazol-1-yl)pyrazolo[1,5-a]pyrimidin-7-yl)morpholine (1.4 g, 84%) as white solid. LCMS (ESI) m/z: 439.1 [M+H]⁺.

### Step 2: Synthesis of 1-methyl-4-(7-morpholino-5-(3-(m-tolyl)-1H-pyrazol-1-yl)pyrazolo[1,5-a]pyrimidin-2-yl)-5,6-dihydropyridin-2(1H)-one

A mixture of 1-methyl-4-(4,4,5,5-tetramethyl-1,3,2-dioxaborolan-2-yl)-5,6-dihydropyridin-2(1H)-one (0.2 g, 1.4 mmol), 4-(2-bromo-5-(4-(m-tolyl)-1H-pyrazol-1-yl)pyrazolo[1,5-a]pyrimidin-7-yl)morpholine (0.3 g, 0.68 mmol), cesium carbonate (0.44 g, 1.4 mmol) and [1,1′-bis(diphenylphosphino)ferrocene]dichloropalladium(II) (0.05 g, 0.07 mmol) in 1,4-dioxane (10 mL) and water (3 mL) was stirred at 100° C. for 4 h under nitrogen atmosphere. The reaction mixture was filtered and the filtrate was concentrated. The residue was subjected to flash column chromatography (dichloromethane: methanol=15:1) to obtain 1-methyl-4-(7-morpholino-5-(3-(m-tolyl)-1H-pyrazol-1-yl)pyrazolo[1,5-a]pyrimidin-2-yl)-5,6-dihydropyridin-2(1H)-one as yellow solid (0.15 g, 47%). LCMS (ESI) m/z: 470.1 [M+H]⁺.

### Step 3: Synthesis of 1-methyl-4-(7-morpholino-5-(3-(m-tolyl)-1H-pyrazol-1-yl)pyrazolo[1,5-a]pyrimidin-2-yl)piperidin-2-one

A mixture of 1-methyl-4-(7-morpholino-5-(3-(m-tolyl)-1H-pyrazol-1-yl)pyrazolo[1,5-a]pyrimidin-2-yl)-5,6-dihydropyridin-2(1H)-one (150 mg, 0.34 mmol) and palladium on activated carbon (10%, 160 mg) in methanol (20 mL) was stirred at 25° C. for 16 h under hydrogen atmosphere. The reaction mixture was then filtered, and the filtrate was concentrated. The residue was subjected to prep-HPLC (BOSTON pHlex ODS 10 μm 21.2×250 mm 120 A. The mobile phase was acetonitrile/0.1% ammonium bicarbonate) to obtain 1-methyl-4-(7-morpholino-5-(3-(m-tolyl)-1H-pyrazol-1-yl)pyrazolo[1,5-a]pyrimidin-2-yl)piperidin-2-one (70.7 mg, 44%) as white solid. ¹H NMR (400 MHz, DMSO-d₆) δ 8.68 (d, J=2.6 Hz, 1H), 7.80 (d, J=11.1 Hz, 2H), 7.37 (t, J=7.5 Hz, 1H), 7.22 (d, J=7.5 Hz, 1H), 7.11 (d, J=2.6 Hz, 1H), 6.93 (s, 1H), 6.45 (s, 1H), 3.86 (s, 8H), 3.36 (d, J=9.5 Hz, 2H), 3.31-3.23 (m, 1H), 2.84 (s, 3H), 2.64 (dd, J=16.9, 5.2 Hz, 1H), 2.57 (d, J=9.4 Hz, 1H), 2.40 (s, 3H), 2.23 (d, J=9.1 Hz, 1H), 2.04-1.91 (m, 1H). LCMS (ESI) m/z: 472.0 [M+H]⁺.

### Synthesis of 1-(5-(3-(1-methyl-1H-pyrazol-3-yl)phenyl)-7-morpholinopyrazolo[1,5-a]pyrimidin-2-yl)piperidin-4-ol (Compound 152)

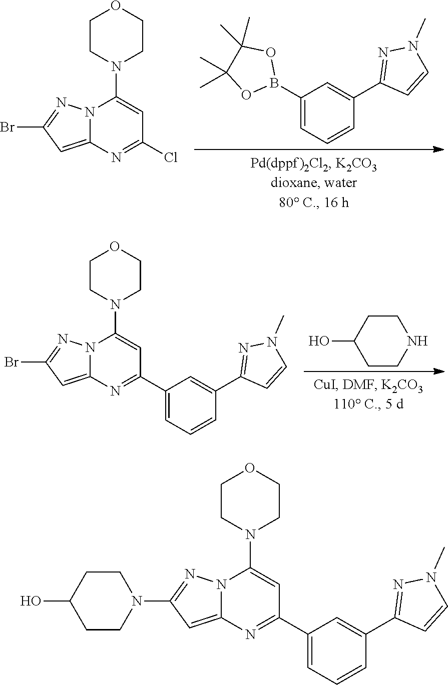 [SMILES: Clc1cc(N2CCOCC2)n2nc(Br)cc2n1 ; Cn1ccc(-c2cccc(B3OC(C)(C)C(C)(C)O3)c2)n1 ; Cn1ccc(-c2cccc(-c3cc(N4CCOCC4)n4nc(Br)cc4n3)c2)n1 ; OC1CCNCC1 ; Cn1ccc(-c2cccc(-c3cc(N4CCOCC4)n4nc(N5CCC(O)CC5)cc4n3)c2)n1]

### Step 1: Synthesis of 4-(2-bromo-5-(3-(1-methyl-1H-pyrazol-3-yl)phenyl)pyrazolo[1,5-a]pyrimidin-7-yl)morpholine

A mixture of 4-(2-bromo-5-chloropyrazolo[1,5-a]pyrimidin-7-yl)morpholine (456 mg, 1.44 mmol), [1,1′-bis(diphenylphosphino) ferrocene]dichloropalladium(II) (102 mg, 0.14 mmol), potassium carbonate(397 mg, 2.88 mmol) and 1-methyl-3-(3-(4,4,5,5-tetramethyl-1,3,2-dioxaborolan-2-yl)phenyl)-1H-pyrazole (450 mg, 1.58 mol) in 1,4-dioxane (10 mL) and water(1 mL) was stirred at 80° C. for 16 h under argon atmosphere. The reaction mixture was concentrated and the crude product thus obtained was purified by silica gel chromatography (dichloromethane/methanol 20:1->10:1) to afford 4-(2-bromo-5-(3-(1-methyl-1H-pyrazol-3-yl)phenyl)pyrazolo[1,5-a]pyrimidin-7-yl)morpholine (400 mg, 63%) as white solid.

LCMS (ESI) m/z: 441.0 [M+H]⁺.

### Step 2: Synthesis of 1-(5-(3-(1-methyl-1H-pyrazol-3-yl)phenyl)-7-morpholinopyrazolo[1,5-a]pyrimidin-2-yl)piperidin-4-ol

A mixture of 4-(2-bromo-5-(3-(1-methyl-1H-pyrazol-3-yl)phenyl)pyrazolo[1,5-a]pyrimidin-7-yl)morpholine (88 mg, 0.2 mmol), cuprous iodide (114 mg, 0.6 mmol), potassium carbonate (82 mg, 0.6 mmol) and piperidin-4-ol (101 mg, 1 mol) in N,N-dimethylacetamide (5 mL) was stirred at 110° C. for 5 d. The mixture was filtered and the filtrate was purified by prep-HPLC (BOSTON pHlex ODS 10 μm 21.2×250 mm 120 A, with mobile phase acetonitrile/0.1% ammonium bicarbonate) to obtain 1-(5-(3-(1-methyl-1H-pyrazol-3-yl)phenyl)-7-morpholinopyrazolo[1,5-a]pyrimidin-2-yl)piperidin-4-ol (15.7 mg, 17%) as yellow solid. ¹H NMR (400 MHz, DMSO-d₆) δ 8.52 (s, 1H), 8.03 (d, J=7.2 Hz, 1H), 7.87 (s, 1H), 7.78 (s, 1H), 7.51 (s, 1H), 6.80 (s, 1H), 6.67 (s, 1H), 6.04 (s, 1H), 4.72 (s, 1H), 3.92 (s, 3H), 3.82 (d, J=9.4 Hz, 8H), 3.75 (s, 2H), 3.67 (s, 1H), 3.00 (d, J=10.5 Hz, 2H), 1.82 (s, 2H), 1.47 (s, 2H). LCMS (ESI) m/z: 460.2 [M+H]⁺.

### PIKfyve Inhibitory Activity, Metabolic Stability, Permeability, and Solubility

PIKfyve Biochemical Assay. The biochemical PIKFyve inhibition assays were run by Carna Biosciences according to proprietary methodology based on the Promega ΔDP-Glo™ Kinase assay. A full-length human PIKFYVE [1-2098(end) amino acids and S696N, L932S, Q995L, T998S, S1033 A and Q1183K of the protein having the sequence set forth in NCBI Reference Sequence No. NP_055855.2] was expressed as N-terminal GST-fusion protein (265 kDa) using baculovirus expression system. GST-PIKFYVE was purified by using glutathione sepharose chromatography and used in an ADP-Glo™ Kinase assay (Promega). Reactions were set up by adding the test compound solution, substrate solution, ATP solution and kinase solution, each at 4× final concentrations. Reactions were prepared with assay buffer (50 mM MOPS, 1 mM DTT, pH7.2), mixed, and incubated in black 384 well polystyrene plates for 1 hour at room temperature. ADP-Glo^{T}M reagent was then added for 40 minutes, followed by kinase detection reagent for an additional 40 minutes. The kinase activity was evaluated by detecting relative light units on a luminescence plate reader. Samples were run in duplicate from 10 μM to 3 nM. Data was analyzed by setting the control wells (+PIKfyve, no compound) to 0% inhibition and the readout value of background (no PIKfyve) set to 100% inhibition, then the % inhibition of each test solution calculated. IC50 values were calculated from concentration vs % inhibition curves by fitting to a four-parameter logistic curve.

NanoBRETrM TE Intracellular Kinase Assay, K-8 (Promega) Cell-Based Assay. Intracellular inhibition of PIKfyve was assayed using Promega's NanoBRET™ TE Intracellular Kinase Assay, K-8 according to manufacturer's instructions. A dilution series of test compounds was added for 2 hours to HEK293 cells transfected for a minimum of 20 hours with PIKFYVE-NanoLuc® Fusion Vector (Promega) containing a full-length PIKfyve according to manufacturer's specifications in a 96-well plate. Kinase activity was detected by addition of a NanoBRET™ tracer reagent, which was a proprietary PIKfyve inhibitor appended to a fluorescent probe (BRET, bioluminescence resonance energy transfer). Test compounds were tested at concentrations of 10, 3, 1, 0.3, 0.1, 0.03, 0.01, 0.003 μM. BRET signals were measured by a GloMax® Discover Multimode Microplate Reader (Promega) using 0.3 sec/well integration time, 450BP donor filter and 600LP acceptor filters. Active test compounds that bound PIKfyve and displaced the tracer reduced BRET signal. I050 values were then calculated by fitting the data to the normalized BRET ratio.

Compound 79 in the below tables has the following structure:

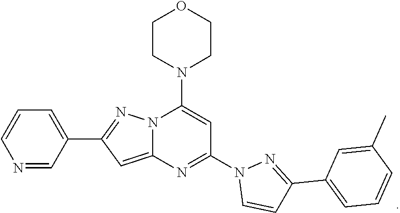 [SMILES: Cc1cccc(-c2ccn(-c3cc(N4CCOCC4)n4nc(-c5cccnc5)cc4n3)n2)c1]

Compound 148 in the below tables has the following structure:

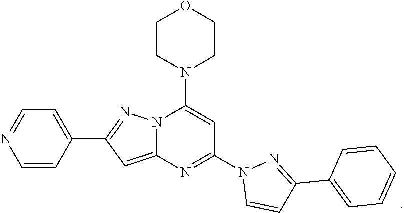 [SMILES: c1ccc(-c2ccn(-c3cc(N4CCOCC4)n4nc(-c5ccncc5)cc4n3)n2)cc1]

The results of the PIKfyve inhibition assays are summarized in the Table below.

| # | PIKFyve IC₅₀ (μM) |
|---|---|
| 1 | +++ |
| 2 | + |
| 3 | ++ |
| 4 | + |
| 5 | ++ |
| 6 | + |
| 7 | + |
| 8 | |
| 9 | |
| 10 | |
| 11 | |
| 12 | + |
| 13 | +++ |
| 14 | +++ |
| 15 | + |
| 16 | + |
| 17 | + |
| 18 | + |
| 19 | + |
| 20 | |
| 21 | |
| 22 | + |
| 23 | ++ |
| 24 | + |
| 25 | + |
| 26 | + |
| 27 | + |
| 28 | + |
| 29 | ++ |
| 30 | |
| 31 | |
| 32 | + |
| 33 | + |
| 34 | + |
| 35 | + |
| 36 | + |
| 37 | + |
| 38 | + |
| 39 | + |
| 40 | + |
| 41 | ++ |
| 42 | ++ |
| 43 | |
| 44 | ++ |
| 45 | |
| 46 | +++ |
| 47 | |
| 48 | |
| 49 | ++ |
| 50 | ++ |
| 51 | ++++ |
| 52 | |
| 53 | +++ |
| 54 | ++ |
| 55 | ++ |
| 56 | ++ |
| 57 | +++ |
| 58 | +++ |
| 59 | +++ |
| 60 | ++ |
| 61 | + |
| 62 | ++ |
| 63 | ++ |
| 64 | +++ |
| 65 | +++ |
| 66 | − |
| 67 | ++ |
| 68 | ++ |
| 69 | ++ |
| 70 | ++ |
| 71 | +++ |
| 72 | ++ |
| 73 | ++ |
| 74 | + |
| 75 | ++ |
| 76 | ++ |
| 77 | − |
| 78 | |
| 79 | ++++ |
| 80 | ++ |
| 81 | ++ |
| 82 | ++ |
| 83 | ++ |
| 84 | + |
| 85 | ++ |
| 86 | + |
| 87 | + |
| 88 | ++ |
| 89 | ++ |
| 90 | +++ |
| 91 | ++ |
| 92 | + |
| 93 | +++ |
| 94 | ++ |
| 95 | − |
| 96 | ++ |
| 97 | +++ |
| 98 | ++ |
| 99 | − |
| 100 | − |
| 101 | |
| 102 | + |
| 103 | − |
| 104 | |
| 105 | |
| 106 | |
| 107 | |
| 108 | |
| 109 | + |
| 110 | |
| 111 | ++ |
| 112 | + |
| 113 | ++ |
| 114 | + |
| 115 | ++ |
| 116 | + |
| 117 | + |
| 118 | + |
| 119 | |
| 120 | |
| 121 | − |
| 122 | |
| 123 | |
| 124 | + |
| 125 | + |
| 126 | + |
| 127 | +++ |
| 128 | +++ |
| 129 | |
| 130 | + |
| 131 | |
| 132 | ++ |
| 133 | + |
| 134 | |
| 135 | + |
| 136 | + |
| 137 | ++ |
| 138 | ++ |
| 139 | ++ |
| 140 | ++ |
| 141 | 1 |
| 142 | +++ |
| 143 | ++ |
| 144 | +++ |
| 145 | |
| 146 | ++ |
| 147 | 1.57 |
| 148 | +++ |
| 149 | |
| 150 | |

++++ stands for <10 nM, +++ stands for 10-100 nM, ++ stands for 100-1000 nM, + stands for 1-10 μM, and − stands for >10 μM.

EEA1 Assay. Genetic or pharmacological disruption of PIKfyve activity results in enlargement of endosomal vesicles. This enlargement was utilized as a surrogate readout of PIKFyve inhibition for routine triage of PIKfyve inhibitors. U2OS cells grown in 96-well assay plates were treated with compound diluted in DMEM media containing 10% fetal bovine serum. After 3 hours of treatment, cells were fixed with paraformaldehyde, permeabilized with 0.2% Triton-X in phosphate buffered saline and stained against EEA1. During the secondary antibody staining, cells were also stained with CellMask DeepRed and Hoechst to detect cytoplasms and nuclei respectively. Endosomal structures were visualized using a high content imager at 40× magnification. Images were analyzed using a linear classifier algorithm integrating EEA1 spot size, intensity and texture trained on images of cells treated with the potent reference compound APY0201. Compound activity was calculated by subtracting the DMSO signal and calculating percentage activity relative to maximal APY0201 activity. IC50s were then calculated from concentration vs. % inhibition data by logistic regression.

The results of PIKFyve EEA1 assays are shown below.

| # | EEA1 IC₅₀ (μM) |
|---|---|
| 1 | |
| 2 | |
| 3 | |
| 4 | |
| 5 | |
| 6 | |
| 7 | |
| 8 | |
| 9 | |
| 10 | |
| 11 | |
| 12 | |
| 13 | |
| 14 | |
| 15 | |
| 16 | |
| 17 | |
| 18 | |
| 19 | |
| 20 | |
| 21 | |
| 22 | |
| 23 | |
| 24 | |
| 25 | |
| 26 | |
| 27 | |
| 28 | |
| 29 | |
| 30 | |
| 31 | |
| 32 | |
| 33 | |
| 34 | |
| 35 | |
| 36 | |
| 37 | |
| 38 | |
| 39 | |
| 40 | |
| 41 | |
| 42 | |
| 43 | |
| 44 | |
| 45 | |
| 46 | |
| 47 | |
| 48 | |
| 49 | |
| 50 | |
| 51 | + |
| 52 | |
| 53 | |
| 54 | |
| 55 | |
| 56 | |
| 57 | |
| 58 | |
| 59 | |
| 60 | |
| 61 | |
| 62 | |
| 63 | |
| 64 | |
| 65 | ++ |
| 66 | >3.00 |
| 67 | |
| 68 | |
| 69 | |
| 70 | |
| 71 | ++ |
| 72 | |
| 73 | |
| 74 | |
| 75 | |
| 76 | |
| 77 | + |
| 78 | + |
| 79 | ++ |
| 80 | |
| 81 | |
| 82 | |
| 83 | |
| 84 | |
| 85 | |
| 86 | |
| 87 | |
| 88 | |
| 89 | |
| 90 | |
| 91 | |
| 92 | |
| 93 | |
| 94 | |
| 95 | |
| 96 | |
| 97 | + |
| 98 | |
| 99 | |
| 100 | |
| 101 | |
| 102 | |
| 103 | |
| 104 | |
| 105 | |
| 106 | |
| 107 | |
| 108 | |
| 109 | |
| 110 | |
| 111 | |
| 112 | |
| 113 | |
| 114 | |
| 115 | |
| 116 | |
| 117 | |
| 118 | |
| 119 | |
| 120 | |
| 121 | |
| 122 | |
| 123 | |
| 124 | |
| 125 | |
| 126 | |
| 127 | |
| 128 | |
| 129 | |
| 130 | |
| 131 | |
| 132 | |
| 133 | |
| 134 | |
| 135 | |
| 136 | |
| 137 | |
| 138 | |
| 139 | |
| 140 | |
| 141 | |
| 142 | + |
| 143 | |
| 144 | |
| 145 | + |
| 146 | |
| 147 | |
| 148 | |
| 149 | ++ |
| 150 | + |
| 151 | + |
| 152 | + |

++++ stands for <10 nM, +++ stands for 10-100 nM, ++ stands for 100-1000 nM, + stands for 1-10 μM, and − stands for >10 μM

### Viability Assay to Assess TDP-43 Toxicity in FAB1 TDP-43 and PIKfyve TDP-43 Yeast Cells

Generation of TDP-43 yeast model expressing human PIKfyve. Human PIKFYVE (“entry clone”) was cloned into pAG416GPDccdB (“destination vector”) according to standard Gateway cloning protocols (Invitrogen, Life Technologies). The resulting pAG416GPD-PIKFYVE plasmids were amplified in E. coli and plasmid identity confirmed by restriction digest and Sanger sequencing. Lithium acetate/polyethylene glycol-based transformation was used to introduce the above PIKFYVE plasmid into a BY4741 yeast strain auxotrophic for the ura3 gene and deleted for two transcription factors that regulate the xenobiotic efflux pumps, a major efflux pump, and FAB1, the yeast ortholog of PIKFYVE (MATa, snq2::KILeu2; pdr3::Klura3;pdrl::NATMX; fab1::G41_{8R}, his3;leu2;ura3;metl5;LYS2+) (FIG. 2).

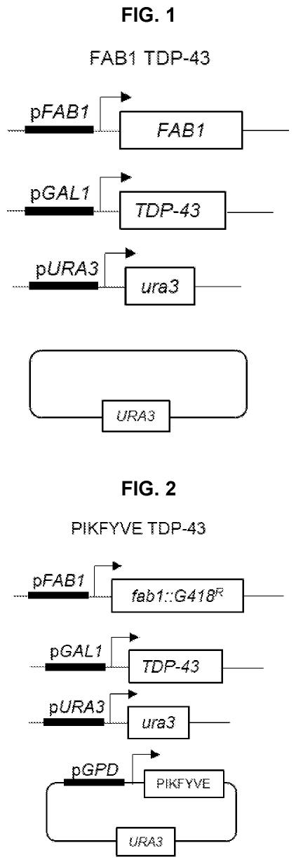

Transformed yeast were plated on solid agar plates with complete synthetic media lacking uracil (CSM-ura) and containing 2% glucose. Individual colonies harboring the control or PIKFYVE TDP-43 plasmids were recovered. A plasmid containing wild-type TDP-43 under the transcriptional control of the GAL1 promoter and containing the hygromycin-resistance gene as a selectable marker was transformed into the fab1::G[^{R}pAG416GPD-PIKFYVE yeast strain (FIG. 1). Transformed yeast were plated on CSM-ura containing 2% glucose and 200 μg/mL G418 after overnight recovery in media lacking antibiotic. Multiple independent isolates were further evaluated for cytotoxicity and TDP-43 expression levels.

Viability Assay. A control yeast strain with the wild-type yeast FAB1 gene and TDP-43 (“FAB1 TDP-43”, carries empty pAG416 plasmid), and the “PIKFYVE TDP-43” yeast strain, were assessed for toxicity using a propidium iodide viability assay. Both yeast strains were transferred from solid CSM-ura/2% glucose agar plates into 3 mL of liquid CSM-ura/2% glucose media for 6-8 hours at 30° C. with aeration. Yeast cultures were then diluted to an optical density at 600 nm wavelength (OD₆₀₀) of 0.005 in 3 mL of CSM-ura/2% raffinose and grown overnight at 30° C. with aeration to an OD₆₀₀ of 0.3-0.8. Log-phase overnight cultures were diluted to OD₆₀₀ of 0.005 in CSM-ura containing either 2% raffinose or galactose and 150 μL dispensed into each well of a flat bottom 96-well plates. Compounds formulated in 100% dimethyl sulfoxide (DMSO) were serially diluted in DMSO and 1.5 μL diluted compound transferred to the 96-well plates using a multichannel pipet. Wells containing DMSO alone were also evaluated as controls for compound effects. Tested concentrations ranged from 15 μM to 0.11 μM. Cultures were immediately mixed to ensure compound distribution and covered plates incubated at 30° C. for 24 hours in a stationary, humified incubator.

Upon the completion of incubation, cultures were assayed for viability using propidium iodide (PI) to stain for dead/dying cells. A working solution of PI was made where, for each plate, 1 μL of 10 mM PI was added to 10 mL of CSM-ura (raffinose or galactose). The final PI solution (50 μL/well) was dispensed into each well of a new round bottom 96-well plate. The overnight 96-well assay plate was then mixed with a multichannel pipet and 50 μL transferred to the PI-containing plate. This plate was then incubated for 30 minutes at 30° C. in the dark. A benchtop flow cytometer (Miltenyi MACSquant) was then used to assess red fluorescence (B2 channel), forward scatter, and side scatter (with following settings: gentle mix, high flow rate, fast measurement, 10,000 events). Intensity histograms were then gated for “PI-positive” or “PI-negative” using the raffinose and galactose cultures treated with DMSO as controls. The DMSO controls for raffinose or galactose-containing cultures were used to determine the window of increased cell death and this difference set to 100. All compounds were similarly gated and then compared to this maximal window to establish the percent reduction in PI-positive cells. IC50 values were then calculated for compounds that demonstrated a concentration-dependent enhancement of viability by fitting a logistic regression curve.

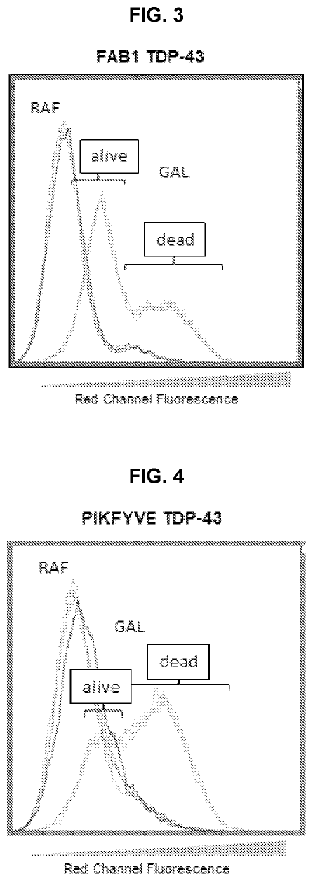

Upon induction of TDP-43 in both strains, there was a marked increase in inviable cells (rightmost population) with both FAB1 TDP-43 and PIKFYVE TDP-43, with a more pronounced effect in PIKFYVE TDP-43 (FIGS. 3 and 4).

PIKfyve Inhibition Suppresses Toxicity in PIKfyve TDP-43 Model. The biochemical PIKFyve inhibition assays were run by Carna Biosciences according to proprietary methodology based on the Promega ADP-Glo™ Kinase assay. A full-length human PIKFYVE [1-2098(end) amino acids and S696N, L932S, Q995L, T998S, S1033 A and Q1183K of accession number NP_055855.2] was expressed as N-terminal GST-fusion protein (265 kDa) using baculovirus expression system. GST-PIKFYVE was purified by using glutathione sepharose chromatography and used in an ADP-GIo™ Kinase assay (Promega). Reactions were set up by adding the test compound solution, substrate solution, ATP solution and kinase solution, each at 4× final concentrations. Reactions were prepared with assay buffer (50 mM MOPS, 1 mM DTT, pH7.2), mixed, and incubated in black 384 well polystyrene plates for 1 hour at room temperature. ADP-GIo™ reagent was then added for 40 minutes, followed by kinase detection reagent for an additional 40 minutes. The kinase activity was evaluated by detecting relative light units on a luminescence plate reader. Samples were run in duplicate from 10 uM to 3 nM. Data was analyzed by setting the control wells (+PIKfyve, no compound) to 0% inhibition and the readout value of background (no PIKfyve) set to 100% inhibition, then the % inhibition of each test solution calculated. IC50 values were calculated from concentration vs % inhibition curves by fitting to a four-parameter logistic curve.

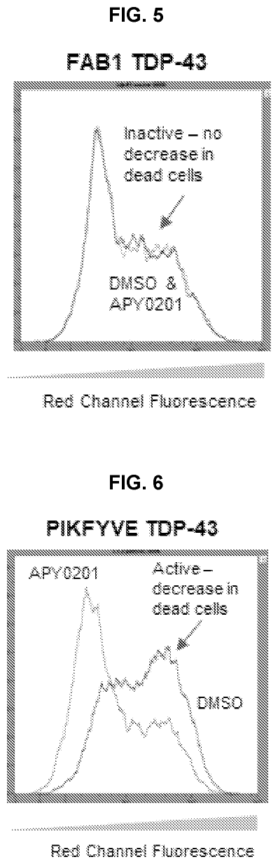

Activity of APYO201, a known PIKFYVE inhibitor, in FAB1 TDP-43 (FIG. 5) and PIKFYVE TDP-43 (FIG. 6). There was no increase in viable cells in FAB1 TDP-43 across a range of compound concentrations as evidenced by a lack in reduction of the right most population of propidium iodide-positive cells (only 0.23 μM is shown). In the PIKFYVE TDP-43 model, 0.23 μM reduced the population of propidium iodide-positive dead cells, indicating PIKFYVE inhibition ameliorated TDP-43 toxicity. Concentrations ranging from 0.5 mM to less than 100 nM afforded increased viability.

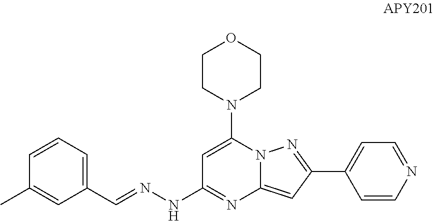 [SMILES: Cc1cccc(/C=N/Nc2cc(N3CCOCC3)n3nc(-c4ccncc4)cc3n2)c1]

A panel of compounds was tested in a biochemical PIKFYVE assay (ADP-Glo™ with full-length PIKfyve) and IC50's determined (nM) (see the Table below). The same compounds were also tested in both FAB1 and PIKFYVE TDP-43 yeast models. Their activity is reported here as “active” or “inactive.” Compounds with low nanomolar potency in the biochemical assay were active in the PIKFYVE TDP-43 yeast model. Compounds that were less potent or inactive in the biochemical assay were inactive in the PIKFYVE TDP-43 model. Compounds that were inactive in the biochemical or PIKFYVE TDP-43 assays were plotted with the highest concentrations tested in that assay.

| | PIKfyve IC₅₀ | FAB1 TDP-43 | PIKfyve TDP-43 |
|---|---|---|---|
| Structure | (nM) | (active/inactive) | (active/inactive) |
| 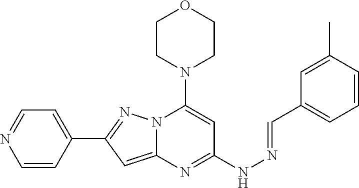 [SMILES: Cc1cccc(/C=N/Nc2cc(N3CCOCC3)n3nc(-c4ccncc4)cc3n2)c1] | 7.5 | Inactive | Active |
| 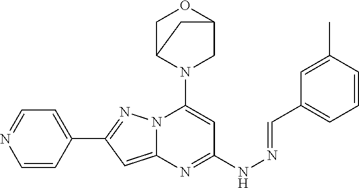 [SMILES: Cc1cccc(/C=N/Nc2cc(N3CC4CC3CO4)n3nc(-c4ccncc4)cc3n2)c1] | 12 | Inactive | Active |
| 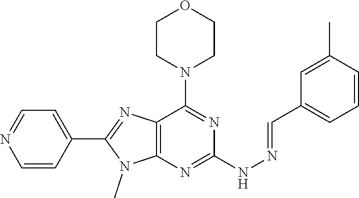 [SMILES: Cc1cccc(/C=N/Nc2nc(N3CCOCC3)c3nc(-c4ccncc4)n(C)c3n2)c1] | 4.9 | Inactive | Active |
| 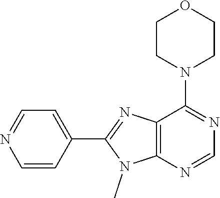 [SMILES: Cn1c(-c2ccncc2)nc2c(N3CCOCC3)ncnc21] | 640 | Inactive | Inactive |
| 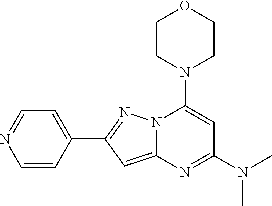 [SMILES: CN(C)c1cc(N2CCOCC2)n2nc(-c3ccncc3)cc2n1] | 2007 | Inactive | Inactive |
| 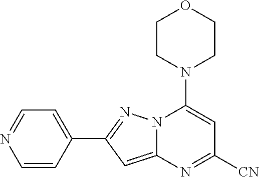 [SMILES: N#Cc1cc(N2CCOCC2)n2nc(-c3ccncc3)cc2n1] | >10000 | Inactive | Inactive |

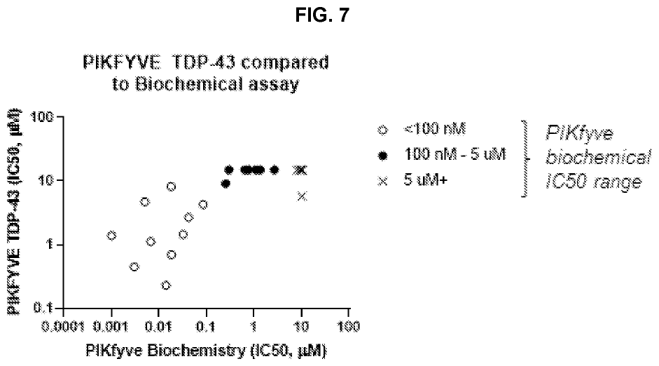

Biochemical and Efficacy Assays. A larger set of PIKfyve inhibitors were evaluated in both a PIKfyve kinase domain binding assay (nanobret) and in the PIKFYVE TDP-43 yeast strain. IC50 values (μM) were plotted. Data points are formatted based on binned potency from the nanobret assay as indicated in the legend (FIG. 7). Below is a table of compounds and their biochemical and PIKFYVE TDP-43 IC50 values plotted in FIG. 7.

| | PIKFYVE Biochemistry | PIKFYVE TDP-43 |
|---|---|---|
| Structure | (IC50, μM) | (IC50, μM) |
| 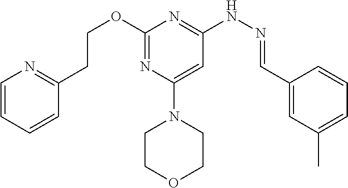 [SMILES: Cc1cccc(/C=N/Nc2cc(N3CCOCC3)nc(OCCc3ccccn3)n2)c1] | 0.003 | 0.450 |
| 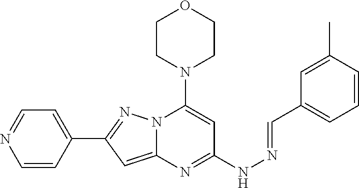 [SMILES: Cc1cccc(/C=N/Nc2cc(N3CCOCC3)n3nc(-c4ccncc4)cc3n2)c1] | 0.001 | 1.390 |
| 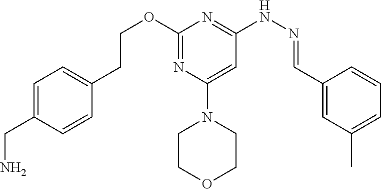 [SMILES: Cc1cccc(/C=N/Nc2cc(N3CCOCC3)nc(OCCc3ccc(CN)cc3)n2)c1] | 0.007 | 1.120 |
| 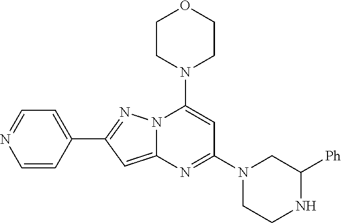 [SMILES: c1ccc(C2CN(c3cc(N4CCOCC4)n4nc(-c5ccncc5)cc4n3)CCN2)cc1] | 2.660 | >15 |
| 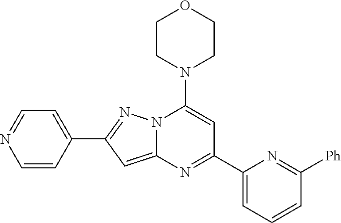 [SMILES: c1ccc(-c2cccc(-c3cc(N4CCOCC4)n4nc(-c5ccncc5)cc4n3)n2)cc1] | 0.014 | 0.230 |
| 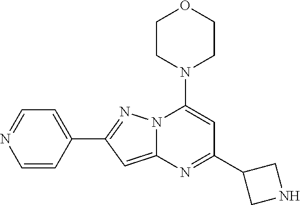 [SMILES: c1cc(-c2cc3nc(C4CNC4)cc(N4CCOCC4)n3n2)ccn1] | 8.020 | >15 |
| 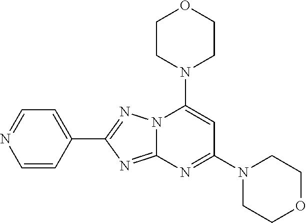 [SMILES: c1cc(-c2nc3nc(N4CCOCC4)cc(N4CCOCC4)n3n2)ccn1] | 9.200 | >15 |
| 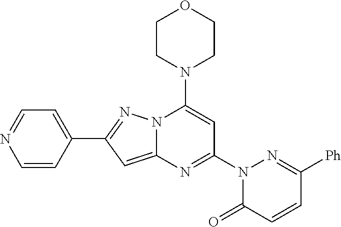 [SMILES: O=c1ccc(-c2ccccc2)nn1-c1cc(N2CCOCC2)n2nc(-c3ccncc3)cc2n1] | 0.295 | >15 |
| 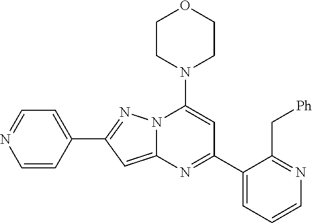 [SMILES: c1ccc(Cc2ncccc2-c2cc(N3CCOCC3)n3nc(-c4ccncc4)cc3n2)cc1] | 1.090 | >15 |
| 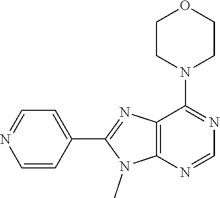 [SMILES: Cn1c(-c2ccncc2)nc2c(N3CCOCC3)ncnc21] | 0.640 | >15 |
| 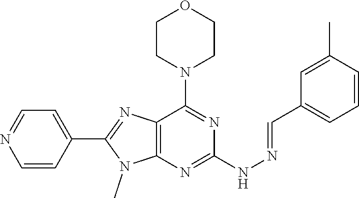 [SMILES: Cc1cccc(/C=N/Nc2nc(N3CCOCC3)c3nc(-c4ccncc4)n(C)c3n2)c1] | 0.005 | 4.720 |
| 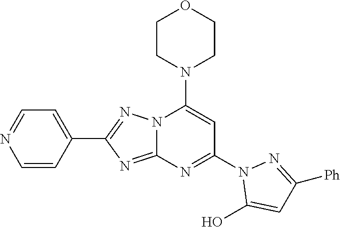 [SMILES: Oc1cc(-c2ccccc2)nn1-c1cc(N2CCOCC2)n2nc(-c3ccncc3)nc2n1] | 0.018 | 0.693 |
| 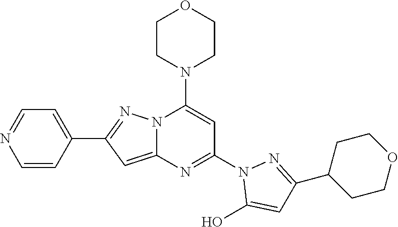 [SMILES: Oc1cc(C2CCOCC2)nn1-c1cc(N2CCOCC2)n2nc(-c3ccncc3)cc2n1] | 0.253 | 9.105 |
| 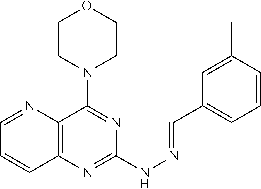 [SMILES: Cc1cccc(/C=N/Nc2nc(N3CCOCC3)c3ncccc3n2)c1] | 0.018 | 8.214 |
| 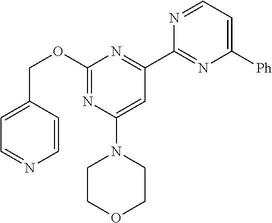 [SMILES: c1ccc(-c2ccnc(-c3cc(N4CCOCC4)nc(OCc4ccncc4)n3)n2)cc1] | 0.032 | 1.447 |
| 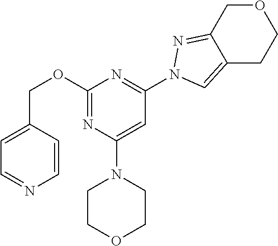 [SMILES: c1cc(COc2nc(N3CCOCC3)cc(-n3cc4c(n3)COCC4)n2)ccn1] | 1.343 | >15 |
| 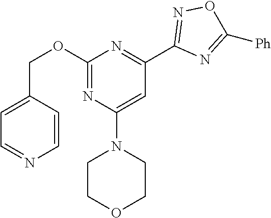 [SMILES: c1ccc(-c2nc(-c3cc(N4CCOCC4)nc(OCc4ccncc4)n3)no2)cc1] | >10 | >15 |
| 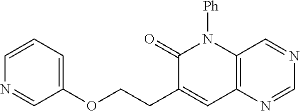 [SMILES: O=c1c(CCOc2cccnc2)cc2ncncc2n1-c1ccccc1] | >10 | >15 |
| 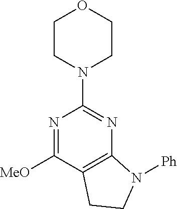 [SMILES: COc1nc(N2CCOCC2)nc2c1CCN2c1ccccc1] | 0.085 | 4.273 |
| 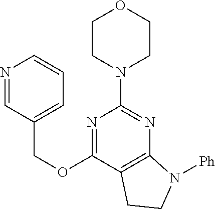 [SMILES: c1ccc(N2CCc3c(OCc4cccnc4)nc(N4CCOCC4)nc32)cc1] | 0.042 | 2.685 |
| 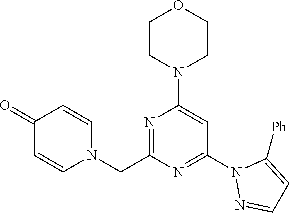 [SMILES: O=c1ccn(Cc2nc(N3CCOCC3)cc(-n3nccc3-c3ccccc3)n2)cc1] | >10 | >15 |
| 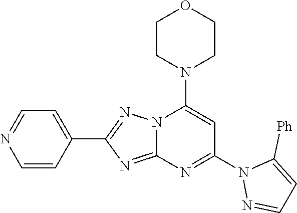 [SMILES: c1ccc(-c2ccnn2-c2cc(N3CCOCC3)n3nc(-c4ccncc4)nc3n2)cc1] | 0.767 | >15 |
| 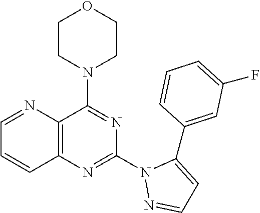 [SMILES: Fc1cccc(-c2ccnn2-c2nc(N3CCOCC3)c3ncccc3n2)c1] | >10 | 5.754 |

### Other Embodiments

Various modifications and variations of the described invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention that are obvious to those skilled in the art are intended to be within the scope of the invention.

Other embodiments are in the claims.

1. A compound having the structure: 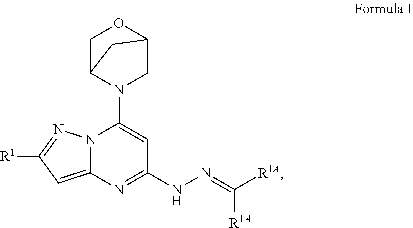
or pharmaceutically acceptable salt thereof,
wherein:
R¹ is optionally substituted C₂-C₉ heteroaryl; and
each R^{IA} is independently H, optionally substituted C₁-C₆ alkyl, optionally substituted C₆-C₁₀ aryl, or optionally substituted C₂-C₉ heteroaryl; and the remaining R¹B is optionally substituted C₁-C₆ alkyl, optionally substituted C₆-C₁₀ aryl, or optionally substituted C₂-C₉ heteroaryl.

2. The compound of claim 1, wherein one R^{1A} is hydrogen, and the remaining R^{1A} is optionally substituted C₆-C₁₀ aryl.

3. The compound of claim 1 or 2, wherein R¹ is pyrid-4-yl.

4. A compound having the structure: 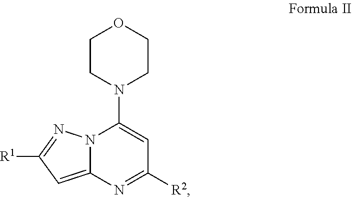
or pharmaceutically acceptable salt thereof,
wherein:
R¹ is optionally substituted pyridin-4-yl;
R² is optionally substituted C₂-C₉ heterocyclyl, optionally substituted C₁-C₆ alkyl, optionally substituted C₁-C₆ alkenyl, optionally substituted pyridin-2-yl, optionally substituted pyridin-3-yl, optionally substituted pyrimidn-4-yl, optionally substituted thiadiazolyl, optionally substituted oxadiazolyl, optionally substituted dialkylamino, optionally substituted 6-oxo-1,5-dihydropyridazin-1-yl, optionally substituted pyrazinyl, fluoro, cyano, optionally substituted pyrazol-3-yl, optionally substituted pyrazol-5-yl, optionally substituted oxazole, optionally substituted N-tetrahydropyranopyrazolyl, optionally substituted N-tetrahydroindazolyl, optionally substituted C₁-C₆ heteroalkyl, optionally substituted C₆-C₁₀ aryl C₁-C₆ alkyl, optionally substituted C₆-C₁₀ aryl C₁-C₆ alkenyl, optionally substituted C₆-C₁₀aryl C₁-C₆ heteroalkyl, CONH₂, —CONH—NHR^{1A}, 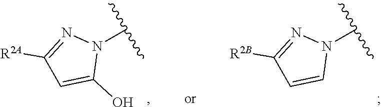
R^{1A} is H or optionally substituted C₂-C₁₀acyl;
R^{2A} is optionally substituted C₆-C₁₀aryl, optionally substituted C₁-C₆ alkyl, optionally substituted C₂-C₅ heteroaryl, or optionally substituted C₃-C₆ cycloalkyl; and
R^{2B} is pyridizin-4-yl, phenyl substituted with fluoro or methoxy, piperidinyl optionally substituted with C₁-C₆ alkyl, optionally substituted pyrimidin-5-yl, optionally substituted pyridin-2-yl, optionally substituted pyridine-3-yl, optionally substituted C₃ carbocyclyl, azetidin-3-yl, optionally substituted C₁-C₆ hydroxyalkyl, or C₃ heteroalkyl.

5. (canceled)

6. The compound of claim 4, wherein R² is optionally substituted azetidine-3-yl, optionally substituted azetidine-1-yl, optionally substituted piperazin-1-yl optionally substituted piperidin-1-yl optionally substituted morpholin-1-yl, optionally substituted pyrrolidine-2-yl, optionally substituted pyridin-2-yl, optionally substituted pyridin-3-yl, optionally substituted pyrimidin-4-yl, optionally substituted thiadiazolyl, optionally substituted oxadiazolyl, optionally substituted 6-oxo-1,5-dihydropyridazin-1-yl, optionally substituted dialkylamino, optionally substituted pyrazinyl, optionally substituted pyrazol-3-yl, optionally substituted pyrazol-5-yl, optionally substituted oxazolyl, optionally substituted N-tetrahydropyranopyrazolyl, optionally substituted N-tetrahydroindazolyl, or optionally substituted imidazolyl.

7-31. (canceled)

32. The compound of claim 4, wherein R² is substituted with oxo, optionally substituted phenyl, optionally substituted benzyl, optionally substituted phenoxy, 4-fluorophenoxy, 3-fluorophenoxy, optionally substituted amino, ═NH, optionally substituted C₁-C₆ alkyl, methyl, halo, bromo, optionally substituted C₁-C₆ heteroalkyl, methoxy, optionally substituted pyridin-3-yl, optionally substituted C₂-C₉ heterocyclyl, optionally substituted piperidin-3-yl, optionally substituted 1,2,3,6-tetrahydropyridin-3-yl, hydroxyl, or nitro.

33-49. (canceled)

50. The compound of claim 4, wherein R² is

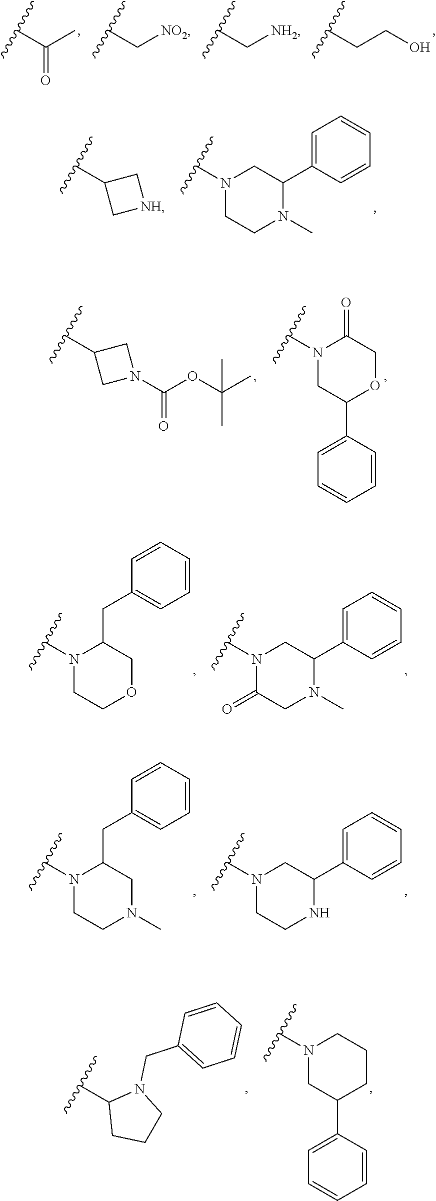

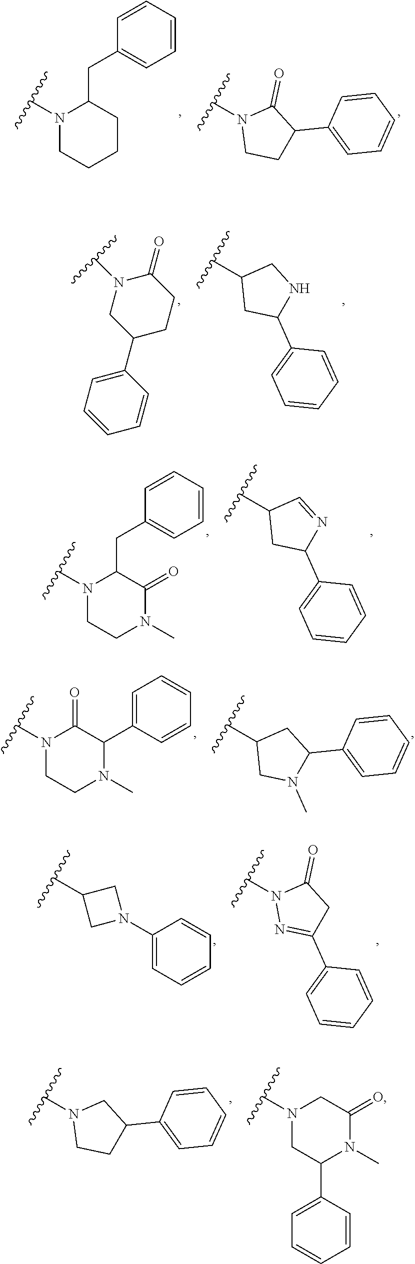

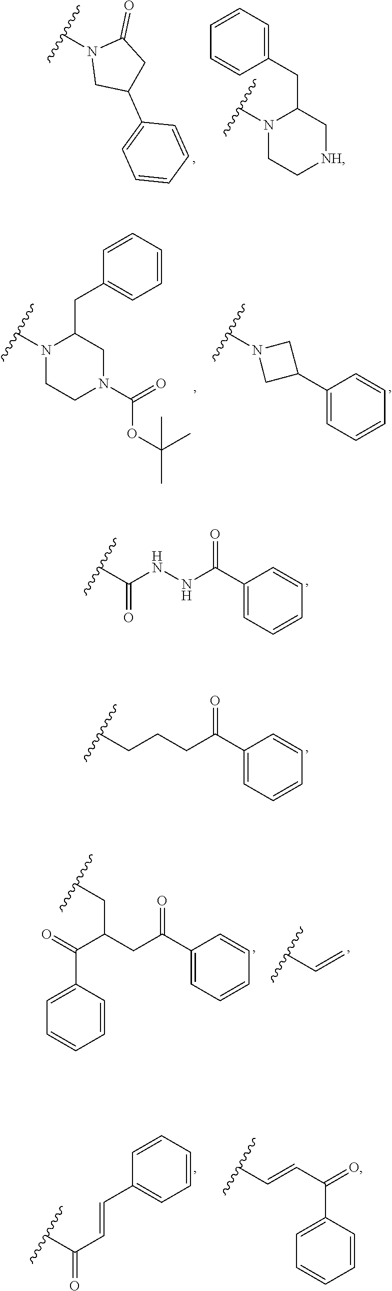

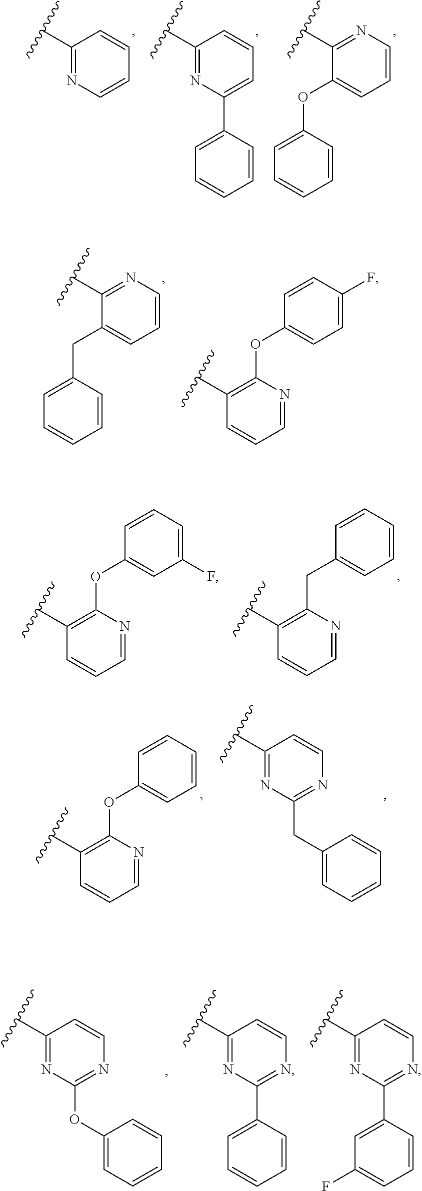

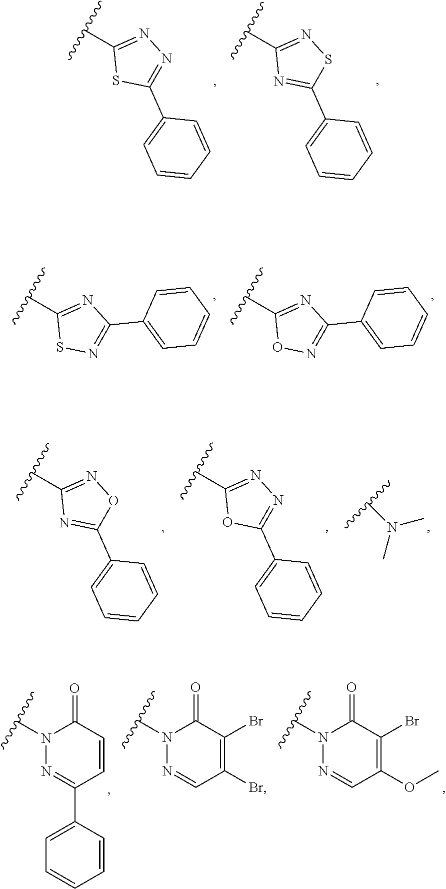

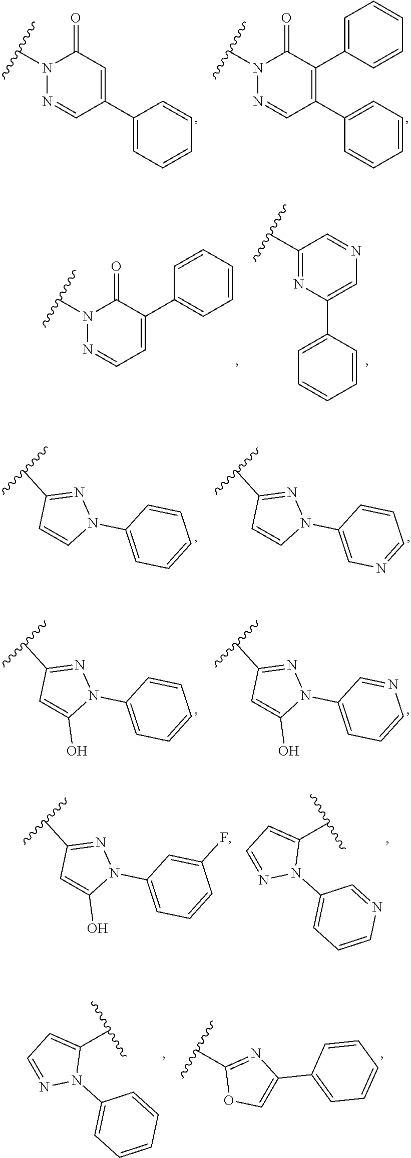

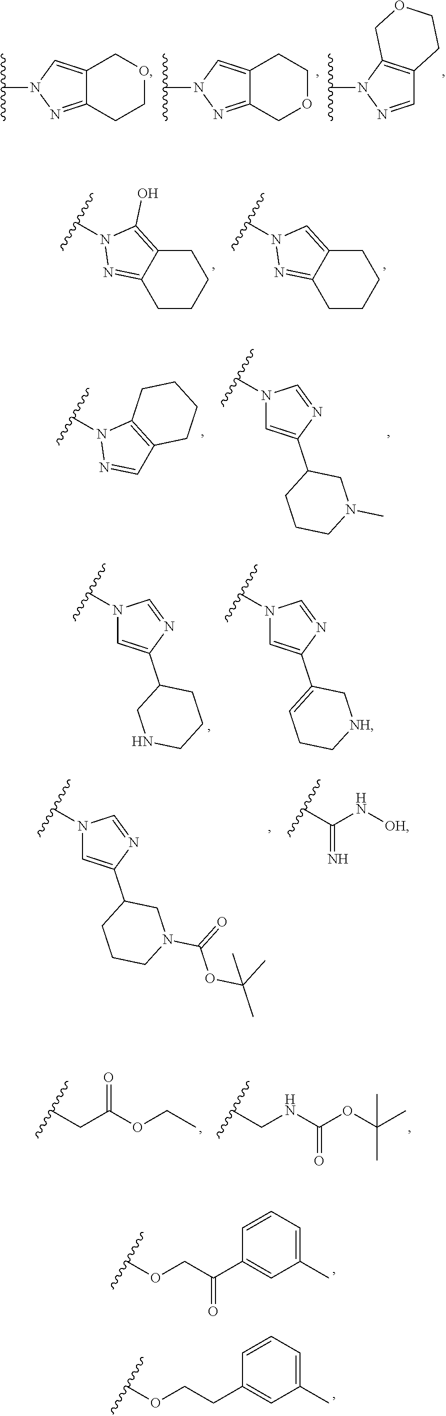

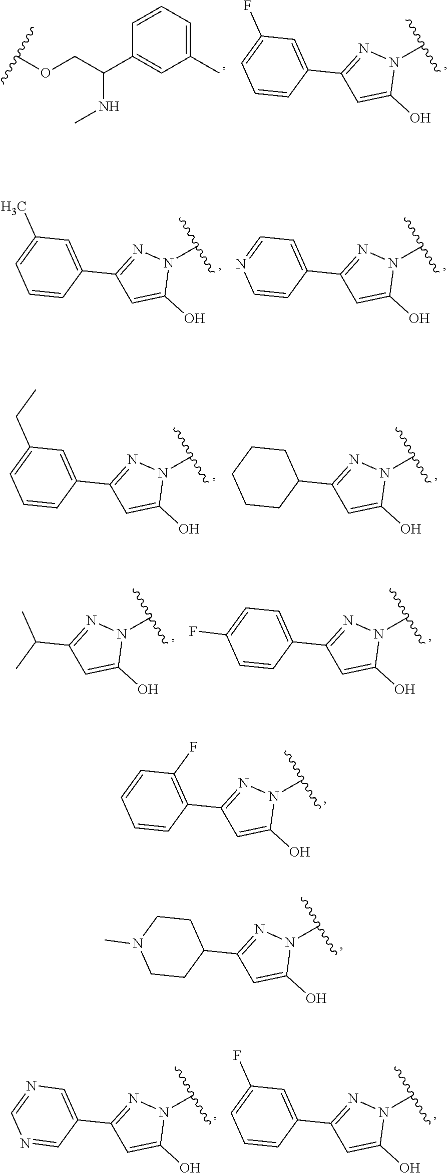

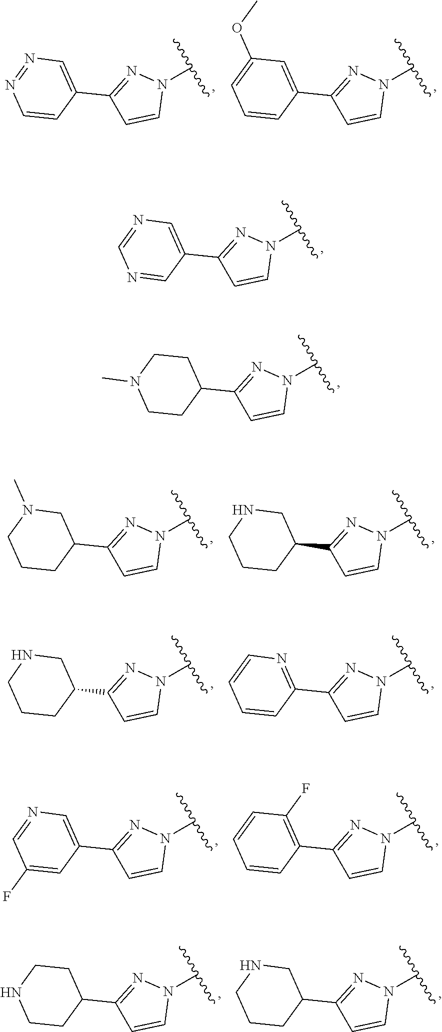

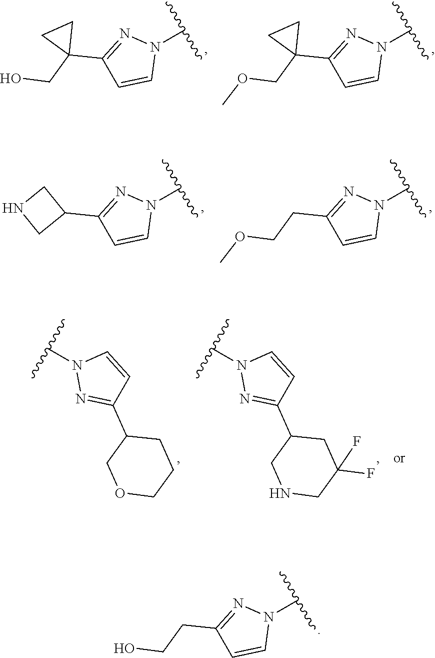

51-74. (canceled)

75. A compound, or pharmaceutically acceptable salt thereof, wherein the compound has the structure: 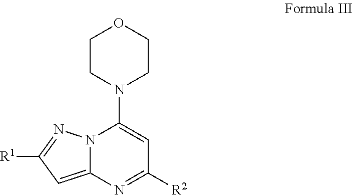
wherein:
R¹ is optionally substituted C₁₋₆ alkenyl, optionally substituted C₁-C₆ hydroxyalkyl, C₁-C₆ alkyl substituted with dialkyl amino, hydrogen, or optionally substituted C₂-C₉ heterocyclyl; and
R² is optionally substituted pyrazol-1-yl, optionally substituted pyrazol-3-yl, optionally substituted N-tetrahydropyranopyrazolyl, C₆-C₁₀ aryl optionally substituted with optionally substituted C₂-C₉ heteroaryl, or optionally substituted pyrimidin-4-yl.

76-78. (canceled)

79. The compound of claim 75, wherein R¹ is

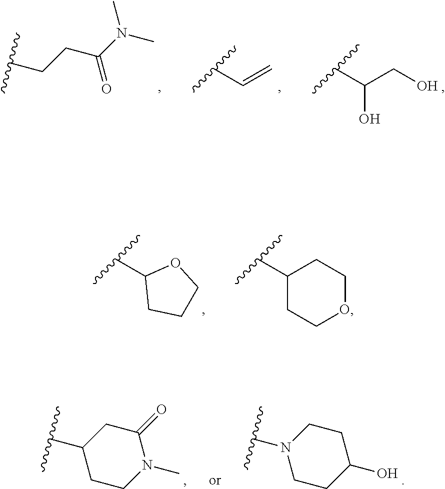

80-84. (canceled)

85. The compound of claim 75, wherein R² is an optionally substituted pyrazol-3-yl, optionally substituted pyrazol-1-yl, optionally substituted N-tetrahydropyranopyrazolyl, or optionally substituted pyrimidin-4-yl.

86-88. (canceled)

89. The compound of claim 75, wherein R² is substituted with optionally substituted phenyl, 3-methoxy-phenyl, 2-fluorophenyl, or pyrimidin-3-yl.

90-92. (canceled)

93. The compound of claim 75, wherein R²

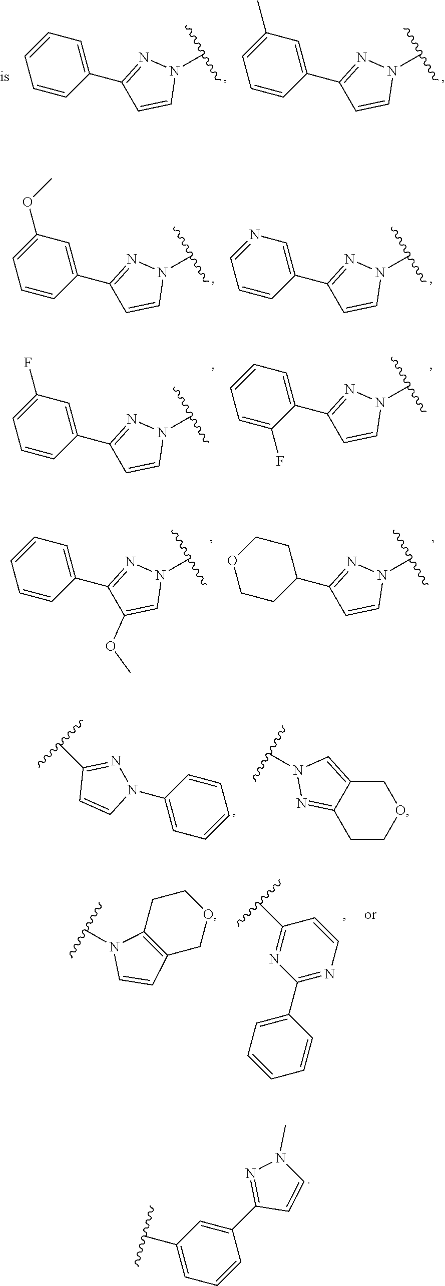

94-97. (canceled)

98. A compound having the structure of any one of compounds 1-152 in Table 1, or a pharmaceutically acceptable salt thereof.

99. A pharmaceutical composition comprising the compound of claim 1, or a pharmaceutically acceptable salt thereof, and a pharmaceutically acceptable excipient.

100. A method of treating a neurological disorder in a subject in need thereof, the method comprising administering to the subject an effective amount of the compound of claim 1, or a pharmaceutically acceptable salt thereof.

101. The method of claim 100, wherein the neurological disorder is FTLD-TDP, chronic traumatic encephalopathy, ALS, Alzheimer's disease, LATE, or frontotemporal lobar degeneration.

102. (canceled)

103. A method of inhibiting toxicity in a cell related to a protein, the method comprising contacting the cell with the compound of claim 1 or a pharmaceutically acceptable salt thereof.

104. The method of claim 103, wherein the toxicity is TDP-43-related toxicity, or C9orf72-related toxicity.

105. (canceled)

106. A method of inhibiting PIKfyve in a cell expressing PIKfyve protein, the method comprising contacting the cell with the compound of claim 1 or a pharmaceutically acceptable salt thereof.

107-109. (canceled)

110. A method of treating a TDP-43 associate disorder in a subject, the method comprising administering to the subject in need an effective amount of the compound of claim 1.
